(19) United States
(12) Patent Application Publication
(10) Pub. No.: US 2023/0348561 A1
(43) Pub. Date: Nov. 2, 2023

(54) DOMINANT NEGATIVE TGFBETA RECEPTOR POLYPEPTIDES, CD8 POLYPEPTIDES, CELLS, COMPOSITIONS, AND METHODS OF USING THEREOF

(71) Applicant: IMMATICS US, INC., Houston, TX (US)

(72) Inventors: Erik FARRAR, Houston, TX (US); Justin GUNESCH, Houston, TX (US); Melinda MATA, Houston, TX (US); Mohammad HOSSAIN, Houston, TX (US); Mamta KALRA, Houston, TX (US); Gagan BAJWA, Houston, TX (US)

(21) Appl. No.: 18/309,062

(22) Filed: Apr. 28, 2023

Related U.S. Application Data
(60) Provisional application No. 63/336,062, filed on Apr. 28, 2022.

(51) Int. Cl. C07K 14/725; C07K 14/705; C12N 15/63; A61P 37/04

(52) U.S. Cl. CPC C07K 14/7051; C07K 14/70517; C12N 15/63; A61P 37/04

(57) ABSTRACT

The present disclosure relates to T cells capable of co-expressing T cell receptors (“TCR”) together with dominant negative TGFβ Receptor (“dnTGFβR”) polypeptides and/or CD8 polypeptides and the use thereof in adoptive cellular therapy. The present disclosure further provides for dnTGFβR polypeptides, vectors, and associated methods thereof. The present disclosure further provides for modified CD8 polypeptides, vectors, and associated methods of making and using the same.

### RELATED APPLICATIONS

The present application is an U.S. Non-Provisional application claiming priority to U.S. Provisional Patent Application No. 63/336,062, filed on Apr. 28, 2022, the entire contents of which are hereby incorporated by reference for all purposes.

### REFERENCE TO SEQUENCE LISTING SUBMITTED ELECTRONICALLY

The official copy of the sequence listing is submitted concurrently via EFS-Web as an ASCII-formatted sequence listing with a file named “3000011-031001_Sequence-Listing_ST26” created on Apr. 26, 2023, and having a size of 521,214 bytes, and is filed concurrently with the specification. The sequence listing contained in this ASCII-formatted document is part of the specification and is herein incorporated by reference in its entirety.

### BACKGROUND

### Field

The present disclosure relates to cells capable of co-expressing one or any combination of T cell receptors (“TCR”), CD8 polypeptides, and/or dnTGFβR (dominant negative TGFβ Receptor) polypeptides and the use thereof in adoptive cellular therapy (“ACT”). The present disclosure further provides for modified CD8 sequences, dnTGFβRII sequences, vectors, compositions, transformed cells, and associated methods thereof.

### Background

CD8 and CD4 are transmembrane glycoproteins characteristic of distinct populations of T lymphocytes whose antigen responses are restricted by class I and class II MHC molecules, respectively. They play major roles both in the differentiation and selection of T cells during thymic development and in the activation of mature T lymphocytes in response to antigen presenting cells. Both CD8 and CD4 are immunoglobulin superfamily proteins. They determine antigen restriction by binding to MHC molecules at an interface distinct from the region presenting the antigenic peptide, but the structural basis for their similar functions appears to be very different. Their sequence similarity is low and, whereas CD4 is expressed on the cell surface as a monomer, CD8 is expressed as an αα homodimer (e.g., FIG. 55C) or an αβ heterodimer (e.g., FIG. 55A). In humans, this CD8αα homodimer may functionally substitute for the CD8αβ heterodimer. CD8 contacts an acidic loop in the α3 domain of Class I MHC, thereby increasing the avidity of the T cell for its target. CD8 is also involved in the phosphorylation events leading to CTL activation through the association of its a chain cytoplasmic tail with the tyrosine kinase p56^{lck}.

Transforming Growth Factor β (TGFβ or TGF-β) is a cytokine having important roles in immune cell function. Transforming Growth Factor β Receptor I (TGFβRI) and Transforming Growth Factor β Receptor II (TGFβRII) are receptor important in TGFβ signalling. TGFβRII is a transmembrane serine/threonine kinase protein that, in a complex with Transforming Growth Factor β Receptor I (TGFβRI), binds TGFβ. After TGFβ is bound, TGFβRII phosphorylates TGFβRI, which then activates further signalling.

Adoptive cell therapy (ACT) is a promising approach to treatment of diseases such as cancer. T-cell therapy has been successful in treating various cancers. Li et al. Signal Transduction and Targeted Therapy 4(35): (2019), the content of which is incorporated by reference in its entirety. However, cells used in ACT often fail to persist in the tumor microenvironment and quickly lose their ability to kill tumor cells. Accordingly, there is a need for T cells and natural killer cells that exhibit longer persistence in the tumor microenvironment and/or sustained capability to kill tumor cells. It is also desirable to develop methods of manufacturing T cells and natural killer cells with enhanced, specific cytotoxic activity for immunotherapy.

### BRIEF SUMMARY

In embodiments, a dominant negative TGFβ Receptor (dnTGFβR) polypeptide may be provided. In embodiments, a dominant negative TGFβ Receptor I (dnTGFβRI) polypeptide may be provided. In embodiments, a dominant negative TGFβ Receptor II (dnTGFβRII) polypeptide may be provided. In embodiments, isolated nucleic acid sequences encoding one or more dnTGFβRI polypeptides and/or dnTGFβRII polypeptides may be provided. In embodiments, isolated vectors comprising one or more nucleic acid sequences comprising one or more nucleic acid sequences encoding one or more dnTGFβRI polypeptides and/or dnTGFβRII polypeptides may be provided. In embodiments, cells expressing comprising or expressing one or more dnTGFβRI polypeptides and/or dnTGFβRII polypeptides may be provided. In embodiments, cells comprising or expressing one or more nucleic acid sequences comprising one or more nucleic acid sequences encoding one or more dnTGFβRI polypeptides and/or dnTGFβRII polypeptides may be provided. In embodiments, cells comprising or expressing one or more vectors comprising one or more nucleic acid sequences encoding one or more dnTGFβRI polypeptides and/or dnTGFβRII polypeptides may be provided. In embodiments, cells described herein may comprise one or more dnTGFβRI polypeptides and/or dnTGFβRII polypeptides, one or more CD8 polypeptide, one or more cell receptor (TCR) comprising an α chain and a β chain, one or more TCR comprising an γ chain and a δ chain, one or more chimeric antigen receptor (CAR), or any combination thereof. In embodiments, a cell may comprise an αβ T cell, an γδ T cell, a natural killer cell, a natural killer T cell, a CD4+ cell, a CD8+ cell, a CD4+/CD8+ cell, or any combination thereof. In embodiments, compositions comprising such polypeptides, nucleic acids, vectors, and/or cells may be provided. In embodiments, such polypeptides, nucleic acids, vectors, and/or cells may be isolated, recombinant, and/or engineered.

In embodiments, isolated polypeptide(s) may be encoded by nucleic acids described herein or, due, for example, to codon degeneration, by nucleic acids encoding the same polypeptide.

In embodiments, a dnTGFβRII polypeptide may comprise a mutated and/or truncated TGFβ Receptor II (TGFβRII). In embodiments, a dnTGFβRII polypeptide may comprise a truncated TGFβRII. In embodiments, a dnTGFβRII polypeptide may comprise a C-terminally truncated TGFβRII. In embodiments, a dnTGFβRII polypeptide may comprise a TGFβRII truncated to remove all or a portion of an intracellular signaling portion of TGFβRII. In embodiments, a dnTGFβRII polypeptide may comprise a TGFβRII mutated to fully or partially disable an intracellular signaling portion of TGFβRII. TGFβRIIvar1 and TGFβRIIvar2 disclosed herein each lack the cytoplasmic domain necessary for downstream signaling. Without being bound by theory, in embodiments, dnTGFβRII may function, for example, as follows: Truncated TGFβRII retains the ability to bind TGF-β and to form heteromeric complexes with TGFβRI, however the lack of the cytoplasmic domain prevents the phosphorylation of TGFβRI and subsequent activation of downstream elements. Moreover, the inclusion of a single truncated TGFβRII protein within the heteromeric TGF-β receptor complex is sufficient to ablate signaling, suggesting that it performs in a dominant-negative fashion.

In embodiments, a dnTGFβRII polypeptide may comprise an extracellular domain, a transmembrane domain, and/or a cytoplasmic domain. In embodiments, the cytoplasmic domain may be truncated, mutated, or absent.

In embodiments, dnTGFβRII variant 1 (dnTGFβRIIvar1) and/or dnTGFβRII variant 2 (dnTGFβRIIvar2) are provided and are examples of dnTGFβRII polypeptides. dnTGFβRIIvar1 may comprise or consist of SEQ ID NO: 305 and may be encoded by a nucleic acid comprising or consisting of SEQ ID NO: 306. dnTGFβRIIvar1 may comprise or consist of SEQ ID NO: 307 and may be encoded by a nucleic acid comprising or consisting of SEQ ID NO: 308. TGFβRIIvar1 and TGFβRIIvar2 disclosed herein each lack the cytoplasmic domain necessary for downstream signaling; in each, the remaining transmembrane and extracellular regions contain slight differences in size/sequence.

In embodiments, cells described herein may comprise an dnTGFβRII polypeptide and a CD8 polypeptide as described herein. In embodiments, cells described herein may comprise a dnTGFβRII polypeptide, a CD8 polypeptide, a T cell receptor (TCR) comprising an α chain and a β chain, a TCR comprising an γ chain and a S chain, a chimeric antigen receptor (CAR), or any combinations thereof. In embodiments, a cell may comprise an αβ T cell, an γδ T cell, a natural killer cell, a natural killer T cell, a CD4+ cell, a CD8+ cell, a CD4+/CD8+ cell, or combinations thereof.

In embodiments, CD8 polypeptides described herein may comprise a CD8α immunoglobulin (Ig)-like domain, a CD8β region, a CD8α transmembrane domain, and a CD8α cytoplasmic domain. In embodiments, a CD8β region may be a CD8β stalk region or domain.

In embodiments, CD8 polypeptides described herein may comprise (a) an immunoglobulin (Ig)-like domain comprising at least about 80%, 85%, 90%, 91%, 92%, 93%, 94%, 95%, 96%, 97%, 98%, 99%, or 100% sequence identity to the amino acid sequence of SEQ ID NO: 1, (b) a CD8β region comprising at least about 80%, 85%, 90%, 91%, 92%, 93%, 94%, 95%, 96%, 97%, 98%, 99%, or 100% identity sequence identity to the amino acid sequence of SEQ ID NO: 2, (c) a transmembrane domain comprising at least about 80%, 85%, 90%, 91%, 92%, 93%, 94%, 95%, 96%, 97%, 98%, 99%, or 100% sequence identity to the amino acid sequence of SEQ ID NO: 3, and (d) a cytoplasmic domain comprising at least about 80%, 85%, 90%, 91%, 92%, 93%, 94%, 95%, 96%, 97%, 98%, 99%, or 100% sequence identity to the amino acid sequence of SEQ ID NO: 4.

In embodiments, CD8 polypeptides described herein have at least about 80%, 85%, 90%, 91%, 92%, 93%, 94%, 95%, 96%, 97%, 98%, 99%, or 100% sequence identity to the amino acid sequence of SEQ ID NO: 5.

In embodiments, CD8 polypeptides described herein have at least about 80%, 85%, 90%, 91%, 92%, 93%, 94%, 95%, 96%, 97%, 98%, 99%, or 100% sequence identity to the amino acid sequence of SEQ ID NO: 7.

In embodiments, CD8 polypeptides described herein may comprise one or more signal peptide with at least about 80%, 85%, 90%, 91%, 92%, 93%, 94%, 95%, 96%, 97%, 98%, 99%, or 100% sequence identity to the amino acid sequence of any one of SEQ ID NO: 6, SEQ ID NO: 293, or SEQ ID NO: 294 fused to the N-terminus or to the C-terminus of CD8 polypeptides described herein.

In embodiments, CD8 polypeptides described herein may comprise (a) SEQ ID NO: 1 comprising one, two, three, four, or five amino acid substitutions; (b) SEQ ID NO: 2 comprising one, two, three, four, or five amino acid substitutions; (c) SEQ ID NO: 3 comprising one, two, three, four, or five amino acid substitutions, and (d) SEQ ID NO: 4 comprising one, two, three, four, or five amino acid substitutions. In embodiments, amino acid substitutions may be conservative or non-conservative. In embodiments, amino acid substitution(s) may be conservative amino acid substitution(s).

In embodiments, CD8 polypeptides described herein may be CD8α or modified CD8α polypeptides.

In embodiments, CD8 polypeptides described herein may be CD8αβ or modified CD8α polypeptides.

In embodiments, a CD8β polypeptide may comprise the amino acid sequence of any one of SEQ ID NO: 8, 9, 10, 11, 12, 13, or 14.

In embodiments, a TCR α chain and a TCR β chain may be selected from SEQ ID NO: 15 and 16; 17 and 18; 19 and 20; 21 and 22; 23 and 24; 25 and 26; 27 and 28; 29 and 30; 31 and 32; 33 and 34; 35 and 36; 37 and 38; 39 and 40; 41 and 42; 43 and 44; 45 and 46; 47 and 48; 49 and 50; 51 and 52; 53 and 54; 55 and 56; 57 and 58; 59 and 60; 61 and 62; 63 and 64; 65 and 66; 67 and 68; 69 and 70; 71 and 303; 304 and 74; 75 and 76; 77 and 78; 79 and 80; 81 and 82; 83 and 84; 85 and 86; 87 and 88; 89 and 90; and 91 and 92.

In embodiments, an isolated nucleic acid may comprise a nucleic acid encoding a T-cell receptor comprising an α chain and a β chain and a CD8 polypeptide comprising an α chain and a β chain. In embodiments, a CD8 polypeptide may be modified or unmodified. An isolated nucleic acid may comprise a nucleic acid at least about 80% identical to the nucleic acid of SEQ ID NO: 267, 269, 271, 273, 275, 277, 279, 281, 283, 285, 287, 289, 291, 295, 297, 299, or 301. An isolated nucleic acid may be at least about 90%, 91%, 92%, 93%, 94%, 95%, 96%, 97%, 98%, 99%, or 100% identical to the nucleic acid of SEQ ID NO: 267, 269, 271, 273, 275, 277, 279, 281, 283, 285, 287, 289, 291, 295, 297, 299, or 301.

In an aspect, polypeptide sequences and/or nucleic acid sequences described herein may be isolated and/or recombinant sequences.

In embodiments, an isolated nucleic acid comprises the nucleic acid of SEQ ID NO: 267.

In embodiments, an isolated nucleic acid comprises the nucleic acid of SEQ ID NO: 279.

In embodiments, isolated polypeptide(s) may be encoded by nucleic acids described herein or, due, for example, to codon degeneration, by nucleic acids encoding the same polypeptide.

In embodiments, an isolated polypeptide may comprise an amino acid sequence at least about 80% identical to the amino acid sequence of SEQ ID NO: 268, 270, 272, 274, 276, 278, 280, 282, 284, 286, 288, 290, 292, 296, 298, 300, or 302. An amino acid sequence may be at least about 90%, 91%, 92%, 93%, 94%, 95%, 96%, 97%, 98%, 99%, or 100% identical to the amino acid sequence of SEQ ID NO: 268, 270, 272, 274, 276, 278, 280, 282, 284, 286, 288, 290, 292, 296, 298, 300, or 302. In another aspect, SEQ ID NO: 268, 270, 272, 274, 276, 278, 280, 282, 284, 286, 288, 290, 292, 296, 298, 300, or 302 comprise 1, 2, 3, 4, 5, 10, 15, or 20 or more amino acid substitutions or deletions. In yet another aspect, SEQ ID NO: 268, 270, 272, 274, 276, 278, 280, 282, 284, 286, 288, 290, 292, 296, 298, 300, or 302 comprise at most 1, 2, 3, 4, 5, 10, 15, or 20 amino acid substitutions or deletions.

In embodiments, an isolated polypeptide may comprise the amino acid sequence of SEQ ID NO: 268.

In embodiments, an isolated polypeptide may comprise the amino acid sequence of SEQ ID NO: 280.

In embodiments, the disclosure provides for nucleic acid(s) encoding polypeptide(s) described herein.

In embodiments, the disclosure provides for vectors comprising nucleic acids encoding polypeptide(s) described herein.

In embodiments, one or more vector may comprise a nucleic acid encoding a dnTGFβRII polypeptide.

In embodiments, one or more vector may comprise a nucleic acid encoding a CD8 polypeptide.

In embodiments, one or more vector may comprise a nucleic acid encoding a CD8α polypeptide.

In embodiments, one or more vector may comprise a nucleic acid encoding a CD8β polypeptide.

In embodiments, a CD8 polypeptide may comprise a CD8α chain and/or a CD8β chain, and the CD8α chain and/or CD8β chain may independently be modified or unmodified.

In embodiments, one or more vector may comprise one or more nucleic acid encoding a T cell receptor (TCR) comprising an α chain and a β chain. In embodiments, one or more vector may comprise one or more nucleic acid encoding a T cell receptor (TCR) comprising an γ chain and a δ chain. In embodiments, one or more vector may comprise one or more nucleic acid encoding a chimeric antigen receptor (CAR).

In embodiments, a vector or vectors comprising one or more nucleic acid(s) encoding one or any combination of a TCR comprising an α chain and a β chain, a TCR comprising a γ chain and a δ chain, a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding one or any combination of a TCR comprising an α chain and a β chain, a dnTGFβRII polypeptide, and/or a CD8 polypeptide may be provided. In embodiments, a vector or vectors comprising one or more nucleic acid(s) encoding one or any combination of a TCR comprising a γ chain and a δ chain, a dnTGFβRII polypeptide, and/or a CD8 polypeptide may be provided. In embodiments, a vector or vectors comprising one or more nucleic acid(s) encoding one or any combination of a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding a TCR comprising an α chain and a β chain, a dnTGFβRII polypeptide, and a CD8 polypeptide may be provided. In embodiments, a vector or vectors comprising one or more nucleic acid(s) encoding a TCR comprising a γ chain and a δ chain, a dnTGFβRII polypeptide, and a CD8 polypeptide may be provided. In embodiments, a vector or vectors comprising one or more nucleic acid(s) encoding a CAR, a dnTGFβRII polypeptide, and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding a TCR comprising an α chain and a β chain and a dnTGFβRII polypeptide may be provided. In embodiments, a vector or vectors comprising one or more nucleic acid(s) encoding a TCR comprising a γ chain and a δ chain and a dnTGFβRII polypeptide may be provided. In embodiments, a vector or vectors comprising one or more nucleic acid(s) encoding a CAR and a dnTGFβRII polypeptide may be provided.

In embodiments, a vector or vectors comprising one or more nucleic acid(s) encoding a TCR comprising an α chain and a β chain and a CD8 polypeptide may be provided. In embodiments, a vector or vectors comprising one or more nucleic acid(s) encoding a TCR comprising a γ chain and a δ chain and a CD8 polypeptide may be provided. In embodiments, a cell or cells comprising one or more nucleic acid(s) encoding a CAR and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a nucleic acid encoding a polypeptide comprising (i) SEQ ID NO: 305 or a sequence at least about 95%, at least about 96%, at least about 97%, at least about 98%, at least about 99%, or about 100% identical to SEQ ID NO: 305; (ii) SEQ ID NO: 307 or a sequence at least about 95%, at least about 96%, at least about 97%, at least about 98%, at least about 99%, or about 100% identical to SEQ ID NO: 307; or (iii) both (i) and (ii) may be provided.

In embodiments, a nucleic acid comprising (i) SEQ ID NO: 306 or a sequence at least about 80%, at least about 85%, at least about 90%, at least about 95%, at least about 96%, at least about 97%, at least about 98%, at least about 99%, or about 100% identical to SEQ ID NO: 306; (ii) SEQ ID NO: 308 or a sequence at least about 80%, at least about 85%, at least about 90%, at least about 95%, at least about 96%, at least about 97%, at least about 98%, at least about 99%, or about 100% identical to SEQ ID NO: 308; or (iii) both (i) and (ii) may be provided.

In embodiments, a nucleic acid comprising (i) SEQ ID NO: 312 or a sequence at least about 80%, at least about 85%, at least about 90%, at least about 95%, at least about 96%, at least about 97%, at least about 98%, at least about 99%, or about 100% identical to SEQ ID NO: 312; (ii) SEQ ID NO: 313 or a sequence at least about 80%, at least about 85%, at least about 90%, at least about 95%, at least about 96%, at least about 97%, at least about 98%, at least about 99%, or about 100% identical to SEQ ID NO: 313; or (iii) both (i) and (ii) may be provided.

In embodiments, a nucleic acid described herein may further comprise a nucleic acid sequence encoding at least one TCR polypeptide, at least one CD8 polypeptide, or at least one TCR polypeptide and at least one CD8 polypeptide.

In embodiments, a nucleic acid comprising: (a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) a nucleic acid sequence encoding at least one dominant negative TGFβ Receptor II (dnTGFβRII) polypeptide, wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92; wherein the CD8 α chain is SEQ ID NO: 7, 258, 259, 262, or a variant thereof; wherein the CD8 β chain is SEQ ID NO: 8, 9, 10, 11, 12, 13, or 14; and wherein at least one of the at least one dnTGFβRII polypeptide is selected from (i) SEQ ID NO: 305 or a sequence at least about 95%, at least about 96%, at least about 97%, at least about 98%, at least about 99%, or about 100% identical thereto or (ii) SEQ ID NO: 307 or a sequence at least about 95%, at least about 96%, at least about 97%, at least about 98%, at least about 99%, or about 100% identical thereto may be provided. In embodiments, the nucleic acid may comprise a nucleic acid sequence encoding dnTGFβRIIvar1 and a nucleic acid sequence encoding dnTGFβRIIvar2.

In embodiments, a nucleic acid comprising: (a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) a nucleic acid sequence encoding at least one dominant negative TGFβ Receptor II (dnTGFβRII) polypeptide, wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303; wherein the CD8 α chain is SEQ ID NO: 7, 258, 259, 262, or a variant thereof; wherein the CD8 β chain is SEQ ID NO: 8, 9, 10, 11, 12, 13, or 14; and wherein the at least one dnTGFβRII polypeptide is selected from (i) SEQ ID NO: 305 or a sequence at least about 95%, at least about 96%, at least about 97%, at least about 98%, at least about 99%, or about 100% identical thereto or (ii) SEQ ID NO: 307 or a sequence at least about 95%, at least about 96%, at least about 97%, at least about 98%, at least about 99%, or about 100% identical thereto may be provided. In embodiments, the nucleic acid may comprise a nucleic acid sequence encoding dnTGFβRIIvar1 and a nucleic acid sequence encoding dnTGFβRIIvar2.

In embodiments, a nucleic acid comprising: (a) a nucleic acid sequence at least about 80% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide may be provided.

In embodiments, a nucleic acid comprising: (a) a nucleic acid sequence at least about 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide may be provided.

In embodiments, the nucleic acid sequence or sequences encoding at least one of the at least one dominant negative TGFβ Receptor II (dnTGFβRII) polypeptide may be selected from (i) SEQ ID NO: 306 or a sequence at least about 90%, 91%, 92%, 93%, 94%, 95%, 96%, 97%, 98%, 99%, or 100% identical thereto or (ii) SEQ ID NO: 308 or a sequence at least about 90%, 91%, 92%, 93%, 94%, 95%, 96%, 97%, 98%, 99%, or 100% identical thereto.

In embodiments, vector comprising N1, N2, N3, N4, N5, L1, L2, L3, and L4, in any order, wherein N1 comprises a nucleic acid sequence encoding a CD8β chain and is present or absent, N2 comprises a nucleic acid sequence encoding a CD8α chain, N3 comprises a nucleic acid sequence encoding a TCRβ chain, N4 comprises a nucleic acid sequence encoding a TCRα chain, and N5 comprises a nucleic acid sequence encoding at least one dominant negative TGFβ Receptor II (dnTGFβRII) polypeptide; and wherein L1-L4 each comprises a nucleic acid sequence encoding at least about one linker, wherein each of L1-L4 is independently the same or different, and wherein each of L1-L4 is independently present or absent may be provided.

In embodiments, a vector comprising Formula I or Formula II:

[I]
5′-N1-L1-N2-L2-N3-L3-N4-L4-N5-3′
[II]
5′-N5-L1-N1-L2-N2-L3-N3-L4-N4-3′.

may be provided.

In embodiments, N1 may comprise a nucleic acid sequence encoding SEQ ID NO: 8, 9, 10, 11, 12, 13, or 14.

In embodiments, N2 may comprise a nucleic acid sequence encoding SEQ ID NO: 7, 258, 259, 262, or a variant thereof.

In embodiments, N4 and N3 may comprise a nucleic acid sequence encoding SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, or 91 and 92.

In embodiments, N5 may comprise a nucleic acid sequence encoding (i) SEQ ID NO: 305 or a sequence at least about 95%, at least about 96%, at least about 97%, at least about 98%, at least about 99%, or about 100% identical to SEQ ID NO: 305 or (ii) SEQ ID NO: 307 or a sequence at least about 95%, at least about 96%, at least about 97%, at least about 98%, at least about 99%, or about 100% identical to SEQ ID NO: 307.

In embodiments, the vector may further comprise (i) a nucleic acid encoding a 2A peptide or an internal ribosome entry site (IRES) positioned between N1 and L1, between L1 and N2, between N2 and L2, between L2 and N3, between N3 and L3, between L3 and N4, between N4 and L4, between L4 and N5, or any combination thereof or (ii) a nucleic acid encoding a 2A peptide or an internal ribosome entry site (IRES) positioned between N5 and L1, between L1 and N1, between N1 and L2, between L2 and N2, between N2 and L3, between L3 and N3, between N3 and L4, between L4 and N4, or any combination thereof. In embodiments, the 2A peptide may be P2A (SEQ ID NO: 93), T2A (SEQ ID NO: 94), E2A (SEQ ID NO: 95), or F2A (SEQ ID NO: 96). In embodiments, the IRES may be selected from the group consisting of IRES from picornavirus, IRES from flavivirus, IRES from pestivirus, IRES from retrovirus, IRES from lentivirus, IRES from insect RNA virus, and IRES from cellular mRNA.

In embodiments, the vector may further comprise (i) a nucleic acid encoding a furin positioned between N1 and L1, between L1 and N2, between N2 and L2, between L2 and N3, between N3 and L3, between L3 and N4, between N4 and L4, between L4 and N5, or any combination thereof or (ii) a nucleic acid encoding a furin positioned between N5 and L1, between L1 and N1, between N1 and L2, between L2 and N2, between N2 and L3, between L3 and N3, between N3 and L4, between L4 and N4, or any combination thereof.

In embodiments, a T cell and/or natural killer (NK) cell comprising: (a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) at least one dominant negative TGFβ Receptor II (dnTGFβRII) polypeptide, wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92; wherein the CD8 α chain is SEQ ID NO: 7, 258, 259, 262, or a variant thereof; wherein the CD8 β chain is SEQ ID NO: 8, 9, 10, 11, 12, 13, or 14; and wherein at least one of the at least one dominant negative TGFβ Receptor II (dnTGFβRII) polypeptide is selected from (i) SEQ ID NO: 305 or a sequence at least about 95%, at least about 96%, at least about 97%, at least about 98%, at least about 99%, or about 100% identical to SEQ ID NO: 305 or (ii) SEQ ID NO: 307 or a sequence at least about 95%, at least about 96%, at least about 97%, at least about 98%, at least about 99%, or about 100% identical to SEQ ID NO: 307 may be provided. In embodiments, a T cell and/or natural killer call may comprise a dnTGFβRIIvar1 and dnTGFβRIIvar2.

In embodiments, a T cell and/or natural killer (NK) cell comprising: (a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) at least one dominant negative TGFβ Receptor II (dnTGFβRII) polypeptide, wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303; wherein the CD8 α chain is SEQ ID NO: 7, 258, 259, 262, or a variant thereof; and wherein, if present, the CD8 β chain is SEQ ID NO: 8, 9, 10, 11, 12, 13, or 14 may be provided. In embodiments, a T cell and/or natural killer call may comprise a dnTGFβRIIvar1 and dnTGFβRIIvar2. In embodiments, at least one of the at least one dominant negative TGFβ Receptor II (dnTGFβRII) polypeptide may comprise SEQ ID NO: 305 or a sequence at least about 95%, at least about 96%, at least about 97%, at least about 98%, at least about 99%, or about 100% identical thereto. In embodiments, at least one of the at least one dominant negative TGFβ Receptor II (dnTGFβRII) polypeptide may comprise SEQ ID NO: 307 or a sequence at least about 95%, at least about 96%, at least about 97%, at least about 98%, at least about 99%, or about 100% identical thereto.

In embodiments, a nucleic acid comprising: (a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) a nucleic acid sequence encoding at least one dominant negative TGFβ Receptor II (dnTGFβRII) polypeptide, wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92; wherein the CD8 α chain is SEQ ID NO: 7, 258, 259, 262, or a variant thereof; wherein the CD8 β chain is SEQ ID NO: 8, 9, 10, 11, 12, 13, or 14; and wherein at least one of the at least one dnTGFβRII polypeptide is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical to thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto may be provided. In embodiments, the nucleic acid may comprise a nucleic acid sequence encoding dnTGFβRIIvar1 and a nucleic acid sequence encoding dnTGFβRIIvar2.

In embodiments, a nucleic acid comprising: (a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) a nucleic acid sequence encoding at least one dominant negative TGFβ Receptor II (dnTGFβRII) polypeptide, wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303; wherein the CD8 α chain is SEQ ID NO: 7, 258, 259, 262, or a variant thereof; wherein the CD8 β chain is SEQ ID NO: 8, 9, 10, 11, 12, 13, or 14; and wherein at least one of the at least one dnTGFβRII polypeptide is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical or (ii) SEQ ID NO: 313 or a sequence at least about 80%, at least about 85%, at least about 90%, at least about 95%, at least about 96%, at least about 97%, at least about 98%, at least about 99%, or about 100% identical thereto may be provided. In embodiments, the nucleic acid may comprise a nucleic acid sequence encoding dnTGFβRIIvar1 and a nucleic acid sequence encoding dnTGFβRIIvar2.

In embodiments, a nucleic acid comprising: (a) a nucleic acid sequence at least about 80% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide may be provided.

In embodiments, a nucleic acid comprising: (a) a nucleic acid sequence at least about 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide may be provided. In embodiments, the nucleic acid sequence encoding at least one of the at least one dominant negative TGFβ Receptor II (dnTGFβRII) polypeptide may also comprise an MSCV promoter and a WPRE sequence and may be selected from (i) SEQ ID NO: 312 or a sequence at least about 80%, at least about 85%, at least about 90%, at least about 95%, at least about 96%, at least about 97%, at least about 98%, at least about 99%, or about 100% identical thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto.

In embodiments, a T cell and/or natural killer (NK) cell comprising: (a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) at least one dominant negative TGFβ Receptor II (dnTGFβRII) polypeptide, wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92; wherein the CD8 α chain is SEQ ID NO: 7, 258, 259, 262, or a variant thereof; wherein the CD8 β chain is SEQ ID NO: 8, 9, 10, 11, 12, 13, or 14; and is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical to thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto may be provided. In embodiments, a T cell and/or natural killer call may comprise a dnTGFβRIIvar1 and dnTGFβRIIvar2.

In embodiments, a T cell and/or natural killer (NK) cell comprising: (a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and (b) at least one dominant negative TGFβ Receptor II (dnTGFβRII) polypeptide, wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303; wherein the CD8 α chain is SEQ ID NO: 7, 258, 259, 262, or a variant thereof; and wherein, if present, the CD8 β chain is SEQ ID NO: 8, 9, 10, 11, 12, 13, or 14 may be provided. In embodiments, a T cell and/or natural killer call may comprise a dnTGFβRIIvar1 and dnTGFβRIIvar2. In embodiments, at least one of the at least one dominant negative TGFβ Receptor II (dnTGFβRII) polypeptide may be encoded by a nucleic acid sequence that also comprises an MSCV promoter and a WPRE sequence and that is selected from SEQ ID NO: 312 or a sequence at least about 80%, at least about 85%, at least about 90%, at least about 95%, at least about 96%, at least about 97%, at least about 98%, at least about 99%, or about 100% identical to thereto. In embodiments, at least one of the at least one dominant negative TGFβ Receptor II (dnTGFβRII) polypeptide may be encoded by a nucleic acid sequence that also comprises an MSCV promoter and a WPRE sequence and that is selected from SEQ ID NO: 313 or a sequence at least about 80%, at least about 85%, at least about 90%, at least about 95%, at least about 96%, at least about 97%, at least about 98%, at least about 99%, or about 100% identical to thereto.

In embodiments, a method of preparing T cells and/or natural killer cells for immunotherapy may be provided, the method comprising: isolating T cells and/or natural killer cells from a blood sample of a human subject, activating the isolated T cells and/or natural killer cells, transducing the activated T cells and/or natural killer cells with a nucleic acid of described herein or a vector described herein, and expanding the transduced T cells and/or natural killer cells. In embodiments, the method may further comprise isolating CD4+CD8+ T cells from the transduced T cells and/or natural killer cells and expanding the isolated CD4+CD8+ transduced T cells. In embodiments, the blood sample may comprise peripheral blood mononuclear cells (PMBC). In embodiments, the activating may comprise contacting the T cells and/or natural killer cells with an anti-CD3 and an anti-CD28 antibody. In embodiments, the T cell may be a CD4+ T cell. In embodiments, the T cell may be a CD8+ T cell. In embodiments, the T cell may be a γδ T cell or an αβ T cell. In embodiments, the activation and/or expanding may be in the presence of a combination of IL-2 and IL-15 and optionally with zoledronate.

In embodiments, a method of increasing persistence, longevity, functionality, naivety, capacity to kill antigen-presenting cells, or a combination thereof, of T cells and/or natural killer (NK) cell may be provided, the method comprising: isolating T cells and/or natural killer (NK) cells from a blood sample of a human subject, activating the isolated T cells and/or natural killer (NK) cells, transducing the activated T cells and/or natural killer (NK) cells with a nucleic acid described herein, a vector described herein, or a combination thereof, to obtain transduced T cells and/or natural killer (NK) cells, and obtaining the transduced T cells or natural killer (NK) cells, wherein the persistence, longevity, functionality, naivety, capacity to kill antigen-presenting cells, or a combination thereof of the transduced T cells and/or natural killer (NK) cells is increased as compared with that of control cells. In embodiments, the method may further comprise expanding the transduced T cells and/or natural killer (NK) cells. In embodiments, the control cells may comprise non-transduced T cells and/or natural killer (NK) cells, T cells and/or natural killer (NK) cells transduced with TCR only, or a combination thereof. In embodiments, the control cells may comprise non-transduced T cells and/or natural killer (NK) cells, T cells and/or natural killer (NK) cells transduced with TCR only, T cells and/or natural killer (NK) cells transduced with TCR and CD8 only, or a combination thereof. In embodiments, the persistence, longevity, functionality, naivety, capacity to kill antigen-presenting cells, or a combination thereof of the transduced T cells and/or natural killer (NK) cells and the control cells may be determined after one challenge with antigen-presenting cells, two challenges with antigen-presenting cells, three challenges with antigen-presenting cells, four challenges with antigen-presenting cells, five challenges with antigen-presenting cells, six challenges with antigen-presenting cells, seven challenges with antigen-presenting cells, or more challenges with antigen-presenting cells. In embodiments, the persistence, longevity, functionality, naivety, capacity to kill antigen-presenting cells, or a combination thereof of the transduced T cells and/or natural killer (NK) cells and the control cells may be determined after two challenges with antigen-presenting cells, after three challenges with antigen-presenting cells, or after more challenges with antigen-presenting cells. In embodiments, the transduced T cells and/or natural killer (NK) cells and the control cells may be cultured in the presence of exogenous TGF-β, optionally TGF-β1. In embodiments, the exogenous TGF-β, optionally TGF-β1, may be added to cell cultures daily. In embodiments, the exogenous TGF-β, optionally TGF-β1, is added to cell cultures at the same time or times that tumor cells may be added to cell cultures.

In embodiments, method of increasing interferon γ (IFNγ) secretion by T cells and/or natural killer (NK) cells may be provided, the method comprising: isolating T cells and/or natural killer (NK) cells from a blood sample of a human subject, activating the isolated T cells and/or natural killer (NK) cells, transducing the activated T cells and/or natural killer (NK) cells with a nucleic acid described herein, a vector described herein, or a combination thereof, to obtain transduced T cells and/or natural killer (NK) cells, and obtaining the transduced T cells or natural killer (NK) cells, wherein the IFNγ secretion of the transduced T cells and/or natural killer (NK) cells is increased as compared with that of control cells. In embodiments, the method may further comprise expanding the transduced T cells and/or natural killer (NK) cells. In embodiments, the control cells may comprise non-transduced T cells and/or natural killer (NK) cells, T cells and/or natural killer (NK) cells transduced with TCR only, or a combination thereof. In embodiments, the control cells may comprise non-transduced T cells and/or natural killer (NK) cells, T cells and/or natural killer (NK) cells transduced with TCR only, T cells and/or natural killer (NK) cells transduced with TCR and CD8 only, or a combination thereof. In embodiments, the IFNγ secretion by the transduced T cells and/or natural killer (NK) cells and the control cells may be determined after one challenge with antigen-presenting cells, two challenges with antigen-presenting cells, three challenges with antigen-presenting cells, four challenges with antigen-presenting cells, five challenges with antigen-presenting cells, six challenges with antigen-presenting cells, seven challenges with antigen-presenting cells, or more challenges with antigen-presenting cells. In embodiments, the IFNγ secretion by the transduced T cells and/or natural killer (NK) cells and the control cells may be determined after two challenges with antigen-presenting cells, after three challenges with antigen-presenting cells, or after more challenges with antigen-presenting cells. In embodiments, the transduced T cells and/or natural killer (NK) cells and the control cells may be cultured in the presence of exogenous TGF-β, optionally TGF-β1. In embodiments, the exogenous TGF-β, optionally TGF-β1, may be added to cell cultures daily. In embodiments, the exogenous TGF-β, optionally TGF-β1, may be added to cell cultures at the same time or times that tumor cells are added to cell cultures.

In embodiments, the antigen presenting cells may present an antigen on a cell surface, and the transduced T cells and/or natural killer (NK) cells and the control cells may be capable of killing the antigen presenting cells. In embodiments, the antigen may comprise a peptide. In embodiments, the antigen comprising a peptide may be in a complex with an MHC molecule on the cell surface.

In embodiments, nucleic acid encoding a fusion polypeptide of Formula III:

N-terminus-P6-PL-P7-C-terminus [III],

wherein P6 and P7 are each independently a first and second polypeptides and PL is a linker, wherein PL comprises SEQ ID NO: 320 or 322 or a sequence at least about 95%, at least about 96%, at least about 97%, at least about 98%, at least about 99%, or about 100% identical to SEQ ID NO: 320 or 322 may be provided.

In embodiments, nucleic acid comprising formula IV:

5′-N6-NL-N7-3′ [IV],

wherein N6 and N7 each independently encode a first and second polypeptides and NL encodes a linker, wherein NL comprises SEQ ID NO: 321 or 323 or a sequence at least about 95%, at least about 96%, at least about 97%, at least about 98%, at least about 99%, or about 100% identical to SEQ ID NO: 321 or 323 may be provided.

In embodiments, polypeptide, polypeptides, or fusion polypeptide encoded by a nucleic acid described herein may be provided.

In embodiments, polypeptide, polypeptides, or fusion polypeptide herein may be isolated, recombinant, or both isolated and recombinant.

In embodiments, a T cell and/or natural killer (NK) cell comprising a polypeptide comprising an amino acid sequence at least about 95%, 96%, 97%, 98%, 99%, or 100% identical to the amino acid sequence of SEQ ID NO: 305 or 307 and (a) at least one TCR polypeptide comprising an α chain and a β chain, (b) at least one CD8 polypeptide comprising (i) an α chain, (ii) a β chain, or (iii) an α chain and a β chain or (c) at least one TCR polypeptide comprising an α chain and a β chain and at least one CD8 polypeptide comprising (i) an α chain, (ii) a β chain, or (iii) an α chain and a β chain may be provided. In embodiments, the T cell may be an αβ T cell, a γδ T cell, and/or a natural killer T cell. In embodiments, the αβ T cell may be a CD4+ T cell. In embodiments, the αβ T cell may be a CD8+ T cell. In embodiments, the γδ T cell may be a Vγ9Vδ2+ T cell.

In embodiments, a nucleic acid described herein may be isolated, recombinant, or both isolated and recombinant.

In embodiments, a vector described herein may be isolated, recombinant, or both isolated and recombinant.

In embodiments, a T cell and/or natural killer (NK) cell described herein may be isolated, recombinant, engineered, or any combination thereof.

In embodiments, a vector comprising a nucleic acid described herein may be provided. In embodiments, a vector described herein may further comprise a nucleic acid encoding a 2A peptide or an internal ribosome entry site (IRES) positioned between a nucleic acid encoding a CD8 α chain and a nucleic acid encoding a CD8 β chain. In embodiments, the vector may further comprise a nucleic acid encoding a 2A peptide or an IRES positioned between a nucleic acid encoding a TCR α chain and a nucleic acid encoding a TCR β chain. In embodiments, the vector may further comprise a nucleic acid encoding a 2A peptide or an IRES positioned between a nucleic acid encoding a TCR chain or a CD8 chain and a nucleic acid encoding a dominant negative TGFβRII. In embodiments, the 2A peptide may be P2A (SEQ ID NO: 93), T2A (SEQ ID NO: 94), E2A (SEQ ID NO: 95), or F2A (SEQ ID NO: 96). In embodiments, the IRES may be selected from the group consisting of IRES from picornavirus, IRES from flavivirus, IRES from pestivirus, IRES from retrovirus, IRES from lentivirus, IRES from insect RNA virus, and IRES from cellular mRNA. In embodiments, the vector may further comprise a post-transcriptional regulatory element (PRE) sequence selected from a Woodchuck PRE (WPRE) (SEQ ID NO: 264), Woodchuck PRE (WPRE) mutant 1 (SEQ ID NO: 256), Woodchuck PRE (WPRE) mutant 2 (SEQ ID NO: 257), or hepatitis B virus (HBV) PRE (HPRE) (SEQ ID NO: 437). In embodiments, the post-transcriptional regulatory element (PRE) sequence may be a Woodchuck PRE (WPRE) mutant 1 comprising the nucleic acid sequence of SEQ ID NO: 256. In embodiments, the post-transcriptional regulatory element (PRE) sequence may be a Woodchuck PRE (WPRE) mutant 2 comprising the nucleic acid sequence of SEQ ID NO: 257. In embodiments, the vector may further comprise a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, or Murine Stem Cell Virus (MSCV) promoter. In embodiments, the promoter may be a Murine Stem Cell Virus (MSCV) promoter. In embodiments, vector may be a viral vector or a non-viral vector. In embodiments, the vector may be a viral vector. In embodiments, the viral vector may be selected from adenoviruses, poxviruses, alphaviruses, arenaviruses, flaviviruses, rhabdoviruses, retroviruses, lentiviruses, herpesviruses, paramyxoviruses, picornaviruses, and any combination thereof. In embodiments, the viral vector may be pseudotyped with an envelope protein of a virus selected from the native feline endogenous virus (RD114), a version of RD114 (RD114TR), gibbon ape leukemia virus (GALV), a version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), and lymphocytic choriomeningitis virus (LCMV). In embodiments, the vector may be a lentiviral vector. In embodiments, the vector may further comprise a nucleic acid encoding a chimeric antigen receptor (CAR).

In embodiments, a T cell and/or natural killer cell expressing a polypeptide as described herein and/or comprising a vector described herein and/or produced by a method described herein may be provided. In embodiments, a T cell described herein may be an αβ T cell, a γδ T cell, a natural killer T cell, or any combination thereof. In embodiments, the αβ T cell may be a CD4+ T cell. In embodiments, the αβ T cell may be a CD8+ T cell. In embodiments, the γδ T cell may be a Vγ9Vδ2+ T cell.

In embodiments, a composition comprising a T cell and/or natural killer cell described herein may be provided. In embodiments, the composition may be a pharmaceutical composition. In embodiments, the composition may further comprise an adjuvant, excipient, carrier, diluent, buffer, stabilizer, or a combination thereof. In embodiments, the adjuvant may be an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof. In embodiments, the adjuvant may be IL-2, IL-7, IL-12, IL-15, IL-21, or any combination thereof.

In embodiments, a method of treating a patient who has cancer may be provided, the method comprising administering to the patient a composition described herein, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer. In embodiments, a method of eliciting an immune response in a patient who has cancer may be provided, the method comprising administering to the patient a composition described herein, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer. In embodiments, the T cell and/or natural killer cell may kill cancer cells that present a peptide in a complex with an MHC molecule on a cell surface.

In embodiments, expression of dnTGFβRII polypeptide may improve immune cell, such as but not limited to, T cell and/or natural killer cell, persistence, functionality, growth, viability, expansion, or combinations thereof, as compared to cells not expressing dnTGFβRII polypeptide. In embodiments, expression of dnTGFβRII polypeptide may improve immune cell, such as but not limited to, T cell and/or natural killer cell, persistence, functionality, growth, viability, expansion, or combinations thereof, in a tumor microenvironment, as compared to cells not expressing dnTGFβRII polypeptide. In embodiments, expression of dnTGFβRII polypeptide may increase efficacy of immune cells, such as, but not limited to, T cells and/or natural killer cells, in killing tumor cells, as compared to cells not expressing dnTGFβRII polypeptide. In embodiments, expression of dnTGFβRII polypeptide may increase ability of immune cells, such as, but not limited to, T cells and/or natural killer cells, to survive in a tumor microenvironment, to persist in killing tumor cells, or combinations thereof, as compared to cells not expressing dnTGFβRII polypeptide. In embodiments, expression of dnTGFβRII polypeptide may increase ability of immune cells, such as, but not limited to, T cells and/or natural killer cells, to maintain a naïve phenotype.

Persistence may be assessed, as a non-limiting example, by the length of time cells are detectable in an individual (e.g., patient) after infusion. As non-limiting examples, persistence may be measured at days, weeks, months, or years after infusion, as non-limiting examples, at about 1 week, about 2 weeks, about 4 weeks, about 1 month, about 2 months, about 3 months, about 6 months, about 9 months, about 12 months, about 18 months, about 24 months, and/or about 30 months after infusion. Persistence may be assessed, as non-limiting examples, by PCR of peripheral blood sample(s), by flow cytometry of peripheral blood samples(s), and/or by analysis of tumor biopsy sample(s). Persistence of cells expressing dnTGFβRII polypeptide may be compared, as non-limiting examples, to typical persistence of infused ACT cells or persistence of similar cells not expressing dnTGFβRII polypeptide.

Continued ability to kill tumor cells may be measured, as non-limiting examples, via (i) serial killing assays using an IncuCyte (wherein ability to kill/impair tumor growth as measured by fold growth during repeated tumor stimulations over a duration of time is assessed), and/or (ii) via cytokine/effector molecule production (IFNγ via ELISAs and other pro-inflammatory cytokines via Luminex (cytokines measured may include, as non-limiting examples, IFNγ, TNFα, Granzyme B, perforin, IL-2, IL-6, MIP-1β, MIP-1α, GM-CSF, RANTES, IL-18, IL-4, IL-10, and IP10)). Continued ability of cells expressing dnTGFβRII polypeptide to kill tumor cells may be compared, as non-limiting examples, to continued ability of similar cells not expressing dnTGFβRII polypeptide to kill tumor cells or continued ability other control cells to kill tumor cells.

Naivety of phenotype may be assessed, as a non-limiting example, via Tmem panel assay via flow cytometry. Typically, flow cytometer gating is off of CD8+ TCR+ cells. Typically, a more naïve phenotype may be indicated by higher frequencies of the T memory subsets Tnaïve/scm (CD45RA+CCR7+), and Tcm (CD45RA−CCR7+) and an increase or retention of the CD39−CD69− and CD27+CD28+ populations. Low CD57 expression may also be desirable.

When assessing the persistence, functionality, growth, viability, expansion, tumor killing efficacy, naivety, or other characteristics of cells expressing dnTGFRβRII, cells such as non-transduced cells, cells transduced with TCR only, cells transduced with CD8 and TCR, or a combination thereof, may serve as control cells, as non-limiting examples. As dnTGFβRII may act to reduce or ablate signalling of TGFβ, assessment of the persistence, functionality, growth, viability, expansion, tumor killing efficacy, naivety, or other characteristics of cells expressing dnTGFRβRII may be performed in the presence of exogenous TGFβ, such as TGF-β1.

In embodiments, dnTGFβRII polypeptide may act in a cis manner (e.g., affecting cells in which it is expressed), in a trans manner (e.g., affecting cells in which it is not expressed), or combinations thereof. In embodiments in which dnTGFβRII polypeptide acts in trans, cells adjacent to or near (e.g., within the tumor microenvironment) cells expressing dnTGFβRII polypeptide may exhibit any or combinations of improvements the same or similar to those described for cells expressing dnTGFβRII polypeptide, as compared to cells not adjacent to or near cells expressing dnTGFβRII polypeptide. Without being bound by theory, dnTGFβRII may act to reduce the amount of TGF-β in the tumor microenvironment; also, cells expressing dnTGFβRII may exhibit an improved ability to secrete cytokines in response to target antigen in the presence of TGF-β, as compared to cells that do not express dnTGFβRII.

In embodiments, the disclosure provides for nucleic acid(s) encoding polypeptide(s) described herein. In embodiments, the disclosure provides for vectors comprising nucleic acids encoding polypeptide(s) described herein. In embodiments, one or more vector may comprise a nucleic acid encoding a dnTGFβRII polypeptide. In embodiments, one or more vector may comprise a nucleic acid encoding a CD8 polypeptide. In embodiments, one or more vector may comprise a nucleic acid encoding a CD8α polypeptide. In embodiments, one or more vector may comprise a nucleic acid encoding a CD8β polypeptide.

In embodiments, one or more vector may comprise one or more nucleic acid encoding a T cell receptor (TCR) comprising an α chain and a β chain. In embodiments, one or more vector may comprise one or more nucleic acid encoding a T cell receptor (TCR) comprising an γ chain and a δ chain. In embodiments, one or more vector may comprise one or more nucleic acid encoding a chimeric antigen receptor (CAR).

In embodiments, a vector or vectors comprising one or more nucleic acid(s) encoding one or any combination of a TCR comprising an α chain and a β chain, a TCR comprising a γ chain and a δ chain, a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding one or any combination of a TCR comprising an α chain and a β chain, a dnTGFβRII polypeptide, and/or a CD8 polypeptide may be provided. In embodiments, a vector or vectors comprising one or more nucleic acid(s) encoding one or any combination of a TCR comprising a γ chain and a δ chain, a dnTGFβRII polypeptide, and/or a CD8 polypeptide may be provided. In embodiments, a vector or vectors comprising one or more nucleic acid(s) encoding one or any combination of a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding a TCR comprising an α chain and a β chain, a dnTGFβRII polypeptide, and a CD8 polypeptide may be provided. In embodiments, a vector or vectors comprising one or more nucleic acid(s) encoding a TCR comprising a γ chain and a δ chain, a dnTGFβRII polypeptide, and a CD8 polypeptide may be provided. In embodiments, a vector or vectors comprising one or more nucleic acid(s) encoding a CAR, a dnTGFβRII polypeptide, and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a vector or vectors comprising one or more nucleic acid(s) encoding a TCR comprising an α chain and a β chain and a dnTGFβRII polypeptide may be provided. In embodiments, a vector or vectors comprising one or more nucleic acid(s) encoding a TCR comprising a γ chain and a δ chain and a dnTGFβRII polypeptide may be provided. In embodiments, a vector or vectors comprising one or more nucleic acid(s) encoding a CAR and a dnTGFβRII polypeptide may be provided.

In embodiments, a vector or vectors comprising one or more nucleic acid(s) encoding a TCR comprising an α chain and a β chain and a CD8 polypeptide may be provided. In embodiments, a vector or vectors comprising one or more nucleic acid(s) encoding a TCR comprising a γ chain and a δ chain and a CD8 polypeptide may be provided. In embodiments, a cell or cells comprising one or more nucleic acid(s) encoding a CAR and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, more than one vector may be co-transduced into one or more cells, co-expressed in one or more cells, or combinations thereof. In embodiments, a cell or cells may comprise an αβ T cell, a γδ T cell, a natural killer (NK) cell, a natural killer T cell, a CD4+ T cell, CD8+ T cell, a CD4+/CD8+ cell, or combinations thereof.

In embodiments, more than one vector may comprise a nucleic acid or nucleic acids encoding one or any combination of a dnTGFβRII polypeptide, a CD8 polypeptide, a TCR comprising an α chain and a β chain, a TCR comprising an γ chain and a S chain, and/or a CAR. In embodiments, a CD8 polypeptide may comprise a CD8α chain and/or a CD8β chain, and the CD8α chain and/or CD8β chain may independently be modified or unmodified.

In embodiments, a single vector may comprise a nucleic acid or nucleic acids encoding one or any combination of a dnTGFβRII polypeptide, a CD8 polypeptide, a TCR comprising an α chain and a β chain, a TCR comprising an γ chain and a S chain, and/or a CAR. In embodiments, a CD8 polypeptide may comprise a CD8α chain and/or a CD8β chain, and the CD8α chain and/or CD8β chain may independently be modified or unmodified.

In embodiments, nucleic acids may be polycistronic, and one or more polycistronic nucleic acids may be utilized. Expression of multiple (e.g., 2, 3, 4, 5, or more) polypeptides from polycistronic nucleic acid may be achieved by any suitable method, such as i) pre-mRNA splicing; ii) proteolytic cleavage sites; iii) fusion proteins; iv) inclusion of one or more 2A peptide-encoding nucleic acid(s) (such as, but not limited to P2A, T2A, E2A, and F2A), v) inclusion of one or more internal ribosome entry site (IRES). Each of these approaches has some advantages and disadvantages to provide multiple transcription units. Among the five approaches, the most widely used are the self-cleaving 2A peptides and IRESs. In embodiments, nucleic acids may be monocistronic, and one or more monocistronic nucleic acid(s) may be utilized.

In embodiments, a 2A peptide may be selected from P2A (SEQ ID NO: 93), T2A (SEQ ID NO: 94), E2A (SEQ ID NO: 95), or F2A (SEQ ID NO: 96).

In embodiments, an IRES may be selected from the group consisting of IRES from picornavirus, IRES from flavivirus, IRES from pestivirus, IRES from retrovirus, IRES from lentivirus, IRES from insect RNA virus, and IRES from cellular mRNA.

In embodiments, a vector may comprise nucleic acid encoding a 2A peptide or an internal ribosome entry site (IRES) positioned between a nucleic acid encoding a modified CD8α polypeptide and a nucleic acid encoding a CD8β polypeptide.

In embodiments, a vector may comprise nucleic acid encoding a 2A peptide or an IRES positioned between a nucleic acid encoding a TCR α chain and a nucleic acid encoding a TCR β chain.

In embodiments, a vector may comprise nucleic acid encoding a 2A peptide or an IRES positioned between a nucleic acid encoding a TCR α chain or a nucleic acid encoding a TCR β chain and a nucleic acid encoding a dnTGFβRII polypeptide.

In embodiments, a single vector may comprise a nucleic acid or nucleic acids encoding one or any combination of a dnTGFβRII polypeptide, a CD8 polypeptide, a TCR comprising an α chain and a β chain, a TCR comprising an γ chain and a δ chain, and/or a CAR, and a vector may comprise a nucleic acid encoding a 2A peptide or an internal ribosome entry site (IRES) positioned between any or each of the nucleic acids encoding polypeptides or fusion polypeptides. In embodiments, a CD8 polypeptide may comprise a CD8α chain and/or a CD8β chain, and the CD8α chain and/or CD8β chain may independently be modified or unmodified.

In embodiments, a vector may further comprise a post-transcriptional regulatory element (PRE) sequence. In embodiments, the post-transcriptional regulatory element (PRE) sequence may be selected from a Woodchuck hepatitis virus PRE (WPRE) (such as, but not limited to wild type WPRE, such as but not limited to SEQ ID NO: 264, or a mutated WPRE, such as but not limited to WPREmut1 (SEQ ID NO: 256) or WPREmut2 (SEQ ID NO: 257)) or a hepatitis B virus (HBV) PRE (HPRE) (SEQ ID NO: 366), or variant(s) thereof, or any combination thereof.

In embodiments, a vector may further comprise one or more promoter. In embodiments, a promoter(s) may be selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, Murine Stem Cell Virus (MSCV) promoter, the promoter from CD69, nuclear factor of activated T-cells (NFAT) promoter, IL-2 promoter, minimal IL-2 promoter, or a combination thereof.

In embodiments, a vector may be a viral vector or a non-viral vector.

In embodiments, a vector may be selected from adenoviruses, poxviruses, alphaviruses, arenaviruses, flaviviruses, rhabdoviruses, retroviruses, lentiviruses, herpesviruses, paramyxoviruses, picornaviruses, or a combination thereof.

In embodiments, a vector may be pseudotyped with an envelope protein of a virus selected from the native feline endogenous virus (RD114), a chimeric version of RD114 (RD114TR), gibbon ape leukemia virus (GALV), a chimeric version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), lymphocytic choriomeningitis virus (LCMV), or a combination thereof.

In embodiments, a vector may comprise one or more Kozak sequence. In embodiments, a Kozak sequence may initiate, increase, or facilitate translation, or a combination thereof. In embodiments, the Kozak sequence may be GCCACC. In embodiments, the Kozak sequence may be ACCATGG. In embodiments, the Kozak sequence may be GCCNCCATGG, where N is a purine (A or G) (SEQ ID NO:365).

In embodiments, a vector may comprise one or more Factor Xa sites.

In embodiments, a vector may comprise one or more enhancer. In embodiments, an enhancer may comprise Conserved Non-Coding Sequence (CNS) 0, CNS 1, CNS2, CNS 3, CNS 4, or portions or combinations thereof.

In embodiments, the disclosure provides for one or more cells transduced with and/or expressing one or more vectors comprising nucleic acids encoding polypeptide(s).

In embodiments, a cell may comprise an αβ T cell, a γδ T cell, a natural killer cell, a natural killer T cell, a CD4+ T cell, CD8+ T cell, a CD4+/CD8+ cell, or any combination thereof.

In embodiments, a T cell may be a CD4+ T cell. In embodiments, a T cell may be a CD8+ T cell. In embodiments, a T cell may be a CD4+/CD8+ T cell. In embodiments, a T cell may be a αβ T cell. In embodiments, a T cell may be a γδ T cell.

In embodiments, a T cell may be an αβ T cell and may express a CD8 polypeptide described herein. In embodiments, a T cell may be an αβ T cell and may express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B). In embodiments, a T cell may be an αβ T cell and may express one or any combination of a dnTGFβRII polypeptide, a modified CD8 polypeptide, and/or a CAR.

In embodiments, a T cell may be a γδ T cell and may express a CD8 polypeptide described herein. In embodiments, a T cell may be a γδ T cell and may express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B). In embodiments, a T cell may be a γδ T cell and may express one or any combination of a dnTGFβRII polypeptide, a modified CD8 polypeptide, and/or a CAR.

In embodiments, a cell or cells comprising, or comprising one or more nucleic acid(s) encoding, one or any combination of a TCR comprising an α chain and a β chain, a TCR comprising a γ chain and a S chain, a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a cell or cells comprising, or comprising one or more nucleic acid(s) encoding, one or any combination of a TCR comprising an α chain and a β chain, a dnTGFβRII polypeptide, and/or a CD8 polypeptide may be provided. In embodiments, a cell or cells comprising, or comprising one or more nucleic acid(s) encoding, one or any combination of a TCR comprising a γ chain and a S chain, a dnTGFβRII, and/or a CD8 polypeptide may be provided. In embodiments, a cell or cells comprising, or comprising one or more nucleic acid(s) encoding, one or any combination of a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a cell or cells comprising, or comprising one or more nucleic acid(s) encoding, a TCR comprising an α chain and a β chain, a dnTGFβRII polypeptide, and a CD8 polypeptide may be provided. In embodiments, a cell or cells comprising, or comprising one or more nucleic acid(s) encoding, a TCR comprising a γ chain and a S chain, a dnTGFβRII polypeptide, and a CD8 polypeptide may be provided. In embodiments, a cell or cells comprising, or comprising one or more nucleic acid(s) encoding, a CAR, a dnTGFβRII polypeptide, and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, a cell or cells comprising, or comprising one or more nucleic acid(s) encoding, a TCR comprising an α chain and a β chain and a dnTGFβRII polypeptide may be provided. In embodiments, a cell or cells comprising, or comprising one or more nucleic acid(s) encoding, a TCR comprising a γ chain and a δ chain and a dnTGFβRII polypeptide may be provided. In embodiments, a cell or cells comprising, or comprising one or more nucleic acid(s) encoding, a CAR and a dnTGFβRII polypeptide may be provided.

In embodiments, a cell or cells comprising, or comprising one or more nucleic acid(s) encoding, a TCR comprising an α chain and a β chain and a CD8 polypeptide may be provided. In embodiments, a cell or cells comprising, or comprising one or more nucleic acid(s) encoding, a TCR comprising a γ chain and a δ chain and a CD8 polypeptide may be provided. In embodiments, a cell or cells comprising, or comprising one or more nucleic acid(s) encoding, a CAR and a CD8 polypeptide may be provided. In embodiments, a CD8 polypeptide may comprise a CD8α chain and/or a CD8β chain, and the CD8α chain and/or CD8β chain may independently be modified or unmodified.

In embodiments, one or more nucleic acid(s) may be comprised in and/or expressed from a vector or vectors.

In embodiments, a cell or cells may comprise an αβ T cell, a γδ T cell, a natural killer cell, a natural killer T cell, a CD4+ T cell, CD8+ T cell, a CD4+/CD8+ cell, or combinations thereof.

In embodiments, populations of cells as described herein may be provided. As a non-limiting example, the disclosure provides for a population of modified cells comprising, or comprising one or more nucleic acid(s) encoding one or any combination of an exogenous CD8 co-receptor comprising a polypeptide described herein, for example, amino acid sequences at least about 80%, at least about 85%, at least about 90%, at least about 95%, at least about 99%, or about 100% to SEQ ID NO: 5, 7, 258, 259, 8, 9, 10, 11, 12, 13, or 14; a dnTGFβRII polypeptide, for example, amino acid sequences at least about 80%, at least about 85%, at least about 90%, at least about 95%, at least about 99%, or about 100% to SEQ ID NO: 305 or SEQ ID NO: 307; and/or a T cell receptor. In embodiments, populations of cells may comprise αβ T cells, γδ T cells, natural killer cells, CD4+ T cells, CD8+ T cells, CD4+/CD8+ cells, or combinations thereof.

In embodiments, a method of preparing cells for immunotherapy may comprise isolating cells from a blood sample of a human subject, activating the isolated cells, transducing the activated cells with one or more vector, and expanding the transduced cells. In embodiments, a cell may comprise an αβ T cell, a γδ T cell, a natural killer cell, a natural killer T cell, a CD4+ T cell, CD8+ T cell, a CD4+/CD8+ cell, or combinations thereof.

In embodiments, a method of treating a patient who has cancer may comprise administering to the patient a composition comprising the population of expanded cells, wherein the cells kill cancer cells that present a peptide in a complex with an MHC molecule on the surface, wherein the peptide is selected from SEQ ID NO: 98-255, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, prostate cancer, or a combination thereof. In embodiments, a cell may comprise an αβ T cell, a γδ T cell, a natural killer cell, a natural killer T cell, a CD4+ T cell, CD8+ T cell, a CD4+/CD8+ cell, or combinations thereof.

In embodiments, the composition may further comprise an adjuvant.

In embodiments, an adjuvant may be selected from anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, IL-23, or combinations thereof.

In embodiments, a method of eliciting an immune response in a patient who has cancer may comprise administering to the patient a composition comprising the population of expanded cells, wherein the cells kill cancer cells that present a peptide in a complex with an MHC molecule on the surface, wherein the peptide is selected from SEQ ID NO: 98-255, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, prostate cancer, or a combination thereof. In embodiments, a cell may comprise an αβ T cell, a γδ T cell, a natural killer cell, a natural killer T cell, a CD4+ T cell, CD8+ T cell, a CD4+/CD8+ cell, or combinations thereof.

### BRIEF DESCRIPTION OF THE DRAWINGS

FIG. 1 shows a representative CD8α subunit, e.g., SEQ ID NO: 258 (CD8 α1). CD8 α1 includes five domains: (1) signal peptide, (2) Ig-like domain-1, (3) a stalk region, (4) transmembrane (TM) domain, and (5) a cytoplasmic tail (Cyto) comprising a lck-binding motif.

FIG. 2 shows a sequence alignment between CD8 α1 (SEQ ID NO: 258) and m1CD8α (SEQ ID NO: 7).

FIG. 3 shows a sequence alignment between CD8α2 (SEQ ID NO: 259) and m2CD8α (SEQ ID NO: 262), in which the cysteine substitution at position 112 is indicated by an arrow.

FIG. 4 shows exemplary vectors according to an aspect of the disclosure. In embodiments, vectors may also comprise additional elements, such as those described herein, such as, but not limited to one or more promoter or one or more post-transcriptional regulatory element. In FIG. 4, the lines may represent direct linkages, with no intervening sequences, or may represent intervening sequences, such as, but not limited to, a linker, a furin, a sequence encoding a 2A polypeptide, a factor Xa site, an untranslated sequence, a translated sequence, a sequence comprising one or more restriction endonuclease sites, or a combination thereof.

FIG. 5A shows titers of viral vectors shown in FIG. 4.

FIG. 5B shows titers of further viral vectors in accordance with the present disclosure. Construct #13; Construct #14; Construct #15; Construct #16; Construct #17; Construct #18; Construct #19; Construct #21; Construct #10n; Construct #11n; and TCR: R11KEA (SEQ ID NO: 15 and SEQ ID NO: 16) (Construct #8), which binds PRAME-004 (SLLQHLIGL) (SEQ ID NO: 147). Note that Constructs #10 and #10n are different batches of the same construct (SEQ ID NO: 291 and 292) and Constructs #11 and #11n are different batches of the same construct (SEQ ID NO: 285 and 286).

FIG. 6 shows T cell manufacturing.

FIG. 7A shows expression of activation markers before and after activation in CD3+CD8+ cells.

FIG. 7B shows expression of activation markers before and after activation in CD3+CD4+ cells.

FIG. 8A shows fold expansion of cells transduced with various constructs from Donor #1. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control). Note that Constructs #9 and #9b are different batches of the same construct (SEQ ID NO: 287 and 288).

FIG. 8B shows fold expansion of cells transduced with various constructs from Donor #2. The constructs are as follows: Construct #9b; Construct #10; Construct #11; Construct #12; Construct #1; Construct #2; TCR=R11KEA.WPRE^{wt} (TCR with wild type WPRE) (Construct #8); NT=Non-transduced T cells (as a negative control).

FIG. 9A shows flow plots of cells transduced with Construct #9.

FIG. 9B shows flow plots of cells transduced with Construct #10 in accordance with the present disclosure.

FIG. 9C shows flow plots of cells transduced with Construct #11.

FIG. 9D shows flow plots of cells transduced with Construct #12.

FIG. 10 shows % CD8+CD4+ of cells transduced with various constructs for Donor #1 and Donor #2. The constructs are as follows: Construct #9b; Construct #10; Construct #11; Construct #12; Construct #1; Construct #2; TCR=R11KEA.WPRE^{wt} TCR with wild type WPRE); NT=Non-transduced T cells (as a negative control).

FIG. 11 shows % Tet of CD8+CD4+ of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 12 shows Tet MFI (CD8+CD4+ Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 13 shows CD8α MFI (CD8+CD4+ Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 14 shows % CD8+CD4 (of CD3+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 15 shows % CD8+ Tet+ (of CD3+) of cells transduced with various constructs. The constructs are as follows: Construct #9b; Construct #10; Construct #11; Construct #12; Construct #1; Construct #2; TCR=R11KEA.WPRE^{wt} TCR with wild type WPRE); NT=Non-transduced T cells (as a negative control).

FIG. 16 shows Tet MFI (CD8+ Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 17 shows CD8α MFI (CD8+ Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 18 shows % Tet+ (of CD3+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 19 shows VCN (upper panel) and CD3+Tet+/VCN (lower panel) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 20A-20C depicts data showing that constructs (#10, #11, & #12) are comparable to TCR-only in mediating cytotoxicity against target positive cells lines expressing antigen at different levels (UACC257 at 1081 copies per cell and A375 at 50 copies per cell).

FIG. 21A-21B depict data showing that IFNγ secretion in response to UACC257 is comparable among constructs, however with A375, #10 expressing is the highest among all constructs. However, comparing #9 with #11 expressing wild type and modified CD8 coreceptor sequences respectively, T cells transduced with #11 induced stronger cytokine response measured as IFNγ quantified in the supernatants from Incucyte plates. Construct #9; Construct #10; Construct #11; Construct #12; Construct #1; Construct #2; Construct #8=R11KEA TCR only.

FIG. 22 depicts an exemplary experiment design to assess DC maturation and cytokine secretion by PBMC-derived product in response to UACC257 and A375 targets. N=2.

FIG. 23A-23B depicts data showing that the IFNγ secretion in response to A375 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with #11 induced stronger cytokine response measured as IFNγ quantified in the culture supernatants of three-way cocultures using donor D600115, E:T:iDC::1:1/10:1/4. Construct #9; Construct #10; Construct #11; Construct #12; Construct #1; Construct #2; Construct #8=R11KEA TCR only.

FIG. 24A-24B depicts data showing that IFNγ secretion in response to A375 increases in the presence of iDCs. In the tri-cocultures with iDCs, IFNγ secretion was higher in Construct #10 compared to the other constructs. IFNγ quantified in the culture supernatants of three-way cocultures using donor D150081, E:T:iDC::1:1/10:1/4. Construct #9; Construct #10; Construct #11; Construct #12; Construct #1; Construct #2; Construct #8=R11KEA TCR only.

FIG. 25A-25B depicts data showing that IFNγ secretion in response to UACC257 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with Construct #11 induced stronger cytokine response measured as IFNγ quantified in the culture supernatants of three-way cocultures using donor D600115, E:T:iDC::1:1/10:1/4. Construct #9; Construct #10; Construct #11; Construct #12; Construct #1; Construct #2; Construct #8=R11KEA TCR only.

FIG. 26 shows T cell manufacturing in accordance with the present disclosure.

FIG. 27A shows expression of activation markers before and after activation in CD3+CD8+ cells.

FIG. 27B shows expression of activation markers before and after activation in CD3+CD4+ cells in accordance the present disclosure.

FIG. 28 shows fold expansion of cells transduced with various constructs.

FIG. 29A-29B show % CD8+CD4+ of cells transduced with various constructs in accordance with the present disclosure.

FIG. 30A-30B show % Tet of CD8+CD4+ of cells transduced with various constructs in accordance with the present disclosure.

FIG. 31A-31B show Tet MFI (CD8+CD4+ Tet+) of cells transduced with various constructs in accordance with the present disclosure.

FIG. 32A-32B show % CD8+CD4− (of CD3+) of cells transduced with various constructs in accordance with the present disclosure.

FIG. 33A-33B show % CD8+ Tet+ (of CD3+) of cells transduced with various constructs in accordance with the present disclosure.

FIG. 34A-34B show Tet MFI (CD8+ Tet+) of cells transduced with various constructs in accordance with the present disclosure.

FIG. 35A-35B show % Tet+ (of CD3+) of cells transduced with various constructs in accordance with the present disclosure.

FIG. 36A-36B show VCN of cells transduced with various constructs in accordance with the present disclosure.

FIG. 37 shows T cell manufacturing in accordance with the present disclosure.

FIG. 38 shows % Tet of CD8+CD4+ of cells transduced with various constructs.

FIG. 39 shows Tet MFI of CD8+CD4+ Tet+ of cells transduced with various constructs.

FIG. 40 shows Tet MFI of CD8+ Tet+ of cells transduced with various constructs.

FIG. 41 shows % Tet+ of CD3+ cells transduced with various constructs.

FIG. 42 shows vector copy number (VCN) of cells transduced with various constructs.

FIG. 43 shows the % T cell subsets in cells transduced with various constructs. FACS analysis was gated on CD3+TCR+.

FIG. 44A-44B show % T cell subsets in cells transduced with various constructs. FACS analysis was gated on CD4+CD8+ for FIG. 44A and on CD4−CD8+ TCR+ for FIG. 44B.

FIG. 45A-45B depict data showing that Constructs #13 and #10 are comparable to TCR-only in mediating cytotoxicity against UACC257 target positive cells lines expressing high levels of antigen (1081 copies per cell). Construct #15 was also effective but slower in killing compared to Constructs #13 and #10. The effector:target ratio used to generate these results was 4:1.

FIG. 46 shows IFNγ secretion in response in UACC257 cell line was higher with Construct #13 compared to Construct #10. IFNγ quantified in the supernatants from Incucyte plates. The effector:target ratio used to generate these results was 4:1.

FIG. 47 shows ICI marker frequency (2B4, 41BB, LAG3, PD-1, TIGIT, TIM3, CD39+CD69+, and CD39−CD69−).

FIG. 48A-48G show increased expression of IFNγ, IL-2, and TNFα with CD4+CD8+ cells transduced with Construct #10 (WT signal peptide, CD8β1) compared to other constructs. FACS analysis was gated on CD3+CD4+CD8+ cells against UACC257, 4:1 E:T.

FIG. 49A-49G show increased expression of IFNγ, IL-2, MIP-1β, and TNFα with CD4−CD8+ cells transduced with Construct #10 (WT signal peptide, CD8β1) compared to other constructs. FACS analysis was gated on CD3+CD4−CD8+ cells against UACC257, 4:1 E:T.

FIG. 50A-50G show increased expression of IL-2 and TNFα with CD3+TCR+ cells transduced with Construct #10 (WT signal peptide, CD8β1) compared to other constructs. FACS analysis was gated on CD3+TCR+ cells against UACC257, 4:1 E:T.

FIG. 51A-51C show results from FACS analysis gated on CD4+CD8+ cells against A375, 4:1 E:T.

FIG. 52A-52C show results from FACS analysis gated on CD4−CD8+ cells against A375, 4:1 E:T.

FIG. 53A-53C show results from FACS analysis gated on CD3+TCR+ cells against A375, 4:1 E:T.

FIG. 54 shows T cell manufacturing in accordance with the present disclosure.

FIG. 55A-55C show interaction between peptide/MHC complex of antigen-presenting cell (APC) with T cell by binding a complex of TCR and CD8αβ heterodimer (FIG. 55A, e.g., produced by transducing T cells with Constructs #2, #3, #4, #10, #13, #14, #15, #16, #17, #18, or #21), a complex of TCR and homodimer CD8α having its stalk region replaced with CD8β stalk region (CD8αα*) (FIG. 55B, e.g., produced by transducing T cells with Construct #11, #12, or #19), and a complex of TCR and CD8α homodimer (FIG. 55C, e.g., produced by transducing T cells with Constructs #1, #5, #6, #7, or #9).

FIG. 56 shows the levels of IL-12 secretion by dendritic cells (DC) in the presence of CD4+ T cells transduced with Construct #10 or #11 and immature dendritic cells (iDCs) in accordance with the present disclosure.

FIG. 57 shows the levels of TNF-α secretion by dendritic cells (DC) in the presence of CD4+ T cells transduced with Construct #10 or #11 and immature dendritic cells (iDCs) in accordance with the present disclosure.

FIG. 58 shows the levels of IL-6 secretion by dendritic cells (DC) in the presence of CD4+ T cells transduced with Construct #10 or #11 and immature dendritic cells (iDCs) in accordance with the present disclosure.

FIG. 59 shows a scheme of determining the levels of cytokine secretion by dendritic cells (DC) in the presence of PBMCs transduced with various constructs and target cells in accordance with the present disclosure.

FIG. 60 shows the levels of IL-12 secretion by dendritic cells (DC) in the presence of PBMCs transduced with various constructs and target cells in accordance with the present disclosure.

FIG. 61 shows the levels of TNF-α secretion by dendritic cells (DC) in the presence of PBMCs transduced with various constructs and target cells in accordance with the present disclosure

FIG. 62 shows the levels of IL-6 secretion by dendritic cells (DC) in the presence of PBMCs transduced with various constructs and target cells in accordance with the present disclosure.

FIG. 63A-63C show IFNγ production from the transduced CD4+ selected T cells obtained from Donor #1 (FIG. 63A), Donor #2 (FIG. 63B), and Donor #3 (FIG. 63C) in accordance to the present disclosure.

FIG. 63D shows EC50 values (ng/ml) in FIG. 63A-63C.

FIG. 64A-64C show IFNγ production from the transduced PBMC obtained from Donor #4 (FIG. 64A), Donor #1 (FIG. 64B), and Donor #3 (FIG. 64C) and their respective EC50 values (ng/ml) in accordance to the present disclosure.

FIG. 64D shows comparison of EC50 values (ng/ml) among different donors in FIG. 64A-64C.

FIG. 65A-65C show IFNγ production from the transduced PBMC (FIG. 65A), CD8+ selected T cells (FIG. 65B), and CD4+ selected T cells (FIG. 65C) and their respective EC50 values (ng/ml) from a single donor in accordance to the present disclosure.

FIG. 66 schematically depicts exemplary dnTGFβRII polypeptides bound to the membrane of a T cell, as may be provided in embodiments. dnTGFβRII binds TGFβ but may block signaling in response to TGFβ binding, as a non-limiting example.

FIG. 67A shows an exemplary nucleic acid comprising an MSCV promoter, a sequence encoding a dnTGFβRIIvar1 polypeptide, and a sequence encoding a WPRE, as may be provided in embodiments.

FIG. 67B shows an exemplary nucleic acid comprising an MSCV promoter, a sequence encoding a dnTGFβRIIvar2 polypeptide, and a sequence encoding a WPRE, as may be provided in embodiments. In embodiments, in FIGS. 67A and 67B, the lines connecting the MSCV promoter to the dnTGFβRII and the dnTGFβRII to the WPRE may represent direct linkages, with no intervening sequences, or may represent intervening sequences, such as, but not limited to, a linker, an untranslated sequence, a translated sequence, a sequence comprising one or more restriction endonuclease sites, or a combination thereof.

FIG. 68 depicts exemplary vector constructs, which may be provided in embodiments. In embodiments, vectors may also comprise additional elements, such as those described herein, such as, but not limited to one or more promoter or one or more post-transcriptional regulatory element. In FIG. 68, the lines may represent direct linkages, with no intervening sequences, or may represent intervening sequences, such as, but not limited to, a linker, a furin, a sequence encoding a 2A polypeptide, a factor Xa site, an untranslated sequence, a translated sequence, a sequence comprising one or more restriction endonuclease sites, or a combination thereof.

FIG. 69 shows vector copy number in cells transduced with vectors encoding dnTGFβRIIvar1 (“var1”) or dnTGFβRIIvar2 (“var2”) in an exemplary transduction. Vector copy number (copies per cell) (Y axis) is plotted against the volume (in μL per 1×10⁶ cells) of lentivirus vector used to transduce the cells (X axis)

FIG. 70 shows vector copy number in non-transduced cells (“NT”) and in cells transduced (i) only with vector encoding TCR at 2.5 μL per million cells (“TCR only”, both bars), (ii) with vector encoding TCR at 2.5 μL per million cells and with vector encoding dnTGFβRIIvar1 at 2.5 μL per million cells (“TCR/DNR(2.5)”, white bar), (iii) with vector encoding TCR at 2.5 μL per million cells and with vector encoding dnTGFβRIIvar1 at 5.0 μL per million cells (“TCR/DNR(5.0)”, white bar), (iv) with vector encoding TCR at 2.5 μL per million cells and with vector encoding dnTGFβRIIvar1 at 10.0 μL per million cells (“TCR/DNR(10.0)”, white bar), (v) with vector encoding TCR at 2.5 μL per million cells and with vector encoding dnTGFβRIIvar2 at 2.5 μL per million cells (“TCR/DNR(2.5)”, black bar), (vi) with vector encoding TCR at 2.5 μL per million cells and with vector encoding dnTGFβRIIvar2 at 5.0 μL per million cells (“TCR/DNR(5.0)”, black bar), or (vii) with vector encoding TCR at 2.5 μL per million cells and with vector encoding dnTGFβRIIvar2 at 10.0 μL per million cells (“TCR/DNR(10.0)”, black bar), in an exemplary transduction. Vector copy number (copies per cell) (Y axis) is shown.

FIG. 71A shows percentage of CD3+ cells positive for TCR (black bars) or for dnTGFβRIIvar1 (white bars), in an exemplary transduction. Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar1 at 2.5 μL per million cells (“TCR/DNR(2.5)”), (iii) with vector encoding TCR at 2.5 μL per million cells and with vector encoding dnTGFβRIIvar1 at 5.0 μL per million cells (“TCR/DNR(5.0)”), or (iv) with vector encoding TCR at 2.5 μL per million cells and with vector encoding dnTGFβRIIvar1 at 10.0 μL per million cells (“TCR/DNR(10.0)”).

FIG. 71B shows percentage of CD3+ cells positive for TCR (black bars) or for dnTGFβRIIvar2 (white bars), in an exemplary transduction. Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar2 at 2.5 μL per million cells (“TCR/DNR(2.5)”), (iii) with vector encoding TCR at 2.5 μL per million cells and with vector encoding dnTGFβRIIvar2 at 5.0 μL per million cells (“TCR/DNR(5.0)”), or (iv) with vector encoding TCR at 2.5 μL per million cells and with vector encoding dnTGFβRIIvar2 at 10.0 μL per million cells (“TCR/DNR(10.0)”).

FIG. 72 shows percentage of CD3+ cells double-positive for TCR and dnTGFβRIIvar1 (white bars) or for TCR and dnTGFβRIIvar2 (black bars), in an exemplary transduction. Cells were non-transduced (“NT”) or transduced (i) only with vector encoding TCR at 2.5 μL per million cells (“TCR Only”, both bars), (ii) with vector encoding TCR at 2.5 μL per million cells and with vector encoding dnTGFβRIIvar1 at 2.5 μL per million cells (“TCR/DNR(2.5)”, white bar), (iii) with vector encoding TCR at 2.5 μL per million cells and with vector encoding dnTGFβRIIvar1 at 5.0 μL per million cells (“TCR/DNR(5.0)”, white bar), (iv) with vector encoding TCR at 2.5 μL per million cells and with vector encoding dnTGFβRIIvar1 at 10.0 μL per million cells (“TCR/DNR(10.0)”, white bar), (v) with vector encoding TCR at 2.5 μL per million cells and with vector encoding dnTGFβRIIvar2 at 2.5 μL per million cells (“TCR/DNR(2.5)”, black bar), (vi) with vector encoding TCR at 2.5 μL per million cells and with vector encoding dnTGFβRIIvar2 at 5.0 μL per million cells (“TCR/DNR(5.0)”, black bar), or (vii) with vector encoding TCR at 2.5 μL per million cells and with vector encoding dnTGFβRIIvar2 at 10.0 μL per million cells (“TCR/DNR(10.0)”, black bar).

FIG. 73 shows fold expansion of non-transduced cells (“NT”, both bars) and of cells transduced with (i) only with vector encoding TCR at 2.5 μL per million cells (“TCR Only”, both bars), (ii) with vector encoding TCR at 2.5 μL per million cells and with vector encoding dnTGFβRIIvar1 at 0.31 μL per million cells (“TCR/DNR(0.31)”, white bar), (iii) with vector encoding TCR at 2.5 μL per million cells and with vector encoding dnTGFβRIIvar1 at 0.63 μL per million cells (“TCR/DNR(0.63)”, white bar), (iv) with vector encoding TCR at 2.5 μL per million cells and with vector encoding dnTGFβRIIvar1 at 1.25 μL per million cells (“TCR/DNR(1.25)”, white bar), (v) with vector encoding TCR at 2.5 μL per million cells and with vector encoding dnTGFβRIIvar1 at 2.50 μL per million cells (“TCR/DNR(2.50)”, white bar), (vi) with vector encoding TCR at 2.5 μL per million cells and with vector encoding dnTGFβRIIvar2 at 0.31 μL per million cells (“TCR/DNR(0.31)”, black bar), (vii) with vector encoding TCR at 2.5 μL per million cells and with vector encoding dnTGFβRIIvar2 at 0.63 μL per million cells (“TCR/DNR(0.63)”, black bar), (viii) with vector encoding TCR at 2.5 μL per million cells and with vector encoding dnTGFβRIIvar2 at 1.25 μL per million cells (“TCR/DNR(1.25)”, black bar), or (ix) with vector encoding TCR at 2.5 μL per million cells and with vector encoding dnTGFβRIIvar2 at 2.50 μL per million cells (“TCR/DNR(2.50)”, black bar), in an exemplary transduction.

FIG. 74A shows percentage of CD3+ cells positive for TCR (black bars) or for dnTGFβRIIvar1 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar1 at 0.31 μL per million cells (“TCR/DNR(0.31)”), (iii) with vector encoding TCR at 2.5 μL per million cells and with vector encoding dnTGFβRIIvar1 at 0.63 μL per million cells (“TCR/DNR(0.63)”), (iv) with vector encoding TCR at 2.5 μL per million cells and with vector encoding dnTGFβRIIvar1 at 1.25 μL per million cells (“TCR/DNR(1.25)”), or (v) with vector encoding TCR at 2.5 μL per million cells and with vector encoding dnTGFβRIIvar1 at 2.50 μL per million cells (“TCR/DNR(2.50)”), in an exemplary transduction.

FIG. 74B shows percentage of CD3+ cells positive for TCR (black bars) or for dnTGFβRIIvar2 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar2 at 0.31 μL per million cells (“TCR/DNR(0.31)”), (iii) with vector encoding TCR at 2.5 μL per million cells and with vector encoding dnTGFβRIIvar2 at 0.63 μL per million cells (“TCR/DNR(0.63)”), (iv) with vector encoding TCR at 2.5 μL per million cells and with vector encoding dnTGFβRIIvar2 at 1.25 μL per million cells (“TCR/DNR(1.25)”), or (v) with vector encoding TCR at 2.5 μL per million cells and with vector encoding dnTGFβRIIvar2 at 2.50 μL per million cells (“TCR/DNR(2.50)”), in an exemplary transduction.

FIG. 75 shows percentage of CD3+ cells double-positive for TCR and dnTGFβRIIvar1 (white bars) or for TCR and dnTGFβRIIvar2 (black bars), in an exemplary transduction. Cells were non-transduced (“NT”) or transduced (i) only with vector encoding TCR at 2.5 μL per million cells (“TCR Only”, both bars), (ii) with vector encoding TCR at 2.5 μL per million cells and with vector encoding dnTGFβRIIvar1 at 0.313 μL per million cells (“TCR/DNR(0.313)”, white bar), (iii) with vector encoding TCR at 2.5 μL per million cells and with vector encoding dnTGFβRIIvar1 at 0.625 μL per million cells (“TCR/DNR(0.625)”, white bar), (iv) with vector encoding TCR at 2.5 μL per million cells and with vector encoding dnTGFβRIIvar1 at 1.25 μL per million cells (“TCR/DNR(1.25)”, white bar), (v) with vector encoding TCR at 2.5 μL per million cells and with vector encoding dnTGFβRIIvar1 at 2.50 μL per million cells (“TCR/DNR(1.25)”, white bar), (vi) with vector encoding TCR at 2.5 μL per million cells and with vector encoding dnTGFβRIIvar2 at 0.313 μL per million cells (“TCR/DNR(0.313)”, black bar), (vii) with vector encoding TCR at 2.5 μL per million cells and with vector encoding dnTGFβRIIvar2 at 0.625 μL per million cells (“TCR/DNR(0.625)”, black bar), or (viii) with vector encoding TCR at 1.25 μL per million cells and with vector encoding dnTGFβRIIvar2 at 10.0 μL per million cells (“TCR/DNR(1.25)”, black bar), or (ix) with vector encoding TCR at 1.25 μL per million cells and with vector encoding dnTGFβRIIvar2 at 2.5 μL per million cells (“TCR/DNR(2.5)”, black bar).

FIG. 76 shows fold expansion of non-transduced cells (“PS NT”) and cells transduced with TCR (2.5 μL per million cells) and dnTGFβRIIvar1 (2.5 μL per million cells) (all other bars). “PS NT” non-transduced cells were incubated with protamine sulfate (PS). “PS Mixed” transduced cells were transduced with TCR and dnTGFβRIIvar1 simultaneously with PS added. “PS Sequential” transduced cells were transduced with TCR and dnTGFβRIIvar1 sequentially with PS added. “LB Mixed” transduced cells were transduced with TCR and dnTGFβRIIvar1 simultaneously with LentiBOOST® (LB) added. “LB Sequential” transduced cells were transduced with TCR and dnTGFβRIIvar1 sequentially with LB added, in an exemplary transduction. “None Mixed” transduced cells were transduced with TCR and dnTGFβRIIvar1 simultaneously with no enhancer added. “PS Mixed (Spin)” transduced cells were transduced with TCR and dnTGFβRIIvar1 simultaneously with PS added and were spinoculated. “LB Mixed (Spin)” transduced cells were transduced with TCR and dnTGFβRIIvar1 simultaneously with LB added and were spinoculated. “None (Spin)” transduced cells were transduced with TCR and dnTGFβRIIvar1 simultaneously with no enhancer added and were spinoculated.

FIG. 77A shows percentage of CD3+ cells positive for TCR (black bars) or for dnTGFβRIIvar1 (white bars), in an exemplary transduction. Non-transduced cells (“NT”) are shown as a control. “None-Mixed” transduced cells were transduced with TCR and dnTGFβRIIvar1 simultaneously with no enhancer added. “PS Mixed” transduced cells were transduced with TCR and dnTGFβRIIvar1 simultaneously with PS added. “PS Serial” transduced cells were transduced with TCR and dnTGFβRIIvar1 sequentially with PS added. “LB Mixed” transduced cells were transduced with TCR and dnTGFβRIIvar1 simultaneously with LB added. “LB Serial” transduced cells were transduced with TCR and dnTGFβRIIvar1 sequentially with LB added. “None (Spin)” transduced cells were transduced with TCR and dnTGFβRIIvar1 simultaneously with no enhancer added and were spinoculated. “PS (Spin)” transduced cells were transduced with TCR and dnTGFβRIIvar1 simultaneously with PS added and were spinoculated. “LB (Spin)” transduced cells were transduced with TCR and dnTGFβRIIvar1 simultaneously with LB added and were spinoculated.

FIG. 77B shows percentage of CD3+ cells double-positive for TCR and dnTGFβRIIvar1, in an exemplary transduction. Non-transduced cells (“NT”) are shown as a control. “None-Mixed” transduced cells were transduced with TCR and dnTGFβRIIvar1 simultaneously with no enhancer added. “PS Mixed” transduced cells were transduced with TCR and dnTGFβRIIvar1 simultaneously with PS added. “PS Serial” transduced cells were transduced with TCR and dnTGFβRIIvar1 sequentially with PS added. “LB Mixed” transduced cells were transduced with TCR and dnTGFβRIIvar1 simultaneously with LB added. “LB Serial” transduced cells were transduced with TCR and dnTGFβRIIvar1 sequentially with LB added. “None (Spin)” transduced cells were transduced with TCR and dnTGFβRIIvar1 simultaneously with no enhancer added and were spinoculated. “PS (Spin)” transduced cells were transduced with TCR and dnTGFβRIIvar1 simultaneously with PS added and were spinoculated. “LB (Spin)” transduced cells were transduced with TCR and dnTGFβRIIvar1 simultaneously with LB added and were spinoculated.

FIG. 78 shows fold expansion (Y Axis) of non-transduced (“NT”) cells and cells transduced with TCR (2.5 μL/million cells) and dnTGFβRIIvar1 (2.5 μL/million cells) (“2.50”), in an exemplary transduction.

FIG. 79A shows percentage of non-transduced CD3+ cells (“NT”) and CD3+ cells transduced with TCR and dnTGFβRIIvar1 (“TCR/DNR”) that were positive for TCR (black bar) or for dnTGFβRIIvar1 (white bar), in an exemplary transduction.

FIG. 79B shows percentage of non-transduced CD3+ cells (“NT”) and CD3+ cells transduced with TCR and dnTGFβRIIvar1 (“TCR/DNR”) that were double-positive for TCR and dnTGFβRIIvar1, in an exemplary transduction.

FIGS. 80A-80E show an exemplary cell sorting scheme and plots of resultant cell fractions for one donor (D120). It can be seen that the Sort 1 input (FIG. 80A) comprised a mix of TCR+dnTGFβRII+ cells, TCR+dnTGFβRII− cells, TCR-dnTGFβRII+ cells, and TCR-dnTGFβRII− cells. Collected Sort 1 output (FIG. 80B) comprised predominantly TCR+dnTGFβRII+ cells. The flow-through cells from Sort 1 (“Sort 1 Waste”) (FIG. 80C) were sorted a second time, producing Sort 2 output cells (FIG. 80D) comprising predominantly TCR+dnTGFβRII− cells, which were collected. The flow-through cells from Sort 2 (“Sort 2 Waste” or “waste”) (FIG. 80E) comprised predominantly TCR-dnTGFβRII− cells. For each of FIGS. 80A-80E, the left plot (FSC×SSC) shows the parent gate for the plot on the right; TCR/DNR expression as a proportion of “all lymphocytes”. Tet+ cells are TCR+ cells, while Tet− cells are TCR− cells.

FIG. 81 shows tumor fold growth of UACC257-RFP cells, normalized to 0 hours, in an exemplary co-culture assay. Shown are UACC257 cells only (“UACC257 Only”, open downward triangles) and UACC257 cells co-cultured with TCR-only transduced cells, where the co-cultures were treated with (i) 8 ng/mL TGF-β1 (“TGF-b 8 ng/mL”, open octagons), (ii) 128 ng/mL TGF-β1 (“TGF-b 128 ng/mL”, solid circles), (iii) no addition of TGF-β1 or GAL (“TGF-b 0 ng/mL”, open circles), (iv) 8 ng/mL TGF-β1 plus 5 μM GAL (“TGF-b 8 ng/mL+GAL”, open upward triangles), (v) 128 ng/mL TGF-β1 plus 5 μM GAL (“TGF-b 128 ng/mL+GAL”, open squares), or (vi) 5 μM GAL only (“TGF-b 0 ng/mL+GAL”, X's).

FIG. 82 shows the Division Index (average number of divisions per cell) of effector T cells (transduced with TCR only), in an exemplary co-culture assay. Division index is inclusive of all CD3+ T cells co-cultured with UACC257 cells in the presence (“+GAL”) or absence (“−GAL”) of GAL, and the absence of TGF-β1 (0 ng/mL) or the presence of TGF-β1 at 2, 4, 8, 16, 32, 64, or 128 ng/mL.

FIG. 83 shows the proliferation of effector cells (transduced with TCR only) cocultured with UACC257 tumor cells with no addition of TGF-β1 or GAL (“TGF-β 0 ng/mL −GAL”), with the addition of 8 ng/mL TGF-β1 and no addition of GAL (“TGF-β 8 ng/mL −GAL”), and with the addition of 8 ng/mL TGF-β1 and 5 μM GAL (“TGF-β 8 ng/mL+GAL 5 μM”). Cell count (on the Y axis) ranges from 0 to 500 for the “TGF-β 0 ng/mL −GAL” plot, from 0 to 300 for the “TGF-β 8 ng/mL −GAL” plot, and from 0 to 500 for the “TGF-β 8 ng/mL +GAL 5 μM” plot, in an exemplary co-culture assay.

FIG. 84A shows the percentage of all lymphocytes that were positive for pSMAD (Y axis), in an exemplary assay. Lymphocytes are from a first donor. Results are shown for non-transduced cells (“NT”) and for cells transduced with vector encoding dnTGFβRIIvar1 at 7.5 μL per million cells (“7.5”). Cells were untreated (white bars), treated with 5 ng/mL TGFβ (ticked bars), or treated with treated with 5 ng/mL TGFβ and 5 μM GAL (black bars).

FIG. 84B shows the percentage of all lymphocytes that were positive for pSMAD (Y axis), in an exemplary assay. Lymphocytes are from the first donor. Results are shown for non-transduced cells (“NT”) and for cells transduced with vector encoding dnTGFβRIIvar2 at 7.5 μL per million cells (“7.5”). Cells were untreated (white bars), treated with 5 ng/mL TGFβ (ticked bars), or treated with treated with 5 ng/mL TGFβ and 5 μM GAL (black bars).

FIG. 84C shows the percentage of all lymphocytes that were positive for pSMAD (Y axis), in an exemplary assay. Lymphocytes are from a second donor. Results are shown for non-transduced cells (“NT”) and for cells transduced with vector encoding dnTGFβRIIvar2 at 10 μL per million cells (“10”). Cells were untreated (white bars), treated with 5 ng/mL TGFβ (ticked bars), or treated with treated with 5 ng/mL TGFβ and 5 μM GAL (black bars).

FIG. 85 shows fold growth of UACC257-RFP tumor cells normalized to 0 hours, in an exemplary co-culture assay. Tumor cells were added back to the co-cultures at approximately 60 hours and at approximately 134 hours after the initiation of co-culture. Growth of UACC257-RFP cells cultured alone (“UACC257 (No Cyto)”) is shown as a control (open squares). Growth of UACC257-RFP cells co-cultured with cells depleted of TCR+dnTGFβRII+ cells and TCR+dnTGFβRII− cells (“Waste (No Cyto)”) is shown (solid diamonds). Growth of UACC257-RFP cells co-cultured with TCR+ cells depleted of TCR+dnTGFβRII+ cells (“TCR+(No Cyto)”) is shown (open circles). Growth of UACC257-RFP cells co-cultured with TCR+dnTGFβRII+ cells (“TCR+DNR+ (No Cyto)”) is shown (solid triangles). Effector:Target (E:T) ratio was 4:1. No TGF-β1 was added to the cell cultures.

FIG. 86 shows fold growth of UACC257-RFP tumor cells normalized to 0 hours, in an exemplary co-culture assay. Tumor cells were added back to the co-cultures at approximately 60 hours and at approximately 134 hours after the initiation of co-culture. Growth of UACC257-RFP cells cultured alone (“UACC257 (TGFb)”) is shown as a control (open squares). Growth of UACC257-RFP cells co-cultured with cells depleted of TCR+dnTGFβRII+ cells and TCR+dnTGFβRII− cells (“Waste (TGFb)”) is shown (solid diamonds). Growth of UACC257-RFP cells co-cultured with TCR+ cells depleted of TCR+dnTGFβRII+ cells (“TCR+(TGFb)”) is shown (open circles). Growth of UACC257-RFP cells co-cultured with TCR+dnTGFβRII+ cells (“TCR+DNR+(TGFb)”) is shown (solid triangles). Effector:Target (E:T) ratio was 4:1. TGF-β1 at a concentration of 10 ng/mL was added to each of cell cultures at the initiation of culture and at the times that tumor cells were added back.

FIG. 87 shows fold growth of UACC257-RFP tumor cells normalized to 0 hours, in an exemplary co-culture assay. Tumor cells were added back to the co-cultures at approximately 60 hours and at approximately 134 hours after the initiation of co-culture. Growth of UACC257-RFP cells cultured alone (“UACC257 (TGFb Daily)”) is shown as a control (open squares). Growth of UACC257-RFP cells co-cultured with cells depleted of TCR+dnTGFβRII+ cells and TCR+dnTGFβRII− cells (“Waste (TGFb Daily)”) is shown (solid diamonds). Growth of UACC257-RFP cells co-cultured with TCR+ cells depleted of TCR+dnTGFβRII+ cells (“TCR+(TGFb Daily)”) is shown (open circles). Growth of UACC257-RFP cells co-cultured with TCR+dnTGFβRII+ cells (“TCR+DNR+(TGFb Daily)”) is shown (solid triangles). Effector:Target (E:T) ratio was 4:1. TGF-β1 at a concentration of 10 ng/mL was added to each of cell cultures daily, including on the day of co-culture initiation.

FIG. 88 shows percentage of cells divided, in an exemplary assay. Shown are (i) TCR+dnTGFβRII+ cells untreated with TGF-β1 (“TCR+DNR+(Untreated)”), (ii) TCR+ cells depleted of TCR+dnTGFβRII+ cells, untreated with TGF-β1 (“TCR+(Untreated)”), (iii) TCR-dnTGFβRII− cells untreated with TGF-β1 (“Waste (Untreated)”), (iv) TCR+dnTGFβRII+ cells treated with 10 ng/mL TGF-β1 (“TCR+DNR+(TGF-b)”), (v) TCR+ cells depleted of TCR+dnTGFβRII+ cells and treated with 10 ng/mL TGF-β1 (“TCR+(TGF-b)”), and (vi) TCR-dnTGFβRII− cells treated with 10 ng/mL TGF-β1 (“Waste (TGF-b)”). An average of cells from two donors is shown.

FIG. 89 shows fold expansion of non-transduced (“NT”) cells and cells transduced with TCR (2.5 μL/million cells), CD8βα.TCR (“CDba.TCR”, 2.5 μL/million cells??) and CD8βα.TCR.dnTGFβRII (“CD8ba.TCR.dnTGFbRII”) at concentrations of 20, 10, 5, 2.5 or 1.25 μL/million cells) in an exemplary assay.

FIG. 90 shows vector copy numbers in cells prepared as described for FIG. 89 in an exemplary assay.

FIG. 91 shows CD8 and CD4 expression in cells prepared as described for FIG. 89 in an exemplary assay.

FIG. 92 shows TCR and TGFbRII expression in cells prepared as described for FIG. 89 in an exemplary assay.

FIG. 93 shows fold expansion of non-transduced (“NT”) cells and cells transduced with TCR (2.5 μL/million cells) and dnTGFβRII.TCR (“dnTGFbRII-TCR”) at 2.5 μL/million cells) in an exemplary assay.

FIG. 94 shows construct expression in cells prepared as described for FIG. 93 in an exemplary assay.

FIG. 95A-95C shows SMAD phosphorylation in TCR+ and TCR+dnTGFβRII+ cells in an exemplary assay in the absence (“untreated”) or presence (“TGFb”) of TGFβ. Non-transduced cells (“NT”) are shown as control. Phosphorylated SMAD+ percentages are shown as a proportion of all live, CD3+CD8+ T cells. Representative flow plots for one donor are shown.

FIG. 96A-96B shows proliferation of cells transduced with TCR+(“TCR”) or TCR+dnTGFβRII+(“TCR+DNR”) in the absence (“untreated”) or presence (“TGF-β”) of TGFβ in an exemplary co-culture assay. Tumor cells were added to the co-cultures on D+0, D+3, and D+6 (Tumor Challenges, #1, #2, and #3, respectively). Cells were harvested for flow analysis after 3 days in culture with tumor cells. Proliferation index values are shown for TCR+ cells.

FIG. 97A-97B shows fold growth of UACC257-RFP tumor cells normalized to 0 hours, in an exemplary co-culture assay with TCR+ or TCR+dnTGFβRII+ cells from three different donors (D645, D776, D897). Tumor cells were added to the co-cultures at hours 0, 68, 140, 212, and 284 hours, or approximately every 3 days. Growth of UACC257-RFP cells cultured alone (“UACC257”) or in co-culture with non-transduced cells (“NT”) is shown as a control.

FIG. 98A-98K show the cytokine release from TCR+ and TCR+dnTGFβRII+ cells in the absence (“untreated”) or presence (“TGFb”) of TGFβ in an exemplary assay.

### DETAILED DESCRIPTION

### Dominant Negative TGFβR

In embodiments, one or more TGF-β signaling pathway may be fully or partially disrupted in cells expressing one or more dominant negative TGF-β receptor (dnTGFβR). In embodiments, one or more dnTGFβRI polypeptides and/or dnTGFβRII polypeptides is provided. In embodiments, nucleic acids described herein comprise and/or encode one or more dnTGFβRI polypeptides and/or dnTGFβRII polypeptides. In embodiments, vectors described herein comprise and/or encode one or more dnTGFβRI polypeptides and/or dnTGFβRII polypeptides. In embodiments, cells described herein comprise and/or express one or more dnTGFβRI polypeptides and/or dnTGFβRII polypeptides. In embodiments, compositions described herein comprise one or more dnTGFβRI polypeptides and/or dnTGFβRII polypeptides or comprise cells comprising and/or expressing one or more dnTGFβRI polypeptides and/or dnTGFβRII polypeptides. In embodiments, TGFβR is rendered dominant negative, as non-limiting examples, by truncating and/or mutating TGFβR to remove and/or render inoperable all or a portion of at least one signaling portion of TGFβR.

In embodiments, TGFβRII is rendered dominant negative, as non-limiting examples, by truncating and/or mutating TGFβRII to remove and/or render inoperable all or a portion of an intracellular signaling portion of TGFβRII. In embodiments, a dnTGFβRII polypeptide may comprise an extracellular domain, a transmembrane domain, and/or a cytoplasmic domain. In embodiments, the cytoplasmic domain may be truncated, mutated, or absent.

In embodiments, dnTGFβRII variant 1 (dnTGFβRIIvar1) and/or dnTGFβRII variant 2 (dnTGFβRIIvar2) are provided and are examples of dnTGFβRII polypeptides. dnTGFβRIIvar1 may comprise or consist of SEQ ID NO: 305 and may be encoded by a nucleic acid comprising or consisting of SEQ ID NO: 306. dnTGFβRIIvar1 may comprise or consist of SEQ ID NO: 307 and may be encoded by a nucleic acid comprising or consisting of SEQ ID NO: 308. TGFβRIIvar1 and TGFβRIIvar2 disclosed herein each lack the cytoplasmic domain necessary for downstream signaling. Without being bound by theory, in embodiments, dnTGFβRII may function, for example, as follows: Truncated TGFβRII retains the ability to bind TGF-β and to form heteromeric complexes with TGFβRI, however the lack of the cytoplasmic domain prevents the phosphorylation of TGFβRI and subsequent activation of downstream elements. Moreover, the inclusion of a single truncated TGFβRII protein within the heteromeric TGF-β receptor complex may be sufficient to ablate signaling, suggesting that it performs in a dominant-negative fashion.

In embodiments, nucleic acid sequences encoding a dnTGFβRII polypeptide operatively coupled to a promoter are provided. In embodiments, nucleic acid sequences encoding a dnTGFβRII polypeptide operatively coupled to a post-transcriptional regulatory element are provided. In embodiments, the promoter is an MSCV promoter and/or the post-transcriptional regulatory element is a WPRE, optionally a mutated WPRE, optionally WPREmut2. FIG. 67A shows dnTGFβRIIvar1 coupled to an MSCV promoter and WPRE. In embodiments, the WPRE is WPRE, and such a construct is encoded by, for example, SEQ ID NO: 312. FIG. 67B shows dnTGFβRIIvar2 coupled to an MSCV promoter and WPRE. In embodiments, the WPRE is WPRE, and such a construct is encoded by, for example, SEQ ID NO: 313. In FIGS. 67A and 67B, the lines connecting the MSCV promoter to the dnTGFβRII and the dnTGFβRII to the WPRE may represent direct linkages, with no intervening sequences, or may represent intervening sequences, such as, but not limited to, a linker, an untranslated sequence (in the case of a nucleic acid sequence), a translated sequence, a sequence comprising one or more restriction endonuclease sites (in the case of a nucleic acid sequence), or a combination thereof.

In embodiments, isolated dnTGFβRII polypeptides are provided. In embodiments, isolated nucleic acid sequences comprising one or more nucleic acid sequences encoding one or more dnTGFβRII polypeptides are provided. In embodiments, isolated vectors comprising one or more nucleic acid sequences comprising one or more nucleic acid sequences encoding one or more dnTGFβRII polypeptides are provided. In embodiments, isolated cells expressing comprising or expressing one or more dnTGFβRII polypeptides are provided. In embodiments, isolated cells comprising or expressing one or more nucleic acid sequences comprising one or more nucleic acid sequences encoding one or more dnTGFβRII polypeptides are provided. In embodiments, isolated cells comprising or expressing one or more vectors comprising one or more nucleic acid sequences comprising one or more nucleic acid sequences encoding one or more dnTGFβRII polypeptides are provided. In embodiments, compositions comprising such polypeptides, nucleic acids, vectors, and/or cells are provided.

In an aspect, polypeptide sequences and/or nucleic acid sequences described herein may be isolated and/or recombinant sequences.

In embodiments, a dnTGFβRII polypeptide has a sequence comprising at least about 80%, 85%, 90%, 91%, 92%, 93%, 94%, 95%, 96%, 97%, 98%, 99%, or 100% sequence identity to the amino acid sequence of SEQ ID NO: 305. In embodiments, a dnTGFβRII polypeptide has a sequence comprising at least about 80%, 85%, 90%, 91%, 92%, 93%, 94%, 95%, 96%, 97%, 98%, 99%, or 100% sequence identity to the amino acid sequence of SEQ ID NO: 307. In embodiments, (i) function(s) of dnTGFβRII, such as, but not limited to, the ability of dnTGFβRII to bind TGFβ, are preserved and/or enhanced in a mutated dnTGFβRII polypeptide; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a mutated dnTGFβRII polypeptide; or (iii) any combinations thereof may be found in a mutated dnTGFβRII polypeptide.

In embodiments, a dnTGFβRII polypeptide comprises (a) SEQ ID NO: 305 comprising one, two, three, four, or five amino acid substitutions or (b) SEQ ID NO: 307 comprising one, two, three, four, or five amino acid substitutions. In embodiments, (i) function(s) of dnTGFβRII, such as, but not limited to, the ability of dnTGFβRII to bind TGFβ, are preserved and/or enhanced in a mutated dnTGFβRII polypeptide; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a mutated dnTGFβRII polypeptide; or (iii) any combinations thereof may be found in a mutated dnTGFβRII polypeptide.

In embodiments, a dnTGFβRII polypeptide is encoded by a nucleic acid comprising at least about 80%, 85%, 90%, 91%, 92%, 93%, 94%, 95%, 96%, 97%, 98%, 99%, or 100% sequence identity to the nucleic acid of SEQ ID NO: 306. In embodiments, a dnTGFβRII polypeptide is encoded by a nucleic acid comprising at least about 80%, 85%, 90%, 91%, 92%, 93%, 94%, 95%, 96%, 97%, 98%, 99%, or 100% sequence identity to the nucleic acid of SEQ ID NO: 308. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid; or (iii) any combinations thereof may be found in a dnTGFβRII polypeptide encoded by a mutated nucleic acid.

In embodiments, a dnTGFβRII polypeptide is encoded by a nucleic acid comprising (a) SEQ ID NO: 306 comprising one, two, three, four, or five nucleic acid substitutions or (b) SEQ ID NO: 308 comprising one, two, three, four, or five nucleic acid substitutions. One or more nucleic acid substitution in a codon may result in a codon encoding the same amino acid or may result in a codon encoding a different amino acid. In embodiments, one or more nucleic acid substitution in a codon may result in a codon encoding a conservative amino acid substitution. One or more nucleic acid substitution in a codon may result in a codon encoding the same amino acid. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid; or (iii) any combinations thereof may be found in a dnTGFβRII polypeptide encoded by a mutated nucleic acid.

In embodiments, a nucleic acid encoding a dnTGFβRII polypeptide may comprise a stop codon (such as TAA, TAG, or TGA), positioned at, as a non-limiting example, at the 3′ end of a nucleotide encoding the dnTGFβRII polypeptide.

In embodiments, a dnTGFβRII polypeptide may be encoded by a nucleic acid also comprising and/or encoding one or more MSCV promoter and/or one or more post transcriptional regulatory element. In embodiments, the post-transcriptional regulatory element (PRE) sequence may be selected from a Woodchuck hepatitis virus PRE (WPRE) (such as, but not limited to wild type WPRE, such as but not limited to SEQ ID NO: 264, or a mutated WPRE, such as but not limited to WPREmut1 (SEQ ID NO: 256) or WPREmut2 (SEQ ID NO: 257)) or a hepatitis B virus (HBV) PRE (HPRE) (SEQ ID NO: 366), or variant(s) thereof, or any combination thereof. In embodiments, such a construct is encoded by SEQ ID NO: 312. In embodiments, such a construct is encoded by SEQ ID NO: 313.

In embodiments, a dnTGFβRII polypeptide encoded by a nucleic acid also comprising and/or encoding one or more MSCV promoter and one or more WPRE may be encoded by a nucleic acid comprising at least about 80%, 85%, 90%, 91%, 92%, 93%, 94%, 95%, 96%, 97%, 98%, 99%, or 100% sequence identity to the nucleic acid of SEQ ID NO: 312. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid also comprising and/or encoding one or more MSCV promoter and one or more WPRE; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid also comprising and/or encoding one or more MSCV promoter and one or more WPRE; (iii) function(s) of the MSCV promoter are preserved and/or enhanced in a dnTGFβRII polypeptide encoded by a mutated nucleic acid also comprising and/or encoding one or more MSCV promoter and one or more WPRE; (iv) one or more post-translational functions of the WPRE are preserved and/or enhanced in a dnTGFβRII polypeptide encoded by a mutated nucleic acid also comprising and/or encoding one or more MSCV promoter and one or more WPRE; or (v) any combinations thereof may be found in a dnTGFβRII polypeptide encoded by a mutated nucleic acid also comprising and/or encoding one or more MSCV promoter and one or more WPRE.

In embodiments, a dnTGFβRII polypeptide encoded by a nucleic acid also comprising and/or encoding one or more MSCV promoter and one or more WPRE may be encoded by a nucleic acid comprising (a) SEQ ID NO: 312 comprising one, two, three, four, or five nucleic acid substitutions. In embodiments, one or more nucleic acid substitution in a codon may result in a codon encoding the same amino acid or may result in a codon encoding a different amino acid. In embodiments, one or more nucleic acid substitution in a codon may result in a codon encoding a conservative amino acid substitution. In embodiments, one or more nucleic acid substitution in a codon may result in a codon encoding the same amino acid. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid also comprising and/or encoding one or more MSCV promoter and one or more WPRE; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid also comprising and/or encoding one or more MSCV promoter and one or more WPRE; (iii) function(s) of the MSCV promoter are preserved and/or enhanced in a dnTGFβRII polypeptide encoded by a mutated nucleic acid also comprising and/or encoding one or more MSCV promoter and one or more WPRE; (iv) post-translational functions of the WPRE are preserved and/or enhanced in a dnTGFβRII polypeptide encoded by a mutated nucleic acid also comprising and/or encoding one or more MSCV promoter and one or more WPRE; or (v) any combinations thereof may be found in a dnTGFβRII polypeptide encoded by a mutated nucleic acid also comprising and/or encoding one or more MSCV promoter and one or more WPRE.

In embodiments, a dnTGFβRII polypeptide encoded by a nucleic acid also comprising and/or encoding one or more MSCV promoter and one or more WPRE may be encoded by a nucleic acid comprising at least about 80%, 85%, 90%, 91%, 92%, 93%, 94%, 95%, 96%, 97%, 98%, 99%, or 100% sequence identity to the nucleic acid of SEQ ID NO: 313. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid also comprising and/or encoding one or more MSCV promoter and one or more WPRE; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid also comprising and/or encoding one or more MSCV promoter and one or more WPRE; (iii) function(s) of the MSCV promoter are preserved and/or enhanced in a dnTGFβRII polypeptide encoded by a mutated nucleic acid also comprising and/or encoding one or more MSCV promoter and one or more WPRE; (iv) post-translational functions of the WPRE are preserved and/or enhanced in a dnTGFβRII polypeptide encoded by a mutated nucleic acid also comprising and/or encoding one or more MSCV promoter and one or more WPRE; or (v) any combinations thereof may be found in a dnTGFβRII polypeptide encoded by a mutated nucleic acid also comprising and/or encoding one or more MSCV promoter and one or more WPRE.

In embodiments, a dnTGFβRII polypeptide encoded by a nucleic acid also comprising and/or encoding one or more MSCV promoter and one or more WPRE may be encoded by a nucleic acid comprising (a) SEQ ID NO: 313 comprising one, two, three, four, or five nucleic acid substitutions. In embodiments, one or more nucleic acid substitution in a codon may result in a codon encoding the same amino acid or may result in a codon encoding a different amino acid. In embodiments, one or more nucleic acid substitution in a codon may result in a codon encoding a conservative amino acid substitution. In embodiments, one or more nucleic acid substitution in a codon may result in a codon encoding the same amino acid. In embodiments, (i) function(s) of dnTGFβRII, such as, but not limited to, the ability of dnTGFβRII to bind TGFβ, are preserved and/or enhanced in a dnTGFβRII polypeptide encoded by a mutated nucleic acid also comprising and/or encoding one or more MSCV promoter and one or more WPRE; (ii) one or more decrease or lack of function(s) of dnTGFβRII, such as, but not limited to, the decreased or eliminated signaling of the C-terminal portion of dnTGFβRII, one or more mutation in and/or deletion of a C-terminal portion of dnTGFβRII, or any combination thereof, are preserved and/or decreased in a dnTGFβRII polypeptide encoded by a mutated nucleic acid also comprising and/or encoding one or more MSCV promoter and one or more WPRE; (iii) function(s) of the MSCV promoter are preserved and/or enhanced in a dnTGFβRII polypeptide encoded by a mutated nucleic acid also comprising and/or encoding one or more MSCV promoter and one or more WPRE; (iv) post-translational functions of the WPRE are preserved and/or enhanced in a dnTGFβRII polypeptide encoded by a mutated nucleic acid also comprising and/or encoding one or more MSCV promoter and one or more WPRE; or (v) any combinations thereof may be found in a dnTGFβRII polypeptide encoded by a mutated nucleic acid also comprising and/or encoding one or more MSCV promoter and one or more WPRE.

In embodiments, a nucleic acid encoding a dnTGFβRII polypeptide may comprise a stop codon (such as TAA, TAG, or TGA), positioned at, as a non-limiting example, at the 3′ end of a nucleotide encoding the dnTGFβRII polypeptide.

Expression of dnTGFβRII may improve immune cell, such as but not limited to, T cell and/or natural killer cell, persistence, functionality, growth, viability, expansion, or combinations thereof, as compared to cells not expressing dnTGFβRII. Expression of dnTGFβRII may improve immune cell, such as but not limited to, T cell and/or natural killer cells, persistence, functionality, growth, viability, expansion, or combinations thereof, in a tumor microenvironment, as compared to cells not expressing dnTGFβRII. Expression of dnTGFβRII may increase efficacy of immune cells, such as, but not limited to, T cells and/or natural killer cells, in killing tumor cells, as compared to cells not expressing dnTGFβRII. Expression of dnTGFβRII may increase ability of immune cells, such as, but not limited to, T cells and/or natural killer cells, to survive in a tumor microenvironment, to persist in killing tumor cells, or combinations thereof, as compared to cells not expressing dnTGFβRII. In embodiments, expression of dnTGFβRII polypeptide may increase ability of immune cells, such as, but not limited to, T cells and/or natural killer cells, to maintain a naïve phenotype.

Persistence may be assessed, as a non-limiting example, by the length of time cells are detectable in an individual (e.g., patient) after infusion. As non-limiting examples, persistence may be measured at days, weeks, months, or years after infusion, as non-limiting examples, at about 1 week, about 2 weeks, about 4 weeks, about 1 month, about 2 months, about 3 months, about 6 months, about 9 months, about 12 months, about 18 months, about 24 months, and/or about 30 months after infusion. Persistence may be assessed, as non-limiting examples, by PCR of peripheral blood sample(s), by flow cytometry of peripheral blood samples(s), and/or by analysis of tumor biopsy sample(s). Persistence of cells expressing dnTGFβRII polypeptide may be compared, as non-limiting examples, to typical persistence of infused ACT cells or persistence of similar cells not expressing dnTGFβRII polypeptide.

Continued ability to kill tumor cells may be measured, as non-limiting examples, via (i) serial killing assays using an IncuCyte (wherein ability to kill/impair tumor growth as measured by fold growth during repeated tumor stimulations over a duration of time is assessed), and/or (ii) via cytokine/effector molecule production (IFNγ via ELISAs and other pro-inflammatory cytokines via Luminex (cytokines measured may include, as non-limiting examples, IFNγ, TNFα, Granzyme B, perforin, IL-2, IL-6, MIP-1β, MIP-1α, GM-CSF, RANTES, IL-18, IL-4, IL-10, and IP10). Continued ability of cells expressing dnTGFβRII polypeptide to kill tumor cells may be compared, as non-limiting examples, to continued ability of similar cells not expressing dnTGFβRII polypeptide to kill tumor cells or continued ability other control cells to kill tumor cells.

Naivety of phenotype may be assessed, as a non-limiting example, via Tmem panel assay via flow cytometry. Typically, flow cytometer gating is off of CD8+ TCR+ cells. Typically, a more naïve phenotype may be indicated by higher frequencies of the T memory subsets Tnaïve/scm (CD45RA+CCR7+), and Tcm (CD45RA−CCR7+) and an increase or retention of the CD39−CD69− and CD27+CD28+ populations. Low CD57 expression may also be desirable.

When assessing the persistence, functionality, growth, viability, expansion, tumor killing efficacy, naivety, or other characteristics of cells expressing dnTGFRβRII, cells such as non-transduced cells, cells transduced with TCR only, cells transduced with CD8 and TCR, or a combination thereof, may serve as control cells, as non-limiting examples. As dnTGFβRII may act to reduce or ablate signalling of TGFβ, assessment of the persistence, functionality, growth, viability, expansion, tumor killing efficacy, naivety, or other characteristics of cells expressing dnTGFRβRII may be performed in the presence of exogenous TGFβ, such as TGF-β1.

DnTGFβRII may act in a cis manner (e.g., affecting cells in which it is expressed), in a trans manner (e.g., affecting cells in which it is not expressed), or combinations thereof. Where dnTGFβRII acts in trans, cells adjacent to or near (e.g., within the tumor microenvironment) cells expressing dnTGFβRII may exhibit any or combinations of improvements the same or similar to those described for cells expressing dnTGFβRII, as compared to cells not adjacent to or near cells expressing dnTGFβRII. Without being bound by theory, dnTGFβRII may act to reduce the amount of TGF-β in the tumor microenvironment; also, cells expressing dnTGFβRII may exhibit an improved ability to secrete cytokines in response to target antigen in the presence of TGF-β, as compared to cells that do not express dnTGFβRII.

### Modified CD8 Polypeptides

CD8 polypeptides described herein may comprise the general structure of a N-terminal signal peptide (optional), CD8α immunoglobulin (Ig)-like domain, CD8β stalk region (domain), CD8α transmembrane domain, and a CD8α cytoplasmic domain. The modified CD8 polypeptides described herein shown an unexpected improvement in functionality of T cells co-transduced with a vector expressing a TCR and CD8 polypeptide.

CD8 polypeptides described herein may comprise the general structure of a N-terminal signal peptide (optional), CD8α immunoglobulin (Ig)-like domain, a stalk domain or region, CD8α transmembrane domain, and a CD8α cytoplasmic domain.

In embodiments, CD8 polypeptides described herein may comprise (a) an immunoglobulin (Ig)-like domain comprising at least about 80%, at least about 85%, at least about 90%, at least about 95%, at least about 98%, at least about 99%, or about 100% sequence identity to the amino acid sequence of SEQ ID NO: 1; (b) a region comprising at least about 80%, at least about 85%, at least about 90%, at least about 95%, at least about 98%, at least about 99%, or about 100% sequence identity to the amino acid sequence of SEQ ID NO: 2; (c) a transmembrane domain comprising at least about 80%, at least about 85%, at least about 90%, at least about 95%, at least about 98%, at least about 99%, or about 100% sequence identity to the amino acid sequence of SEQ ID NO: 3, and (d) a cytoplasmic domain comprising at least about 80%, at least about 85%, at least about 90%, at least about 95%, at least about 98%, at least about 99%, or about 100% sequence identity to the amino acid sequence of SEQ ID NO: 4. The CD8 polypeptides described herein may be co-expressed with a T-cell receptor or CAR-T in a T-cell and used in methods of adoptive cell therapy (ACT). The T-cell may be an αβ T-cell or a γδ T-cell.

In embodiments, CD8 polypeptides described herein may comprise (a) at least about 80%, at least 85%, at least 90%, at least 95%, at least 98%, at least 99%, or 100% sequence identity to the amino acid sequence of SEQ ID NO: 1; (b) at least about 80%, at least about 85%, at least about 90%, at least about 95%, at least about 98%, at least about 99%, or about 100% sequence identity to the amino acid sequence of SEQ ID NO: 2; (c) at least about 80%, at least about 85%, at least about 90%, at least about 95%, at least about 98%, at least about 99%, or about 100% sequence identity to the amino acid sequence of SEQ ID NO: 3, and (d) a at least about 80%, at least about 85%, at least about 90%, at least about 95%, at least about 98%, at least about 99%, or about 100% sequence identity to the amino acid sequence of SEQ ID NO: 4. The CD8 polypeptides described herein may be co-expressed with a T-cell receptor or CAR-T in a T-cell and used in methods of adoptive cell therapy (ACT). The T-cell may be an αβ T-cell or a γδ T-cell.

In embodiments, CD8 polypeptides described herein may comprise (a) SEQ ID NO: 1 comprising one, two, three, four, or five amino acid substitutions; (b) SEQ ID NO: 2 comprising one, two, three, four, or five amino acid substitutions; (c) SEQ ID NO: 3 comprising one, two, three, four, or five amino acid substitutions, and (d) SEQ ID NO: 4 comprising one, two, three, four, or five amino acid substitutions. In embodiments, the substitutions may b conservative amino acid substitutions. The CD8 polypeptides described herein may be co-expressed with a T-cell receptor or CAR-T in a T-cell and used in methods of adoptive cell therapy (ACT). The T-cell may be an γδ T-cell or a γδ T-cell.

CD8 is a membrane-anchored glycoprotein that functions as a coreceptor for antigen recognition of the peptide/MHC class I complexes by T cell receptors (TCR) and plays an important role in T cell development in the thymus and T cell activation in the periphery. Functional CD8 is a dimeric protein made of either two a chains (CD8αα) or an α chain and a β chain (CD8αβ), and the surface expression of the β chain may require its association with the coexpressed a chain to form the CD8αβ heterodimer. CD8αα and CD8αβ may be differentially expressed on a variety of lymphocytes. CD8αβ is expressed predominantly on the surface of αβTCR⁺ T cells and thymocytes, and CD8αα on a subset of αβTCR⁺, γδTCR⁺ intestinal intraepithelial lymphocytes, NK cells, dendritic cells, and a small fraction of CD4+ T cells.

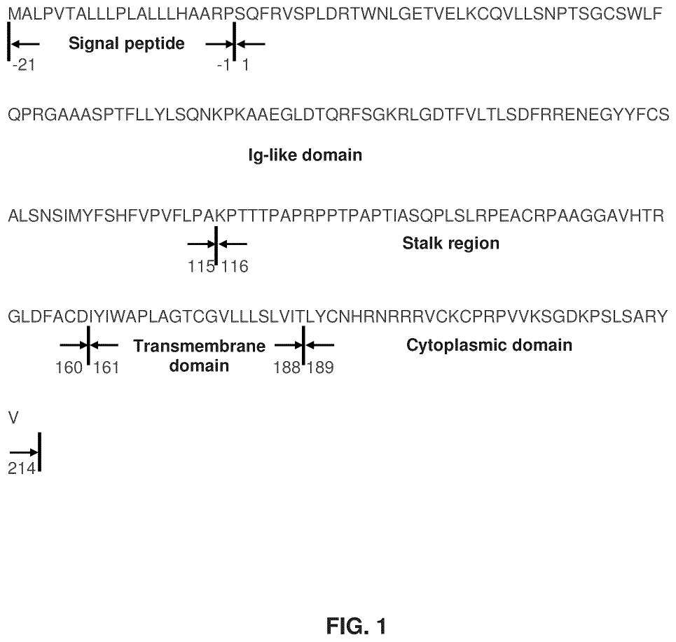

For example, the human CD8 gene may express a protein of 235 amino acids. FIG. 1 shows a CD8α protein (CD8 α1—SEQ ID NO: 258), which in an aspect is divided into the following domains (starting at the amino terminal and ending at the carboxy terminal of the polypeptide): (1) signal peptide (amino acids −21 to −1), which may be cleaved off in human cells during the transport of the receptor to the cell surface and thus may not constitute part of the mature, active receptor; (2) immunoglobulin (Ig)-like domain (in this embodiment, amino acids 1-115), which may assume a structure, referred to as the immunoglobulin fold, which is similar to those of many other molecules involved in regulating the immune system, the immunoglobulin family of proteins. The crystal structure of the CD8αα receptor in complex with the human MHC molecule HLA-A2 has demonstrated how the Ig domain of CD8αα receptor binds the ligand; (3) membrane proximal region (in this embodiment, amino acids 116-160), which may be an extended linker region allowing the CD8αα receptor to “reach” from the surface of the T-cell over the top of the MHC to the a3 domain of the MHC where it binds. The stalk region may be glycosylated and may be inflexible; (4) transmembrane domain (in this embodiment, amino acids 161-188), which may anchor the CD8αα receptor in the cell membrane and is therefore not part of the soluble recombinant protein; and (5) cytoplasmic domain (in this embodiment, amino acids 189-214), which can mediate a signaling function in T-cells through its association with p56^{lck}, which may be involved in the T cell activation cascade of phosphorylation events.

CD8α sequences may generally have a sufficient portion of the immunoglobulin domain to be able to bind to MHC. Generally, CD8α molecules may contain all or a substantial part of immunoglobulin domain of CD8α, e.g., SEQ ID NO: 258, but in an aspect may contain at least about 10, 15, 20, 25, 30, 35, 40, 45, 50, 55, 60, 65, 70, 75, 80, 85, 90, 95, 100, 105, 110 or 115 amino acids of the immunoglobulin domain. The CD8α molecules of the present disclosure may be dimers (e.g., CD8αα or CD8αβ), and CD8α monomer may be included within the scope of the present disclosure. In an aspect, CD8α of the present disclosure may comprise CD8α1 (SEQ ID NO: 258) and CD8α2 (SEQ ID NO: 259). In an aspect, the present disclosure may comprise CD8α1 (SEQ ID NO: 258) encoded by SEQ ID NO: 310.

CD8α and β subunits may have similar structural motifs, including an Ig-like domain, a stalk region of 30-40 amino acids, a transmembrane region, and a short cytoplasmic domain of about 20 amino acids. CD8α and β chains have two and one N-linked glycosylation sites, respectively, in the Ig-like domains where they share <20% identity in their amino acid sequences. The CD8β stalk region is 10-13 amino acids shorter than the CD8α stalk and is highly glycosylated with O-linked carbohydrates. These carbohydrates on the β, but not the α, stalk region appear to be quite heterogeneous due to complex sialylations, which may be differentially regulated during the developmental stages of thymocytes and upon activation of T cells. Glycan adducts have been shown to play regulatory roles in the functions of glycoproteins and in immune responses. Glycans proximal to transmembrane domains can affect the orientation of adjacent motifs. The unique biochemical properties of the CD8β chain stalk region may present a plausible candidate for modulating the coreceptor function.

The CD8α polypeptide may be modified by replacing CD8α stalk region with a CD8β stalk region to generate a modified CD8α polypeptide. In embodiments, the modified CD8α polypeptides described herein may have a CD8β stalk region comprising at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 2. The modified CD8α polypeptides described herein may have an immunoglobulin (Ig)-like domain having at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1. Modified CD8 polypeptides may have a transmembrane domain comprising at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 3. Modified CD8 polypeptides described herein may have a cytoplasmic tail comprising at least at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 4. The CD8 polypeptides described herein may have at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5. The CD8 polypeptides described herein may comprise one or more signal peptide comprising at least about 80%, at least about 85%, at least about 90%, at least about 95%, at least about 99%, or about 100% sequence identity to the amino acid sequence of SEQ ID NO: 6 or SEQ ID NO: 294 directly or indirectly fused to the N-terminus or fused to the C-terminus of mCD8α polypeptide. The CD8 polypeptides described herein may have at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 7.

### T-Cells

T-cells may express dnTGFβRII, modified CD8 polypeptides described herein, or combinations thereof. As a non-limiting example, a T-cell may co-express a T-cell Receptor (TCR) and a dnTGFβRII polypeptide. As another non-limiting example, a T-cell may co-express a T-cell Receptor (TCR) and a modified CD8 polypeptide described herein. As another non-limiting example, a T-cell may co-express a T-cell Receptor (TCR), a dnTGFβRII polypeptide, and a modified CD8 polypeptide described herein. T-cells may also express a chimeric antigen receptor (CAR), CAR-analogues, or CAR derivatives. In embodiments, a CD8 polypeptide may comprise a CD8α chain and/or a CD8β chain, and the CD8α chain and/or CD8β chain may independently be modified or unmodified.

The T-cell may be an αβ T cell, a γδ T cell, a natural killer T cell, a natural killer T cell, or a combination thereof if in a population. The T cell may be a CD4+ T cell, CD8+ T cell, or a CD4+/CD8+ T cell. In embodiments, a cell may comprise an αβ T cell, a γδ T cell, a natural killer cell, a natural killer T cell, a CD4+ T cell, CD8+ T cell, a CD4+/CD8+ cell, or combinations thereof.

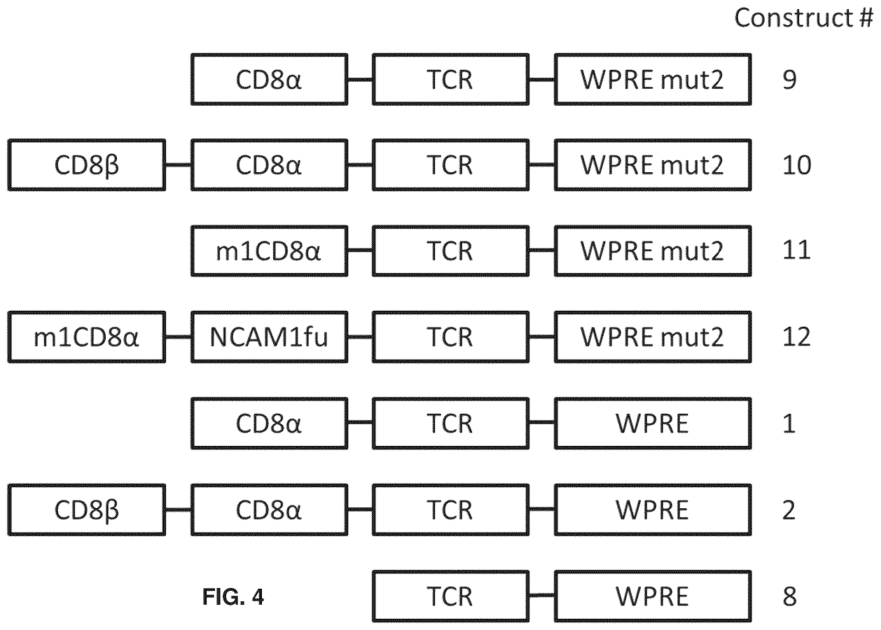

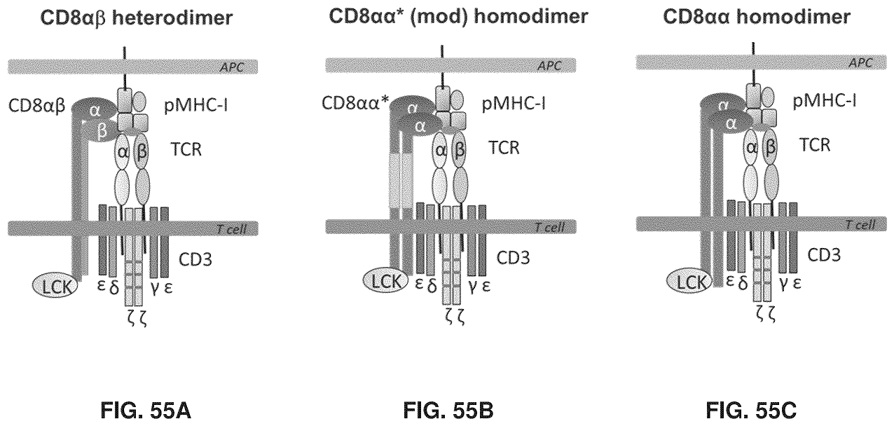

A T cell may be an αβ T cell and may express a CD8 polypeptide described herein. A T cell may be an αβ T cell and may express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B). A T cell may be an αβ T cell and may express one or any combination of a dnTGFβRII polypeptide, a CD8 polypeptide, and/or a CAR. In embodiments, a CD8 polypeptide may comprise a CD8α chain and/or a CD8β chain, and the CD8α chain and/or CD8β chain may independently be modified or unmodified.

A T cell may be a γδ T cell and may express a CD8 polypeptide described herein. In embodiments, a T cell may be a γδ T cell and may express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B). A T cell may be a γδ T cell and may express one or any combination of a dnTGFβRII polypeptide, a CD8 polypeptide, and/or a CAR. In embodiments, a CD8 polypeptide may comprise a CD8α chain and/or a CD8β chain, and the CD8α chain and/or CD8β chain may independently be modified or unmodified.

A T cell or cells comprising, or comprising nucleic acid(s) encoding, one or any combination of a TCR comprising an α chain and a β chain, a TCR comprising a γ chain and a chain, a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

A T cell or cells comprising, or comprising nucleic acid(s) encoding, one or any combination of a TCR comprising an α chain and a β chain, a dnTGFβRII polypeptide, and/or a CD8 polypeptide may be provided. A cell or cells comprising, or comprising nucleic acid(s) encoding, one or any combination of a TCR comprising a γ chain and a S chain, a dnTGFβRII polypeptide, and/or a CD8 polypeptide may be provided. A cell or cells comprising, or comprising nucleic acid(s) encoding, one or any combination of a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

A T cell or cells comprising, or comprising nucleic acid(s) encoding, a TCR comprising an α chain and a β chain, a dnTGFβRII polypeptide, and a CD8 polypeptide may be provided. In embodiments, a cell or cells comprising, or comprising nucleic acid(s) encoding, a TCR comprising a γ chain and a δ chain, a dnTGFβRII polypeptide, and a CD8 polypeptide may be provided. A cell or cells comprising, or comprising nucleic acid(s) encoding, a CAR, a dnTGFβRII polypeptide, and a CD8 polypeptide may be provided. In embodiments, a CD8 polypeptide may comprise a CD8α chain and/or a CD8β chain, and the CD8α chain and/or CD8β chain may independently be modified or unmodified.

A T cell or cells comprising, or comprising nucleic acid(s) encoding, a TCR comprising an α chain and a β chain and a dnTGFβRII polypeptide may be provided. A cell or cells comprising, or comprising nucleic acid(s) encoding, a TCR comprising a γ chain and a δ chain and a dnTGFβRII polypeptide may be provided. A cell or cells comprising, or comprising nucleic acid(s) encoding, a CAR and a dnTGFβRII polypeptide may be provided. In embodiments, a CD8 polypeptide may comprise a CD8α chain and/or a CD8β chain, and the CD8α chain and/or CD8β chain may independently be modified or unmodified.

A T cell or cells comprising, or comprising nucleic acid(s) encoding, a TCR comprising an α chain and a β chain and a CD8 polypeptide may be provided. In embodiments, a cell or cells comprising, or comprising nucleic acid(s) encoding, a TCR comprising a γ chain and a δ chain and a CD8 polypeptide may be provided. A cell or cells comprising, or comprising nucleic acid(s) encoding a CAR and a CD8 polypeptide may be provided. In embodiments, a CD8 polypeptide may comprise a CD8α chain and/or a CD8β chain, and the CD8α chain and/or CD8β chain may independently be modified or unmodified. In embodiments, such cells may also comprise, or comprise nucleic acid(s) encoding, at least one dnTGFβRII polypeptide.

### Natural Killer (NK) Cells

Natural Killer (NK) cells may also be engineered and used in adoptive cell therapy (ACT). See, e.g., Morton L T, et al., “T cell receptor engineering of primary NK cells to therapeutically target tumors and tumor immune evasion”, J Immunother Cancer, Mar. 14, 2022; 10:e003715, which is incorporated by reference herein in its entirety. In embodiments, engineered NK cells are provided.

NK cells may express dnTGFβRII, modified CD8 polypeptides described herein, or combinations thereof. As a non-limiting example, a NK cell may co-express a T-cell Receptor (TCR) and a dnTGFβRII polypeptide. As another non-limiting example, a NK cell may co-express a T-cell Receptor (TCR) and a modified CD8 polypeptide described herein. As another non-limiting example, a NK cell may co-express a T-cell Receptor (TCR), a dnTGFβRII polypeptide, and a modified CD8 polypeptide described herein. NK cells may also express a chimeric antigen receptor (CAR), CAR-analogues, or CAR derivatives. In embodiments, a CD8 polypeptide may comprise a CD8α chain and/or a CD8β chain, and the CD8α chain and/or CD8β chain may independently be modified or unmodified.

The NK cell may express a CD8 polypeptide described herein. A NK cell may express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B). A NK cell may express one or any combination of a dnTGFβRII polypeptide, a CD8 polypeptide, and/or a CAR. In embodiments, a CD8 polypeptide may comprise a CD8α chain and/or a CD8β chain, and the CD8α chain and/or CD8β chain may independently be modified or unmodified.

A NK cell or cells comprising, or comprising nucleic acid(s) encoding, one or any combination of a TCR comprising an α chain and a β chain, a TCR comprising a γ chain and a δ chain, a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

A NK cell or cells comprising, or comprising nucleic acid(s) encoding, one or any combination of a TCR comprising an α chain and a β chain, a dnTGFβRII polypeptide, and/or a CD8 polypeptide may be provided. A cell or cells comprising, or comprising nucleic acid(s) encoding, one or any combination of a TCR comprising a γ chain and a δ chain, a dnTGFβRII polypeptide, and/or a CD8 polypeptide may be provided. A cell or cells comprising, or comprising nucleic acid(s) encoding, one or any combination of a CAR, a dnTGFβRII polypeptide, and/or a CD8 polypeptide may be provided. In embodiments, a CD8 polypeptide may comprise a CD8α chain and/or a CD8β chain, and the CD8α chain and/or CD8β chain may independently be modified or unmodified.

A NK cell or cells comprising, or comprising nucleic acid(s) encoding, a TCR comprising an α chain and a β chain, a dnTGFβRII polypeptide, and a CD8 polypeptide may be provided. In embodiments, a cell or cells comprising, or comprising nucleic acid(s) encoding, a TCR comprising a γ chain and a δ chain, a dnTGFβRII polypeptide, and a CD8 polypeptide may be provided. A cell or cells comprising, or comprising nucleic acid(s) encoding, a CAR, a dnTGFβRII polypeptide, and a CD8 polypeptide may be provided. In embodiments, a CD8 polypeptide may comprise a CD8α chain and/or a CD8β chain, and the CD8α chain and/or CD8β chain may independently be modified or unmodified.

A NK cell or cells comprising, or comprising nucleic acid(s) encoding, a TCR comprising an α chain and a β chain and a dnTGFβRII polypeptide may be provided. A cell or cells comprising, or comprising nucleic acid(s) encoding, a TCR comprising a γ chain and a δ chain and a dnTGFβRII polypeptide may be provided. A cell or cells comprising, or comprising nucleic acid(s) encoding, a CAR and a dnTGFβRII polypeptide may be provided. In embodiments, a CD8 polypeptide may comprise a CD8α chain and/or a CD8β chain, and the CD8α chain and/or CD8β chain may independently be modified or unmodified.

A NK cell or cells comprising, or comprising nucleic acid(s) encoding, a TCR comprising an α chain and a β chain and a CD8 polypeptide may be provided. In embodiments, a cell or cells comprising, or comprising nucleic acid(s) encoding, a TCR comprising a γ chain and a δ chain and a CD8 polypeptide may be provided. A cell or cells comprising, or comprising nucleic acid(s) encoding a CAR and a CD8 polypeptide may be provided. In embodiments, a CD8 polypeptide may comprise a CD8α chain and/or a CD8β chain, and the CD8α chain and/or CD8β chain may independently be modified or unmodified. In embodiments, such cells may also comprise, or comprise nucleic acid(s) encoding, at least one dnTGFβRII polypeptide.

### T-Cell Receptors

A T-cell may co-express a T-cell receptor (TCR), antigen binding protein, or both, with dnTGFβRII polypeptides and/or CD8 polypeptides described herein, including, but are not limited to, those listed in Table 3 (SEQ ID NOs: 15-92). In embodiments, a CD8 polypeptide may comprise a CD8α chain and/or a CD8β chain, and the CD8α chain and/or CD8β chain may independently be modified or unmodified. Further, a T-cell may express one or any combination of dnTGFβRII polypeptides, modified CD8 polypeptides described herein, TCRs, and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. The cell may be a T cell or a natural killer cell. The T-cell may be a CD4+ cell, a CD8+ cell, a CD4+/CD8+ cell, an αβ T cell, a γδ T cell, or a natural killer T cell. In embodiments, TCRs described herein may be single-chain TCRs or soluble TCRs.

Further, the TCRs that may be co-expressed with the dnTGFβRII polypeptides and/or CD8 polypeptides described herein in a T-cell may be TCRs comprised of an alpha chain (TCRα) and a beta chain (TCRβ). In embodiments, a CD8 polypeptide may comprise a CD8α chain and/or a CD8β chain, and the CD8α chain and/or CD8β chain may independently be modified or unmodified. The TCRα chains and TCRβ chains that may be used in TCRs may be selected from R11KEA (SEQ ID NO: 15 and 16), R20P1H7 (SEQ ID NO: 17 and 18), R7P1D5 (SEQ ID NO: 19 and 20), R10P2G12 (SEQ ID NO: 21 and 22), R10P1A7 (SEQ ID NO: 23 and 24), R4P1D10 (SEQ ID NO: 25 and 26), R4P3F9 (SEQ ID NO: 27 and 28), R4P3H3 (SEQ ID NO: 29 and 30), R36P3F9 (SEQ ID NO: 31 and 32), R52P2G11 (SEQ ID NO: 33 and 34), R53P2A9 (SEQ ID NO: 35 and 36), R26P1A9 (SEQ ID NO: 37 and 38), R26P2A6 (SEQ ID NO: 39 and 40), R26P3H1 (SEQ ID NO: 41 and 42), R35P3A4 (SEQ ID NO: 43 and 44), R37P1C9 (SEQ ID NO: 45 and 46), R37P1H1 (SEQ ID NO: 47 and 48), R42P3A9 (SEQ ID NO: 49 and 50), R43P3F2 (SEQ ID NO: 51 and 52), R43P3G5 (SEQ ID NO: 53 and 54), R59P2E7 (SEQ ID NO: 55 and 56), R11P3D3 (SEQ ID NO: 57 and 58), R16P1C10 (SEQ ID NO: 59 and 60), R16P1E8 (SEQ ID NO: 61 and 62), R17P1A9 (SEQ ID NO: 63 and 64), R17P1D7 (SEQ ID NO: 65 and 66), R17P1G3 (SEQ ID NO: 67 and 68), R17P2B6 (SEQ ID NO: 69 and 70), R11P3D3KE (SEQ ID NO: 71 and 303), R39P1C12 (SEQ ID NO: 304 and 74), R39P1F5 (SEQ ID NO: 75 and 76), R40P1C2 (SEQ ID NO: 77 and 78), R41P3E6 (SEQ ID NO: 79 and 80), R43P3G4 (SEQ ID NO: 81 and 82), R44P3B3 (SEQ ID NO: 83 and 84), R44P3E7 (SEQ ID NO: 85 and 86), R49P2B7 (SEQ ID NO: 87 and 88), R55P1G7 (SEQ ID NO: 89 and 90), or R59P2A7 (SEQ ID NO: 91 and 92). The cell may be a T cell or a natural killer cell. The T-cell may be a αβ T cell, γδ T cell, or a natural killer T cell.

Table 1 shows examples of the peptides to which TCRs bind when the peptide is in a complex with an MHC molecule. (MHC molecules in humans may be referred to as HLA, human leukocyte-antigens).

TABLE 1
T-Cell Receptor and Peptides

| | Peptide |
|---|---|
| TCR name | (SEQ ID NO:) |
| R20P1H7, R7P1D5, R10P2G12 | KVLEHVVRV |
| | (SEQ ID NO: 215) |
| R10P1A7 | KIQEILTQV |
| | (SEQ ID NO: 123) |
| R4P1D10, R4P3F9, R4P3H3 | FLLDGSANV |
| | (SEQ ID NO: 238) |
| R36P3F9, R52P2G11, R53P2A9 | ILQDGQFLV |
| | (SEQ ID NO: 193) |
| R26P1A9, R26P2A6, R26P3H1, | KVLEYVIKV |
| R35P3A4, R37P1C9, R37P1H1, | (SEQ ID NO: 202) |
| R42P3A9, R43P3F2, R43P3G5, | |
| R59P2E7 | |
| R11KEA, R11P3D3, R16P1C10, | SLLQHLIGL |
| R16P1E8, R17P1A9, R17P1D7, | (SEQ ID NO: 147) |
| R17P1G3, R17P2B6, R11P3D3KE | |
| R39P1C12, R39P1F5, R40P1C2, | ALSVLRLAL |
| R41P3E6, R43P3G4, R44P3B3, | (SEQ ID NO: 248) |
| R44P3E7, R49P2B7, R55P1G7, | |
| R59P2A7 | |

### Tumor Associated Antigens (TAA)

Tumor associated antigen (TAA) peptides may be used with the dnTGFβRII polypeptides and/or CD8 polypeptides constructs, methods and embodiments described herein. For example, the T-cell receptors (TCRs) described herein may specifically bind to the TAA peptide when bound to a human leukocyte antigen (HLA). This is also known as a major histocompatibility complex (MHC) molecule. The MHC-molecules of the human are also designated as human leukocyte-antigens (HLA).

Tumor associated antigen (TAA) peptides that may be used with the dnTGFβRII polypeptides and/or CD8 polypeptides described herein include, but are not limited to, those listed in Table 3 and those TAA peptides described in U.S. Patent Application Publication No. 2016/0187351; U.S. Patent Application Publication No. 2017/0165335; U.S. Patent Application Publication No. 2017/0035807; U.S. Patent Application Publication No. 2016/0280759; U.S. Patent Application Publication No. 2016/0287687; U.S. Patent Application Publication No. 2016/0346371; U.S. Patent Application Publication No. 2016/0368965; U.S. Patent Application Publication No. 2017/0022251; U.S. Patent Application Publication No. 2017/0002055; U.S. Patent Application Publication No. 2017/0029486; U.S. Patent Application Publication No. 2017/0037089; U.S. Patent Application Publication No. 2017/0136108; U.S. Patent Application Publication No. 2017/0101473; U.S. Patent Application Publication No. 2017/0096461; U.S. Patent Application Publication No. 2017/0165337; U.S. Patent Application Publication No. 2017/0189505; U.S. Patent Application Publication No. 2017/0173132; U.S. Patent Application Publication No. 2017/0296640; U.S. Patent Application Publication No. 2017/0253633; U.S. Patent Application Publication No. 2017/0260249; U.S. Patent Application Publication No. 2018/0051080; U.S. Patent Application Publication No. 2018/0164315; U.S. Patent Application Publication No. 2018/0291082; U.S. Patent Application Publication No. 2018/0291083; U.S. Patent Application Publication No. 2019/0255110; U.S. Pat. Nos. 9,717,774; 9,895,415; U.S. Patent Application Publication No. 2019/0247433; U.S. Patent Application Publication No. 2019/0292520; U.S. Patent Application Publication No. 2020/0085930; U.S. Pat. Nos. 10,336,809; 10,131,703; 10,081,664; 10,081,664; 10,093,715; 10,583,573; and U.S. Patent Application Publication No. 2020/00085930; the contents of each of these publications, sequences, and sequence listings described therein are herein incorporated by reference in their entireties. The Tumor associated antigen (TAA) peptides described herein may be bound to an HLA (MHC molecule). The Tumor associated antigen (TAA) peptides bound to an HLA may be recognized by a TCR described herein, optionally co-expressed with CD8 polypeptides described herein.

T cells may be engineered to express a chimeric antigen receptor (CAR) comprising a ligand binding domain derived from NKG2D, NKG2A, NKG2C, NKG2F, LLT1, AICL, CD26, NKRP1, NKp30, NKp44, NKp46, CD244 (2B4), DNAM-1, and NKp80, or an anti-tumor antibody such as anti-Her2neu or anti-EGFR and a signaling domain obtained from CD3-ζ, Dap 10, CD28, 4-IBB, and CD40L. In some examples, the chimeric receptor binds MICA, MICB, Her2neu, EGFR, mesothelin, CD38, CD20, CD 19, PSA, RON, CD30, CD22, CD37, CD38, CD56, CD33, CD30, CD138, CD123, CD79b, CD70, CD75, CA6, GD2, alpha-fetoprotein (AFP), carcinoembryonic antigen (CEA), CEACAM5, CA-125, MUC-16, 5T4, NaPi2b, ROR1, ROR2, 5T4, PLIF, Her2/Neu, EGFRvIII, GPMNB, LIV-1, glycolipidF77, fibroblast activating protein, PSMA, STEAP-1, STEAP-2, c-met, CSPG4, Nectin-4, VEGFR2, PSCA, folate binding protein/receptor, SLC44A4, Cripto, CTAG1B, AXL, IL-13R, IL-3R, SLTRK6, gp100, MART1, Tyrosinase, SSX2, SSX4, NYESO-1, epithelial tumor antigen (ETA), MAGEA family genes (such as MAGE3A. MAGE4A), KKLC1, mutated ras, βraf, p53, MHC class I chain-related molecule A (MICA), or MHC class I chain-related molecule B (MICB), HPV, or CMV. The cell may be a T cell or a natural killer cell. The T-cell may be a αβ T cell, γδ T cell, or a natural killer T cell.

### Culturing T-Cells

Methods for the activation, transduction, and/or expansion of T cells, e.g., tumor-infiltrating lymphocytes, CD8+ T cells, CD4+ T cells, and T cells, that may be used for transgene expression are described herein. T cells may be activated, transduced, and expanded, while depleting α- and/or β-TCR positive cells. The cell may be a T cell or a natural killer cell. The T-cell may be a αβ T cell, γδ T cell, or a natural killer T cell.

Methods for the ex vivo expansion of a population of engineered γδ T-cells for adoptive transfer therapy are described herein. Engineered γδ T cells of the disclosure may be expanded ex vivo. Engineered T cells described herein can be expanded in vitro without activation by APCs, or without co-culture with APCs, and aminophosphates. Methods for transducing T cells are described in U.S. Patent Application No. 2019/0175650, published on Jun. 13, 2019, the contents of which are incorporated by reference in their entirety. Other methods for transduction and culturing of T-cells may be used.

T cells, including γδ T cells, may be isolated from a complex sample that is cultured in vitro. In embodiments, whole PBMC population, without prior depletion of specific cell populations, such as monocytes, αβ T-cells, B-cells, and NK cells, can be activated and expanded. In embodiments, enriched T cell populations can be generated prior to their specific activation and expansion. In embodiments, activation and expansion of γδ T cells may be performed with or without the presence of native or engineered antigen presenting cells (APCs). In embodiments, isolation and expansion of T cells from tumor specimens can be performed using immobilized T cell mitogens, including antibodies specific to γδ TCR, and other γδ TCR activating agents, including lectins. In embodiments, isolation and expansion of γδ T cells from tumor specimens can be performed in the absence of γδ T cell mitogens, including antibodies specific to γδ TCR, and other γδ TCR activating agents, including lectins.

T cells, including γδ T cells, may be isolated from leukapheresis of a subject, for example, a human subject. In embodiments, γδ T cells are not isolated from peripheral blood mononuclear cells (PBMC). The T cells may be isolated using anti-CD3 and anti-CD28 antibodies, optionally with recombinant human Interleukin-2 (rhIL-2), e.g., between about 50 and 150 U/mL rhIL-2.

The isolated T cells can rapidly expand in response to contact with one or more antigens. Some γδ T cells, such as Vγ9Vδ2+ T cells, can rapidly expand in vitro in response to contact with some antigens, like prenyl-pyrophosphates, alkyl amines, and metabolites or microbial extracts during tissue culture. Stimulated T-cells can exhibit numerous antigen-presentation, co-stimulation, and adhesion molecules that can facilitate the isolation of T-cells from a complex sample. T cells within a complex sample can be stimulated in vitro with at least one antigen for 1 day, 2 days, 3 days, 4 days, 5 days, 6 days, 7 days, or another suitable period of time. Stimulation of T cells with a suitable antigen can expand T cell population in vitro.

Activation and expansion of γδ T cells can be performed using activation and co-stimulatory agents described herein to trigger specific γδ T cell proliferation and persistence populations. In embodiments, activation and expansion of γδ T-cells from different cultures can achieve distinct clonal or mixed polyclonal population subsets. In embodiments, different agonist agents can be used to identify agents that provide specific γδ activating signals. In embodiments, agents that provide specific γδ activating signals can be different monoclonal antibodies (MAbs) directed against the γδ TCRs. In embodiments, companion co-stimulatory agents to assist in triggering specific γδ T cell proliferation without induction of cell energy and apoptosis can be used. These co-stimulatory agents can include ligands binding to receptors expressed on γδ cells, such as NKG2D, CD161, CD70, JAML, DNAX accessory molecule-1 (DNAM-1), ICOS, CD27, CD137, CD30, HVEM, SLAM, CD122, DAP, and CD28. In embodiments, co-stimulatory agents can be antibodies specific to unique epitopes on CD2 and CD3 molecules. CD2 and CD3 can have different conformation structures when expressed on αβ or γδ T-cells. In embodiments, specific antibodies to CD3 and CD2 can lead to distinct activation of γδ T cells.

Non-limiting examples of antigens that may be used to stimulate the expansion of T cells, including γδ T cells, from a complex sample in vitro may comprise, prenyl-pyrophosphates, such as isopentenyl pyrophosphate (IPP), alkyl-amines, metabolites of human microbial pathogens, metabolites of commensal bacteria, methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates.

A population of T-cells, including γδ T cells, may be expanded ex vivo prior to engineering of the T-cells. Non-limiting example of reagents that can be used to facilitate the expansion of a T-cell population in vitro may comprise anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), Les Culinaris Agglutinin (LCA), Pisum sativum Agglutinin (PSA), Helix pomatia agglutinin (HPA), Vicia graminea Lectin (VGA), or another suitable mitogen capable of stimulating T-cell proliferation. Further, the T-cells may be expanded using MCSF, IL-6, eotaxin, IFN-alpha, IL-7, gamma-induced protein 10, IFN-gamma, IL-1RA, IL-12, MIP-1alpha, IL-2, IL-13, MIP-1beta, IL-2R, IL-15, and combinations thereof.

The ability of γδ T cells to recognize a broad spectrum of antigens can be enhanced by genetic engineering of the γδ T cells. The γδ T cells can be engineered to provide a universal allogeneic therapy that recognizes an antigen of choice in vivo. Genetic engineering of the γδ T-cells may comprise stably integrating a construct expressing a tumor recognition moiety, such as αβ TCR, γδ TCR, chimeric antigen receptor (CAR), which combines both antigen-binding and T-cell activating functions into a single receptor, an antigen binding fragment thereof, or a lymphocyte activation domain into the genome of the isolated γδ T-cell(s), a cytokine (for example, IL-15, IL-12, IL-2. IL-7. IL-21, IL-18, IL-19, IL-33, IL-4, IL-9, IL-23, or IL1β) to enhance T-cell proliferation, survival, and function ex vivo and in vivo. Genetic engineering of the isolated γδ T-cell may also include deleting or disrupting gene expression from one or more endogenous genes in the genome of the isolated γδ T-cells, such as the MHC locus (loci).

Engineered (or transduced) T cells, including γδ T cells, can be expanded ex vivo without stimulation by an antigen presenting cell or aminobisphosphonate. Antigen reactive engineered T cells of the present disclosure may be expanded ex vivo and in vivo. In embodiments, an active population of engineered T cells may be expanded ex vivo without antigen stimulation by an antigen presenting cell, an antigenic peptide, a non-peptide molecule, or a small molecule compound, such as an aminobisphosphonate but using certain antibodies, cytokines, mitogens, or fusion proteins, such as IL-17 Fc fusion, MICA Fc fusion, and CD70 Fc fusion. Examples of antibodies that can be used in the expansion of a γδ T-cell population include anti-CD3, anti-CD27, anti-CD30, anti-CD70, anti-OX40, anti-NKG2D, or anti-CD2 antibodies, examples of cytokines may comprise IL-2, IL-15, IL-12, IL-21, IL-18, IL-9, IL-7, and/or IL-33, and examples of mitogens may comprise CD70 the ligand for human CD27, phytohaemagglutinin (PHA), concavalin A (ConA), pokeweed mitogen (PWM), protein peanut agglutinin (PNA), soybean agglutinin (SBA), les culinaris agglutinin (LCA), Pisum sativum agglutinin (PSA), Helix pomatia agglutinin (HPA), Vicia graminea Lectin (VGA) or another suitable mitogen capable of stimulating T-cell proliferation.

A population of engineered T cells, including γδ T cells, can be expanded in less than 60 days, less than 48 days, less than 36 days, less than 24 days, less than 12 days, or less than 6 days. In embodiments, a population of engineered T cells can be expanded from about 7 days to about 49 days, about 7 days to about 42 days, from about 7 days to about 35 days, from about 7 days to about 28 days, from about 7 days to about 21 days, or from about 7 days to about 14 days. The T-cells may be expanded for between about 1 and 21 days. For example, the T-cells may be expanded for about at least 1, 2, 3, 4, 5, 6, 7, 8, 9, 10, 11, 12, 13, 14, 15, 16, 17, 18, 19, or 20 days.

In embodiments, the same methodology may be used to isolate, activate, and expand αβ T cells.

In embodiments, the same methodology may be used to isolate, activate, and expand γδ T cells.

### Vectors

Engineered cells may be generated using various methods, including those recognized in the literature. For example, a polynucleotide encoding an expression cassette that comprises a tumor recognition, or another type of recognition moiety, can be stably introduced into the T-cell by a transposon/transposase system or a viral-based gene transfer system, such as a lentiviral or a retroviral system, or another suitable method, such as transfection, electroporation, transduction, lipofection, calcium phosphate (CaPO₄), nanoengineered substances, such as Ormosil, viral delivery methods, including adenoviruses, retroviruses, lentiviruses, adeno-associated viruses, or another suitable method. A number of viral methods have been used for human gene therapy, such as the methods described in WO 1993/020221, the content of which is incorporated herein in its entirety. Non-limiting examples of viral methods that can be used to engineer cells may comprise γ-retroviral, adenoviral, lentiviral, herpes simplex virus, vaccinia virus, pox virus, or adeno-virus associated viral methods. A cell may comprise an αβ T cell, a γδ T cell, a natural killer cell, a natural killer T cell, a CD4+ T cell, CD8+ T cell, a CD4+/CD8+ cell, or any combination thereof.

Viruses used for transfection of cells include naturally occurring viruses as well as artificial viruses. Viruses may be either an enveloped or non-enveloped virus. Parvoviruses (such as AAVs) are examples of non-enveloped viruses. The viruses may be enveloped viruses. The viruses used for transfection of cells may be retroviruses and in particular lentiviruses. Viral envelope proteins that can promote viral infection of eukaryotic cells may comprise HIV-1 derived lentiviral vectors (LVs) pseudotyped with envelope glycoproteins (GPs) from the vesicular stomatitis virus (VSV-G), the modified feline endogenous retrovirus (RD114TR) (SEQ ID NO: 97), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; the content of which is incorporated herein by reference), RD114 env, chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235: 1269-1276; the content of which is incorporated herein by reference), baculovirus GP64 env (such as described in Wang et al. J. Virol. 81:10869-10878, 2007; the content of which is incorporated herein by reference), or GALV env (such as described in Merten et al., J. Virol. 79:834-840, 2005; the content of which is incorporated herein by reference), or derivatives thereof.

A single lentiviral cassette can be used to create a single lentiviral vector, expressing at least four individual monomer proteins of two distinct dimers from a single multi-cistronic mRNA so as to co-express the dimers on the cell surface. For example, the integration of a single copy of the lentiviral vector was sufficient to transform T cells to co-express TCRαβ and CD8αβ, optionally αβ T cells or γδ T cells.

Vectors may comprise a multi-cistronic cassette within a single vector capable of expressing more than one, more than two, more than three, more than four genes, more than five genes, or more than six genes, in which the polypeptides encoded by these genes may interact with one another or may form dimers. The dimers may be homodimers, e.g., two identical proteins forming a dimer, or heterodimers, e.g., two structurally different proteins forming a dimer.

Additionally, multiple vectors may be used to transfect cells with the constructs and sequences described herein. One or more vectors may comprise combinations of TCR transgene(s), dnTGFβRII polypeptide transgene(s), and CD8 transgene(s) in any order. As a non-limiting example, a first vector may comprise a transgene encoding a TCR, a second vector may comprise a transgene encoding a dnTGFβRII polypeptide, and a third vector may comprise a transgene encoding a CD8 polypeptide described herein, and the vectors may be transfected into cells either simultaneously or sequentially in any order, using recognized methods. As another non-limiting example, a single vector may encode two transgenes in any order, or a single vector may encode three or more transgenes in any order. As another non-limiting example, a cell line that is stably transfected with one or more transgene(s) may then be transfected with one or more other transgene(s). In embodiments, a CD8 polypeptide may comprise a CD8α chain and/or a CD8β chain, and the CD8α chain and/or CD8β chain may independently be modified or unmodified.

One or more vector may comprise a nucleic acid encoding a dnTGFβRII polypeptide. One or more vector may comprise a nucleic acid encoding a CD8 polypeptide. One or more vector may comprise a nucleic acid encoding a CD8α polypeptide. One or more vector may comprise a nucleic acid encoding a CD8β polypeptide. In embodiments, a CD8 polypeptide may comprise a CD8α chain and/or a CD8β chain, and the CD8α chain and/or CD8β chain may independently be modified or unmodified.

One or more vector may comprise a nucleic acid encoding a T cell receptor (TCR) comprising an α chain and a β chain. One or more vector may comprise a nucleic acid encoding a T cell receptor (TCR) comprising an γ chain and a δ chain. One or more vector may comprise a nucleic acid encoding a chimeric antigen receptor (CAR).

More than one vector may comprise a nucleic acid or nucleic acids encoding one or any combination of a dnTGFβRII polypeptide, a CD8 polypeptide, a TCR comprising an α chain and a β chain, a TCR comprising an γ chain and a δ chain, and/or a CAR. In embodiments, a CD8 polypeptide may comprise a CD8α chain and/or a CD8β chain, and the CD8α chain and/or CD8β chain may independently be modified or unmodified.

A single vector may comprise a nucleic acid or nucleic acids encoding one or any combination of a dnTGFβRII polypeptide, a CD8 polypeptide, a TCR comprising an α chain and a β chain, a TCR comprising an γ chain and a δ chain, and/or a CAR. In embodiments, a CD8 polypeptide may comprise a CD8α chain and/or a CD8β chain, and the CD8α chain and/or CD8β chain may independently be modified or unmodified.

As used herein, the term “cistron” refers to a section of a nucleic acid molecule that specifies the formation of one polypeptide chain, i.e. coding for one polypeptide chain. For example, “mono-cistron” refers to one section of a nucleic acid molecule that specifies the formation of one polypeptide chain, i.e. coding for one polypeptide chain; “bi-cistron” refers to two sections of a nucleic acid molecule that specify the formation of two polypeptide chains, i.e. coding for two polypeptide chains; “tri-cistron” refers to three sections of a nucleic acid molecule that specify the formation of three polypeptide chains, i.e. coding for three polypeptide chains; etc.; “multicistron” refers two or more sections of a nucleic acid molecule that specify the formation of two or more polypeptide chains, i.e. coding for two or more polypeptide chains.

As used herein, the term “arranged in tandem” refers to the arrangement of the genes contiguously, one following or behind the other, in a single file on a nucleic acid sequence. The genes are ligated together contiguously on a nucleic acid sequence, with the coding strands (sense strands) of each gene ligated together on a nucleic acid sequence.

A transgene may further include one or more multicistronic element(s) and the multicistronic element(s) may be positioned, as non-limiting examples, between any, some, or each of a nucleic acid encoding a TCRα or a portion thereof, a nucleic acid encoding a TCRβ or a portion thereof, a nucleic acid encoding a CD8α or a portion thereof, a nucleic acid encoding a CD8β or a portion thereof, and/or a nucleic acid encoding a dnTGFβRII polypeptide or a portion thereof. The multicistronic element(s) may be positioned, as non-limiting examples, between any two nucleic acid sequences encoding of TCRα, TCRβ, CD8α, CD8β, and/or dnTGFβRII polypeptide, and these coding sequences may be in any order. The multicistronic element(s) may include a sequence encoding a ribosome skip element selected from among a T2A, a P2A, a E2A or a F2A or an internal ribosome entry site (IRES).

As used herein, the term “self-cleaving 2A peptide” refers to relatively short peptides (of the order of 20 amino acids long, depending on the virus of origin) acting co-translationally, by preventing the formation of a normal peptide bond between the glycine and last proline, resulting in the ribosome skipping to the next codon, and the nascent peptide cleaving between the Gly and Pro. After cleavage, the short 2A peptide remains fused to the C-terminus of the ‘upstream’ protein, while the proline is added to the N-terminus of the ‘downstream’ protein. Self-cleaving 2A peptide may be selected from porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, the content of which including 2A nucleic acid and amino acid sequences are incorporated herein by reference in their entireties). By adding one or more linker sequences (such as, but not limited to, GSG, SGSG (SEQ ID NO: 266)) before the self-cleaving 2A sequence, this may enable efficient synthesis of biologically active proteins, e.g., TCRs.

As used herein, the term “internal ribosome entry site (IRES)” refers to a nucleotide sequence located in a messenger RNA (mRNA) sequence, which can initiate translation without relying on the 5′ cap structure. IRES is usually located in the 5′ untranslated region (5′UTR) but may also be located in other positions of the mRNA. In embodiments IRES may be selected from IRES from viruses, IRES from cellular mRNAs, in particular IRES from picornavirus, such as polio, EMCV and FMDV, flavivirus, such as hepatitis C virus (HCV), pestivirus, such as classical swine fever virus (CSFV), retrovirus, such as murine leukaemia virus (MLV), lentivirus, such as simian immunodeficiency virus (SIV), and insect RNA virus, such as cricket paralysis virus (CRPV), and IRES from cellular mRNAs, e.g. translation initiation factors, such as eIF4G, and DAPS, transcription factors, such as c-Myc, and NF-κB-repressing factor (NRF), growth factors, such as vascular endothelial growth factor (VEGF), fibroblast growth factor 2 (FGF-2), platelet-derived growth factor B (PDGF-B), homeotic genes, such as antennapedia, survival proteins, such as X-linked inhibitor of apoptosis (XIAP), and Apaf-1, and other cellular mRNA, such as BiP.

Constructs and vectors described herein may be used with the methodology described in U.S. Patent Application Publication No. 2019/0175650, published on Jun. 13, 2019, the contents of which are incorporated by reference in their entirety.

In embodiments, a vector may further comprise a post-transcriptional regulatory element (PRE) sequence. In embodiments, the post-transcriptional regulatory element (PRE) sequence may be selected from a Woodchuck hepatitis virus PRE (WPRE) (such as, but not limited to wild type WPRE, such as but not limited to SEQ ID NO: 264, or a mutated WPRE, such as but not limited to WPREmut1 (SEQ ID NO: 256) or WPREmut2 (SEQ ID NO: 257)) or a hepatitis B virus (HBV) PRE (HPRE) (SEQ ID NO: 366), or variant(s) thereof, or any combination thereof.

In embodiments, a vector may further comprise one or more promoter. In embodiments, the promoter(s) may be selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, Murine Stem Cell Virus (MSCV) promoter, the promoter from CD69, nuclear factor of activated T-cells (NFAT) promoter, IL-2 promoter, minimal IL-2 promoter, or a combination thereof.

In embodiments, a vector may comprise one or more Kozak sequence. In embodiments, the Kozak sequence may be GCCACC. In embodiments, the Kozak sequence may be ACCATGG. In embodiments, the Kozak sequence may be GCCNCCATGG, where N is a purine (A or G) (SEQ ID NO:365).

In embodiments, a vector may comprise one or more Factor Xa sites.

In embodiments, a vector may comprise one or more enhancer. In embodiments, the enhancer may comprise Conserved Non-Coding Sequence (CNS) 0, CNS 1, CNS2, CNS 3, CNS 4, or portions or combinations thereof.

In embodiments, a vector may be a viral vector or a non-viral vector.

In embodiments, a vector may be selected from adenoviruses, poxviruses, alphaviruses, arenaviruses, flaviviruses, rhabdoviruses, retroviruses, lentiviruses, herpesviruses, paramyxoviruses, picornaviruses, or a combination thereof.

In embodiments, a vector may be pseudotyped with an envelope protein of a virus selected from the native feline endogenous virus (RD114), a chimeric version of RD114 (RD114TR), gibbon ape leukemia virus (GALV), a chimeric version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), lymphocytic choriomeningitis virus (LCMV), or a combination thereof.

Non-viral vectors may also be used with the sequences, constructs, and cells described herein.

Cells may be transfected by other means known in the art including lipofection (liposome-based transfection), electroporation, calcium phosphate transfection, biolistic particle delivery (e.g., gene guns), microinjection, or combinations thereof. Various methods of transfecting cells are known in the art. See, e.g., Sambrook & Russell (Eds.) Molecular Cloning: A Laboratory Manual (3^{rd} Ed.) Volumes 1-3 (2001) Cold Spring Harbor Laboratory Press; Ramamoorth & Narvekar “Non Viral Vectors in Gene Therapy—An Overview.” J Clin Diagn Res. (2015) 9(1): GE01-GE06.

### Gene Editing

In embodiments, transgenes (e.g., transgene(s) encoding CD8 α chain and/or β chain, transgene(s) encoding TCR α chain and/or β chain, and/or transgene(s) encoding dominant negative TGFβRII polypeptide may be inserted into a cell(s) using gene addition, gene editing, gene replacement, and/or gene transfer techniques, such as but not limited to knock-in techniques, such as but not limited to targeted knock-in techniques. Cells may be, as non-limiting examples, T cells or natural killer cells or combinations thereof. T cells may be, as non-limiting examples, αβ T cells, γδ T cells, natural killer T cells, CD4+ cells, CD8+ cells, CD4+/CD8+ cells, or combinations thereof. As non-limiting examples, techniques such as Clustered Regularly Interspaced Short Palindromic Repeats (CRISPR) systems (using, as non-limiting examples, Cas9, Cas12, Cas12a, Cas12a2, and/or Cas13), transcription-activator-like effector nuclease (TALEN) systems, and/or transposon-based systems (see, e.g., US Patent Publication No. 2019/0169637, which is incorporated herein in its entirety). Non-limiting examples of transposon-based systems include Sleeping Beauty (see, e.g, U.S. Pat. Nos. 7,985,739; 6,613,752; and 9,228,180 and US Patent Publication Nos. 2005/0003542; 2004/0092471; 2002/0103152; 2016/0264949; 2018/0135032; 2011/0117072; 2019/0169638; 2005/0112764; 2017/0029774; 2021/0139583, each of which is incorporated herein in its entirety), piggyBac (see, e.g., U.S. Pat. Nos. 10,287,559; 11,186,847; 10,131,885; 9,546,382; 8,399,643; 8,592,211; 6,962,810; 7,105,343; and 6,551,825 and US Patent Publication Nos. 2018/0142219; 2017/0166874; 2016/0160235; 2020/0087635; 2018/0195086; 2013/0160152; 2010/0287633; 2022/0064610; 2009/0042297; 2002/0173634; and 2017/0226531, each of which is incorporated herein in its entirety), and/or TcBuster systems (see, e.g., U.S. Pat. Nos. 11,278,570; 11,162,084; and 11,111,483 and US Patent Publication Nos. 2021/0277366; 2020/0339965; and 2020/0323902, each of which is incorporated herein in its entirety)).

### Compositions

Compositions may comprise a dnTGFβRII polypeptide and/or a CD8 polypeptides described herein and/or a TCR described herein. Further, compositions described herein may comprise a T-cell and/or a natural killer cell expressing a dnTGFβRII polypeptide and/or CD8 polypeptides described herein. The compositions described herein may comprise a T-cell and/or a natural killer cell expressing a dnTGFβRII polypeptide and/or CD8 polypeptides described herein and a T-cell and/or a natural killer cell receptor (TCR), optionally a TCR that specifically binds one of the TAA described herein complexed with an antigen presenting protein, e.g., MHC, referred to as HLA in humans, for human leukocyte antigen. In embodiments, a CD8 polypeptide may comprise a CD8α chain and/or a CD8β chain, and the CD8α chain and/or CD8β chain may independently be modified or unmodified.

To facilitate administration, the T cells and/or natural killer cells described herein can be made into a pharmaceutical composition or made into an implant appropriate for administration in vivo, with pharmaceutically acceptable carriers or diluents. The means of making such a composition or an implant are described in the art. See, e.g., Remington's Pharmaceutical Sciences, 16th Ed., Mack, ed. (1980).

The T cells and/or natural killer cells described herein can be formulated into a preparation in semisolid or liquid form, such as a capsule, solution, infusion, or injection. Means known in the art can be utilized to prevent or minimize release and absorption of the composition until it reaches the target tissue or organ, or to ensure timed-release of the composition. Desirably, however, a pharmaceutically acceptable form is employed that does not hinder the cells from expressing the CARs or TCRs. Thus, desirably the T cells and/or natural killer cells described herein can be made into a pharmaceutical composition comprising a carrier. The T cells and/or natural killer cells described herein can be formulated with a physiologically acceptable carrier or excipient to prepare a pharmaceutical composition. The carrier and composition can be sterile. Carriers include, for example, a balanced salt solution, such as Hanks' balanced salt solution, or normal saline. The formulation should suit the mode of administration. Suitable pharmaceutically acceptable carriers include but are not limited to water, salt solutions (e.g., NaCl), saline, buffered saline, as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, that do not deleteriously react with the T-cells and/or natural killer cells. The T-cells and/or natural killer cells may be αβ T cells or γδ T cells that express a dnTGFβRII polypeptide and/or CD8 polypeptides described herein, optionally a TCR described herein. In embodiments, a CD8 polypeptide may comprise a CD8α chain and/or a CD8β chain, and the CD8α chain and/or CD8β chain may independently be modified or unmodified.

A composition of the present disclosure can be provided in unit dosage form wherein each dosage unit, e.g., an injection, contains a predetermined amount of the composition, alone or in appropriate combination with other active agents.

The compositions described herein may be a pharmaceutical composition. Pharmaceutical composition described herein may further comprise an adjuvant selected from the group consisting of colony-stimulating factors, including but not limited to Granulocyte Macrophage Colony Stimulating Factor (GM-CSF, sargramostim), cyclophosphamide, imiquimod, resiquimod, interferon-alpha, or any combination thereof.

Pharmaceutical compositions described herein may comprise an adjuvant selected from the group consisting of colony-stimulating factors, e.g., Granulocyte Macrophage Colony Stimulating Factor (GM-CSF, sargramostim), cyclophosphamide, imiquimod and resiquimod.

Adjuvants include but are not limited to cyclophosphamide, imiquimod or resiquimod. Other exemplary adjuvants include Montanide IMS 1312, Montanide ISA 206, Montanide ISA 50V, Montanide ISA-51, poly-ICLC (Hiltonol®) and anti-CD40 mAB, or combinations thereof.

Other examples for useful adjuvants include, but are not limited to, chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present disclosure can readily be determined by the skilled artisan without undue experimentation.

Other adjuvants include but are not limited to anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), Polyinosinic-polycytidylic acid-poly-1-lysine carboxymethylcellulose (poly-ICLC), virosomes, and/or interleukin-1 (IL-1), IL-2, IL-4, IL-7, IL-12, IL-13, IL-15, IL-18, IL-21, and IL-23. See, e.g., Narayanan et al. J. Med. Chem. (2003) 46(23): 5031-5044; Pohar et al. Scientific Reports 7 14598 (2017); Grajkowski et al. Nucleic Acids Research (2005) 33(11): 3550-3560; Martins et al. Expert Rev Vaccines (2015) 14(3): 447-59.

The compositions described herein may also include one or more adjuvants.

Adjuvants are substances that non-specifically enhance or potentiate the immune response (e.g., immune responses mediated by CD8-positive T cells and helper-T (TH) cells to an antigen and would thus be considered useful in the medicament of the present disclosure). Suitable adjuvants include, but are not limited to, 1018 ISS, aluminium salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. In embodiments, the adjuvant may be Freund's or GM-CSF. Several immunological adjuvants (e.g., MF59) specific for dendritic cells and their preparation have been described previously. Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, incorporated herein by reference in its entirety) and acting as immunoadjuvants (e.g., IL-12, IL-15, IL-23, IL-7, IFN-alpha. IFN-beta).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany). In embodiments, dSLIM may be a component of a pharmaceutical composition described herein. Other TLR binding molecules such as RNA binding TLR 7, TLR 8 and/or TLR 9 may also be used.

### Methods of Treatment and Preparation

Engineered T cells and/or engineered natural killer (NK) cells may express a dnTGFβRII polypeptide(s) and/or CD8 polypeptide(s) described herein. Further, engineered T cells and/or engineered natural killer (NK) cells may express a TCR described herein. The TCR expressed by the engineered T cells and/or engineered natural killer (NK) cells may recognize a TAA bound to an HLA as described herein. Engineered T cells and/or engineered natural killer (NK) cells of the present disclosure can be used to treat a subject in need of treatment for a condition, for example, a cancer described herein. The cells may be αβ T cells, γδ T cells, and/or natural killer (NK) cells that express a dnTGFβRII polypeptide and/or a CD8 polypeptide, and optionally a TCR described herein. In embodiments, a CD8 polypeptide may comprise a CD8α chain and/or a CD8β chain, and the CD8α chain and/or CD8β chain may independently be modified or unmodified.

A method of treating a condition (e.g., ailment) in a subject with T cells and/or natural killer (NK) cells described herein may comprise administering to the subject a therapeutically effective amount of engineered T cells and/or engineered natural killer (NK) cells described herein, optionally γδ T cells. T cells and/or natural killer (NK) cells described herein may be administered at various regimens (e.g., timing, concentration, dosage, spacing between treatment, and/or formulation). A subject can also be preconditioned with, for example, chemotherapy, radiation, or a combination of both, prior to receiving engineered T cells and/or engineered natural killer (NK) cells of the present disclosure. A population of engineered T cells and/or engineered natural killer (NK) cells may also be frozen or cryopreserved prior to being administered to a subject. A population of engineered T cells and/or engineered natural killer (NK) cells can include two or more cells that express identical, different, or a combination of identical and different tumor recognition moieties. For instance, a population of engineered T-cells and/or engineered natural killer (NK) cells can include several distinct engineered T cells and/or engineered natural killer (NK) cells that are designed to recognize different antigens, or different epitopes of the same antigen. The cells may be αβ T cells, γδ T cells, and/or natural killer (NK) cells that express a dnTGFβRII polypeptide and/or a CD8 polypeptide described herein, and optionally a TCR described herein. In embodiments, a CD8 polypeptide may comprise a CD8α chain and/or a CD8β chain, and the CD8α chain and/or CD8β chain may independently be modified or unmodified.

T cells and/or natural killer (NK) cells described herein, including αβ T-cells and γδ T cells, may be used to treat various conditions. The cells may be αβ T cells, γδ T cells, and/or natural killer (NK) cells that express a dnTGFβRII polypeptide and/or a CD8 polypeptide, and optionally a TCR described herein. In embodiments, a CD8 polypeptide may comprise a CD8α chain and/or a CD8β chain, and the CD8α chain and/or CD8β chain may independently be modified or unmodified. T cells and/or natural killer (NK) cells described herein may be used to treat a cancer, including solid tumors and hematologic malignancies. Non-limiting examples of cancers include: non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

The T cells and/or natural killer (NK) cells described herein may be used to treat an infectious disease. The T cells and/or natural killer (NK) cells described herein may be used to treat an infectious disease, an infectious disease may be caused a virus. The T cells and/or natural killer (NK) cells described herein may be used to treat an immune disease, such as an autoimmune disease. The cells may be αβ T cells, γδ T cells, and/or natural killer (NK) cells that express a dnTGFβRII polypeptide and/or a CD8 polypeptide, and optionally a TCR described herein. In embodiments, a CD8 polypeptide may comprise a CD8α chain and/or a CD8β chain, and the CD8α chain and/or CD8β chain may independently be modified or unmodified.

Treatment with T cells and/or natural killer (NK) cells described herein, optionally γδ T cells, may be provided to the subject before, during, and after the clinical onset of the condition. Treatment may be provided to the subject after about 1 day, about 1 week, about 6 months, about 12 months, or about 2 years after clinical onset of the disease. Treatment may be provided to the subject for more than about 1 day, about 1 week, about 1 month, about 6 months, about 12 months, about 2 years, about 3 years, about 4 years, about 5 years, about 6 years, about 7 years, about 8 years, about 9 years, about 10 years or more after clinical onset of disease. Treatment may be provided to the subject for less than about 1 day, about 1 week, about 1 month, about 6 months, about 12 months, or about 2 years after clinical onset of the disease. Treatment may also include treating a human in a clinical trial. A treatment can include administering to a subject a pharmaceutical composition comprising engineered T cells and/or engineered natural killer (NK) cells described herein. The cells may be αβ T cells, γδ T cells, and/or natural killer (NK) cells that express a dnTGFβRII polypeptide and/or a CD8 polypeptide, and optionally a TCR described herein. In embodiments, a CD8 polypeptide may comprise a CD8α chain and/or a CD8β chain, and the CD8α chain and/or CD8β chain may independently be modified or unmodified.

In embodiments, administration of engineered T cells and/or engineered natural killer (NK) cells of the present disclosure to a subject may modulate the activity of endogenous lymphocytes in a subject's body. In embodiments, administration of engineered T cells and/or engineered natural killer (NK) cells to a subject may provide an antigen to an endogenous T-cell and may boost an immune response. In embodiments, the memory T cell may be a CD4+ T-cell. In embodiments, the memory T cell may be a CD8+ T-cell. In embodiments, administration of engineered T cells and/or engineered natural killer (NK) cells of the present disclosure to a subject may activate the cytotoxicity of another immune cell. In embodiments, the other immune cell may be a CD8+ T-cell. In embodiments, the other immune cell may be a Natural Killer T-cell. In embodiments, administration of engineered γδ T-cells and/or engineered natural killer (NK) cells of the present disclosure to a subject may suppress a regulatory T-cell. In embodiments, the regulatory T-cell may be a FOX3+ Treg cell. In embodiments, the regulatory T-cell may be a FOX3− Treg cell. Non-limiting examples of cells whose activity can be modulated by engineered T cells and/or engineered natural killer (NK) cells of the disclosure may comprise: hematopoietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells. The T cells may be αβ T cells or γδ T cells that express a dnTGFβRII polypeptide and/or a CD8 polypeptide, and optionally a TCR described herein. In embodiments, a CD8 polypeptide may comprise a CD8α chain and/or a CD8β chain, and the CD8α chain and/or CD8β chain may independently be modified or unmodified.

During most bone marrow transplants, a combination of cyclophosphamide with total body irradiation may be conventionally employed to prevent rejection of the hematopoetic stem cells (HSC) in the transplant by the subject's immune system. In embodiments, incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of γδ T-cells into humans may require the co-administration of γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. In embodiments, the disclosure provides a method for administrating engineered T cells and/or engineered natural killer (NK) cells to a subject without the co-administration of a native cytokine or modified versions thereof, such as IL-2, IL-15, IL-12, IL-21. In embodiments, engineered T cells and/or engineered natural killer (NK) cells can be administered to a subject without co-administration with IL-2. In embodiments, engineered T cells and/or engineered natural killer (NK) cells may be administered to a subject during a procedure, such as a bone marrow transplant without the co-administration of IL-2.

In embodiments, the methods may further comprise administering a chemotherapy agent. The dosage of the chemotherapy agent may be sufficient to deplete the patient's T-cell population. The chemotherapy may be administered about 5-7 days prior to administration of T-cells and/or natural killer (NK) cells. The chemotherapy agent may be cyclophosphamide, fludarabine, or a combination thereof. The chemotherapy agent may comprise dosing at about 400-600 mg/m²/day of cyclophosphamide. The chemotherapy agent may comprise dosing at about 10-30 mg/m²/day of fludarabine.

In embodiments, the methods may further comprise pre-treatment of the patient with low-dose radiation prior to administration of the composition comprising T-cells. The low dose radiation may comprise about 1.4 Gy for 1-6 days, such as about 5 days, prior to administration of the composition comprising T-cells.

In embodiments, the patient may be HLA-A*02.

In embodiments, the patient may be HLA-A*06.

In embodiments, the methods may further comprise administering an anti-PD1 antibody. The anti-PD1 antibody may be a humanized antibody. The anti-PD1 antibody may be pembrolizumab. The dosage of the anti-PD1 antibody may be about 200 mg. The anti-PD1 antibody may be administered every 3 weeks following administration of T cells and/or natural killer (NK) cells.

In embodiments, the dosage of T-cells and/or natural killer (NK) cells may be between about 0.8-1.2×10⁹ T cells and/or natural killer (NK) cells. The dosage of the T cells and/or natural killer (NK) cells may be about 0.5×10⁸ to about 10×10⁹ T cells and/or natural killer (NK) cells. The dosage of T-cells and/or natural killer (NK) cells may be about 1.2-3×10⁹ T cells and/or natural killer (NK) cells, about 3-6×10⁹ T cells and/or natural killer (NK) cells, about 10×10⁹ T cells and/or natural killer (NK) cells, about 5×10⁹ T cells and/or natural killer (NK) cells, about 0.1×10⁹ T cells and/or natural killer (NK) cells, about 1×10⁸ T cells and/or natural killer (NK) cells, about 5×10⁸ T cells and/or natural killer (NK) cells, about 1.2-6×10⁹ T cells and/or natural killer (NK) cells, about 1-6×10⁹ T cells and/or natural killer (NK) cells, or about 1-8×10⁹ T cells and/or natural killer (NK) cells.

In embodiments, the T cells and/or natural killer (NK) cells may be administered in 3 doses. The T-cell doses may escalate with each dose. The T-cells and/or natural killer (NK) cells may be administered by intravenous infusion.

In embodiments, the dnTGFβRII and/or CD8 sequences described herein and associated products and compositions may be used autologous or allogenic methods of adoptive cellular therapy. In embodiments, dnTGFβRII sequences, CD8 sequences, T cells and/or natural killer (NK) cells thereof, and compositions may be used in, for example, methods described in U.S. Patent Application Publication 2019/0175650; U.S. Patent Application Publication 2019/0216852; U.S. Patent Application Publication 2019/024743; and U.S. Provisional Patent Application 62/980,844, each of which is incorporated by reference in its entirety.

The disclosure also provides for a population of modified T cells and/or natural killer (NK) cells that express a dnTGFβRII polypeptide and/or present an exogenous CD8 polypeptide described herein and a T cell receptor wherein the population of modified T cells is activated and expanded with a combination of IL-2 and IL-15. In embodiments, the population of modified T cells and/or natural killer (NK) cells are expanded and/or activated with a combination of IL-2, IL-15, and zoledronate. In embodiments, the population of modified T cells and/or natural killer (NK) cells are activated with a combination of IL-2, IL-15, and zoledronate while expanded with a combination of IL-2, IL-15, and without zoledronate. The disclosure further provides for use of other interleukins during activation and/or expansion, such as IL-12, IL-18, IL-21, and any combination thereof.

In an aspect, IL-21, a histone deacetylase inhibitor (HDACi), or any combination thereof may be utilized in the field of cancer treatment, with methods described herein, and/or with ACT processes described herein. In embodiments, the present disclosure provides methods for re-programming effector T cells to a central memory phenotype comprising culturing the effector T cells with at least one HDACi together with IL-21. Representative HDACi include, for example, trichostatin A, trapoxin B, phenylbutyrate, valproic acid, vorinostat (suberanilohydroxamic acid), belinostat, panobinostat, dacinostat, entinostat, tacedinaline, and mocetinostat.

Compositions comprising engineered T cells and/or natural killer (NK) cells described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. An engineered T-cell and/or engineered natural killer (NK) cell can also be administered to lessen a likelihood of developing, contracting, or worsening a condition. Effective amounts of a population of engineered T-cells and/or engineered natural killer (NK) cells for therapeutic use can vary based on the severity and course of the disease or condition, previous therapy, the subject's health status, weight, and/or response to the drugs, and/or the judgment of the treating physician. The cells may be αβ T cells, γδ T cells, and/or natural killer (NK) cells engineered to express a dnTGFβRII polypeptide and/or a modified or unmodified CD8 polypeptides described herein and optionally a TCR described herein.

### Methods of Administration

One or multiple engineered T cell and/or natural killer (NK) cell populations described herein may be administered to a subject in any order or simultaneously. If simultaneously, the multiple engineered T cell and/or natural killer (NK) cell can be provided in a single, unified form, such as an intravenous injection, or in multiple forms, for example, as multiple intravenous infusions, subcutaneous injections or pills. Engineered T-cells and/or engineered natural killer (NK) cells can be packed together or separately, in a single package or in a plurality of packages. One or all of the engineered T cells and/or engineered natural killer (NK) cells can be given in multiple doses. If not simultaneous, the timing between the multiple doses may vary to as much as about a week, about a month, about two months, about three months, about four months, about five months, about six months, or about a year. In embodiments, engineered T cells and/or engineered natural killer (NK) cells can expand within a subject's body, in vivo, after administration to a subject. Engineered T cells and/or engineered natural killer (NK) cells can be frozen to provide cells for multiple treatments with the same cell preparation. Engineered T cells and/or engineered natural killer (NK) cells of the present disclosure, and pharmaceutical compositions comprising the same, can be packaged as a kit. A kit may comprise instructions (e.g., written instructions) on the use of engineered T cells and/or engineered natural killer (NK) cells and compositions comprising the same.

A method of treating a cancer may comprise administering to a subject a therapeutically-effective amount of engineered T cells and/or engineered natural killer (NK) cells, in which the administration treats the cancer. In embodiments, the therapeutically-effective amount of engineered γδ T cells and/or engineered natural killer (NK) cells may be administered for at least about 10 seconds, about 30 seconds, about 1 minute, about 10 minutes, about 30 minutes, about 1 hour, about 2 hours, about 3 hours, about 4 hours, about 5 hours, about 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, or about 1 year. In embodiments, the therapeutically-effective amount of the engineered T cells and/or engineered natural killer (NK) cells may be administered for at least about one week. In embodiments, the therapeutically-effective amount of engineered T cells and/or engineered natural killer (NK) cells may be administered for at least about two weeks.

Engineered T-cells and/or engineered natural killer (NK) cells described herein, optionally γδ T cells, can be administered before, during, or after the occurrence of a disease or condition, and the timing of administering a pharmaceutical composition comprising an engineered T-cell can vary. For example, engineered T cells and/or engineered natural killer (NK) cells can be used as a prophylactic and can be administered continuously to subjects with a propensity to conditions or diseases in order to lessen the likelihood of occurrence of the disease or condition. Engineered T-cells and/or engineered natural killer (NK) cells can be administered to a subject during or as soon as possible after the onset of the symptoms. The administration of engineered T cells and/or engineered natural killer (NK) cells can be initiated immediately within the onset of symptoms, within the about first 3 hours of the onset of the symptoms, about within the first 6 hours of the onset of the symptoms, about within the first 24 hours of the onset of the symptoms, about within 48 hours of the onset of the symptoms, or within any period of time from the onset of symptoms. The initial administration can be via any route practical, such as by any route described herein using any formulation described herein. In embodiments, the administration of engineered T cells and/or engineered natural killer (NK) cells of the present disclosure may be an intravenous administration. One or multiple dosages of engineered T cells and/or engineered natural killer (NK) cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered T cells and/or engineered natural killer (NK) cells can be administered years after onset of the cancer and before or after other treatments. In embodiments, engineered γδ T cells and/or engineered natural killer (NK) cells can be administered for at least about 10 minutes, about 30 minutes, about 1 hour, about 2 hours, about 3 hours, about 4 hours, about 5 hours, about 6 hours, about 12 hours, about 24 hours, at least about 48 hours, at least about 72 hours, at least about 96 hours, at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years at least about 3 years, at least about 4 years, or at least about 5 years. The length of treatment can vary for each subject. The cells may be αβ T cells, γδ T cells, and/or natural killer (NK) cells that express a dnTGFβRII polypeptide and/or a CD8 polypeptide described herein, optionally a TCR described herein.

Engineered T-cells and/or engineered natural killer (NK) cells expressing a dnTGFβRII polypeptide and/or a CD8 polypeptides described herein, optionally αβ T cells and/or γδ T cells, may be present in a composition in an amount of at least about 1×10³ cells/ml, at least about 2×10³ cells/ml, at least about 3×10³ cells/ml, at least about 4×10³ cells/ml, at least about 5×10³ cells/ml, at least about 6×10³ cells/ml, at least about 7×10³ cells/ml, at least about 8×10³ cells/ml, at least about 9×10³ cells/ml, at least about 1×10⁴ cells/ml, at least about 2×10⁴ cells/ml, at least about 3×10⁴ cells/ml, at least about 4×10⁴ cells/ml, at least about 5×10⁴ cells/ml, at least about 6×10⁴ cells/ml, at least about 7×10⁴ cells/ml, at least about 8×10⁴ cells/ml, at least about 9×10⁴ cells/ml, at least about 1×10⁵ cells/ml, at least about 2×10⁵ cells/ml, at least about 3×10⁵ cells/ml, at least about 4×10⁵ cells/ml, at least about 5×10⁵ cells/ml, at least about 6×10⁵ cells/ml, at least about 7×10⁵ cells/ml, at least about 8×10⁵ cells/ml, at least about 9×10⁵ cells/ml, at least about 1×10⁶ cells/ml, at least about 2×10⁶ cells/ml, at least about 3×10⁶ cells/ml, at least about 4×10⁶ cells/ml, at least about 5×10⁶ cells/ml, at least about 6×10⁶ cells/ml, at least about 7×10⁶ cells/ml, at least about 8×10⁶ cells/ml, at least about 9×10⁶ cells/ml, at least about 1×10⁷ cells/ml, at least about 2×10⁷ cells/ml, at least about 3×10⁷ cells/ml, at least about 4×10⁷ cells/ml, at least about 5×10⁷ cells/ml, at least about 6×10⁷ cells/ml, at least about 7×10⁷ cells/ml, at least about 8×10⁷ cells/ml, at least about 9×10⁷ cells/ml, at least about 1×10⁸ cells/ml, at least about 2×10⁸ cells/ml, at least about 3×10⁸ cells/ml, at least about 4×10⁸ cells/ml, at least about 5×10⁸ cells/ml, at least about 6×10⁸ cells/ml, at least about 7×10⁸ cells/ml, at least about 8×10⁸ cells/ml, at least about 9×10⁸ cells/ml, at least about 1×10⁹ cells/ml, or more, from about 1×10³ cells/ml to about at least about 1×10⁸ cells/ml, from about 1×10⁵ cells/ml to about at least about 1×10⁸ cells/ml, or from about 1×10⁶ cells/ml to about at least about 1×10⁸ cells/ml. Uses

T cells, natural killer (NK) cells, and pharmaceutical compositions described herein may be used in therapy, in particular in a method of treating cancer. The present disclosure therefore also provides the use of the T cells, natural killer (NK) cells, and pharmaceutical compositions described herein in the therapy, in particular in a method of treating cancer. Further, the present disclosure also provides the use of the T cells, natural killer (NK) cells, and pharmaceutical compositions described herein in the manufacture of a medicament, in particular a medicament for the treatment of cancer. The cancer may be selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer. The features and aspects described in connection with the methods of treating, preparing and administering above are also applicable to the uses described herein, mutatis mutandis.

### Sequences

The sequences described herein may comprise about 80%, about 85%, about 90%, about 85%, about 96%, about 97%, about 98%, or about 99% or 100% identity to the sequence of any of SEQ ID NO: 1-97, 256-266, 293, or 305-365. The sequences described herein may comprise at least 80%, at least 85%, at least 90%, at least 85%, at least 96%, at least 97%, at least 98%, at least 99% or 100% identity to the sequence of any of SEQ ID NO: 1-97, 256-266, or, or 305-365. A sequence “at least 85% identical to a reference sequence” is a sequence having, on its entire length, 85%, or more, in particular 90%, 91%, 92%, 93%, 94%, 95%, 96%, 97%, 98%, 99%, or 100% sequence identity with the entire length of the reference sequence.

In embodiments, the disclosure provides for sequences at least 80%, at least about 85%, at least about 90%, at least about 85%, at least about 96%, at least about 97%, at least about 98%, at least about 99% or about 100% identity to WPREmut1 (SEQ ID NO: 256), or WPRE version 2, e.g., WPREmut2 (SEQ ID NO: 257). In another aspect, the disclosure provides for sequences at least 1, 2, 3, 4, 5, 10, 15, or 20 amino acid substitutions in WPREmut1 (SEQ ID NO: 256), or WPRE version 2, e.g., WPREmut2 (SEQ ID NO: 257). In yet another aspect, the disclosure provides for sequences at most 1, 2, 3, 4, 5, 10, 15, or 20 amino acid substitutions in WPREmut1 (SEQ ID NO: 256), or WPRE version 2, e.g., WPREmut2 (SEQ ID NO: 257). In another aspect, the sequence substitutions are conservative substitutions.

Percentage of identity may be calculated using a global pairwise alignment (e.g., the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the present disclosure, is calculated using the EMBOSS: needle (global) program with a “Gap Open” parameter equal to 10.0, a “Gap Extend” parameter equal to 0.5, and a Blosum62 matrix.

Proteins comprising or consisting of an amino acid sequence “at least 80%, 85%, 90%, 95%, 96%, 97%, 98% or 99% identical”, “at least about 80%, 85%, 90%, 95%, 96%, 97%, 98% or 99% identical”, or similar recitations, to a reference sequence may comprise mutations such as deletions, insertions and/or substitutions compared to the reference sequence. The reference sequence may be, as non-limiting examples, a wild type sequence, a mature wild type sequence, a native sequence, a truncated wild type sequence, a truncated mature wild type sequence, a truncated native sequence, or a sequence disclosed herein. The reference sequence may be, as non-limiting examples, a wild type sequence, a mature wild type sequence, or a native sequence. In the case of substitutions, the protein consisting of an amino acid sequence at least or at least about 80%, 85%, 90%, 95%, 96%, 97%, 98% or 99% identical to a reference sequence may correspond to a homologous sequence derived from another species than the reference sequence.

Amino acid substitutions may be conservative or non-conservative. In embodiments, substitutions are conservative substitutions, in which one amino acid is substituted for another amino acid with similar structural and/or chemical properties.

Conservative substitutions may comprise those, which are described by Dayhoff in “The Atlas of Protein Sequence and Structure. Vol. 5”, Natl. Biomedical Research, the contents of which are incorporated by reference in their entirety. For example, In embodiments, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In embodiments, a conservative amino acid substitution may be selected from the following of T→A, G→A, T→V, A→M, A→V, T→G, and/or T→S.

A conservative amino acid substitution may comprise the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

Conservative substitutions may be made in accordance with Table A. Methods for predicting tolerance to protein modification may be found in, for example, Guo et al., Proc. Natl. Acad. Sci., USA, 101(25):9205-9210 (2004), the contents of which are incorporated by reference in their entirety.

TABLE A
Conservative Amino Acid Substitution
Conservative Amino Acid Substitutions

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

The sequences described herein may comprise 1, 2, 3, 4, 5, 10, 15, 20, 25, or 30 amino acid or nucleotide mutations, substitutions, deletions. Any one of SEQ ID NO: 1-97, 256-266, 293, 294, and 305-365 may comprise 1, 2, 3, 4, 5, 10, 15, 20, 25, or 30 mutations, substitutions, or deletions. In another aspect, any one of SEQ ID NO: 1-97, 256-266, 293, 294, and 305-365 may comprise at most 1, 2, 3, 4, 5, 10, 15, 20, 25, or 30 mutations, substitutions, or deletions. In an aspect, the mutations or substitutions may be conservative amino acid substitutions.

Conservative substitutions in the polypeptides described herein may be those shown in Table B under the heading of “conservative substitutions.” If such substitutions result in a change in biological activity, then more substantial changes, denominated “exemplary substitutions” in Table B, may be introduced and the products screened if needed.

TABLE B
Amino Acid Substitution
Amino Acid Substitutions

| Original Residue | | |
|---|---|---|
| (naturally | | |
| occurring amino | Conservative | Exemplary |
| acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala, Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Nucleic acids comprising or consisting of a nucleic acid sequence “at least 80%, 85%, 90%, 95%, 96%, 97%, 98% or 99% identical”, “at least about 80%, 85%, 90%, 95%, 96%, 97%, 98% or 99% identical”, or similar recitations, to a reference sequence may comprise mutations such as deletions, insertions and/or substitutions compared to the reference sequence. The reference sequence may be, as non-limiting examples, a wild type sequence, a mature wild type sequence, a native sequence, a truncated wild type sequence, a truncated mature wild type sequence, a truncated native sequence, or a sequence disclosed herein. The reference sequence may be, as non-limiting examples, a wild type sequence, a mature wild type sequence, or a native sequence. Due, for example, to codon degeneracy, mutations or substitutions to a reference nucleic acid sequence may result in a mutated nucleic acid sequence that encodes protein identical to the protein encoded by the reference sequence. Mutated nucleic acid sequences that encode a protein having a different sequence from the protein encoded by the reference sequence are also contemplated. Mutated nucleic acid sequences encoding conservative amino acid mutations are contemplated. In the case of substitutions, the nucleic acid sequence at least, or at least about, 80%, 85%, 90%, 95%, 96%, 97%, 98% or 99% identical to a reference sequence may correspond to a homologous sequence derived from another species than the reference sequence.

Unless otherwise indicated, all terms used herein have the same meaning as they would to one skilled in the art.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific embodiments of this disclosure set forth in the appended claims. The foregoing embodiments are presented by way of example only; the scope of the present disclosure is to be limited only by the following claims.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention. Additional information regarding CD8 polypeptides, TCR polypeptides, and further information, may be found in U.S. patent application Ser. No. 17/563,599, filed Dec. 28, 2021, entitled “CD8 POLYPEPTIDES, COMPOSITIONS, AND METHODS OF USING THEREOF”, which is incorporated by reference herein in its entirety.

Unless otherwise specified herein, ranges of values set forth herein are intended to operate as a scheme for referring to each separate value falling within the range individually, including but not limited to the endpoints of the ranges, and each separate value of each range set forth herein is hereby incorporated into the specification as if it were individually recited.

This specification may include references to “one embodiment”, “an embodiment”, “embodiments”, “one aspect”, “an aspect”, or “aspects”. Each of these words and phrases is not intended to convey a different meaning from the other words and phrases. These words and phrases may refer to the same embodiment or aspect, may refer to different embodiments or aspects, and may refer to more than one embodiment or aspect. Various embodiments and aspects may be combined in any manner consistent with this disclosure.

“Activation” as used herein refers broadly to the state of a T cell that has been sufficiently stimulated to induce detectable cellular proliferation. Activation can also be associated with induced cytokine production, and detectable effector functions. The term “activated T cells” refers to, among other things, T cells that are proliferating.

“Antibodies” as used herein refer broadly to antibodies or immunoglobulins of any isotype, fragments of antibodies, which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins including an antigen-specific targeting region of an antibody and a non-antibody protein. Antibodies are organized into five classes—IgG, IgE, IgA, IgD, and IgM.

“Antigen” or “Antigenic,” as used herein, refers broadly to a peptide or a portion of a peptide capable of being bound by an antibody which is additionally capable of inducing an animal to produce an antibody capable of binding to an epitope of that antigen. An antigen may have one epitope or have more than one epitope. The specific reaction referred to herein indicates that the antigen will react, in a highly selective manner, with its corresponding antibody and not with the multitude of other antibodies which may be evoked by other antigens.

“Chimeric antigen receptor” or “CAR” or “CARs” as used herein refers broadly to genetically modified receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). In certain embodiments, the CSD is optional. In embodiments, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

“Cytotoxic T lymphocyte” (CTL) as used herein refers broadly to a T lymphocyte that expresses CD8 on the surface thereof (e.g., a CD8+ T cell). Such cells may be “memory” T cells (T_{M} cells) that are antigen-experienced.

“Effective amount”, “therapeutically effective amount”, or “efficacious amount” as used herein refers broadly to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

“Genetically modified” as used herein refers broadly to methods to introduce exogenous nucleic acids into a cell, whether or not the exogenous nucleic acids are integrated into the genome of the cell. “Genetically modified cell” as used herein refers broadly to cells that contain exogenous nucleic acids whether or not the exogenous nucleic acids are integrated into the genome of the cell.

“Immune cells” as used herein refers broadly to white blood cells (leukocytes) derived from hematopoietic stem cells (HSC) produced in the bone marrow “Immune cells” include, without limitation, lymphocytes (T cells, B cells, natural killer (NK) (CD3-CD56+) cells) and myeloid-derived cells (neutrophil, eosinophil, basophil, monocyte, macrophage, dendritic cells). “T cells” include all types of immune cells expressing CD3 including T-helper cells (CD4+ cells), cytotoxic T-cells (CD8+ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8+ T cells, natural-killer (NK) cells, NK-T cells, γδ T cells, and neutrophils, which are cells capable of mediating cytotoxicity responses.

“Individual,” “subject,” “host,” and “patient,” as used interchangeably herein, refer broadly to a mammal, including, but not limited to, humans, murines (e.g., rats, mice), lagomorphs (e.g., rabbits), non-human primates, canines, felines, and ungulates (e.g., equines, bovines, ovines, porcines, caprines).

“Peripheral blood mononuclear cells” or “PBMCs” as used herein refers broadly to any peripheral blood cell having a round nucleus. PBMCs include lymphocytes, such as T cells, B cells, and NK cells, and monocytes.

“Polynucleotide” and “nucleic acid”, as used interchangeably herein, refer broadly to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases.

“T cell” or “T lymphocyte,” as used herein, refer broadly to thymocytes, naïve T lymphocytes, immature T lymphocytes, mature T lymphocytes, resting T lymphocytes, or activated T lymphocytes. Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4−CD8− T cell, natural killer T cell, T cells expressing αβ TCR (αβ T cells), T cells expressing γδ TCR (γδ T cells), or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

In the present disclosure, the term “homologous” refers to the degree of identity between sequences of two amino acid sequences, e.g., peptide or polypeptide sequences. The aforementioned “homology” is determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases.

The terms “sequence homology” or “sequence identity” are used interchangeably herein. For the purpose of this disclosure, in order to determine the percentage of sequence homology or sequence identity of two amino acid sequences or of two nucleotide sequences, the sequences are aligned for optimal comparison purposes. In order to optimize the alignment between the two sequences, gaps may be introduced in any of the two sequences that are compared. Such alignment can be carried out over the full-length of the sequences being compared. Alternatively, the alignment may be carried out over a shorter length, for example over about 5, about 10, about 20, about 50, about 100 or more nucleotides or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison. In D. Sankoff and J. B. Kruskal, (ed.), Time warps, string edits and macromolecules: the theory and practice of sequence comparison, Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mal. Biol. 48, 443-453). Both amino acid sequences and nucleotide sequences can be aligned by the algorithm. The Needleman-Wunsch algorithm has been implemented in the computer program NEEDLE. For the purpose of this disclosure, the NEEDLE program from the EMBOSS package was used (version 2.8.0 or higher, EMBOSS: The European Molecular Biology Open Software Suite (2000) Rice, Longden, and Bleasby, Trends in Genetics 16, (6) 276-277, emboss.bioinformatics.nl/). For amino acid sequences, EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the present disclosure is calculated as follows: Number of corresponding positions in the alignment showing an identical amino acid or identical nucleotide in both sequences divided by the total length of the alignment after subtraction of the total number of gaps in the alignment. The identity can be obtained from NEEDLE by using the NOBRIEF option and is labelled in the output of the program as “longest-identity”. The nucleotide and amino acid sequences of the present disclosure can further be used as a “query sequence” to perform a search against sequence databases to, for example, identify other family members or related sequences. Such searches can be performed using the NBLAST and XBLAST programs (version 2.0) of Altschul et al. (1990) J. Mal. Biol. 215:403-10. BLAST nucleotide searches can be performed with the NBLAST program, score=100, word length=12 to obtain nucleotide sequences homologous to polynucleotides of the present disclosure. BLAST protein searches can be performed with the XBLAST program, score=50, word length=3 to obtain amino acid sequences homologous to polypeptides of the present disclosure. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

“T-cell receptor (TCR)” as used herein refers broadly to a protein receptor on T cells that is composed of a heterodimer of an alpha (α) and beta (β) chain, although in some cells the TCR consists of gamma and delta (γ/δ) chains. The TCR may be modified on any cell comprising a TCR, including a helper T cell, a cytotoxic T cell, a memory T cell, regulatory T cell, natural killer T cell, or a gamma delta T cell.

The TCR is generally found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3-γ, CD3δ, and two times CD3ε) in mammals, that associate with molecules known as the T-cell receptor (TCR) and the ζ-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

“Treatment,” “treating,” and the like, as used herein refer broadly to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

The ability of dendritic cells (DC) to activate and expand antigen-specific CD8+ T cells may depend on the DC maturation stage and that DCs may need to receive a “licensing” signal, associated with IL-12 production, in order to elicit cytolytic immune response. In particular, the provision of signals through CD40 Ligand (CD40L)-CD40 interactions on CD4+ T cells and DCs, respectively, may be considered important for the DC licensing and induction of cytotoxic CD8+ T cells. DC licensing may result in the upregulation of co-stimulatory molecules, increased survival and better cross-presenting capabilities of DCs. This process may be mediated via CD40/CD40L interaction [S. R. Bennet et al., “Help for cytotoxic T-cell responses is mediated by CD40 signalling,” Nature 393(6684):478-480 (1998); S. P. Schoenberger et al., “T-cell help for cytotoxic T-cell help is mediated by CD40-CD40L interactions,” Nature 393(6684):480-483 (1998)], but CD40/CD40L-independent mechanisms also exist (CD70, LTβR). In addition, a direct interaction between CD40L expressed on DCs and CD40 on expressed on CD8+ T-cells has also been suggested, providing a possible explanation for the generation of helper-independent CTL responses [S. Johnson et al., “Selected Toll-like receptor ligands and viruses promote helper-independent cytotoxic T-cell priming by upregulating CD40L on dendritic cells,” Immunity 30(2):218-227 (2009)].

### Example 1

### Exemplary Nucleic Acid and Amino Acid Sequences

TABLE 2
CD8-TCR Constructs

| | Nucleic Acid | Amino Acid |
|---|---|---|
| Construct # | (SEQ ID NO) | (SEQ ID NO) |
| 1 | 295 | 296 |
| 2 | 297 | 298 |
| 8 | 299 | 300 |
| 9 | 287 | 288 |
| 9b | 287 | 288 |
| 10 | 291 | 292 |
| 10n | 291 | 292 |
| 11 | 285 | 286 |
| 11n | 285 | 286 |
| 12 | 301 | 302 |
| 13 | 267 | 268 |
| 14 | 269 | 270 |
| 15 | 271 | 272 |
| 16 | 273 | 274 |
| 17 | 275 | 276 |
| 18 | 277 | 278 |
| 19 | 279 | 280 |
| 21 | 281 | 282 |
| 22 | 283 | 284 |
| 25 | 289 | 290 |

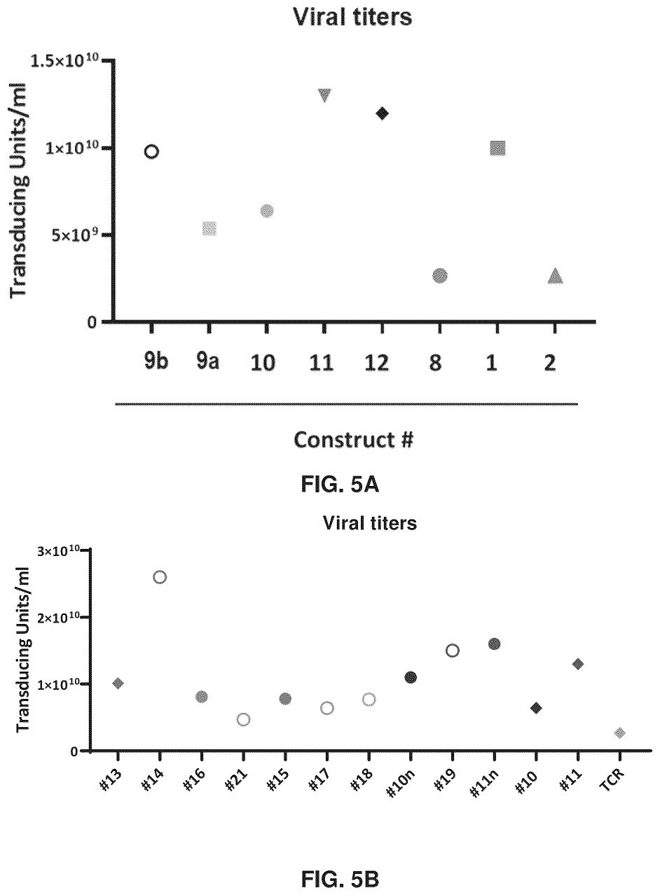

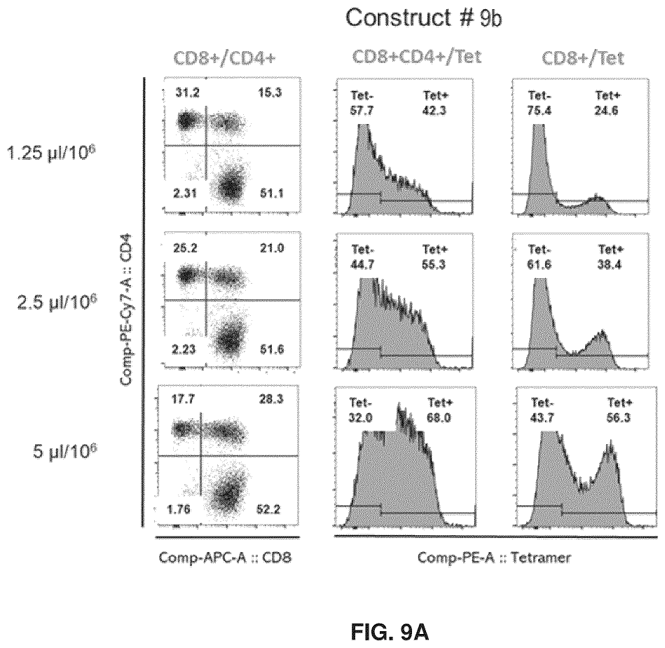

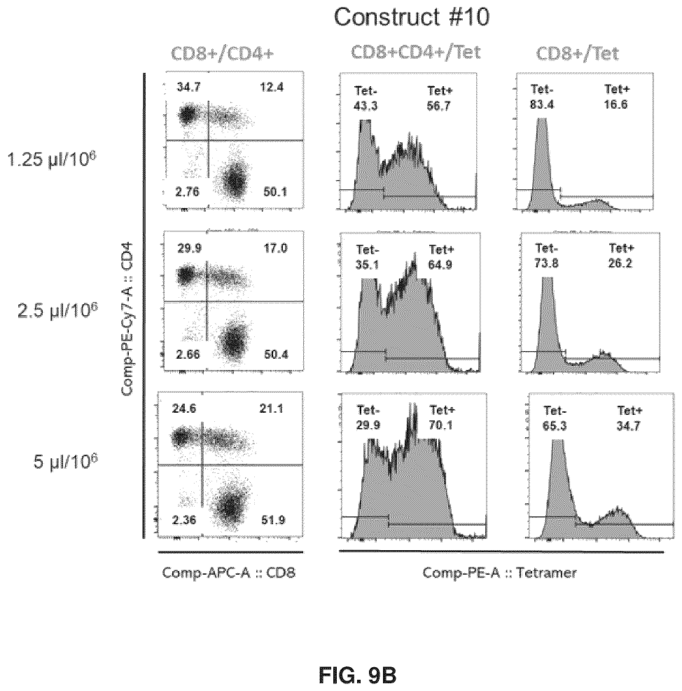

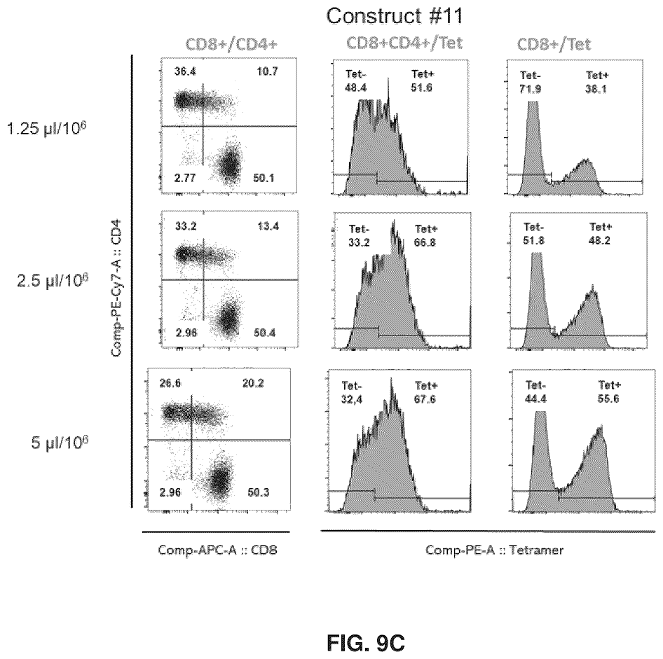

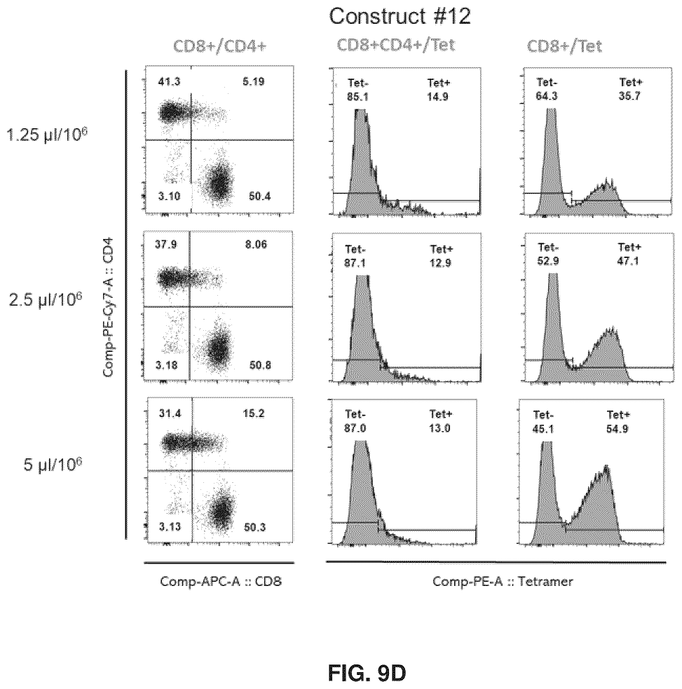

The inventors found that the various CD8 elements in the vector lead to a surprising increase in expression and activity. For example, despite the observation that Construct #10 has lower viral titers than Constructs #9b, #11, and #12 (FIG. 5A), T cells transduced with Construct #10 expressing CD8αβ heterodimer and TCR at the lowest viral volumetric concentration, e.g., 1.25 μl/10⁶ cells, generated higher CD8+CD4+ TCR+ cells (56.7%, FIG. 9B) than that of transduced with Construct #9b expressing CD8α and TCR (42.3%, FIG. 9A), Construct #11 expressing CD8αCD8βstalk with CD8α transmembrane and intracellular domain and TCR (51.6%, FIG. 9C), and Construct #12 expressing CD8αCD8βstalk with Neural Cell Adhesion Molecule 1 (NCAM1) transmembrane and intracellular domain and TCR (14.9%, FIG. 9D).

The inventors also surprisingly found that expressing dnTGFβRII and exogenous TCR in T cells increases the ability of the T cells to maintain their killing ability after multiple stimulations with tumor cells, particularly in the presence of exogenous TGF-β.

A vector may comprise any one or more of nucleic acid sequences of SEQ ID NO: 72, 73, 267, 269, 271, 273, 275, 277, 279, 281, 283, 285, 287, 289, 291, 295, 297, 299, 301, 306, 308, 309-312, or 313.

A T-cell and/or natural killer cell may be transduced to express any one or more of the nucleic acid of SEQ ID NO: 72, 73, 267, 269, 271, 273, 275, 277, 279, 281, 283, 285, 287, 289, 291, 295, 297, 299, 301, 306, 308, 309-312, or 313.

Several of the elements of the constructs in Table 2 are described in Table 3.

TABLE 3
Representative Protein and DNA Sequences

| SEQ ID | | |
|---|---|---|
| NO: | Description | Sequence |
| 1 | CD8α Ig-like | SQFRVSPLDRTWNLGETVELKCQVLLSNPTSGCSWLFQ |
| | domain-1 | PRGAAASPTFLLYLSQNKPKAAEGLDTQRFSGKRLGDT |
| | | FVLTLSDFRRENEGYYFCSALSNSIMYFSHFVPVFLPA |
| 2 | CD8β stalk region | SVVDFLPTTAQPTKKSTLKKRVCRLPRPETQKGPLCSP |
| 3 | CD8α | IYIWAPLAGTCGVLLLSLVIT |
| | transmembrane | |
| | domain | |
| 4 | CD8α | LYCNHRNRRRVCKCPRPVVKSGDKPSLSARYV |
| | cytoplasmic tail | |
| 5 | m1CD8α (signal- | SQFRVSPLDRTWNLGETVELKCQVLLSNPTSGCSWLFQ |
| | less) | PRGAAASPTFLLYLSQNKPKAAEGLDTQRFSGKRLGDT |
| | | FVLTLSDFRRENEGYYFCSALSNSIMYFSHFVPVFLPAS |
| | | VVDFLPTTAQPTKKSTLKKRVCRLPRPETQKGPLCSPIY |
| | | IWAPLAGTCGVLLLSLVITLYCNHRNRRRVCKCPRPVV |
| | | KSGDKPSLSARYV |
| 6 | CD8α Signal | MALPVTALLLPLALLLHAARP |
| | peptide | |
| 7 | m1CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPASVVDFLPTTAQPTKKSTL |
| | | KKRVCRLPRPETQKGPLCSPIYIWAPLAGTCGVLLLSL |
| | | VITLYCNHRNRRRVCKCPRPVVKSGDKPSLSARYV |
| 8 | CD8β1 | MRPRLWLLLAAQLTVLHGNSVLQQTPAYIKVQTNKM |
| | | VMLSCEAKISLSNMRIYWLRQRQAPSSDSHHEFLALW |
| | | DSAKGTIHGEEVEQEKIAVFRDASRFILNLTSVKPEDSG |
| | | IYFCMIVGSPELTFGKGTQLSVVDFLPTTAQPTKKSTLK |
| | | KRVCRLPRPETQKGPLCSPITLGLLVAGVLVLLVSLGV |
| | | AIHLCCRRRRARLRFMKQPQGEGISGTFVPQCLHGYYS |
| | | NTTTSQKLLNPWILKT |
| 9 | CD8β2 | MRPRLWLLLAAQLTVLHGNSVLQQTPAYIKVQTNKM |
| | | VMLSCEAKISLSNMRIYWLRQRQAPSSDSHHEFLALW |
| | | DSAKGTIHGEEVEQEKIAVFRDASRFILNLTSVKPEDSG |
| | | IYFCMIVGSPELTFGKGTQLSVVDFLPTTAQPTKKSTLK |
| | | KRVCRLPRPETQKGLKGKVYQEPLSPNACMDTTAILQP |
| | | HRSCLTHGS |
| 10 | CD8β3 | LQQTPAYIKVQTNKMVMLSCEAKISLSNMRIYWLRQR |
| | | QAPSSDSHHEFLALWDSAKGTIHGEEVEQEKIAVFRDA |
| | | SRFILNLTSVKPEDSGIYFCMIVGSPELTFGKGTQLSVV |
| | | DFLPTTAQPTKKSTLKKRVCRLPRPETQKGPLCSPITLG |
| | | LLVAGVLVLLVSLGVAIHLCCRRRRARLRFMKQFYK |
| 11 | CD8β4 | LQQTPAYIKVQTNKMVMLSCEAKISLSNMRIYWLRQR |
| | | QAPSSDSHHEFLALWDSAKGTIHGEEVEQEKIAVFRDA |
| | | SRFILNLTSVKPEDSGIYFCMIVGSPELTFGKGTQLSVV |
| | | DFLPTTAQPTKKSTLKKRVCRLPRPETQKGPLCSPITLG |
| | | LLVAGVLVLLVSLGVAIHLCCRRRRARLRFMKQLRLH |
| | | PLEKCSRMDY |
| 12 | CD8β5 | LQQTPAYIKVQTNKMVMLSCEAKISLSNMRIYWLRQR |
| | | QAPSSDSHHEFLALWDSAKGTIHGEEVEQEKIAVFRDA |
| | | SRFILNLTSVKPEDSGIYFCMIVGSPELTFGKGTQLSVV |
| | | DFLPTTAQPTKKSTLKKRVCRLPRPETQKGPLCSPITLG |
| | | LLVAGVLVLLVSLGVAIHLCCRRRRARLRFMKQKFNI |
| | | VCLKISGFTTCCCFQILQISREYGFGVLLQKDIGQ |
| 13 | CD8β6 | LQQTPAYIKVQTNKMVMLSCEAKISLSNMRIYWLRQR |
| | | QAPSSDSHHEFLALWDSAKGTIHGEEVEQEKIAVFRDA |
| | | SRFILNLTSVKPEDSGIYFCMIVGSPELTFGKGTQLSVV |
| | | DFLPTTAQPTKKSTLKKRVCRLPRPETQKGPLCSPITLG |
| | | LLVAGVLVLLVSLGVAIHLCCRRRRARLRFMKQKFNI |
| | | VCLKISGFTTCCCFQILQISREYGFGVLLQKDIGQ |
| 14 | CD8β7 | LQQTPAYIKVQTNKMVMLSCEAKISLSNMRIYWLRQR |
| | | QAPSSDSHHEFLALWDSAKGTIHGEEVEQEKIAVFRDA |
| | | SRFILNLTSVKPEDSGIYFCMIVGSPELTFGKGTQLSVV |
| | | DFLPTTAQPTKKSTLKKRVCRLPRPETQKGPLCSPITLG |
| | | LLVAGVLVLLVSLGVAIHLCCRRRRARLRFMKQPQGE |
| | | GISGTFVPQCLHGYYSNTTTSQKLLNPWILKT |
| 15 | R11KEA alpha | MEKNPLAAPLLILWFHLDCVSSILNVEQSPQSLHVQEG |
| | chain | DSTNFTCSFPSSNFYALHWYRKETAKSPEALFVMTLNG |
| | | DEKKKGRISATLNTKEGYSYLYIKGSQPEDSATYLCAL |
| | | YNNNDMRFGAGTRLTVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 16 | R11KEA beta | MDSWTFCCVSLCILVAKHTDAGVIQSPRHEVTEMGQE |
| | chain | VTLRCKPISGHNSLFWYRETMMRGLELLIYFNNNVPID |
| | | DSGMPEDRFSAKMPNASFSTLKIQPSEPRDSAVYFCAS |
| | | SPGSTDTQYFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 17 | R20P1H7 alpha | MEKMLECAFIVLWLQLGWLSGEDQVTQSPEALRLQEG |
| | chain | ESSSLNCSYTVSGLRGLFWYRQDPGKGPEFLFTLYSAG |
| | | EEKEKERLKATLTKKESFLHITAPKPEDSATYLCAVQG |
| | | ENSGYSTLTFGKGTMLLVSPDIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNLL |
| | | MTLRLWSS |
| 18 | R20P1H7 beta | MGPQLLGYVVLCLLGAGPLEAQVTQNPRYLITVTGKK |
| | chain | LTVTCSQNMNHEYMSWYRQDPGLGLRQIYYSMNVEV |
| | | TDKGDVPEGYKVSRKEKRNFPLILESPSPNQTSLYFCAS |
| | | SLGPGLAAYNEQFFGPGTRLTVLEDLKNVFPPEVAVFE |
| | | PSEAEISHTQKATLVCLATGFYPDHVELSWWVNGKEV |
| | | HSGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 19 | R7P1D5 alpha | MKTFAGFSFLFLWLQLDCMSRGEDVEQSLFLSVREGD |
| | chain | SSVINCTYTDSSSTYLYWYKQEPGAGLQLLTYIFSNMD |
| | | MKQDQRLTVLLNKKDKHLSLRIADTQTGDSAIYFCAE |
| | | YSSASKIIFGSGTRLSIRPNIQNPDPAVYQLRDSKSSDKS |
| | | VCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 20 | R7P1D5 beta | MGSWTLCCVSLCILVAKHTDAGVIQSPRHEVTEMGQE |
| | chain | VTLRCKPISGHDYLFWYRQTMMRGLELLIYFNNNVPID |
| | | DSGMPEDRFSAKMPNASFSTLKIQPSEPRDSAVYFCAS |
| | | RANTGELFFGEGSRLTVLEDLKNVFPPEVAVFEPSEAEI |
| | | SHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 21 | R10P2G12 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | EGNSGNTPLVFGKGTRLSVIANIQNPDPAVYQLRDSKS |
| | | SDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 22 | R10P2G12 beta | MGIRLLCRVAFCFLAVGLVDVKVTQSSRYLVKRTGEK |
| | chain | VFLECVQDMDHENMFWYRQDPGLGLRLIYFSYDVKM |
| | | KEKGDIPEGYSVSREKKERFSLILESASTNQTSMYLCAS |
| | | SLSSGSHQETQYFGPGTRLLVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGR |
| | | ADCGFTSESYQQGVLSATILYEILLGKATLYAVLVSAL |
| | | VLMAMVKRKDSRG |
| 23 | R10P1A7 alpha | MKTFAGFSFLFLWLQLDCMSRGEDVEQSLFLSVREGD |
| | chain | SSVINCTYTDSSSTYLYWYKQEPGAGLQLLTYIFSNMD |
| | | MKQDQRLTVLLNKKDKHLSLRIADTQTGDSAIYFCAE |
| | | SKETRLMFGDGTQLVVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGENLL |
| | | MTLRLWSS |
| 24 | R10P1A7 beta | MLLLLLLLGPGISLLLPGSLAGSGLGAWSQHPSVWICK |
| | chain | SGTSVKIECRSLDFQATTMFWYRQFPKQSLMLMATSN |
| | | EGSKATYEQGVEKDKFLINHASLTLSTLTVTSAHPEDS |
| | | SFYICSARAGGHEQFFGPGTRLTVLEDLKNVFPPEVAV |
| | | FEPSEAEISHTQKATLVCLATGFYPDHVELSWVWNGK |
| | | EVHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSESYQQGVLSATILYEILLGKATLYAVLV |
| | | SALVLMAMVKRKDSRG |
| 25 | R4P1D10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNF |
| | | HDKIIFGKGTRLHILPNIQNPDPAVYQLRDSKSSDKSVC |
| | | LFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 26 | R4P1D10 beta | MGFRLLCCVAFCLLGAGPVDSGVTQTPKHLITATGQR |
| | chain | VTLRCSPRSGDLSVYWYQQSLDQGLQFLIHYYNGEER |
| | | AKGNILERFSAQQFPDLHSELNLSSLELGDSALYFCASS |
| | | VASAYGYTFGSGTRLTVVEDLNKVFPPEVAVFEPSEAE |
| | | ISHTQKATLVCLATGFFPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSVSYQQGVLSATILYEILLGKATLYAVLVSALVLM |
| | | AMVKRKDF |
| 27 | R4P3F9 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAYS |
| | | GAGSYQLTFGKGTKLSVIPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 28 | R4P3F9 beta | MGFRLLCCVAFCLLGAGPVDSGVTQTPKHLITATGQR |
| | chain | VTLRCSPRSGDLSVYWYQQSLDQGLQFLIQYYNGEER |
| | | AKGNILERFSAQQFPDLHSELNLSSLELGDSALYFCASS |
| | | VESSYGYTFGSGTRLTVVEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCG |
| | | FTSVSYQQGVLSATILYEILLGKATLYAVLVSALVLMA |
| | | MVKRKDF |
| 29 | R4P3H3 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVKA |
| | | GNQFYFGTGTSLTVIPNIQNPDPAVYQLRDSKSSDKSV |
| | | CLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 30 | R4P3H3 beta | MGTRLLCWVVLGFLGTDHTGAGVSQSPRYKVAKRGQ |
| | chain | DVALRCDPISGHVSLFWYQQALGQGPEFLTYFQNEAQ |
| | | LDKSGLPSDRFFAERPEGSVSTLKIQRTQQEDSAVYLC |
| | | ASSLLTSGGDNEQFFGPGTRLTVLEDLKNVFPPEVAVF |
| | | EPSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSESYQQGVLSATILYEILLGKATLYAVLV |
| | | SALVLMAMVKRKDSRG |
| 31 | R36P3F9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCATVSN |
| | | YQLIWGAGTKLIIKPDIQNPDPAVYQLRDSKSSDKSVC |
| | | LFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 32 | R36P3F9 beta | MGPQLLGYVVLCLLGAGPLEAQVTQNPRYLITVTGKK |
| | chain | LTVTCSQNMNHEYMSWYRQDPGLGLRQIYYSMNVEV |
| | | TDKGDVPEGYKVSRKEKRNFPLILESPSPNQTSLYFCAS |
| | | SSTSGGLSGETQYFGPGTRLLVLEDLKNVFPPEVAVFEP |
| | | SEAEISHTQKATLVCLATGFYPDHVELSWWVNGKEVH |
| | | SGVSTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 33 | R52P2G11 alpha | MKKHLTTFLVILWLYFYRGNGKNQVEQSPQSLIILEGK |
| | chain | NCTLQCNYTVSPFSNLRWYKQDTGRGPVSLTIMTFSEN |
| | | TKSNGRYTATLDADTKQSSLHITASQLSDSASYICVVS |
| | | AYGKLQFGAGTQVVVTPDIQNPDPAVYQLRDSKSSDK |
| | | SVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLM |
| | | TLRLWSS |
| 34 | R52P2G11 beta | MDSWTFCCVSLCILVAKHTDAGVIQSPRHEVTEMGQE |
| | chain | VTLRCKPISGHNSLFWYRQTMMRGLELLIYFNNNVPID |
| | | DSGMPEDRFSAKMPNASFSTLKIQPSEPRDSAVYFCAS |
| | | SLGSPDGNQPQHFGDGTRLSILEDLNKVFPPEVAVFEPS |
| | | EAEISHTQKATLVCLATGFFPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGR |
| | | ADCGFTSVSYQQGVLSATILYEILLGKATLYAVLVSAL |
| | | VLMAMVKRKDF |
| 35 | R53P2A9 alpha | MACPGFLWALVISTCLEFSMAQTVTQSQPEMSVQEAE |
| | chain | TVTLSCTYDTSESDYYLFWYKQPPSRQMILVIRQEAYK |
| | | QQNATENRFSVNFQKAAKSFSLKISDSQLGDAAMYFC |
| | | AYNSYAGGTSYGKLTFGQGTILTVHPNIQNPDPAVYQL |
| | | RDSKSSDKSVCLFTDFDSQTNVSQSKDSDVYITDKTVL |
| | | DMRSMDFKSNSAVAWSNKSDFACANAFNNSIIPEDTFF |
| | | PSPESSCDVKLVEKSFETDTNLNFQNLSVIGFRILLLKV |
| | | AGFNLLMTLRLWSS |
| 36 | R53P2A9 beta | MGPGLLCWVLLCLLGAGPVDAGVTQSPTHLIKTRGQQ |
| | chain | VTLRCSPISGHKSVSWYQQVLGQGPQFIFQYYEKEERG |
| | | RGNFPDRFSARQFPNYSSELNVNALLLGDSALYLCASS |
| | | LDGTSEQYFGPGTRLTVTEDLKNVFPPEVAVFEPSEAEI |
| | | SHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 37 | R26P1A9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCLIGASG |
| | | SRLTFGEGTQLTVNPDIQNPDPAVYQLRDSKSSDKSVC |
| | | LFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 38 | R26P1A9 beta | MGSWTLCCVSLCILVAKHTDAGVIQSPRHEVTEMGQE |
| | chain | VTLRCKPISGHDYLFWYRQTMMRGLELLIYFNNNVPID |
| | | DSGMPEDRFSAKMPNASFSTLKIQPSEPRDSAVYFCAS |
| | | SYFGWNEKLFFGSGTQLSVLEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSVSYQQGVLSATILYEILLGKATLYAVLVSALV |
| | | LMAMVKRKDF |
| 39 | R26P2A6 alpha | MMKSLRVLLVILWLQLSWVWSQQKEVEQDPGPLSVP |
| | chain | EGAIVSLNCTYSNSAFQYFMWYRQYSRKGPELLMYTY |
| | | SSGNKEDGRFTAQVDKSSKYISLFIRDSQPSDSATYLCA |
| | | MSDVSGGYNKLIFGAGTRLAVHPYIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 40 | R26P2A6 beta | MGPQLLGYVVLCLLGAGPLEAQVTQNPRYLITVTGKK |
| | chain | LTVTCSQNMNHEYMSWYRQDPGLGLRQIYYSMNVEV |
| | | TDKGDVPEGYKVSRKEKRNFPLILESPSPNQTSLYFCAS |
| | | TTPDGTDEQFFGPGTRLTVLEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 41 | R26P3H1 alpha | MASAPISMLAMLFTLSGLRAQSVAQPEDQVNVAEGNP |
| | chain | LTVKCTYSVSGNPYLFWYVQYPNRGLQFLLKYITGDN |
| | | LVKGSYGFEAEFNKSQTSFHLKKPSALVSDSALYFCAV |
| | | RDMNRDDKIIFGKGTRLHILPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNLL |
| | | MTLRLWSS |
| 42 | R26P3H1 beta | MSNQVLCCVVLCFLGANTVDGGITQSPKYLFRKEGQN |
| | chain | VTLSCEQNLNHDAMYWYRQDPGQGLRLIYYSQIVNDF |
| | | QKGDIAEGYSVSREKKESFPLTVTSAQKNPTAFYLCAS |
| | | SRAEGGEQYFGPGTRLTVTEDLKNVFPPEVAVFEPSEA |
| | | EISHTQKATLVCLATGFYPDHVELSWWVNGKEVHSGV |
| | | STDPQPLKEQPALNDSRYCLSSRLRVSATFWQNPRNHF |
| | | RCQVQFYGLSENDEWTQDRAKPVTQIVSAEAWGRAD |
| | | CGFTSESYQQGVLSATILYEILLGKATLYAVLVSALVL |
| | | MAMVKRKDSRG |
| 43 | R35P3A4 alpha | MTSIRAVFIFLWLQLDLVNGENVEQHPSTLSVQEGDSA |
| | chain | VIKCTYSDSASNYFPWYKQELGKRPQLIIDIRSNVGEKK |
| | | DQRIAVTLNKTAKHFSLHITETQPEDSAVYFCAASPTG |
| | | GYNKLIFGAGTRLAVHPYIQNPDPAVYQLRDSKSSDKS |
| | | VCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 44 | R35P3A4 beta | MSIGLLCCAALSLLWAGPVNAGVTQTPKFQVLKTGQS |
| | chain | MTLQCAQDMNHEYMSWYRQDPGMGLRLIHYSVGAGI |
| | | TDQGEVPNGYNVSRSTTEDFPLRLLSAAPSQTSVYFCA |
| | | SSLGGASQEQYFGPGTRLTVTEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGR |
| | | ADCGFTSESYQQGVLSATILYEILLGKATLYAVLVSAL |
| | | VLMAMVKRKDSRG |
| 45 | R37P1C9 alpha | MKLVTSITVLLSLGIMGDAKTTQPNSMESNEEEPVHLP |
| | chain | CNHSTISGTDYIHWYRQLPSQGPEYVIHGLTSNVNNRM |
| | | ASLAIAEDRKSSTLILHRATLRDAAVYYCILFNFNKFYF |
| | | GSGTKLNVKPNIQNPDPAVYQLRDSKSSDKSVCLFTDF |
| | | DSQTNVSQSKDSDVYITDKTVLDMRSMDFKSNSAVA |
| | | WSNKSDFACANAFNNSIIPEDTFFPSPESSCDVKLVEKS |
| | | FETDTNLNFQNLSVIGFRILLLKVAGFNLLMTLRLWSS |
| 46 | R37P1C9 beta | MGPGLLHWMALCLLGTGHGDAMVIQNPRYQVTQFGK |
| | chain | PVTLSCSQTLNHNVMYWYQQKSSQAPKLLFHYYDKD |
| | | FNNEADTPDNFQSRRPNTSFCFLDIRSPGLGDAAMYLC |
| | | ATSSGETNEKLFFGSGTQLSVLEDLNKVFPPEVAVFEPS |
| | | EAEISHTQKATLVCLATGFFPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGR |
| | | ADCGFTSVSYQQGVLSATILYEILLGKATLYAVLVSAL |
| | | VLMAMVKRKDF |
| 47 | R37P1H1 alpha | MTRVSLLWAVVVSTCLESGMAQTVTQSQPEMSVQEA |
| | chain | ETVTLSCTYDTSESNYYLFWYKQPPSRQMILVIRQEAY |
| | | KQQNATENRFSVNFQKAAKSFSLKISDSQLGDTAMYF |
| | | CAFGYSGGGADGLTFGKGTHLIIQPYIQNPDPAVYQLR |
| | | DSKSSDKSVCLFTDFDSQTNVSQSKDSDVYITDKTVLD |
| | | MRSMDFKSNSAVAWSNKSDFACANAFNNSIIPEDTFFP |
| | | SPESSCDVKLVEKSFETDTNLNFQNLSVIGFRILLLKVA |
| | | GFNLLMTLRLWSS |
| 48 | R37P1H1 beta | MGPGLLCWALLCLLGAGLVDAGVTQSPTHLIKTRGQQ |
| | chain | VTLRCSPKSGHDTVSWYQQALGQGPQFIFQYYEEEER |
| | | QRGNFPDRFSGHQFPNYSSELNVNALLLGDSALYLCAS |
| | | SNEGQGWEAEAFFGQGTRLTVVEDLNKVFPPEVAVFE |
| | | PSEAEISHTQKATLVCLATGFFPDHVELSWWVNGKEV |
| | | HSGVSTDPQPLKEQPALNDSRYCLSSRLRVSATFWQNP |
| | | RNHFRCQVQFYGLSENDEWTQDRAKPVTQIVSAEAW |
| | | GRADCGFTSVSYQQGVLSATILYEILLGKATLYAVLVS |
| | | ALVLMAMVKRKDF |
| 49 | R42P3A9 alpha | MKRILGALLGLLSAQVCCVRGIQVEQSPPDLILQEGAN |
| | chain | STLRCNFSDSVNNLQWFHQNPWGQLINLFYIPSGTKQN |
| | | GRLSATTVATERYSLLYISSSQTTDSGVYFCAVHNFNK |
| | | FYFGSGTKLNVKPNIQNPDPAVYQLRDSKSSDKSVCLF |
| | | TDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLW |
| | | SS |
| 50 | R42P3A9 beta | MLSPDLPDSAWNTRLLCHVMLCLLGAVSVAAGVIQSP |
| | chain | RHLIKEKRETATLKCYPIPRHDTVYWYQQGPGQDPQFL |
| | | ISFYEKMQSDKGSIPDRFSAQQFSDYHSELNMSSLELG |
| | | DSALYFCASSLLGQGYNEQFFGPGTRLTVLEDLKNVFP |
| | | PEVAVFEPSEAEISHTQKATLVCLATGFYPDHVELSWW |
| | | VNGKEVHSGVSTDPQPLKEQPALNDSRYCLSSRLRVSA |
| | | TFWQNPRNHFRCQVQFYGLSENDEWTQDRAKPVTQIV |
| | | SAEAWGRADCGFTSESYQQGVLSATILYEILLGKATLY |
| | | AVLVSALVLMAMVKRKDSRG |
| 51 | R43P3F2 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | NNNAGNMLTFGGGTRLMVKPHIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 52 | R43P3F2 beta | MLSPDLPDSAWNTRLLCHVMLCLLGAVSVAAGVIQSP |
| | chain | RHLIKEKRETATLKCYPIPRHDTVYWYQQGPGQDPQFL |
| | | ISFYEKMQSDKGSIPDRFSAQQFSDYHSELNMSSLELG |
| | | DSALYFCASSPTGTSGYNEQFFGPGTRLTVLEDLKNVF |
| | | PPEVAVFEPSEAEISHTQKATLVCLATGFYPDHVELSW |
| | | WVNGKEVHSGVSTDPQPLKEQPALNDSRYCLSSRLRV |
| | | SATFWQNPRNHFRCQVQFYGLSENDEWTQDRAKPVT |
| | | QIVSAEAWGRADCGFTSESYQQGVLSATILYEILLGKA |
| | | TLYAVLVSALVLMAMVKRKDSRG |
| 53 | R43P3G5 alpha | MEKNPLAAPLLILWFHLDCVSSILNVEQSPQSLHVQEG |
| | chain | DSTNFTCSFPSSNFYALHWYRWETAKSPEALFVMTLN |
| | | GDEKKKGRISATLNTKEGYSYLYIKGSQPEDSATYLCA |
| | | LNRDDKIIFGKGTRLHILPNIQNPDPAVYQLRDSKSSDK |
| | | SVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLM |
| | | TLRLWSS |
| 54 | R43P3G5 beta | MGIRLLCRVAFCFLAVGLVDVKVTQSSRYLVKRTGEK |
| | chain | VFLECVQDMDHENMFWYRQDPGLGLRLIYFSYDVKM |
| | | KEKGDIPEGYSVSREKKERFSLILESASTNQTSMYLCAS |
| | | RLPSRTYEQYFGPGTRLTVTEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 55 | R59P2E7 alpha | METLLGLLILWLQLQWVSSKQEVTQIPAALSVPEGENL |
| | chain | VLNCSFTDSAIYNLQWFRQDPGKGLTSLLLIQSSQREQ |
| | | TSGRLNASLDKSSGRSTLYIAASQPGDSATYLCAVNSD |
| | | YKLSFGAGTTVTVRANIQNPDPAVYQLRDSKSSDKSV |
| | | CLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 56 | R59P2E7 beta | MLSPDLPDSAWNTRLLCHVMLCLLGAVSVAAGVIQSP |
| | chain | RHLIKEKRETATLKCYPIPRHDTVYWYQQGPGQDPQFL |
| | | ISFYEKMQSDKGSIPDRFSAQQFSDYHSELNMSSLELG |
| | | DSALYFCASSLGLGTGDYGYTFGSGTRLTVVEDLNKV |
| | | FPPEVAVFEPSEAEISHTQKATLVCLATGFFPDHVELSW |
| | | WVNGKEVHSGVSTDPQPLKEQPALNDSRYCLSSRLRV |
| | | SATFWQNPRNHFRCQVQFYGLSENDEWTQDRAKPVT |
| | | QIVSAEAWGRADCGFTSVSYQQGVLSATILYEILLGKA |
| | | TLYAVLVSALVLMAMVKRKDF |
| 57 | R11P3D3 alpha | MEKNPLAAPLLILWFHLDCVSSILNVEQSPQSLHVQEG |
| | chain | DSTNFTCSFPSSNFYALHWYRWETAKSPEALFVMTLN |
| | | GDEKKKGRISATLNTKEGYSYLYIKGSQPEDSATYLCA |
| | | LYNNNDMRFGAGTRLTVKPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNLL |
| | | MTLRLWSS |
| 58 | R11P3D3 beta | MDSWTFCCVSLCILVAKHTDAGVIQSPRHEVTEMGQE |
| | chain | VTLRCKPISGHNSLFWYRQTMMRGLELLIYFNNNVPID |
| | | DSGMPEDRFSAKMPNASFSTLKIQPSEPRDSAVYFCAS |
| | | SPGSTDTQYFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 59 | R16P1C10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAVI |
| | | SNFGNEKLTFGTGTRLTIIPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 60 | R16P1C10 beta | MGSRLLCWVLLCLLGAGPVKAGVTQTPRYLIKTRGQQ |
| | chain | VTLSCSPISGHRSVSWYQQTPGQGLQFLFEYFSETQRN |
| | | KGNFPGRFSGRQFSNSRSEMNVSTLELGDSALYLCASS |
| | | PWDSPNEQYFGPGTRLTVTEDLKNVFPPEVAVFEPSEA |
| | | EISHTQKATLVCLATGFYPDHVELSWWVNGKEVHSGV |
| | | STDPQPLKEQPALNDSRYCLSSRLRVSATFWQNPRNHF |
| | | RCQVQFYGLSENDEWTQDRAKPVTQIVSAEAWGRAD |
| | | CGFTSESYQQGVLSATILYEILLGKATLYAVLVSALVL |
| | | MAMVKRKDSRG |
| 61 | R16P1E8 alpha | MMKSLRVLLVILWLQLSWVWSQQKEVEQDPGPLSVP |
| | chain | EGAIVSLNCTYSNSAFQYFMWYRQYSRKGPELLMYTY |
| | | SSGNKEDGRFTAQVDKSSKYISLFIRDSQPSDSATYLCA |
| | | MSEAAGNKLTFGGGTRVLVKPNIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 62 | R16P1E8 beta | MGTRLLCWAALCLLGAELTEAGVAQSPRYKIIEKRQS |
| | chain | VAFWCNPISGHATLYWYQQILGQGPKLLIQFQNNGVV |
| | | DDSQLPKDRFSAERLKGVDSTLKIQPAKLEDSAVYLCA |
| | | SSYTNQGEAFFGQGTRLTVVEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSVSYQQGVLSATILYEILLGKATLYAVLVSALV |
| | | LMAMVKRKDF |
| 63 | R17P1A9 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVLN |
| | | QAGTALIFGKGTTLSVSSNIQNPDPAVYQLRDSKSSDK |
| | | SVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLM |
| | | TLRLWSS |
| 64 | R17P1A9 beta | MGFRLLCCVAFCLLGAGPVDSGVTQTPKHLITATGQR |
| | chain | VTLRCSPRSGDLSVYWYQQSLDQGLQFLIQYYNGEER |
| | | AKGNILERFSAQQFPDLHSELNLSSLELGDSALYFCASS |
| | | AETGPWLGNEQFFGPGTRLTVLEDLKNVFPPEVAVFEP |
| | | SEAEISHTQKATLVCLATGFYPDHVELSWWVNGKEVH |
| | | SGVSTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 65 | R17P1D7 alpha | MACPGFLWALVISTCLEFSMAQTVTQSQPEMSVQEAE |
| | chain | TVTLSCTYDTSESDYYLFWYKQPPSRQMILVIRQEAYK |
| | | QQNATENRFSVNFQKAAKSFSLKISDSQLGDAAMYFC |
| | | AYRWAQGGSEKLVFGKGTKLTVNPYIQKPDPAVYQLR |
| | | DSKSSDKSVCLFTDFDSQTNVSQSKDSDVYITDKTVLD |
| | | MRSMDFKSNSAVAWSNKSDFACANAFNNSIIPEDTFFP |
| | | SPESSCDVKLVEKSFETDTNLNFQNLSVIGFRILLLKVA |
| | | GFNLLMTLRLWSS |
| 66 | R17P1D7 beta | MTIRLLCYMGFYFLGAGLMEADIYQTPRYLVIGTGKKI |
| | chain | TLECSQTMGHDKMYWYQQDPGMELHLIHYSYGVNST |
| | | EKGDLSSESTVSRIRTEHFPLTLESARPSHTSQYLCATEL |
| | | WSSGGTGELFFGEGSRLTVLEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 67 | R17P1G3 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVGP |
| | | SGTYKYIFGTGTRLKVLANIQNPDPAVYQLRDSKSSDK |
| | | SVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLM |
| | | TLRLWSS |
| 68 | R17P1G3 beta | MGPQLLGYVVLCLLGAGPLEAQVTQNPRYLITVTGKK |
| | chain | LTVTCSQNMNHEYMSWYRQDPGLGLRQIYYSMNVEV |
| | | TDKGDVPEGYKVSRKEKRNFPLILESPSPNQTSLYFCAS |
| | | SPGGSGNEQFFGPGTRLTVLEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 69 | R17P2B6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVVS |
| | | GGGADGLTFGKGTHLIIQPYIQKPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGENLL |
| | | MTLRLWSS |
| 70 | R17P2B6 beta | MLSPDLPDSAWNTRLLCHVMLCLLGAVSVAAGVIQSP |
| | chain | RHLIKEKRETATLKCYPIPRHDTVYWYQQGPGQDPQFL |
| | | ISFYEKMQSDKGSIPDRFSAQQFSDYHSELNMSSLELG |
| | | DSALYFCASSLGRGGQPQHFGDGTRLSILEDLNKVFPP |
| | | EVAVFEPSEAEISHTQKATLVCLATGFFPDHVELSWWV |
| | | NGKEVHSGVSTDPQPLKEQPALNDSRYCLSSRLRVSAT |
| | | FWQNPRNHFRCQVQFYGLSENDEWTQDRAKPVTQIVS |
| | | AEAWGRADCGFTSVSYQQGVLSATILYEILLGKATLY |
| | | AVLVSALVLMAMVKRKDF |
| 71 | R11P3D3KE | MEKNPLAAPLLILWFHLDCVSSILNVEQSPQSLHVQEG |
| | alpha chain | DSTNFTCSFPSSNFYALHWYRKETAKSPEALFVMTLNG |
| | | DEKKKGRISATLNTKEGYSYLYIKGSQPEDSATYLCAL |
| | | YNNNDMRFGAGTRLTVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 72 | R11KEA alpha | atggagaagaatcccctggctgcccccctgctgatcctgtggtttcacctggactgcgt |
| | chain nucleic acid | gtcctctatcctgaatgtggaacagagcccacagagcctgcacgtgcaggagggcga |
| | sequence | ctccaccaacttcacatgctcttttcctagctccaacttctacgccctgcactggtacaga |
| | | aaggagaccgcaaagtccccagaggccctgttcgtgatgacactgaacggcgatga |
| | | gaagaagaagggccgcatcagcgccaccctgaatacaaaggagggctactcctatct |
| | | gtacatcaagggctcccagcctgaggactctgccacctatctgtgcgccctgtacaaca |
| | | ataacgatatgcggtttggcgccggcaccagactgacagtgaagccaaacatccaga |
| | | atccagaccccgccgtgtatcagctgcgggacagcaagtctagcgataagagcgtgt |
| | | gcctgttcaccgactttgattctcagacaaacgtgagccagtccaaggacagcgacgt |
| | | gtacatcaccgacaagacagtgctggatatgagaagcatggacttcaagtctaacagc |
| | | gccgtggcctggtccaataagtctgatttcgcctgcgccaatgcctttaataactccatca |
| | | tccccgaggataccttctttccttctccagagtcctcttgtgacgtgaagctggtggagaa |
| | | gtctttcgagaccgatacaaacctgaattttcagaacctgagcgtgatcggcttcaggat |
| | | cctgctgctgaaggtggccggctttaatctgctgatgaccctgaggctgtggagctcc |
| 73 | R11KEA beta | atggactcttggaccttctgctgcgtgagcctgtgcatcctggtggccaagcacacaga |
| | chain nucleic acid | cgccggcgtgatccagtcccctaggcacgaggtgaccgagatgggccaggaggtga |
| | sequence | cactgcgctgtaagccaatctctggccacaacagcctgttttggtatagggagaccatg |
| | | atgcgcggcctggagctgctgatctacttcaataacaatgtgcccatcgacgattccgg |
| | | catgcctgaggatcggttttctgccaagatgcccaatgccagcttctccacactgaagat |
| | | ccagcctagcgagccaagagactccgccgtgtatttttgcgcctctagcccaggcagc |
| | | accgatacacagtacttcggaccaggaaccaggctgacagtgctggaggacctgaag |
| | | aacgtgttcccccctgaggtggccgtgtttgagccctctgaggccgagatcagccaca |
| | | cccagaaggccaccctggtgtgcctggcaaccggcttctatcctgatcacgtggagct |
| | | gtcctggtgggtgaacggcaaggaggtgcacagcggcgtgtccacagacccacagc |
| | | ccctgaaggagcagccagccctgaatgatagccggtattgcctgtcctctcggctgag |
| | | agtgtccgccaccttttggcagaacccccggaatcacttcagatgtcaggtgcagtttta |
| | | cggcctgtccgagaacgatgagtggacccaggaccgggccaagcctgtgacacaga |
| | | tcgtgtctgccgaggcatggggaagagcagactgtggcttcacctctgagagctacca |
| | | gcagggcgtgctgagcgccaccatcctgtatgagatcctgctgggcaaggccacact |
| | | gtacgccgtcctggtctccgctctggtgctgatggcaatggtcaaaagaaaagatagtc |
| | | gggga |
| 74 | R39P1C12 beta | MGPGLLCWALLCLLGAGLVDAGVTQSPTHLIKTRGQQ |
| | chain | VTLRCSPKSGHDTVSWYQQALGQGPQFIFQYYEEEER |
| | | QRGNFPDRFSGHQFPNYSSELNVNALLLGDSALYLCAS |
| | | SQLNTEAFFGQGTRLTVVEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCG |
| | | FTSVSYQQGVLSATILYEILLGKATLYAVLVSALVLMA |
| | | MVKRKDF |
| 75 | R39P1F5 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNN |
| | | ARLMFGDGTQLVVKPNIQNPDPAVYQLRDSKSSDKSV |
| | | CLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 76 | R39P1F5 beta | MDTWLVCWAIFSLLKAGLTEPEVTQTPSHQVTQMGQE |
| | chain | VILRCVPISNHLYFYWYRQILGQKVEFLVSFYNNEISEK |
| | | SEIFDDQFSVERPDGSNFTLKIRSTKLEDSAMYFCASSG |
| | | QGANEQYFGPGTRLTVTEDLKNVFPPEVAVFEPSEAEI |
| | | SHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 77 | R40P1C2 alpha | MACPGFLWALVISTCLEFSMAQTVTQSQPEMSVQEAE |
| | chain | TVTLSCTYDTSESDYYLFWYKQPPSRQMILVIRQEAYK |
| | | QQNATENRFSVNFQKAAKSFSLKISDSQLGDAAMYFC |
| | | AYLNYQLIWGAGTKLIIKPDIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGENLL |
| | | MTLRLWSS |
| 78 | R40P1C2 beta | MDTWLVCWAIFSLLKAGLTEPEVTQTPSHQVTQMGQE |
| | chain | VILRCVPISNHLYFYWYRQILGQKVEFLVSFYNNEISEK |
| | | SEIFDDQFSVERPDGSNFTLKIRSTKLEDSAMYFCASSE |
| | | MTAVGQYFGPGTRLTVTEDLKNVFPPEVAVFEPSEAEI |
| | | SHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 79 | R41P3E6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFT |
| | | AQLNKASQYVSLLIRDSQPSDSATYLCAAFSGYALNFG |
| | | KGTSLLVTPHIQNPDPAVYQLRDSKSSDKSVCLFTDFD |
| | | SQTNVSQSKDSDVYITDKTVLDMRSMDFKSNSAVAWS |
| | | NKSDFACANAFNNSIIPEDTFFPSPESSCDVKLVEKSFET |
| | | DTNLNFQNLSVIGFRILLLKVAGFNLLMTLRLWSS |
| 80 | R41P3E6 beta | MDTWLVCWAIFSLLKAGLTEPEVTQTPSHQVTQMGQE |
| | chain | VILRCVPISNHLYFYWYRQILGQKVEFLVSFYNNEISEK |
| | | SEIFDDQFSVERPDGSNFTLKIRSTKLEDSAMYFCASSQ |
| | | YTGELFFGEGSRLTVLEDLKNVFPPEVAVFEPSEAEISH |
| | | TQKATLVCLATGFYPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGF |
| | | TSESYQQGVLSATILYEILLGKATLYAVLVSALVLMAM |
| | | VKRKDSRG |
| 81 | R43P3G4 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNG |
| | | GDMRFGAGTRLTVKPNIQNPDPAVYQLRDSKSSDKSV |
| | | CLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 82 | R43P3G4 beta | MDTWLVCWAIFSLLKAGLTEPEVTQTPSHQVTQMGQE |
| | chain | VILRCVPISNHLYFYWYRQILGQKVEFLVSFYNNEISEK |
| | | SEIFDDQFSVERPDGSNFTLKIRSTKLEDSAMYFCASSG |
| | | QGALEQYFGPGTRLTVTEDLKNVFPPEVAVFEPSEAEIS |
| | | HTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCG |
| | | FTSESYQQGVLSATILYEILLGKATLYAVLVSALVLMA |
| | | MVKRKDSRG |
| 83 | R44P3B3 alpha | MAMLLGASVLILWLQPDWVNSQQKNDDQQVKQNSPS |
| | chain | LSVQEGRISILNCDYTNSMFDYFLWYKKYPAEGPTFLIS |
| | | ISSIKDKNEDGRFTVFLNKSAKHLSLHIVPSQPGDSAVY |
| | | FCAASGLYNQGGKLIFGQGTELSVKPNIQNPDPAVYQL |
| | | RDSKSSDKSVCLFTDFDSQTNVSQSKDSDVYITDKTVL |
| | | DMRSMDFKSNSAVAWSNKSDFACANAFNNSIIPEDTFF |
| | | PSPESSCDVKLVEKSFETDTNLNFQNLSVIGFRILLLKV |
| | | AGFNLLMTLRLWSS |
| 84 | R44P3B3 beta | MGCRLLCCVVFCLLQAGPLDTAVSQTPKYLVTQMGN |
| | chain | DKSIKCEQNLGHDTMYWYKQDSKKFLKIMFSYNNKEL |
| | | IINETVPNRFSPKSPDKAHLNLHINSLELGDSAVYFCAS |
| | | SLGDRGYEQYFGPGTRLTVTEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 85 | R44P3E7 alpha | MKTFAGFSFLFLWLQLDCMSRGEDVEQSLFLSVREGD |
| | chain | SSVINCTYTDSSSTYLYWYKQEPGAGLQLLTYIFSNMD |
| | | MKQDQRLTVLLNKKDKHLSLRIADTQTGDSAIYFCAEI |
| | | NNNARLMFGDGTQLVVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 86 | R44P3E7 beta | MLSPDLPDSAWNTRLLCHVMLCLLGAVSVAAGVIQSP |
| | chain | RHLIKEKRETATLKCYPIPRHDTVYWYQQGPGQDPQFL |
| | | ISFYEKMQSDKGSIPDRFSAQQFSDYHSELNMSSLELG |
| | | DSALYFCASSPPDQNTQYFGPGTRLTVLEDLKNVFPPE |
| | | VAVFEPSEAEISHTQKATLVCLATGFYPDHVELSWWV |
| | | NGKEVHSGVSTDPQPLKEQPALNDSRYCLSSRLRVSAT |
| | | FWQNPRNHFRCQVQFYGLSENDEWTQDRAKPVTQIVS |
| | | AEAWGRADCGFTSESYQQGVLSATILYEILLGKATLYA |
| | | VLVSALVLMAMVKRKDSRG |
| 87 | R49P2B7 alpha | MLLLLVPVLEVIFTLGGTRAQSVTQLGSHVSVSEGALV |
| | chain | LLRCNYSSSVPPYLFWYVQYPNQGLQLLLKYTTGATL |
| | | VKGINGFEAEFKKSETSFHLTKPSAHMSDAAEYFCAVR |
| | | IFGNEKLTFGTGTRLTIIPNIQNPDPAVYQLRDSKSSDKS |
| | | VCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 88 | R49P2B7 beta | MGIRLLCRVAFCFLAVGLVDVKVTQSSRYLVKRTGEK |
| | chain | VFLECVQDMDHENMFWYRQDPGLGLRLIYFSYDVKM |
| | | KEKGDIPEGYSVSREKKERFSLILESASTNQTSMYLCAS |
| | | SLMGELTGELFFGEGSRLTVLEDLKNVFPPEVAVFEPSE |
| | | AEISHTQKATLVCLATGFYPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSESYQQGVLSATILYEILLGKATLYAVLVSALV |
| | | LMAMVKRKDSRG |
| 89 | R55P1G7 alpha | MMKSLRVLLVILWLQLSWVWSQQKEVEQDPGPLSVP |
| | chain | EGAIVSLNCTYSNSAFQYFMWYRQYSRKGPELLMYTY |
| | | SSGNKEDGRFTAQVDKSSKYISLFIRDSQPSDSATYLCA |
| | | MMGDTGTASKLTFGTGTRLQVTLDIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 90 | R55P1G7 beta | MGIRLLCRVAFCFLAVGLVDVKVTQSSRYLVKRTGEK |
| | chain | VFLECVQDMDHENMFWYRQDPGLGLRLIYFSYDVKM |
| | | KEKGDIPEGYSVSREKKERFSLILESASTNQTSMYLCAS |
| | | SFGGYEQYFGPGTRLTVTEDLKNVFPPEVAVFEPSEAEI |
| | | SHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 91 | R59P2A7 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVQP |
| | | HDMRFGAGTRLTVKPNIQNPDPAVYQLRDSKSSDKSV |
| | | CLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 92 | R59P2A7 beta | MLCSLLALLLGTFFGVRSQTIHQWPATLVQPVGSPLSL |
| | chain | ECTVEGTSNPNLYWYRQAAGRGLQLLFYSVGIGQISSE |
| | | VPQNLSASRPQDRQFILSSKKLLLSDSGFYLCAWSGLV |
| | | AEQFFGPGTRLTVLEDLKNVFPPEVAVFEPSEAEISHTQ |
| | | KATLVCLATGFYPDHVELSWWVNGKEVHSGVSTDPQ |
| | | PLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMV |
| | | KRKDSRG |
| 93 | P2A | ATNFSLLKQAGDVEENPGP |
| 94 | T2A | EGRGSLLTCGDVEENPGP |
| 95 | E2A | QCTNYALLKLAGDVESNPGP |
| 96 | F2A | VKQTLNFDLLKLAGDVESNPGP |
| 97 | RD114TR | MKLPTGMVILCSLIIVRAGFDDPRKAIALVQKQHGKPC |
| | | ECSGGQVSEAPPNSIQQVTCPGKTAYLMTNQKWKCRV |
| | | TPKISPSGGELQNCPCNTFQDSMHSSCYTEYRQCRRIN |
| | | KTYYTATLLKIRSGSLNEVQILQNPNQLLQSPCRGSINQ |
| | | PVCWSATAPIHISDGGGPLDTKRVWTVQKRLEQIHKA |
| | | MTPELQYHPLALPKVRDDLSLDARTFDILNTTFRLLQM |
| | | SNFSLAQDCWLCLKLGTPTPLAIPTPSLTYSLADSLANA |
| | | SCQIIPPLLVQPMQFSNSSCLSSPFINDTEQIDLGAVTFT |
| | | NCTSVANVSSPLCALNGSVFLCGNNMAYTYLPQNWTR |
| | | LCVQASLLPDIDINPGDEPVPIPAIDHYIHRPKRAVQFIP |
| | | LLAGLGITAAFTTGATGLGVSVTQYTKLSHQLISDVQV |
| | | LSGTIQDLQDQVDSLAEVVLQNRRGLDLLTAEQGGICL |
| | | ALQEKCCFYANKSGIVRNKIRTLQEELQKRRESLASNP |
| | | LWTGLQGFLPYLLPLLGPLLTLLLILTIGPCVFNRLVQF |
| | | VKDRISVVQALVLTQQYHQLKPL |
| 256 | WPREmut1 | cagtctgacgtacgcgtaatcaacctctggattacaaaatttgtgaaagattgactggtat |
| | | tcttaactatgttgctccttttacgctatgtggatacgctgctttaatgcctttgtatcatgcta |
| | | ttgcttcccgtatggctttcattttctcctccttgtataaatcctggttgctgtctctttatgag |
| | | gagttgtggcccgttgtcaggcaacgtggcgtggtgtgcactgtgtttgctgacgcaac |
| | | ccccactggttggggcattgccaccacctgtcagctcctttccgggactttcgctttccc |
| | | cctccctattgccacggcggaactcatcgccgcctgccttgcccgctgctggacaggg |
| | | gctcggctgttgggcactgacaattccgtggtgttgtcggggaaatcatcgtcctttcctt |
| | | ggctgctcgcctgtgttgccacctggattctgcgcgggacgtccttctgctacgtccctt |
| | | cggccctcaatccagcggaccttccttcccgcggcctgctgccggctctgcggcctctt |
| | | ccgcgtcttcgccttcgccctcagacgagtcggatctccctttgggccgcctccccgcc |
| 257 | WPREmut2 | Gagcatcttaccgccatttatacccatatttgttctgtttttcttgatttgggtatacatttaaa |
| | | tgttaataaaacaaaatggtggggcaatcatttacattttttgggatatgtaattactagttc |
| | | aggtgtattgccacaagacaaacttgttaagaaactttcccgttatttacgctctgttcctgt |
| | | taatcaacctctggattacaaaatttgtgaaagattgactgatattcttaactttgttgctcct |
| | | tttacgctgtgtggatttgctgctttattgcctctgtatcttgctattgcttcccgtacggcttt |
| | | cgttttctcctccttgtataaatcctggttgctgtctctttttgaggagttgtggcccgttgtc |
| | | cgtcaacgtggcgtggtgtgctctgtgtttgctgacgcaacccccactggctggggcat |
| | | tgccaccacctgtcaactcctttctgggactttcgctttccccctcccgatcgccacggc |
| | | agaactcatcgccgcctgccttgcccgctgctggacaggggctaggttgctgggcact |
| | | gataattccgtggtgttgtc |
| 258 | CD8α1 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPAKPTTTPAPRPPTPAPTIAS |
| | | QPLSLRPEACRPAAGGAVHTRGLDFACDIYIWAPLAGT |
| | | CGVLLLSLVITLYCNHRNRRRVCKCPRPVVKSGDKPSL |
| | | SARYV |
| 259 | CD8α2 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPAKPTTTPAPRPPTPAPTIAS |
| | | QPLSLRPEACRPAAGGAVHTRGLDFACDIYIWAPLAGT |
| | | CGVLLLSLVITLYCNHRNRRRVCKCPRPVVKSGDKPSL |
| | | SARYV |
| 260 | CD8α stalk | KPTTTPAPRPPTPAPTIASQPLSLRPEACRPAAGGAVHT |
| | | RGLDFACD |
| 261 | CD8α Ig-like | SQFRVSPLDRTWNLGETVELKCQVLLSNPTSGCSWLFQ |
| | domain-2 | PRGAAASPTFLLYLSQNKPKAAEGLDTQRFSGKRLGDT |
| | | FVLTLSDFRRENEGCYFCS2ALSNSIMYFSHFVPVFLPA |
| 262 | m2CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPASVVDFLPTTAQPTKKSTL |
| | | KKRVCRLPRPETQKGPLCSPIYIWAPLAGTCGVLLLSL |
| | | VITLYCNHRNRRRVCKCPRPVVKSGDKPSLSARYV |
| 263 | MSCV promoter | Tgaaagaccccacctgtaggtttggcaagctagcttaagtaacgccattttgcaaggc |
| | | atggaaaatacataactgagaatagagaagttcagatcaaggttaggaacagagagac |
| | | agcagaatatgggccaaacaggatatctgtggtaagcagttcctgccccggctcaggg |
| | | ccaagaacagatggtccccagatgcggtcccgccctcagcagtttctagagaaccatc |
| | | agatgtttccagggtgccccaaggacctgaaaatgaccctgtgccttatttgaactaacc |
| | | aatcagttcgcttctcgcttctgttcgcgcgcttctgctccccgagctcaataaaagagcc |
| | | cacaacccctcact |
| 264 | WPRE | cagtctgacgtacgcgtaatcaacctctggattacaaaatttgtgaaagattgactggtat |
| | | tcttaactatgttgctccttttacgctatgtggatacgctgctttaatgcctttgtatcatgcta |
| | | ttgcttcccgtatggctttcattttctcctccttgtataaatcctggttgctgtctctttatgag |
| | | gagttgtggcccgttgtcaggcaacgtggcgtggtgtgcactgtgtttgctgacgcaac |
| | | ccccactggttggggcattgccaccacctgtcagctcctttccgggactttcgctttccc |
| | | cctccctattgccacggcggaactcatcgccgcctgccttgcccgctgctggacaggg |
| | | gctcggctgttgggcactgacaattccgtggtgttgtcggggaagctgacgtcctttcca |
| | | tggctgctcgcctgtgttgccacctggattctgcgcgggacgtccttctgctacgtccctt |
| | | cggccctcaatccagcggaccttccttcccgcggcctgctgccggctctgcggcctctt |
| | | ccgcgtcttcgccttcgccctcagacgagtcggatctccctttgggccgcctccccgcc |
| 265 | Furin consensus | RXXR |
| 266 | Linker | SGSG |
| 293 | CD8β Signal | MRPRLWLLLAAQLTVLHGNSV |
| | peptide | |
| 294 | S19 Signal | MEFGLSWLFLVAILKGVQC |
| | peptide | |
| 303 | R11P3D3KE beta | MDSWTFCCVSLCILVAKHTDAGVIQSPRHEVTEMGQE |
| | chain | VTLRCKPISGHNSLFWYRETMMRGLELLIYFNNNVPID |
| | | DSGMPEDRFSAKMPNASFSTLKIQPSEPRDSAVYFCAS |
| | | SPGSTDTQYFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRG |
| 304 | R39P1C12 alpha | MKTFAGFSFLFLWLQLDCMSRGEDVEQSLFLSVREGD |
| | chain | SSVINCTYTDSSSTYLYWYKQEPGAGLQLLTYIFSNMD |
| | | MKQDQRLTVLLNKKDKHLSLRIADTQTGDSAIYFCAEI |
| | | DNQGGKLIFGQGTELSVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 305 | Dominant | MGRGLLRGLWPLHIVLWTRIASTIPPHVQKSDVEMEA |
| | Negative | QKDEIICPSCNRTAHPLRHINNDMIVTDNNGAVKFPQL |
| | TGFβRII var1 | CKFCDVRFSTCDNQKSCMSNCSITSICEKPQEVCVAVW |
| | Amino Acid | RKNDENITLETVCHDPKLPYHDFILEDAASPKCIMKEK |
| | Sequence | KKPGETFFMCSCSSDECNDNIIFSEEYNTSNPDLLLVIFQ |
| | | VTGISLLPPLGVAISVIIIFYCYRVNRQQKLSS |
| 306 | Dominant | atgggtcgggggctgctcaggggcctgtggccgctgcacatcgtcctgtggacgcgt |
| | Negative | atcgccagcacgatcccaccgcacgttcagaagtcggatgtggaaatggaggcccag |
| | TGFβRII var1 | aaagatgaaatcatctgccccagctgtaataggactgcccatccactgagacatattaat |
| | Nucleic Acid | aacgacatgatagtcactgacaacaacggtgcagtcaagtttccacaactgtgtaaattt |
| | Sequence | tgtgatgtgagattttccacctgtgacaaccagaaatcctgcatgagcaactgcagcatc |
| | | acctccatctgtgagaagccacaggaagtctgtgtggctgtatggagaaagaatgacg |
| | | agaacataacactagagacagtttgccatgaccccaagctcccctaccatgactttattc |
| | | tggaagatgctgcttctccaaagtgcattatgaaggaaaaaaaaaagcctggtgagact |
| | | ttcttcatgtgttcctgtagctctgatgagtgcaatgacaacatcatcttctcagaagaatat |
| | | aacaccagcaatcctgacttgttgctagtcatatttcaagtgacaggcatcagcctcctg |
| | | ccaccactgggagttgccatatctgtcatcatcatcttctactgctaccgcgttaaccggc |
| | | agcagaagctgagttca |
| 307 | Dominant | MGRGLLRGLWPLHIVLWTRIASTIPPHVQKSVNNDMIV |
| | Negative | TDNNGAVKFPQLCKFCDVRFSTCDNQKSCMSNCSITSI |
| | TGFβRII var2 | CEKPQEVCVAVWRKNDENITLETVCHDPKLPYHDFILE |
| | Amino Acid | DAASPKCIMKEKKKPGETFFMCSCSSDECNDNIIFSEEY |
| | Sequence | NTSNPDLLLVIFQVTGISLLPPLGVAISVIIIFYCYRVNRQ |
| | | QKLSS |
| 308 | Dominant | atgggtcgggggctgctcaggggcctgtggccgctgcacatcgtcctgtggacgcgt |
| | Negative | atcgccagcacgatcccaccgcacgttcagaagtcggttaataacgacatgatagtca |
| | TGFβRII var2 | ctgacaacaacggtgcagtcaagtttccacaactgtgtaaattttgtgatgtgagattttcc |
| | Nucleic Acid | acctgtgacaaccagaaatcctgcatgagcaactgcagcatcacctccatctgtgaga |
| | Sequence | agccacaggaagtctgtgtggctgtatggagaaagaatgacgagaacataacactag |
| | | agacagtttgccatgaccccaagctcccctaccatgactttattctggaagatgctgcttc |
| | | tccaaagtgcattatgaaggaaaaaaaaaagcctggtgagactttcttcatgtgttcctgt |
| | | agctctgatgagtgcaatgacaacatcatcttctcagaagaatataacaccagcaatcct |
| | | gacttgttgctagtcatatttcaagtgacaggcatcagcctcctgccaccactgggagtt |
| | | gccatatctgtcatcatcatcttctactgctaccgcgttaaccggcagcagaagctgagtt |
| | | ca |
| 309 | CD8β1 Nucleic | atgcgcccgagactgtggcttctgctcgccgcgcaactgactgtcctgcacggaaaca |
| | Acid Sequence, | gcgtgctgcagcagacaccggcctacatcaaagtgcagaccaacaagatggtcatgc |
| | codon optimized | tgtcctgcgaggccaagatttccctctccaacatgcggatctattggttgcggcagaga |
| | | caggcgccttcctcggactcccaccatgagttcttggccctgtgggactccgccaagg |
| | | gaactattcacggcgaagaagtggaacaggagaagatcgccgtgtttcgcgatgcctc |
| | | ccgctttatactgaatctgacctccgtgaagcccgaagatagcgggatctacttttgcat |
| | | gattgtgggctcacccgaactgaccttcgggaagggcactcagctgagcgtggtgga |
| | | cttcctccccactaccgcccaacccactaagaagtcaaccctgaagaagcgggtttgc |
| | | agactcccacggccggaaacgcagaagggtccgctgtgttccccgatcaccctggg |
| | | gctccttgtggctggagtgctggtccttctggtgtcccttggcgtcgccattcacctctgc |
| | | tgccggagaaggagggccagactgaggttcatgaagcagcctcagggagagggga |
| | | tcagtggcactttcgtgccacaatgcctccatggctactattccaacaccaccacctcgc |
| | | aaaagctgctgaacccctggatcctgaaaacc |
| 310 | CD8α1 Nucleic | atggcgcttcccgtgaccgcactcctgttgccccttgccctgctgttgcacgccgcacg |
| | Acid Sequence, | accttcccaattccgggtgtcccctctggatcgcacctggaacctcggggaaacggtg |
| | codon optimized | gagctcaagtgtcaagtcctcctgtcgaacccgaccagcggatgcagctggctgttcc |
| | | agccgagaggagctgccgcctcacccaccttcctcctgtacttgagccagaacaagc |
| | | cgaaggccgctgagggtctggacacccagcgcttctcgggcaaacggctgggagac |
| | | acttttgtgctgactctctccgacttccggcgggagaacgagggctactacttctgctct |
| | | gcgctctccaattcaatcatgtacttctcacacttcgtgccggtgttcctgcctgccaagc |
| | | ccaccactactccggcacccagacctccaactcccgctcccaccatcgcgtcccaacc |
| | | cctttcgctgcgccctgaagcgtgtcggcctgctgctggaggagccgtgcatacccgc |
| | | ggtctggacttcgcgtgcgacatctacatttgggcccctttggctggcacctgtggagtg |
| | | ctgctcctgtcccttgtgatcaccctgtactgcaaccaccggaataggcggagagtctg |
| | | caagtgtccgcggcctgtcgtgaagtcaggagataagccgagcctgtccgcacgcta |
| | | cgtg |
| 311 | m1CD8α Nucleic | ATGGCGCTTCCCGTGACCGCACTCCTGTTGCCCCTTG |
| | Acid Sequence, | CCCTGCTGTTGCACGCCGCACGACCTTCCCAATTCCG |
| | codon optimized | GGTGTCCCCTCTGGATCGCACCTGGAACCTCGGGGA |
| | | AACGGTGGAGCTCAAGTGTCAAGTCCTCCTGTCGAA |
| | | CCCGACCAGCGGATGCAGCTGGCTGTTCCAGCCGAG |
| | | AGGAGCTGCCGCCTCACCCACCTTCCTCCTGTACTTG |
| | | AGCCAGAACAAGCCGAAGGCCGCTGAGGGTCTGGA |
| | | CACCCAGCGCTTCTCGGGCAAACGGCTGGGAGACAC |
| | | TTTTGTGCTGACTCTCTCCGACTTCCGGCGGGAGAAC |
| | | GAGGGCTACTACTTCTGCTCTGCGCTCTCCAATTCAA |
| | | TCATGTACTTCTCACACTTCGTGCCGGTGTTCCTGCC |
| | | TGCCAGCGTGGTGGACTTCCTCCCCACTACCGCCCA |
| | | ACCCACTAAGAAGTCAACCCTGAAGAAGCGGGTTTG |
| | | CAGACTCCCACGGCCGGAAACGCAGAAGGGTCCGCT |
| | | GTGTTCCCCGATCTACATTTGGGCCCCTTTGGCTGGC |
| | | ACCTGTGGAGTGCTGCTCCTGTCCCTTGTGATCACCC |
| | | TGTACTGCAACCACCGGAATAGGCGGAGAGTCTGCA |
| | | AGTGTCCGCGGCCTGTCGTGAAGTCAGGAGATAAGC |
| | | CGAGCCTGTCCGCACGCTACGTG |
| 312 | MSCV Pro- | TGAAAGACCCCACCTGTAGGTTTGGCAAGCTAGCTT |
| | Dominant | AAGTAACGCCATTTTGCAAGGCATGGAAAATACATA |
| | Negative | ACTGAGAATAGAGAAGTTCAGATCAAGGTTAGGAA |
| | TGFβRII var1- | CAGAGAGACAGCAGAATATGGGCCAAACAGGATAT |
| | WPRE Nucleic | CTGTGGTAAGCAGTTCCTGCCCCGGCTCAGGGCCAA |
| | Acid Sequence | GAACAGATGGTCCCCAGATGCGGTCCCGCCCTCAGC |
| | | AGTTTCTAGAGAACCATCAGATGTTTCCAGGGTGCC |
| | | CCAAGGACCTGAAAATGACCCTGTGCCTTATTTGAA |
| | | CTAACCAATCAGTTCGCTTCTCGCTTCTGTTCGCGCG |
| | | CTTCTGCTCCCCGAGCTCAATAAAAGAGCCCACAAC |
| | | CCCTCACTAGCGGCCGCCCCGGGTCGACGCTACCAC |
| | | CATGGGTCGGGGGCTGCTCAGGGGCCTGTGGCCGCT |
| | | GCACATCGTCCTGTGGACGCGTATCGCCAGCACGAT |
| | | CCCACCGCACGTTCAGAAGTCGGATGTGGAAATGGA |
| | | GGCCCAGAAAGATGAAATCATCTGCCCCAGCTGTAA |
| | | TAGGACTGCCCATCCACTGAGACATATTAATAACGA |
| | | CATGATAGTCACTGACAACAACGGTGCAGTCAAGTT |
| | | TCCACAACTGTGTAAATTTTGTGATGTGAGATTTTCC |
| | | ACCTGTGACAACCAGAAATCCTGCATGAGCAACTGC |
| | | AGCATCACCTCCATCTGTGAGAAGCCACAGGAAGTC |
| | | TGTGTGGCTGTATGGAGAAAGAATGACGAGAACATA |
| | | ACACTAGAGACAGTTTGCCATGACCCCAAGCTCCCC |
| | | TACCATGACTTTATTCTGGAAGATGCTGCTTCTCCAA |
| | | AGTGCATTATGAAGGAAAAAAAAAAGCCTGGTGAG |
| | | ACTTTCTTCATGTGTTCCTGTAGCTCTGATGAGTGCA |
| | | ATGACAACATCATCTTCTCAGAAGAATATAACACCA |
| | | GCAATCCTGACTTGTTGCTAGTCATATTTCAAGTGAC |
| | | AGGCATCAGCCTCCTGCCACCACTGGGAGTTGCCAT |
| | | ATCTGTCATCATCATCTTCTACTGCTACCGCGTTAAC |
| | | CGGCAGCAGAAGCTGAGTTCATAGACCGGTCCGCAG |
| | | TCTGACGTACGCGTAATCAACCTCTGGATTACAAAA |
| | | TTTGTGAAAGATTGACTGGTATTCTTAACTATGTTGC |
| | | TCCTTTTACGCTATGTGGATACGCTGCTTTAATGCCT |
| | | TTGTATCATGCTATTGCTTCCCGTATGGCTTTCATTTT |
| | | CTCCTCCTTGTATAAATCCTGGTTGCTGTCTCTTTAT |
| | | GAGGAGTTGTGGCCCGTTGTCAGGCAACGTGGCGTG |
| | | GTGTGCACTGTGTTTGCTGACGCAACCCCCACTGGTT |
| | | GGGGCATTGCCACCACCTGTCAGCTCCTTTCCGGGA |
| | | CTTTCGCTTTCCCCCTCCCTATTGCCACGGCGGAACT |
| | | CATCGCCGCCTGCCTTGCCCGCTGCTGGACAGGGGC |
| | | TCGGCTGTTGGGCACTGACAATTCCGTGGTGTTGTCG |
| | | GGGAAGCTGACGTCCTTTCCATGGCTGCTCGCCTGT |
| | | GTTGCCACCTGGATTCTGCGCGGGACGTCCTTCTGCT |
| | | ACGTCCCTTCGGCCCTCAATCCAGCGGACCTTCCTTC |
| | | CCGCGGCCTGCTGCCGGCTCTGCGGCCTCTTCCGCGT |
| | | CTTCGCCTTCGCCCTCAGACGAGTCGGATCTCCCTTT |
| | | GGGCCGCCTCCCCGCC |
| 313 | MSCV Pro- | TGAAAGACCCCACCTGTAGGTTTGGCAAGCTAGCTT |
| | Dominant | AAGTAACGCCATTTTGCAAGGCATGGAAAATACATA |
| | Negative | ACTGAGAATAGAGAAGTTCAGATCAAGGTTAGGAA |
| | TGFβRII var2- | CAGAGAGACAGCAGAATATGGGCCAAACAGGATAT |
| | WPRE Nucleic | CTGTGGTAAGCAGTTCCTGCCCCGGCTCAGGGCCAA |
| | Acid Sequence | GAACAGATGGTCCCCAGATGCGGTCCCGCCCTCAGC |
| | | AGTTTCTAGAGAACCATCAGATGTTTCCAGGGTGCC |
| | | CCAAGGACCTGAAAATGACCCTGTGCCTTATTTGAA |
| | | CTAACCAATCAGTTCGCTTCTCGCTTCTGTTCGCGCG |
| | | CTTCTGCTCCCCGAGCTCAATAAAAGAGCCCACAAC |
| | | CCCTCACTAGCGGCCGCCCCGGGTCGACGCTACCAC |
| | | CATGGGTCGGGGGCTGCTCAGGGGCCTGTGGCCGCT |
| | | GCACATCGTCCTGTGGACGCGTATCGCCAGCACGAT |
| | | CCCACCGCACGTTCAGAAGTCGGTTAATAACGACAT |
| | | GATAGTCACTGACAACAACGGTGCAGTCAAGTTTCC |
| | | ACAACTGTGTAAATTTTGTGATGTGAGATTTTCCACC |
| | | TGTGACAACCAGAAATCCTGCATGAGCAACTGCAGC |
| | | ATCACCTCCATCTGTGAGAAGCCACAGGAAGTCTGT |
| | | GTGGCTGTATGGAGAAAGAATGACGAGAACATAAC |
| | | ACTAGAGACAGTTTGCCATGACCCCAAGCTCCCCTA |
| | | CCATGACTTTATTCTGGAAGATGCTGCTTCTCCAAAG |
| | | TGCATTATGAAGGAAAAAAAAAAGCCTGGTGAGAC |
| | | TTTCTTCATGTGTTCCTGTAGCTCTGATGAGTGCAAT |
| | | GACAACATCATCTTCTCAGAAGAATATAACACCAGC |
| | | AATCCTGACTTGTTGCTAGTCATATTTCAAGTGACAG |
| | | GCATCAGCCTCCTGCCACCACTGGGAGTTGCCATAT |
| | | CTGTCATCATCATCTTCTACTGCTACCGCGTTAACCG |
| | | GCAGCAGAAGCTGAGTTCATAGACCGGTCCGCAGTC |
| | | TGACGTACGCGTAATCAACCTCTGGATTACAAAATT |
| | | TGTGAAAGATTGACTGGTATTCTTAACTATGTTGCTC |
| | | CTTTTACGCTATGTGGATACGCTGCTTTAATGCCTTT |
| | | GTATCATGCTATTGCTTCCCGTATGGCTTTCATTTTCT |
| | | CCTCCTTGTATAAATCCTGGTTGCTGTCTCTTTATGA |
| | | GGAGTTGTGGCCCGTTGTCAGGCAACGTGGCGTGGT |
| | | GTGCACTGTGTTTGCTGACGCAACCCCCACTGGTTG |
| | | GGGCATTGCCACCACCTGTCAGCTCCTTTCCGGGACT |
| | | TTCGCTTTCCCCCTCCCTATTGCCACGGCGGAACTCA |
| | | TCGCCGCCTGCCTTGCCCGCTGCTGGACAGGGGCTC |
| | | GGCTGTTGGGCACTGACAATTCCGTGGTGTTGTCGG |
| | | GGAAGCTGACGTCCTTTCCATGGCTGCTCGCCTGTGT |
| | | TGCCACCTGGATTCTGCGCGGGACGTCCTTCTGCTAC |
| | | GTCCCTTCGGCCCTCAATCCAGCGGACCTTCCTTCCC |
| | | GCGGCCTGCTGCCGGCTCTGCGGCCTCTTCCGCGTCT |
| | | TCGCCTTCGCCCTCAGACGAGTCGGATCTCCCTTTGG |
| | | GCCGCCTCCCCGCC |
| 314 | Linker Amino | GGGGSGGGGSGGGGS |
| | Acid Sequence | |
| 315 | Linker Nucleic | GGCGGCGGCGGCAGCGGCGGCGGCGGCAGCGGCGG |
| | Acid Sequence | CGGCGGCAGC |
| 316 | Linker Amino | KESGSVSSEQLAQFRSLD |
| | Acid Sequence | |
| 317 | Linker Nucleic | AAAGAGTCCGGCTCCGTGTCCTCCGAACAGCTGGCG |
| | Acid Sequence | CAGTTTCGTTCCCTGGAT |
| 318 | Linker Amino | EGKSSGSGSESKST |
| | Acid Sequence | |
| 319 | Linker Nucleic | GAAGGCAAATCCTCCGGCTCCGGCTCCGAATCCAAA |
| | Acid Sequence | TCCACC |
| 320 | Linker Amino | SGGGSGGGGSGGGGSGGGGSGGGGSGGGTLQ |
| | Acid Sequence | |
| 321 | Linker Nucleic | TCTGGTGGTGGTTCTGGTGGGGGTGGCTCTGGCGGC |
| | Acid Sequence | GGGGGATCAGGCGGAGGAGGGTCCGGAGGCGGAGG |
| | | CTCTGGTGGGGGTACTCTACAG |
| 322 | Linker Amino | SGGGSGGGGSGGGGSGGGTLQ |
| | Acid Sequence | |
| 323 | Linker Nucleic | TCTGGTGGTGGTTCTGGTGGGGGTGGCTCTGGCGGC |
| | Acid Sequence | GGGGGATCTGGTGGGGGTACTCTACAG |
| 324 | Linker Amino | SGGGSGGGGSGGGGSGGGGSGGGSLQ |
| | Acid Sequence | |
| 325 | Linker Nucleic | AGCGGCGGCGGCAGCGGCGGCGGCGGCAGCGGCGG |
| | Acid Sequence | CGGCGGCAGCGGCGGCGGCGGCAGCGGCGGCGGCA |
| | | GCCTACAG |
| 326 | Linker Amino | SGGSGGGGSGGGSGGGGSLQ |
| | Acid Sequence | |
| 327 | Linker Amino | GSGSGSGS |
| | Acid Sequence | |
| 328 | Linker Amino | GGSGGSGGSGG |
| | Acid Sequence | |
| 329 | Linker Amino | GGSGG |
| | Acid Sequence | |
| 330 | Linker Amino | GGGGSGGGGSGGGGS |
| | Acid Sequence | |
| 331 | Linker Amino | GGGGGGGG |
| | Acid Sequence | |
| 332 | Linker Amino | GGGGGG |
| | Acid Sequence | |
| 333 | Linker Amino | GGGGS |
| | Acid Sequence | |
| 334 | Linker Amino | GGGGSGGGGS |
| | Acid Sequence | |
| 335 | Linker Amino | GGSGGHMGSGG |
| | Acid Sequence | |
| 336 | Linker Amino | EAAAKEAAAKEAAAK |
| | Acid Sequence | |
| 337 | Linker Amino | EAAAKEAAAK |
| | Acid Sequence | |
| 338 | Linker Amino | EAAAK |
| | Acid Sequence | |
| 339 | Linker Amino | AEAAAKEAAAKEAAAKEAAAKALEAEAAAKEAAAK |
| | Acid Sequence | EAAAK EAAAKA |
| 340 | Linker Amino | PAPAP |
| | Acid Sequence | |
| 341 | Linker Amino | AEAAAKEAAAKA |
| | Acid Sequence | |
| 342 | Linker Amino | VSQTSKLTRAETVFPDV |
| | Acid Sequence | |
| 343 | Linker Amino | PLGLWA |
| | Acid Sequence | |
| 344 | Linker Amino | RVLAEA |
| | Acid Sequence | |
| 345 | Linker Amino | EDVVCCSMSY |
| | Acid Sequence | |
| 346 | Linker Amino | GGIEGRGS |
| | Acid Sequence | |
| 347 | Linker Amino | TRHRQPRGWE |
| | Acid Sequence | |
| 348 | Linker Amino | AGNRVRRSVG |
| | Acid Sequence | |
| 349 | Linker Amino | RRRRRRRRR |
| | Acid Sequence | |
| 350 | Linker Amino | GFLG |
| | Acid Sequence | |
| 351 | Linker Amino | GGGGSLVPRGSGGGGS |
| | Acid Sequence | |
| 352 | Linker Amino | APAPAPAPAPAPAPAPAPAPAPAPAPAPAPAPAP |
| | Acid Sequence | |
| 353 | Linker Amino | APAPAPAPAPAPAPAPAPAPAPAPAPAPAPAP |
| | Acid Sequence | |
| 354 | Linker Amino | APAPAPAPAPAPAPAPAPAPAPAPAPAPAP |
| | Acid Sequence | |
| 355 | Linker Amino | APAPAPAPAPAPAPAPAPAPAPAPAPAP |
| | Acid Sequence | |
| 356 | Linker Amino | APAPAPAPAPAPAPAPAPAPAPAPAP |
| | Acid Sequence | |
| 357 | Linker Amino | APAPAPAPAPAPAPAPAPAPAPAP |
| | Acid Sequence | |
| 358 | Linker Amino | APAPAPAPAPAPAPAPAPAPAP |
| | Acid Sequence | |
| 359 | Linker Amino | APAPAPAPAPAPAPAPAPAP |
| | Acid Sequence | |
| 360 | Linker Amino | APAPAPAPAPAPAPAPAP |
| | Acid Sequence | |
| 361 | Linker Amino | APAPAPAPAPAPAPAP |
| | Acid Sequence | |
| 362 | Linker Amino | APAPAPAPAPAPAP |
| | Acid Sequence | |
| 363 | Linker Amino | APAPAPAPAPAP |
| | Acid Sequence | |
| 364 | Linker Amino | APAPAPAPAP |
| | Acid Sequence | |
| 365 | Kozak Sequence | GCCNCCATGG, where N is a purine (A or G) |
| 366 | Hepatitis B Virus | TAAACAGGCCTATTGATTGGAAAGTTTGTCAACGAA |
| | (HBV) Post- | TTGTGGGTCTTTTGGGGTTTGCTGCCCCTTTTACGCA |
| | Transcriptional | ATGTGGATATCCTGCTTTAATGCCTTTATATGCATGT |
| | Regulatory | ATACAAGCAAAACAGGCTTTTACTTTCTCGCCAACTT |
| | Element Nucleic | ACAAGGCCTTTCTCAGTAAACAGTATATGACCCTTT |
| | Acid Sequence | ACCCCGTTGCTCGGCAACGGCCTGGTCTGTGCCAAG |
| | (HPRE) | TGTTTGCTGACGCAACCCCCACTGGTTGGGGCTTGG |
| | | CCATAGGCCATCAGCGCATGCGTGGAACCTTTGTGT |
| | | CTCCTCTGCCGATCCATACTGCGGAACTCCTAGCCGC |
| | | TTGTTTTGCTCGCAGCAGGTCTGGAGCAAACCTCATC |
| | | GGGACCGACAATTCTGTCGTACTCTCCCGCAAGTAT |
| | | ACATCGTTTCCATGGCTGCTAGGCTGTGCTGCCAACT |
| | | GGATCCTGCGCGGGACGTCCTTTGTTTACGTCCCGTC |
| | | GGCGCTGAATCCCGCGGACGACCCCTCCCGGGGCCG |
| | | CTTGGGGCTCTACCGCCCGCTTCTCCGTCTGCCGTAC |
| | | CGTCCGACCACGGGGCGCACCTCTCTTTACGCGGAC |
| | | TCCCCGTCTGTGCCTTCTCATCTGCCGGACCGTGTGC |
| | | ACTTCGCTTCACCTCTGCACGTCGCATGGAGACCAC |
| | | CGTGAACGCCCACCGGAACCTGCCCAAGGTCTTGCA |
| | | TAAGAGGACTCTTGGACTTTCAGCAATGTC |
| 367 | Wild Type (“wt”) | MGRGLLRGLWPLHIVLWTRIASTIPPHVQKSDVEMEA |
| | TGFβRII Amino | QKDEIICPSCNRTAHPLRHINNDMIVTDNNGAVKFPQL |
| | Acid Sequence | CKFCDVRFSTCDNQKSCMSNCSITSICEKPQEVCVAVW |
| | | RKNDENITLETVCHDPKLPYHDFILEDAASPKCIMKEK |
| | | KKPGETFFMCSCSSDECNDNIIFSEEYNTSNPDLLLVIFQ |
| | | VTGISLLPPLGVAISVIIIFYCYRVNRQQKLSSTWETGKT |
| | | RKLMEFSEHCAIILEDDRSDISSTCANNINHNTELLPIEL |
| | | DTLVGKGRFAEVYKAKLKQNTSEQFETVAVKIFPYEE |
| | | YASWKTEKDIFSDINLKHENILQFLTAEERKTELGKQY |
| | | WLITAFHAKGNLQEYLTRHVISWEDLRKLGSSLARGIA |
| | | HLHSDHTPCGRPKMPIVHRDLKSSNILVKNDLTCCLCD |
| | | FGLSLRLDPTLSVDDLANSGQVGTARYMAPEVLESRM |
| | | NLENVESFKQTDVYSMALVLWEMTSRCNAVGEVKDY |
| | | EPPFGSKVREHPCVESMKDNVLRDRGRPEIPSFWLNHQ |
| | | GIQMVCETLTECWDHDPEARLTAQCVAERFSELEHLD |
| | | RLSGRSCSEEKIPEDGSLNTTK |
| | Linker Amino | LE |
| | Acid Sequence | |
| | Kozak Sequence | GCCACC |
| | Kozak Sequence | ACCATGG |

The constructs in Table 2 may be assemblages of the individual components described in Table 3. The inventors found that the combination, order, and inclusion of transcription enhancers from Table 3 as described in Table 2 provided unexpected improvements in transfection efficiency, expression levels, and induction of cytotoxic T-cell activities, e.g., IL-12 secretion, IFN-γ secretion, TNF-α secretion, granzyme A secretion, MIP-1 a secretion, IP-10 secretion, granzyme B secretion, and any combination thereof.

### Tumor Associated Antigens (TAA)

In the MHC class I dependent immune reaction, peptides not only have to be able to bind to certain MHC class I molecules expressed by tumor cells, they subsequently also have to be recognized by T cells bearing specific T cell receptors (TCR).

For proteins to be recognized by T-lymphocytes as tumor-specific or -associated antigens, and to be used in a therapy, particular prerequisites must be fulfilled. The antigen should be expressed mainly by tumor cells and not, or in comparably small amounts, by normal healthy tissues. In embodiments, the peptide may be over-presented by tumor cells as compared to normal healthy tissues. It is furthermore desirable that the respective antigen is not only present in a type of tumor, but also in high concentrations (e.g., copy numbers of the respective peptide per cell). Tumor-specific and tumor-associated antigens are often derived from proteins directly involved in transformation of a normal cell to a tumor cell due to their function, e.g., in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach. Singh-Jasuja et al. Cancer Immunol. Immunother. 53 (2004): 187-195. Epitopes are present in the amino acid sequence of the antigen, making the peptide an “immunogenic peptide”, and being derived from a tumor associated antigen, leads to a T-cell-response, both in vitro and in vivo.

Any peptide able to bind an MHC molecule may function as a T-cell epitope. For the induction of a T-cell-response, the TAA must be presented a T cell having a corresponding TCR and the host must not have immunological tolerance for this particular epitope. Exemplary Tumor Associated Antigens (TAA) that may be used with the CD8 polypeptides described herein are disclosed herein.

TABLE 4
TAA Peptide sequences

| SEQ | Amino |
|---|---|
| ID | Acid |
| NO: | Sequence |
| 98 | YLYDSETKNA |
| 99 | HLMDQPLSV |
| 100 | GLLKKINSV |
| 101 | FLVDGSSAL |
| 102 | FLFDGSANLV |
| 103 | FLYKIIDEL |
| 104 | FILDSAETTTL |
| 105 | SVDVSPPKV |
| 106 | VADKIHSV |
| 107 | IVDDLTINL |
| 108 | GLLEELVTV |
| 109 | TLDGAAVNQV |
| 110 | SVLEKEIYSI |
| 111 | LLDPKTIFL |
| 112 | YTFSGDVQL |
| 113 | YLMDDFSSL |
| 114 | KVWSDVTPL |
| 115 | LLWGHPRVALA |
| 116 | KIWEELSVLEV |
| 117 | LLIPFTIFM |
| 118 | FLIENLLAA |
| 119 | LLWGHPRVALA |
| 120 | FLLEREQLL |
| 121 | SLAETIFIV |
| 122 | TLLEGISRA |
| 123 | KIQEILTQV |
| 124 | VIFEGEPMYL |
| 125 | SLFESLEYL |
| 126 | SLLNQPKAV |
| 127 | GLAEFQENV |
| 128 | KLLAVIHEL |
| 129 | TLHDQVHLL |
| 130 | TLYNPERTITV |
| 131 | KLQEKIQEL |
| 132 | SVLEKEIYSI |
| 133 | RVIDDSLVVGV |
| 134 | VLFGELPAL |
| 135 | GLVDIMVHL |
| 136 | FLNAIETAL |
| 137 | ALLQALMEL |
| 138 | ALSSSQAEV |
| 139 | SLITGQDLLSV |
| 140 | QLIEKNWLL |
| 141 | LLDPKTIFL |
| 142 | RLHDENILL |
| 143 | YTFSGDVQL |
| 144 | GLPSATTTV |
| 145 | GLLPSAESIKL |
| 146 | KTASINQNV |
| 147 | SLLQHLIGL |
| 148 | YLMDDFSSL |
| 149 | LMYPYIYHV |
| 150 | KVWSDVTPL |
| 151 | LLWGHPRVALA |
| 152 | VLDGKVAVV |
| 153 | GLLGKVTSV |
| 154 | KMISAIPTL |
| 155 | GLLETTGLLAT |
| 156 | TLNTLDINL |
| 157 | VIIKGLEEI |
| 158 | YLEDGFAYV |
| 159 | KIWEELSVLEV |
| 160 | LLIPFTIFM |
| 161 | ISLDEVAVSL |
| 162 | KISDFGLATV |
| 163 | KLIGNIHGNEV |
| 164 | ILLSVLHQL |
| 165 | LDSEALLTL |
| 166 | VLQENSSDYQSNL |
| 167 | HLLGEGAFAQV |
| 168 | SLVENIHVL |
| 169 | YTFSGDVQL |
| 170 | SLSEKSPEV |
| 171 | AMFPDTIPRV |
| 172 | FLIENLLAA |
| 173 | FTAEFLEKV |
| 174 | ALYGNVQQV |
| 175 | LFQSRIAGV |
| 176 | ILAEEPIYIRV |
| 177 | FLLEREQLL |
| 178 | LLLPLELSLA |
| 179 | SLAETIFIV |
| 180 | AILNVDEKNQV |
| 181 | RLFEEVLGV |
| 182 | YLDEVAFML |
| 183 | KLIDEDEPLFL |
| 184 | KLFEKSTGL |
| 185 | SLLEVNEASSV |
| 186 | GVYDGREHTV |
| 187 | GLYPVTLVGV |
| 188 | ALLSSVAEA |
| 189 | TLLEGISRA |
| 190 | SLIEESEEL |
| 191 | ALYVQAPTV |
| 192 | KLIYKDLVSV |
| 193 | ILQDGQFLV |
| 194 | SLLDYEVSI |
| 195 | LLGDSSFFL |
| 196 | VIFEGEPMYL |
| 197 | ALSYILPYL |
| 198 | FLFVDPELV |
| 199 | SEWGSPHAAVP |
| 200 | ALSELERVL |
| 201 | SLFESLEYL |
| 202 | KVLEYVIKV |
| 203 | VLLNEILEQV |
| 204 | SLLNQPKAV |
| 205 | KMSELQTYV |
| 206 | ALLEQTGDMSL |
| 207 | VIIKGLEEITV |
| 208 | KQFEGTVEI |
| 209 | KLQEEIPVL |
| 210 | GLAEFQENV |
| 211 | NVAEIVIHI |
| 212 | ALAGIVTNV |
| 213 | NLLIDDKGTIKL |
| 214 | VLMQDSRLYL |
| 215 | KVLEHVVRV |
| 216 | LLWGNLPEI |
| 217 | SLMEKNQSL |
| 218 | KLLAVIHEL |
| 219 | ALGDKFLLRV |
| 220 | FLMKNSDLYGA |
| 221 | KLIDHQGLYL |
| 222 | GPGIFPPPPPQP |
| 223 | ALNESLVEC |
| 224 | GLAALAVHL |
| 225 | LLLEAVWHL |
| 226 | SIIEYLPTL |
| 227 | TLHDQVHLL |
| 228 | SLLMWITQC |
| 229 | FLLDKPQDLSI |
| 230 | YLLDMPLWYL |
| 231 | GLLDCPIFL |
| 232 | VLIEYNFSI |
| 233 | TLYNPERTITV |
| 234 | AVPPPPSSV |
| 235 | KLQEELNKV |
| 236 | KLMDPGSLPPL |
| 237 | ALIVSLPYL |
| 238 | FLLDGSANV |
| 239 | ALDPSGNQLI |
| 240 | ILIKHLVKV |
| 241 | VLLDTILQL |
| 242 | HLIAEIHTA |
| 243 | SMNGGVFAV |
| 244 | MLAEKLLQA |
| 245 | YMLDIFHEV |
| 246 | ALWLPTDSATV |
| 247 | GLASRILDA |
| 248 | ALSVLRLAL |
| 249 | SYVKVLHHL |
| 250 | VYLPKIPSW |
| 251 | NYEDHFPLL |
| 252 | VYIAELEKI |
| 253 | VHFEDTGKTLLF |
| 254 | VLSPFILTL |
| 255 | HLLEGSVGV |

### Example 2

### CD8α Molecules and dnTGFβRII Polypeptides

### CD8 Polypeptides

CD8α homodimer (CD8αα) may be composed of two α subunits held together by two disulfide bonds at the stalk regions. FIG. 1 shows a CD8α polypeptide, e.g., SEQ ID NO: 258 (CD8α1), that includes five domains: (1) one signal peptide (from −21 to −1), e.g., SEQ ID NO: 6, (2) one Ig-like domain-1 (from 1 to 115), e.g., SEQ ID NO: 1, (3) one stalk region (from 116 to 160), e.g., SEQ ID NO: 260, (4) one transmembrane (TM) domain (from 161-188), e.g., SEQ ID NO: 3, and (5) one cytoplasmic tail (Cyto) comprising a lck-binding motif (from 189 to 214), e.g., SEQ ID NO: 4. Another example of CD8α subunit, e.g., CD8α2 (SEQ ID NO: 259), differs from CD8α1 at position 112, at which CD8α2 contains a cysteine (C), whereas CD8α1 contains a tyrosine (Y).

Modified CD8 polypeptides

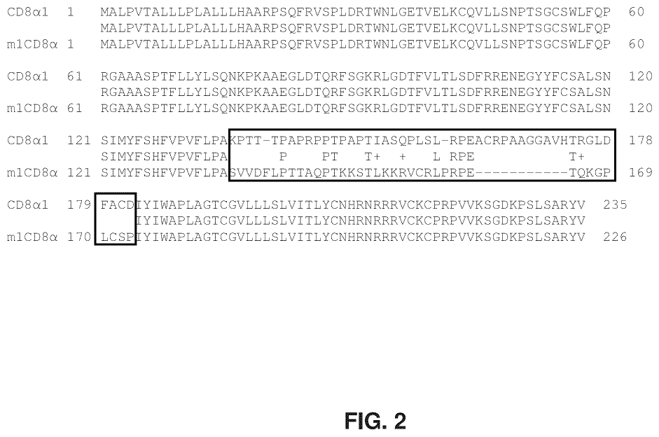

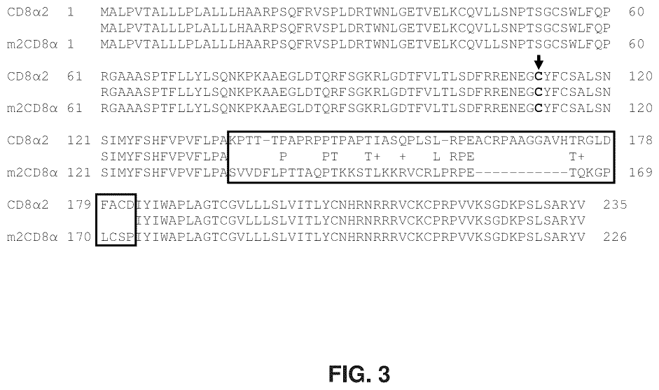

Different from CD8α polypeptide, e.g., CD8α1 (SEQ ID NO: 258) and CD8α2 (SEQ ID NO: 259), a modified CD8α polypeptide, e.g., m1CD8α (SEQ ID NO: 7) and m2CD8α (SEQ ID NO: 262), may contain additional regions, such as sequence stretches from a CD8β polypeptide. In embodiments, SEQ ID NO: 2 or variants thereof are used with a CD8α polypeptide. In other embodiments, a portion of a CD8α polypeptide, e.g., SEQ ID NO: 260, is removed or not included in modified CD8 polypeptides described herein. FIG. 2 shows a sequence alignment between CD8α1 (SEQ ID NO: 258) and m1CD8α (SEQ ID NO: 7). FIG. 3 shows a sequence alignment between CD8α2 (SEQ ID NO: 259) and m2CD8α (SEQ ID NO: 262), in which the cysteine substitution is indicated by an arrow. The stalk regions are shown within the boxes.

Modified CD8 expressing cells showed improved functionality in terms of cytotoxicity and cytokine response as compared to original CD8 expressing T cells transduced with the TCR.

dnTGFβRII Polypeptides

A dnTGFβRII polypeptide may comprise or consist of appropriate amino acid sequences identified herein. A dnTGFβRII polypeptide may be encoded by one or more nucleic acids comprising or consisting of appropriate nucleic acid sequences identified herein. For example, In embodiments, dnTGFβRII variant 1 (dnTGFβRIIvar1) and/or dnTGFβRII variant 2 (dnTGFβRIIvar2) are provided and are examples of dnTGFβRII polypeptides. dnTGFβRIIvar1 may comprise or consist of SEQ ID NO: 305 and may be encoded by a nucleic acid comprising or consisting of SEQ ID NO: 306. dnTGFβRIIvar1 may comprise or consist of SEQ ID NO: 307 and may be encoded by a nucleic acid comprising or consisting of SEQ ID NO: 308. In embodiments, a dnTGFβRII polypeptide encoded by a nucleic acid also comprising and/or encoding a MSCV promoter and a WPRE may be encoded by a nucleic acid comprising or consisting of SEQ ID NO: 312 or SEQ ID NO: 313.

### Example 3

### Lentiviral Viral Vectors

The lentiviral vectors used herein contain several elements that enhance vector function, including a central polypurine tract (cPPT) for improved replication and nuclear import, a promoter from the murine stem cell virus (MSCV) (SEQ ID NO: 263), which lessens vector silencing in some cell types, a woodchuck hepatitis virus posttranscriptional responsive element (WPRE) (SEQ ID NO: 264) for improved transcriptional termination, and the backbone was a deleted 3′-LTR self-inactivating (SIN) vector design that improves safety, sustained gene expression and anti-silencing properties. Yang et al. Gene Therapy (2008) 15, 1411-1423.

In embodiments, vectors, constructs, or sequences described herein comprise mutated forms of WPRE. In embodiments, sequences or vectors described herein comprise mutations in WPRE version 1, e.g., WPREmut1 (SEQ ID NO: 256), or WPRE version 2, e.g., WPREmut2 (SEQ ID NO: 257). Construct #9 and Construct #9b represent two LV production batches with the same construct containing SEQ ID NO: 257 as WPREmut2, with the difference between Construct #9 and Construct #9b being the titer consistent with Table 4. In embodiments, WPRE mutants comprise at most one mutation, at most two mutations, at most three mutations, at least four mutations, or at most five mutations. In embodiments, vectors, constructs, or sequences described herein do not comprise WPRE. In an aspect, WPRE sequences described in U.S. 2021/0285011, the content of which is incorporated by reference in its entirety, may be used together with vectors, sequences, or constructs described herein.

In embodiments, vectors, constructs, or sequences described herein do not include an X protein promoter. The WPRE mutants described herein do not express an X protein. WPRE promotes accumulation of mRNA, theorized to promote export of mRNA from nucleosome to cytoplasm to promote translation of the transgene mRNA.

To obtain optimal co-expression levels of TCRαβ, mCD8α (e.g., m1CD8α (SEQ ID NO: 7 (which may be encoded by SEQ ID NO: 311)) and m2CD8α (SEQ ID NO: 262)) and CD8β (e.g., any one of CD8β1-7 (SEQ ID NO: 8-14)) and/or dnTGFβRII (e.g., any one or both of SEQ ID NO: 305 or 307) in the transduced CD4+ T cells, CD8+ T cells, and/or γδ T cells, lentiviral vectors with various designs were generated. T cells may be transduced with two separate lentiviral vectors (2-in-1), e.g., one expressing TCRα and TCRβ and the other expressing mCD8α and CD8β, for co-expression of TCRαβ and CD8αβ heterodimer, or one expressing TCRα and TCRβ and the other expressing mCD8α for co-expression of TCRαβ and mCD8α homodimer. Alternatively, T cells may be transduced with a single lentiviral vector (4-in-1) co-expressing TCRα, TCRβ, mCD8α, and CD8β for co-expression of TCRαβ and CD8αβ heterodimer. In the 4-in-1 vector, the nucleotides encoding TCRα chain, TCRβ chain, mCD8α chain, and CD8β chain may be shuffled in various orders, e.g., from 5′ to 3′ direction, TCRα-TCRβ-mCD8α-CD8β, TCRα-TCRβ-CD8β-mCD8α, TCRβ-TCRα-mCD8α-CD8β, TCRβ-TCRα-CD8β-mCD8α, mCD8α-CD8β-TCRα-TCRβ, mCD8α-CD8β-TCRβ-TCRα, CD8β-mCD8α-TCRα-TCRβ, and CD8β-mCD8α-TCRβ-TCRα. Various 4-in-1 vectors, thus generated, may be used to transduce CD4+ T cells, CD8+ T cells, and/or γδ T cells, followed by measuring TCRαβ/mCD8α/CD8β co-expression levels of the transduced cells using techniques known in the art, e.g., flow cytometry. Similarly, T cells may be transduced with a single lentiviral vector (3-in-1) co-expressing TCRα, TCRβ, and mCD8α (e.g., m1CD8α and m2CD8α) for co-expression of TCRαβ and mCD8α homodimer. In the 3-in-1 vector, the nucleotides encoding TCRα chain, TCRβ chain, mCD8α chain may be shuffled in various orders, e.g., TCRα-TCRβ-mCD8α, TCRβ-TCRα-mCD8α, mCD8α-TCRα-TCRβ, and mCD8α-TCRβ-TCRα. Various 3-in-1 vectors, thus generated, may be used to transduce CD4+ T cells, CD8+ T cells, and/or γδ T cells, followed by measuring TCRαβ/mCD8α co-expression levels of the transduced cells using techniques known in the art. Similarly, one or more dnTGFβRII polypeptide may be encoded by a separate vector or by a vector also encoding one or more CD8 and/or one or more TCR. Vectors co-expressing any combination of TCRα, TCRβ, mCD8α, CD8β, and/or dnTGFβRII, in any order, may be generated.

To generate lentiviral vectors co-expressing TCRαβ and mCD8α and/or CD8β, a nucleotide encoding furin-linker (GSG or SGSG (SEQ ID NO: 266))-2A peptide may be positioned between TCRα chain and TCRβ chain, between mCD8α chain and CD8β chain, and between a TCR chain and a CD8 chain, and/or between a CD8 or TCR and a dnTGFβRII to enable highly efficient gene expression. The 2A peptide may be selected from P2A (SEQ ID NO: 93), T2A (SEQ ID NO: 94), E2A (SEQ ID NO: 95), or F2A (SEQ ID NO: 96).

Lentiviral viral vectors may also contain post-transcriptional regulatory element (PRE), such as WPRE (SEQ ID NO: 264), WPREmut1 (SEQ ID NO: 256), or WPREmut2 (SEQ ID NO: 257), which may function to enhance the expression of one or more transgene by increasing both nuclear and cytoplasmic mRNA levels. One or more regulatory elements including mouse RNA transport element (RTE), the constitutive transport element (CTE) of the simian retrovirus type 1 (SRV-1), and the 5′ untranslated region of the human heat shock protein 70 (Hsp70 5′UTR) may also be used and/or in combination with WPRE to increase transgene expression. The WPREmut1 and WPREmut2 do not express an X protein, but still act to enhance translation of the transgene mRNA.

Lentiviral vectors may be pseudotyped with RD114TR (for example, SEQ ID NO: 97), which is a chimeric glycoprotein comprising an extracellular and transmembrane domain of feline endogenous virus (RD114) fused to cytoplasmic tail (TR) of murine leukemia virus. Other viral envelop proteins, such as VSV-G env, MLV 4070A env, RD114 env, chimeric envelope protein RD114pro, baculovirus GP64 env, or GALV env, or derivatives thereof, may also be used. RD114TR variants comprising at least 85%, at least 90%, at least 95%, at least 98%, at least 99%, or 100% to SEQ ID NO: 97 also provided for.

For example, FIG. 4 shows exemplary vectors, which include two 4-in-1 vectors, e.g., Constructs #10 and #2, co-expressing TCR (TCRα chain and TCRβ chain), CD8α, and CD8β; three 3-in-1 vectors expressing TCR and CD8α, e.g., Constructs #1 and #9, two 3-in-1 vectors expressing TCR and m1CD8α (SEQ ID NO: 7), e.g., Constructs #11 and #12, and Construct #8 expressing TCR only. To improve transcriptional termination, wild type WPRE (WPRE) (SEQ ID NO: 264) is included in Constructs #1, #2, and #8; WPREmut (SEQ ID NO: 257) is included in Constructs #9, #10, #11, and #12.

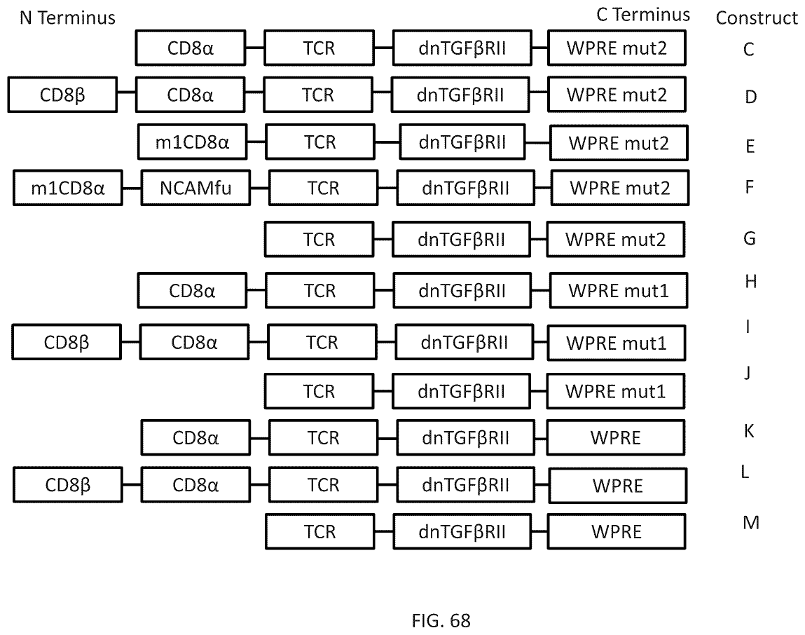

As another example, FIG. 68 depicts exemplary vectors that are provided in embodiments. For example, Constructs C-L depicted in FIG. 68 are provided in embodiments. The TCRs in FIG. 68 may be, for example, TCRβ directly or indirectly fused to TCRα with or without a linker and/or other elements therebetween or TCRα directly or indirectly fused to TCRβ with or without a linker and/or other elements therebetween. The dnTGFβRII polypeptides in FIG. 68 may, as nonlimiting examples, comprise or consist of dnTGFβRIIvar1 (SEQ ID NO: 305, which may be encoded by SEQ ID NO: 306) and or dnTGFβRIIvar2 (SEQ ID NO: 307, which may be encoded by SEQ ID NO: 308). The CD8α, CD8β, and TCR polypeptides in FIG. 68 may independently be as described herein and/or may independently by modified or unmodified. In embodiments, CD8α may comprise or consist of CD8α1 (SEQ ID NO: 258, which may be encoded by SEQ ID NO: 310). In embodiments, CD8α may comprise or consist of m1CD8α (SEQ ID NO: 7, which may be encoded by SEQ ID NO: 311). In embodiments, CD8β may comprise or consist of CD8β1 (SEQ ID NO: 8, which may be encoded by SEQ ID NO: 309).

Further exemplary constructs (Constructs #13-#19 and #21-#26) are described in Table 2 above. In particular, Constructs #13, #14, and #16 are 4-in-1 constructs co-expressing TCR, CD8α, and CD8β3 with various combinations of signal peptides (SEQ ID NO: 6 [WT CD8α signal peptide]; SEQ ID NO: 293 [WT CD8β signal peptide]; and SEQ ID NO: 294 [S19 signal peptide]) and differing element order. Constructs #15 and #17 are 4-in-1 constructs co-expressing TCR, CD8α, and CD8β5. Construct #15 comprises the WT CD8α signal peptide (SEQ ID NO: 6) and WT CD8β signal peptide (SEQ ID NO: 293), whereas Construct #17 comprises the S19 signal peptide (SEQ ID NO: 294) at the N-terminal end of both CD8α and CD8β5. Construct #21 is a 4-in-1 constructs co-expressing TCR, CD8α, and CD8β2 comprising WT CD8α signal peptide (SEQ ID NO: 6) and WT CD8β3 signal peptide (SEQ ID NO: 293). Construct #18 is a variant of Construct #10 in which the WT signal peptides for CD8α and CD8β1 (SEQ ID NOs: 6 and 293, respectively) were replaced with S19 signal peptide (SEQ ID NO: 294). Construct #19 is a variant of Construct #11 in which the WT CD8α signal peptide (SEQ ID NO: 6) was replaced with the S19 signal peptide (SEQ ID NO: 294). Construct #22 is a variant of Construct #11 in which the CD4 transmembrane and intracellular domains are fused to the C-terminus of the CD8β stalk sequence in place of the CD8α transmembrane and intracellular domains. Construct #25 is a variant of Construct #22 in which the CD8β stalk sequence (SEQ ID NO: 2) is replaced with the CD8α stalk sequence (SEQ ID NO: 260).

### Example 4

### Vector Screening (Constructs #1, #2, #8, #9, #10, #11, and #12)

### Viral Titers

FIG. 5A shows viral titer of Constructs #1, #2, #8, #9, #10, #11, and #12. Table 5 shows viral titers and lentiviral P24 ELISA data for Constructs #9, #10, #11, and #12.

TABLE 5

| Constructs # | Titer | Lentiviral P24 |
|---|---|---|
| 9 | 5.40 × 10⁹ | 6556 |
| 9b | 9.80 × 10⁹ | 16196 |
| 10 | 6.40 × 10⁹ | 9525 |
| 11 | 1.30 × 10¹⁰ | 16797 |
| 12 | 1.20 × 10¹⁰ | 17996 |

For construct 12, NCAMfu refers to NCAMFusion protein expressing modified CD8a extracellular and Neural cell adhesion molecule 1 (CD56) intracellular domain.

For Table 5, the WPREmut2 portion refers to SEQ ID NO: 257.

### T Cell Manufacturing

### Activation

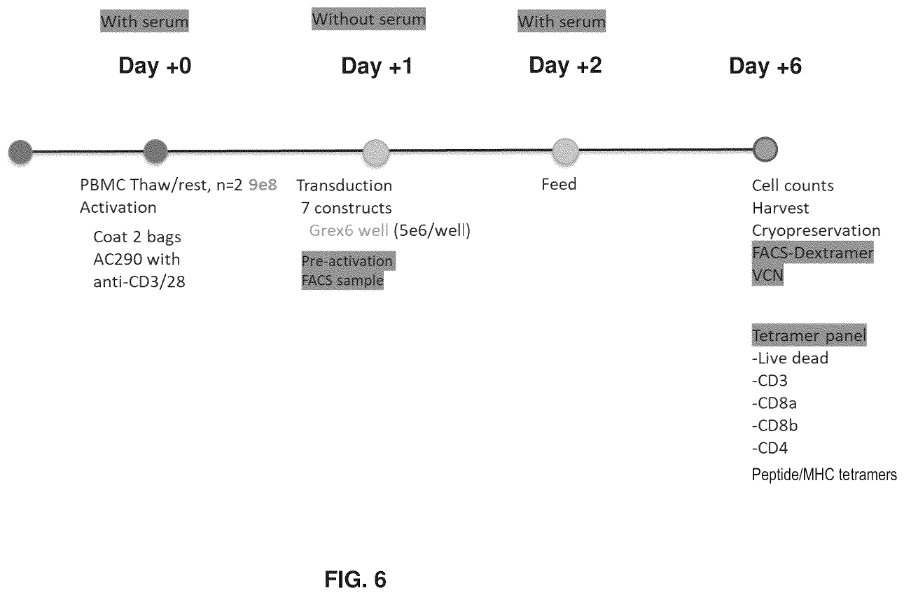

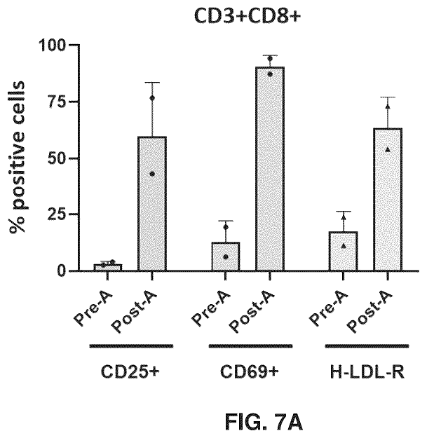

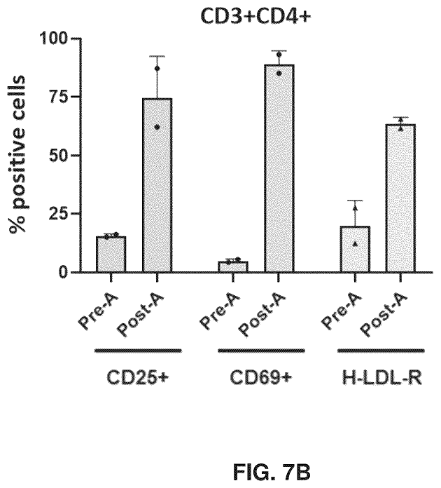

FIG. 6 shows that, on Day +0, PBMCs (about 9×10⁸ cells) obtained from two donors (Donor #1 and Donor #2) were thawed and rested. Cells were activated in bags (AC290) coated with anti-CD3 and anti-CD28 antibodies in the presence of serum. Activation markers, e.g., CD25, CD69, and human low density lipoprotein receptor (H-LDL-R) are in CD8+ and CD4+ cells, were subsequently measured. FIG. 7A shows that % CD3+CD8+CD25+ cells, % CD3+CD8+CD69+ cells, and % CD3+CD8+H-LDL-R+ cells increase after activation (Post-A) as compared with that before activation (Pre-A). Similarly, FIG. 7B shows that % CD3+CD4+CD25+ cells, % CD3+CD4+CD69+ cells, and % CD3+CD4+H-LDL-R+ cells increase after activation (Post-A) as compared with that before activation (Pre-A). These results support the activation of PBMCs.

### Transduction

FIG. 6 shows that, on Day +1, activated PBMCs were transduced with viral vectors, e.g., Constructs #1, #2, #8, #9, #10, #11, and #12, in G-Rex® 6 well plates at about 5×10⁶ cells/well in the absence of serum. The amounts of virus used for transduction are shown in Table 6.

TABLE 6

| Constructs | Virus Volume/1 × 10⁶ cells | |
|---|---|---|
| #9, #10, #11, #12 | 1.25 μl, 2.5 μl, 5 μl | |
| #1 | 1.25 | μl |
| #2 | 5 | μl |
| #8 (TCR) | 2.5 | μl |

### Expansion

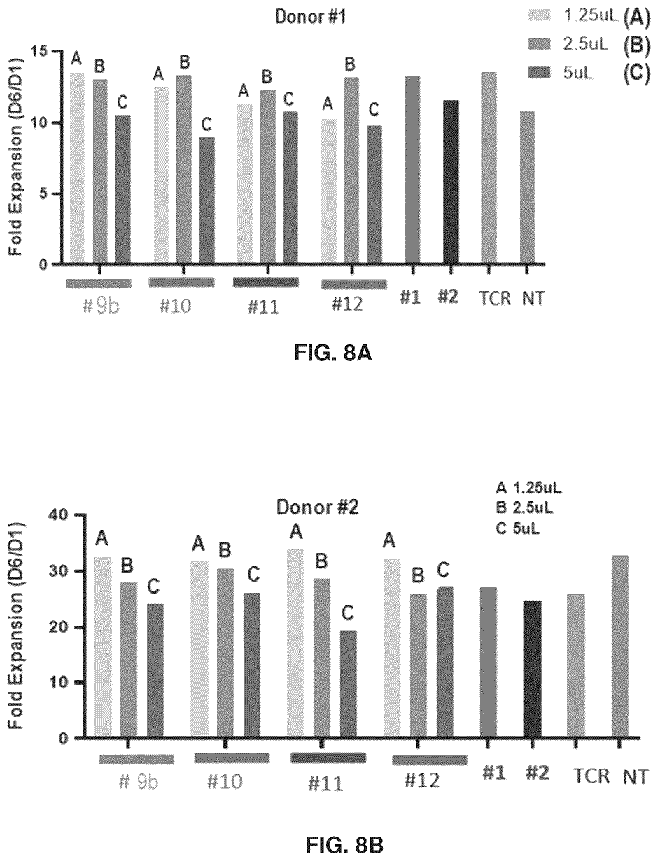

FIG. 6 shows that, on Day +2, transduced PBMCs were expanded in the presence of serum. On Day +6, cells were harvested for subsequent analysis, e.g., FACS-Dextramer and vector copy number (VCN) and were cryopreserved. FIGS. 8A and 8B show fold expansion on Day +6 of transduced T cell products obtained from Donor #1 and donor #2, respectively. Viabilities of cells is greater than 90% on Day +6.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+ Tetramer(Tet)+ and CD8+ Tet+.

FIGS. 9A, 9B, 9C, and 9D show representative flow plots of cells obtained from Donor #1 indicating % CD8, CD4, and PRAME-004/MHC tetramer (Tet) of cells transduced with Construct #9b, #10, #11, or #12, respectively.

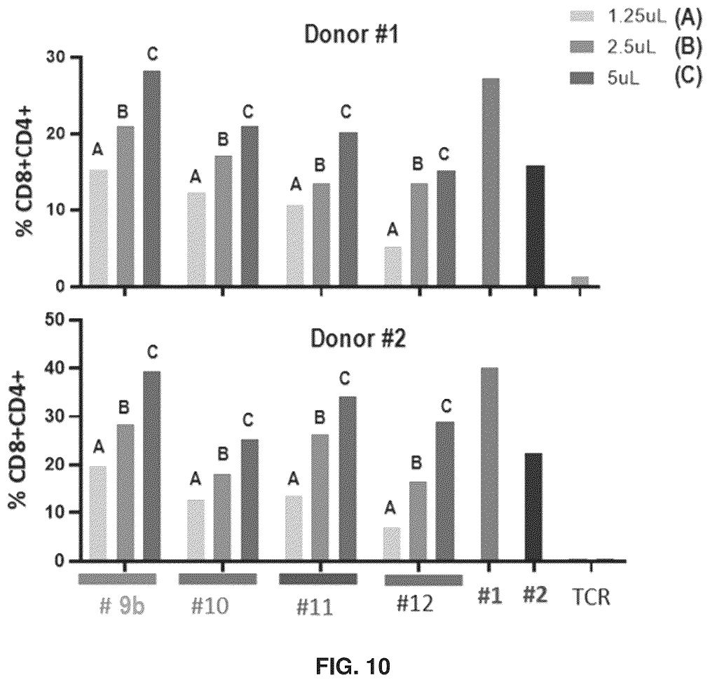

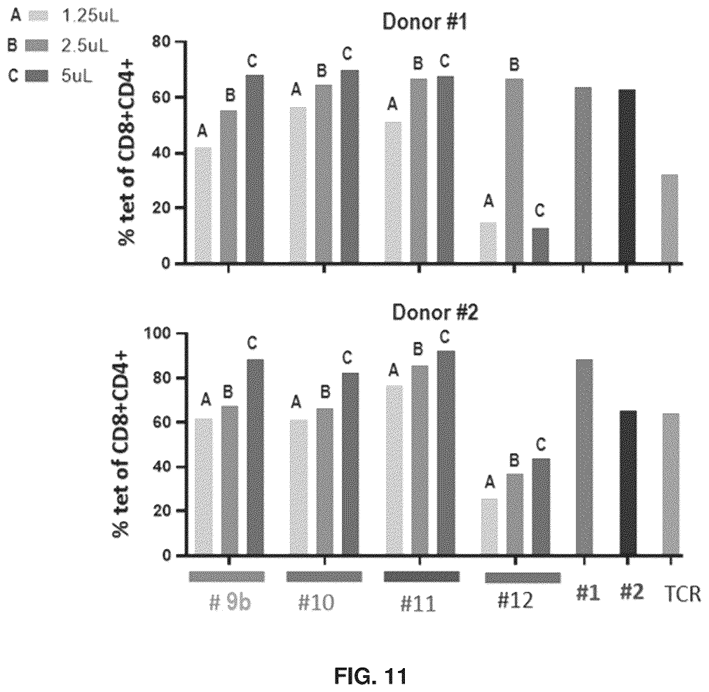

FIG. 10 shows % CD8+CD4+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that higher % CD8+CD4+ cells were obtained by transduction with vectors expressing CD8α and TCR with wild type WPRE (Construct #1) and WPREmut2 (Construct #9) than that transduced with Constructs #10, #11, or #12. Construct #8 (TCR only) serves as negative control. FIG. 11 shows % Tet of CD8+CD4+ cells from Donor #1 (upper panel) and Donor #2 (lower panel) transduced with Constructs #1, #2, #8 (TCR), #9, #10, #11, and #12 at 1.25 μl, 2.5 μl, or 5 μl per 1×10⁶ cells. These results show that % Tet of CD8+CD4+ cells appear comparable among cells transduced with Constructs #9, #10, and #11, and seems greater than that transduced with Construct #12. FACS analysis was gated on live singlets, followed by CD3+, followed by CD4+CD8+, and followed by CD4+CD8+ Tet+.

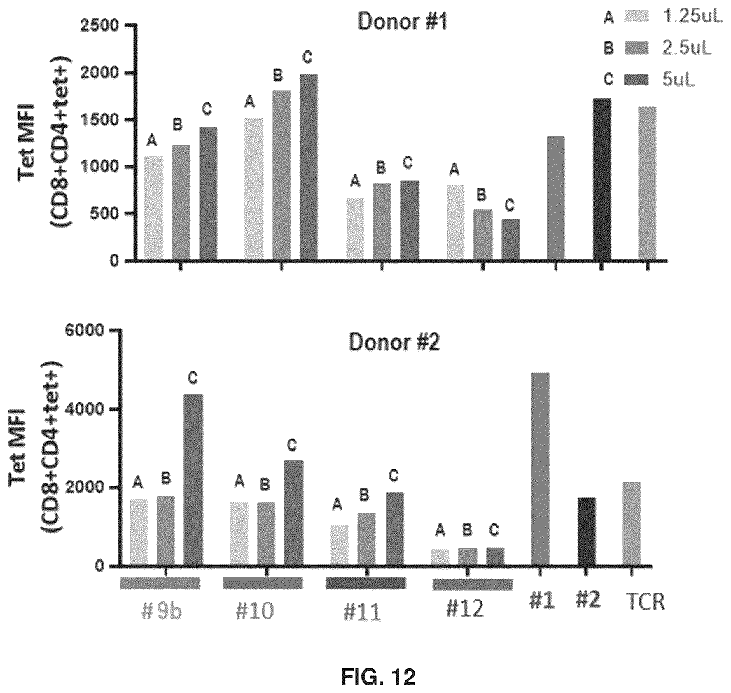

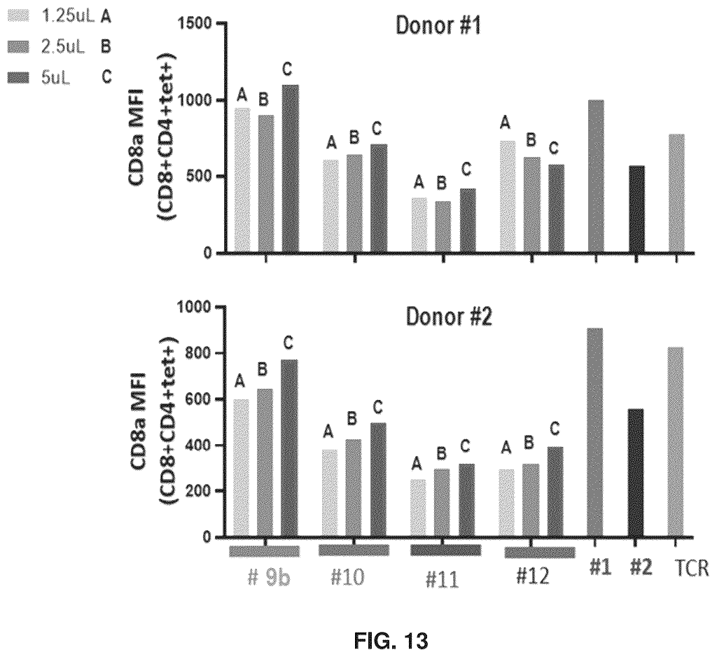

FIG. 12 shows Tet MFI of CD8+CD4+ Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that tetramer MFI on CD4+CD8+ Tet+ varies among donors. FIG. 13 shows CD8α MFI of CD8+CD4+ Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher CD8α MFI in cells transduced with vectors expressing CD8α and TCR with wild type WPRE (Construct #1) and WPREmut2 (Construct #9) than that transduced with the other constructs. Transduction volume of 5 μl/10⁶ appears to yield better results than 1.25 μl/10⁶ and 2.5 μl/10⁶. FACS analysis was gated on live singlets, followed by CD3+, followed by CD4+CD8+, followed by CD4+CD8+ Tet+, and followed by Tet MFI/CD8α MFI.

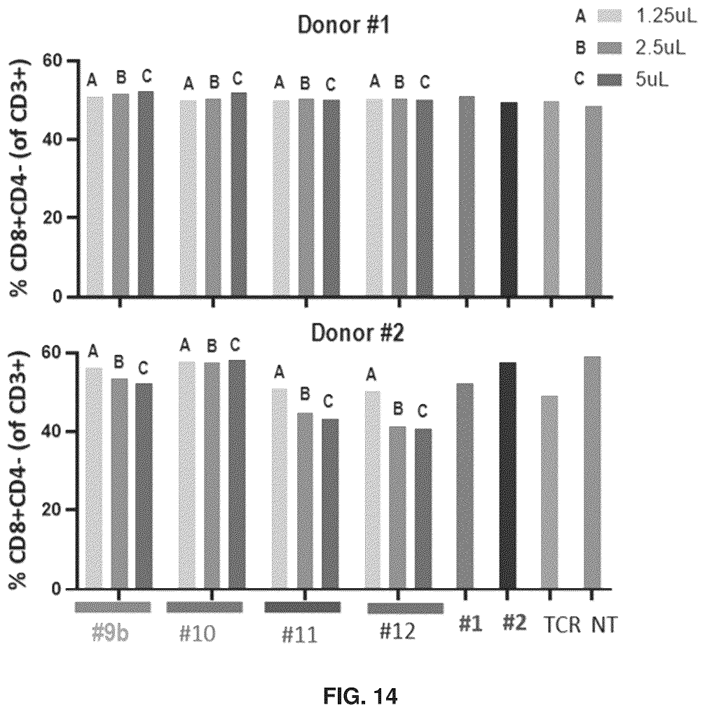

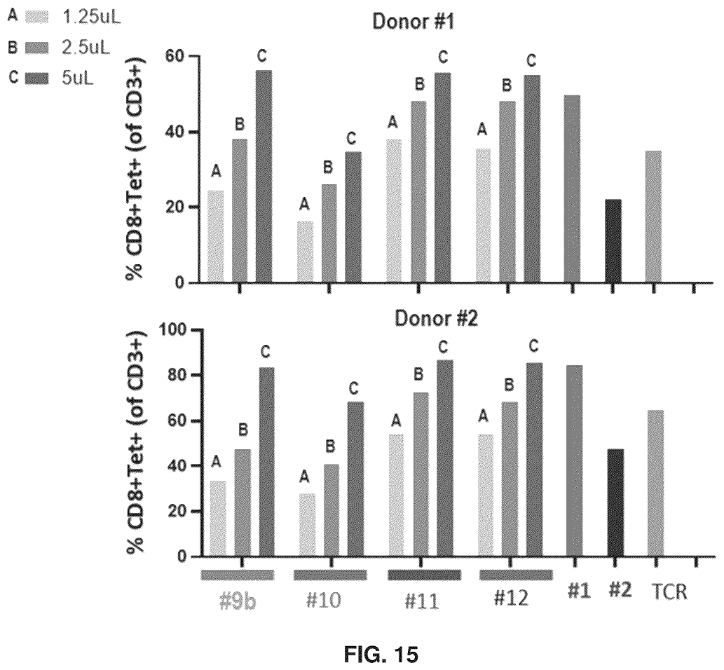

FIG. 14 shows CD8 frequencies (% CD8+CD4− of CD3+) in cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show no difference in the CD8 frequencies among the constructs. Non-transduction (NT) serves as negative control. FIG. 15 shows % CD8+ Tet+ (of CD3+) cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher frequencies of CD8+ Tet+ (of CD3+) in cells transduced with Constructs #9, #11, and #12 than that transduced with Construct #10. FACS analysis was gated on live singlets, followed by CD3+, followed by CD8+CD4−, and followed by CD8+ Tet+.

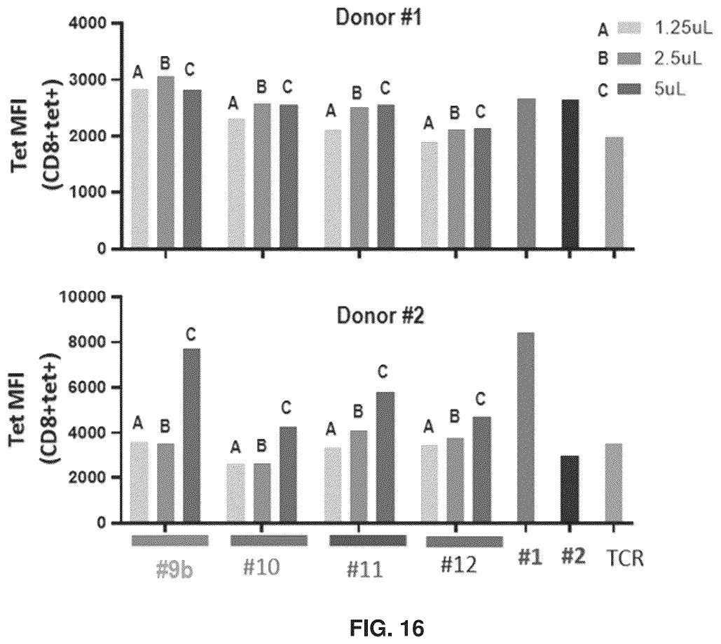

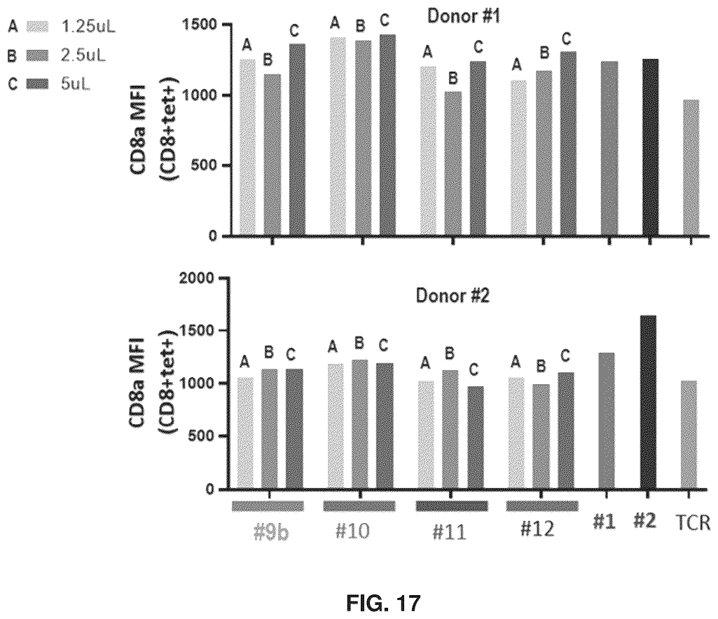

FIG. 16 shows Tet MFI of CD8+ Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show tetramer MFI of CD8+tet+ cells varies among donors. FIG. 17 shows CD8α MFI of CD8+ Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that CD8α MFI of CD8+ Tet+ are comparable among cells transduced with different constructs. FACS analysis was gated on live singlets, followed by CD3+, followed by CD4+CD8+, followed by CD4+CD8+ Tet+, and followed by Tet MFI/CD8α MFI.

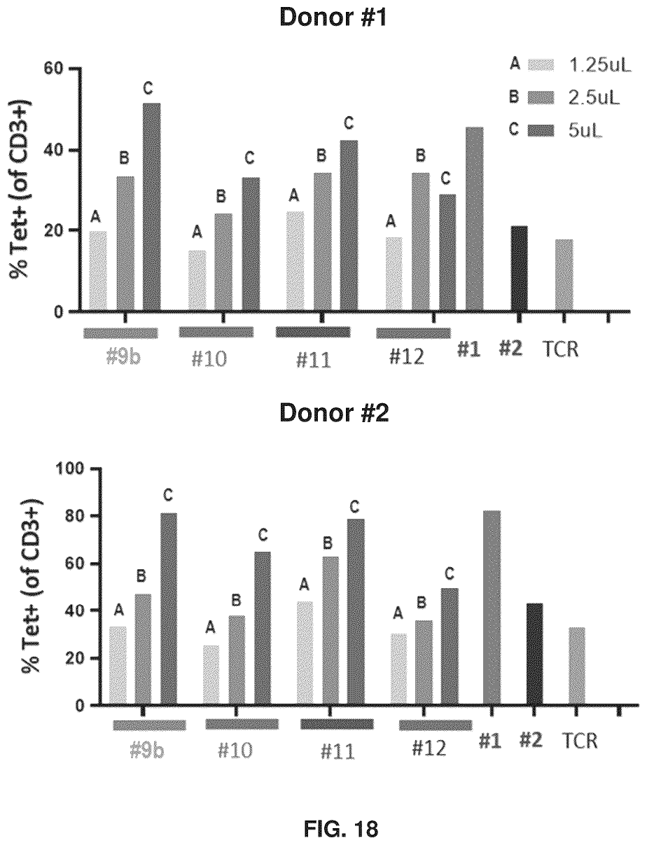

FIG. 18 shows % Tet+ of CD3+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher frequencies of CD3+Tet+ in cells transduced with Construct #9 or #11 than that transduced with Construct #10 or #12. It appears more % Tet+CD3+ cells in cells transduced with Construct #10 (WPREmut2) than that transduced with Construct #2 (wild type WPRE) at 5 μl per 1×10⁶ cells. FACS analysis was gated on live singlets, followed by CD3+, followed by CD3+, and followed by Tet+.

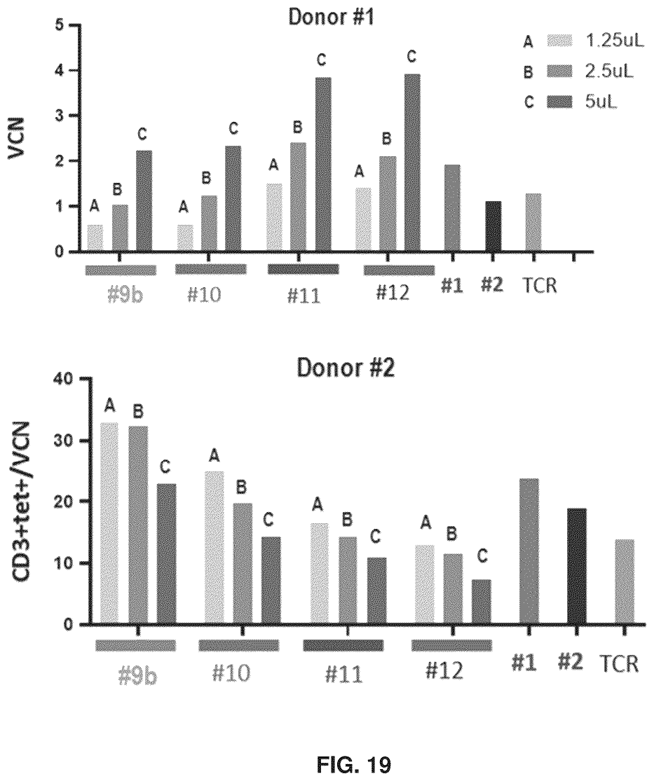

FIG. 19 (upper panel) shows vector copy number (VCN) of cells from Donor #1 transduced with Construct #1, #2, #8 (TCR), #9, #10, #11, or #12 at 1.25 μl, 2.5 μl, or 5 μl per 1×10⁶ cells. These results show higher VCN for cells transduced with Constructs #11 or #12 (may be due to higher titers) than that transduced with Construct #9 or #10. FIG. 19 (lower panel) shows CD3+Tet+/VCN of cells from Donor #1 transduced with Construct #1, #2, #8 (TCR), #9, #10, #11, or #12 at 1.25 μl, 2.5 μl, or 5 μl per 1×10⁶ cells. These results show higher CD3+Tet+/VCN in cells transduced with Construct #9 than that transduced with Construct #10, #11, or #12.

In sum, these results show (1) higher % CD8+CD4+ cells obtained by transducing cells with vectors expressing CD8α and TCR with wild type WPRE (Construct #1) and WPREmut2 (Construct #9) than that transduced with Construct #10, #11 or #12; (2) % CD8+CD4+ Tet+ cells was comparable among cells transduced with different constructs; (3) dose dependent increase in % tetramer, e.g., 5 μl per 1×10⁶ cells showed better results than 1.25 μl and 2.5 μl per 1×10⁶ cells; (4) % CD8+ cells comparable among cells transduced with different constructs; (5) higher frequencies of CD8+ Tet+ in cells transduced with Construct #9, #11, or #12 than that transduced with Construct #10; (6) higher frequencies of CD3+Tet+ in cells transduced with Construct #9 or #11 than that transduced with Construct #10 or #12; (7) higher VCN in cells transduced with Construct #11 or #12 than that transduced with Construct #9 or #10; and (8) higher CD3+tet+/VCN in cells transduced with Construct #9 than that transduced with Construct #10, #11, or #12.

T cell products transduced with viral vector expressing a transgenic TCR and modified CD8 co-receptor showed superior cytotoxicity and increased cytokine production against target positive cell lines.

### Example 5

### Tumor Death Assay

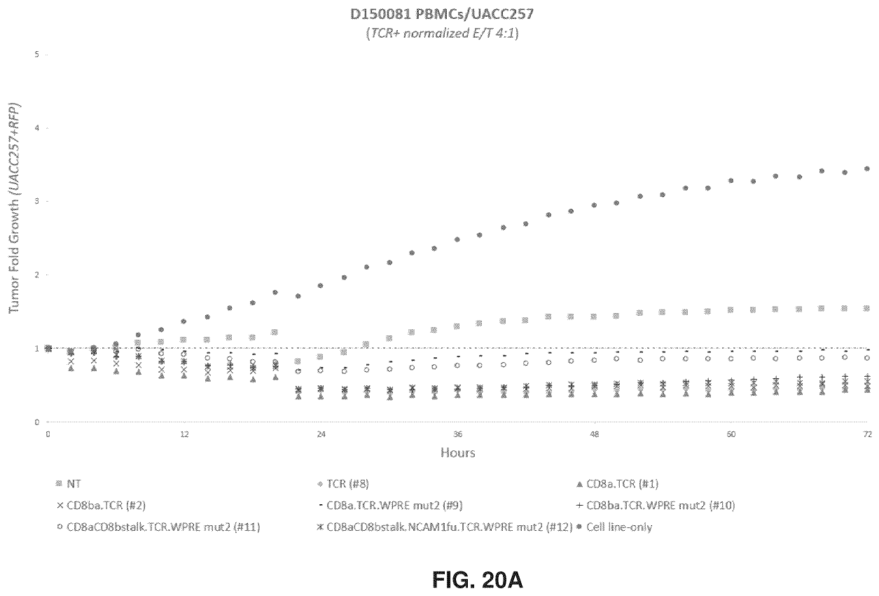

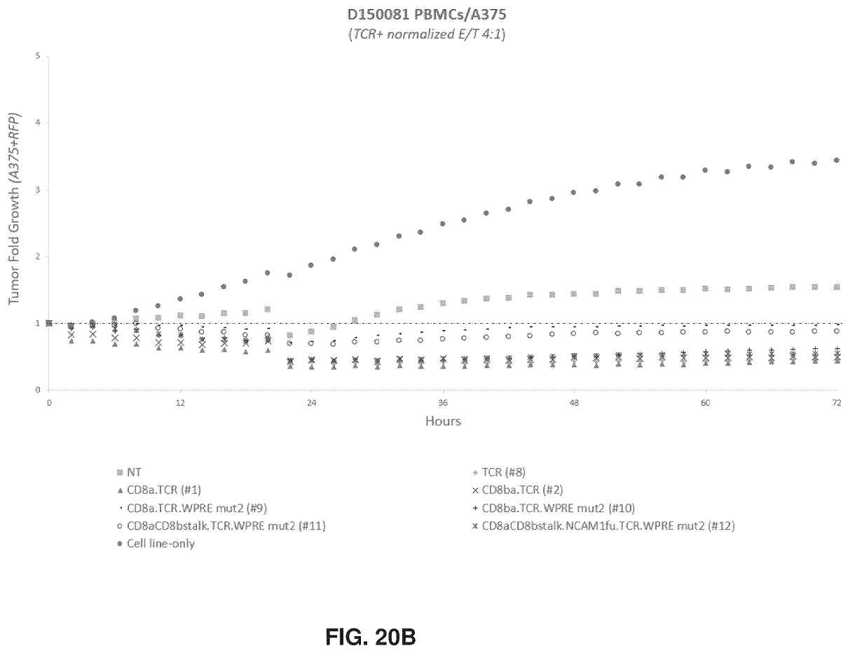

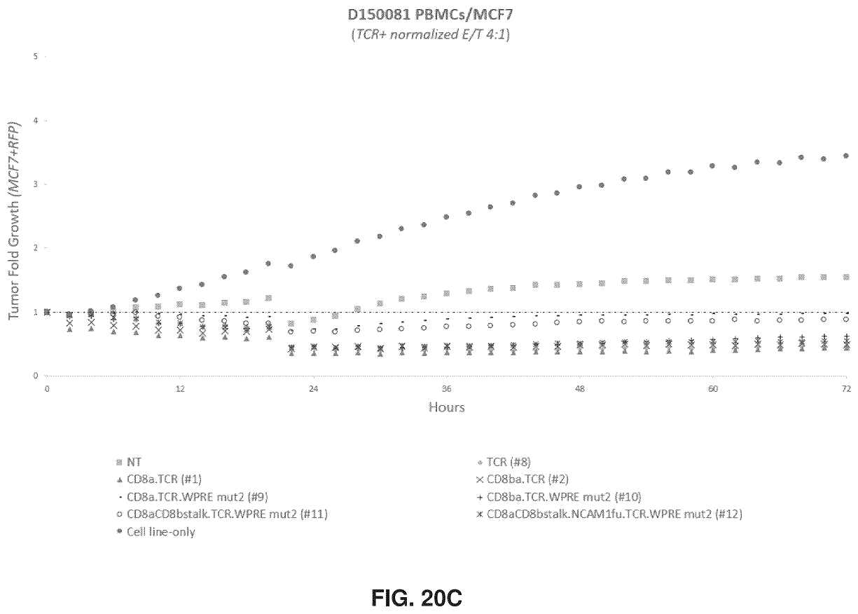

FIG. 20A-C depicts data showing that constructs (#10, #11, & #12) are comparable to TCR-only in mediating cytotoxicity against target positive cells lines expressing antigen at different levels (UACC257 at 1081 copies per cell and A375 at 50 copies per cell).

TABLE 7

| Tumor Cell Line | Antigen Positivity |
|---|---|
| UACC257 | High |
| A375 | Low |
| MCF7 | Negative |

Construct #9 loses tumor control over time against the low target antigen expressing A375 cell line.

### Example 6

### IFNγ Secretion Assay

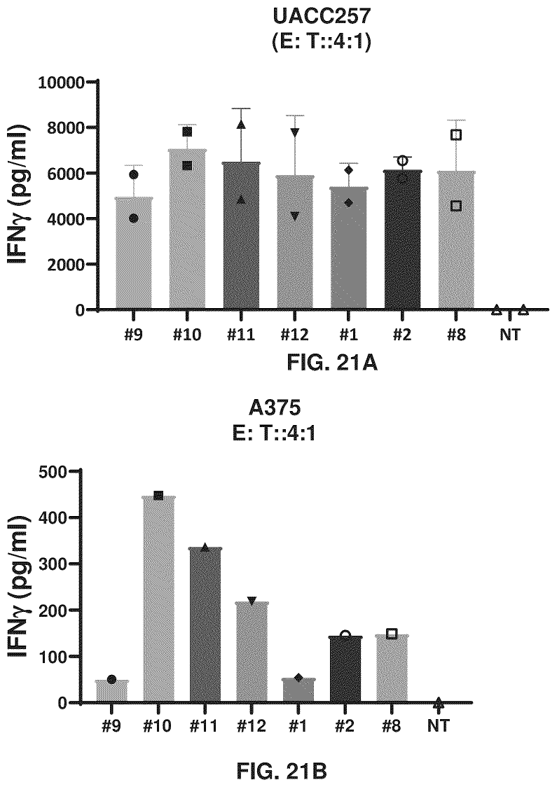

IFNγ secretion was measured in UACC257 and A375 cells lines. IFNγ secretion in response in UACC257 cell line was comparable among constructs. However, in the A375 cell line, Construct #10 showed higher IFNγ secretion than other constructs. IFNγ quantified in the supernatants from Incucyte plates. FIG. 21A-B.

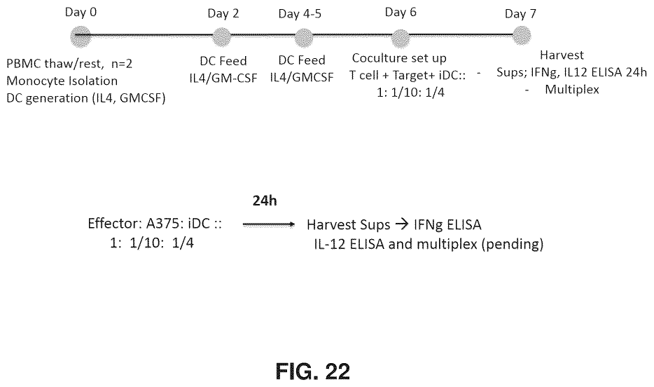

FIG. 22 depicts an exemplary experiment design to assess Dendritic Cell (DC) maturation and cytokine secretion by PBMC-derived T cell products in response to exposure to target positive tumor cell lines UACC257 and A375.

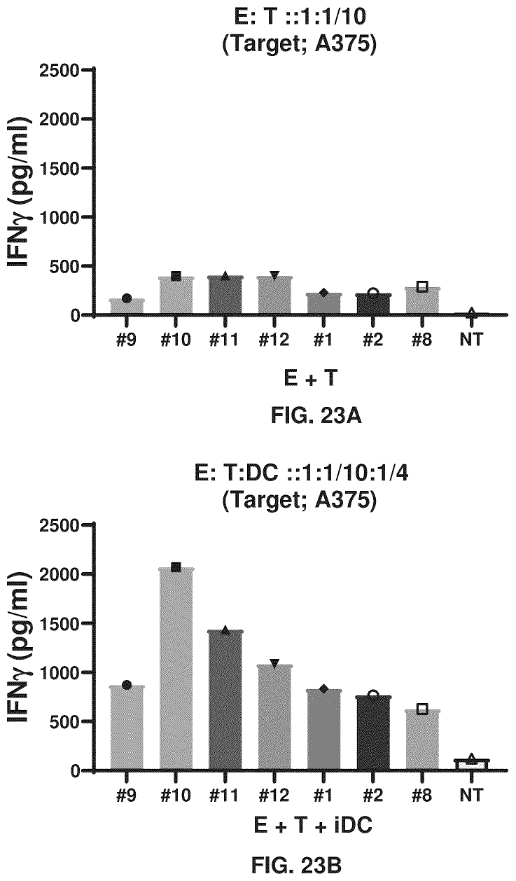

IFNγ secretion in response to A375 increases in the presence of immature DC (iDCs). In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with #11 induced stronger cytokine response measured as IFNγ quantified in the culture supernatants of three-way cocultures using donor D600115, E:T:iDC::1:1/10:1/4. FIG. 23A-B.

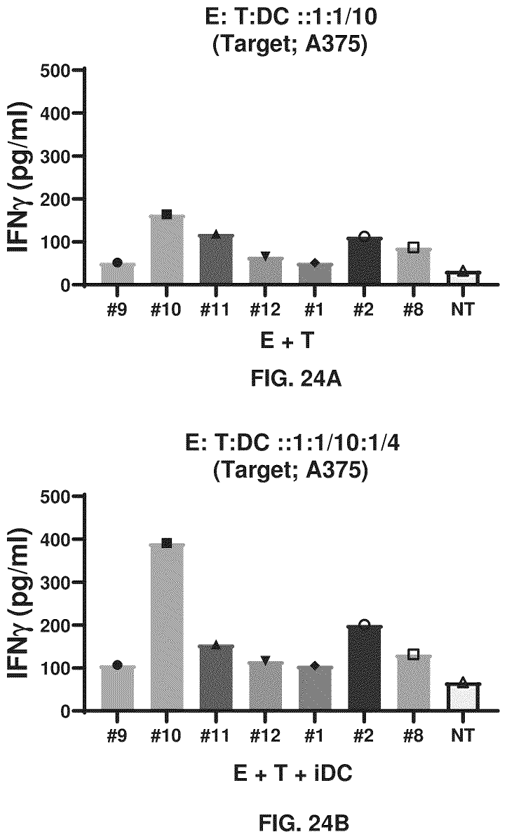

IFNγ secretion in response to A375 increases in the presence of iDCs. In the tri-cocultures with iDCs, IFNγ secretion was higher in Construct #10 compared to the other constructs. IFNγ quantified in the supernatants from DC cocultures D150081, E:T:iDC::1:1/10:1/4. FIG. 24A-B

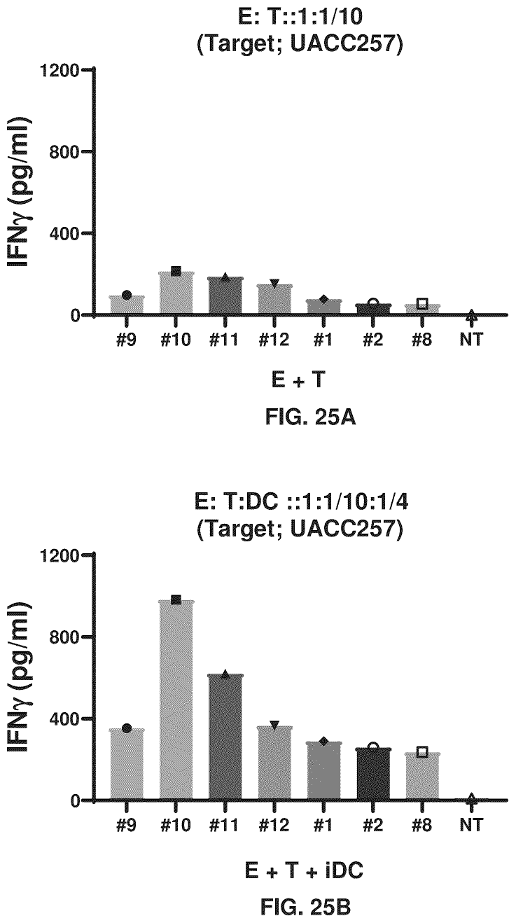

IFNγ secretion in response to UACC257 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with Construct #11 induced stronger cytokine response measured as IFNγ quantified in the culture supernatants of three-way cocultures using donor D600115, E:T:iDC::1:1/10:1/4. FIG. 25A-B. These results demonstrate that T cell products co-expressing a transgenic TCR and CD8 co-receptor (αβ heterodimer or modified CD8α homodimer) are able to license DCs in the microenvironment through antigen cross presentation and therefore hold the potential to mount a stronger anti-tumor response and modulate the tumor microenvironment.

### Example 7

### Vector Screening (Constructs #13-#21)

### Viral Titers

FIG. 5B shows viral titer of Constructs #10, #10n (new batch), #11, #11n (new batch), #13-#21, and TCR only as a control.

### T Cell Manufacturing

### Activation

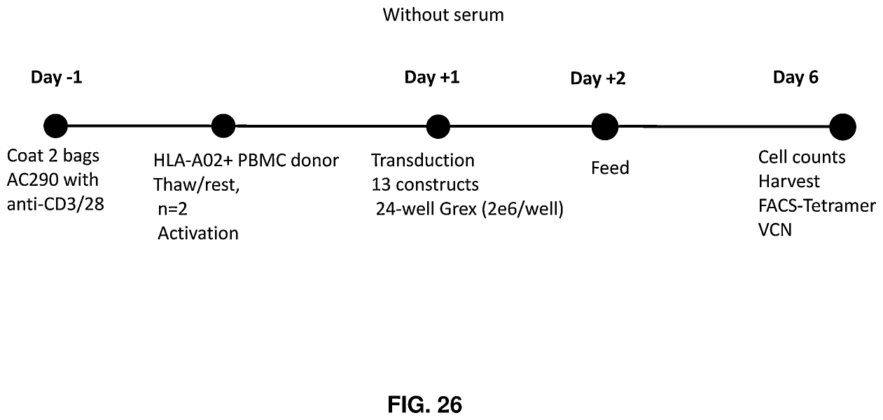

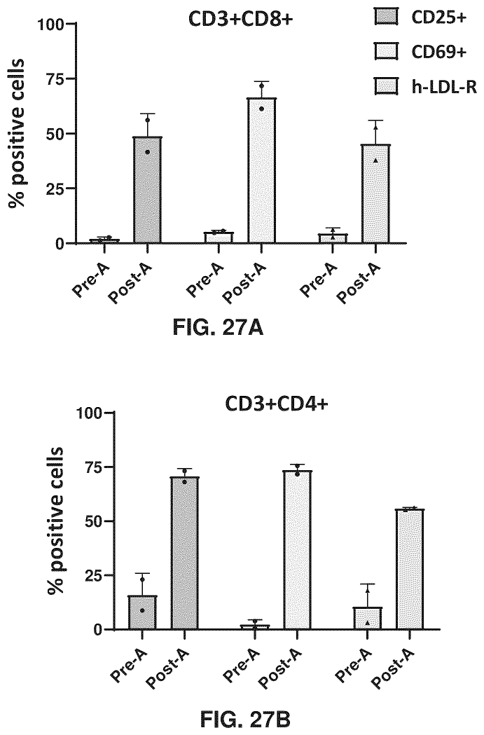

FIG. 26 shows that, on Day +0, PBMCs obtained from two HLA-A02+ donors (Donor #1 and Donor #2) were thawed and rested. Cells were activated in bags (AC290) coated with anti-CD3 and anti-CD28 antibodies in the absence of serum. Activation markers, e.g., CD25, CD69, and human low density lipoprotein receptor (H-LDL-R) are in CD8+ and CD4+ cells, were subsequently measured. FIG. 27A shows that % CD3+CD8+CD25+ cells, % CD3+CD8+CD69+ cells, and % CD3+CD8+H-LDL-R+ cells increase after activation (Post-A) as compared with that before activation (Pre-A). Similarly, FIG. 27B shows that % CD3+CD4+CD25+ cells, % CD3+CD4+CD69+ cells, and % CD3+CD4+H-LDL-R+ cells increase after activation (Post-A) as compared with that before activation (Pre-A). These results support the activation of PBMCs.

### Transduction

FIG. 26 shows that, on Day +1, activated PBMCs were transduced with viral vectors, e.g., Constructs #8, #10, #10n, #11, #11n, and #13-#21, in G-Rex® 24-well plates at about 2×10⁶ cells/well in the absence of serum. The amounts of virus used for transduction are shown in Table 8.

TABLE 8

| Constructs | Virus Volume/1 × 10⁶ cells |
|---|---|
| #10n, #11n, #13-#21 | 0.3 μl, 1.1 μl, 3.3 μl, 10 μl, 30 μl |
| #8 (TCR), #10 | 2.5 μl |
| #11 | 1.25 μl |
| NT | — |

### Expansion

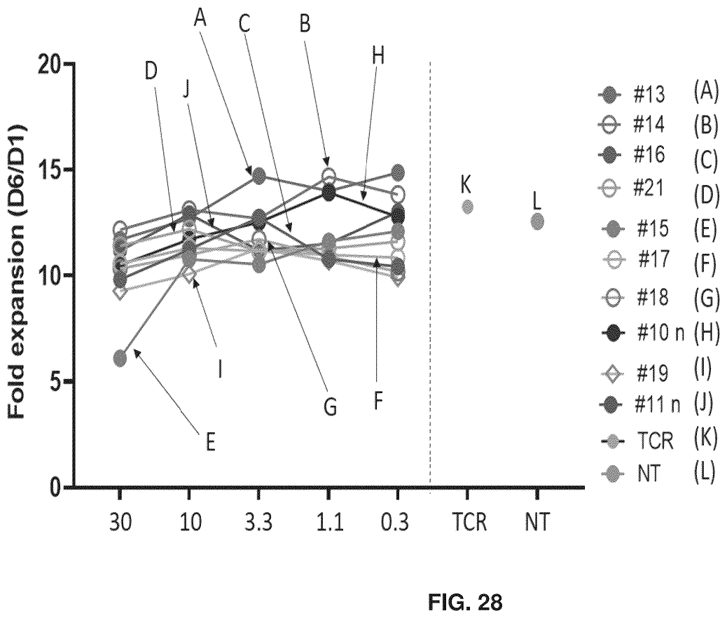

FIG. 26 shows that, on Day +2, transduced PBMCs were expanded in the absence of serum. On Day +6, cells were harvested for subsequent analysis, e.g., FACS-Tetramer and vector copy number (VCN) and were cryopreserved. FIG. 28 shows fold expansion on Day +6 of transduced T cell products. Viabilities of cells is greater than 90% on Day +6.

### Characterization of T Cell Products

Cell counts, FACS-dextramers, and vector copy numbers (VCN) were determined.

Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+ Tetramer(Tet)+ and CD8+ Tet+.

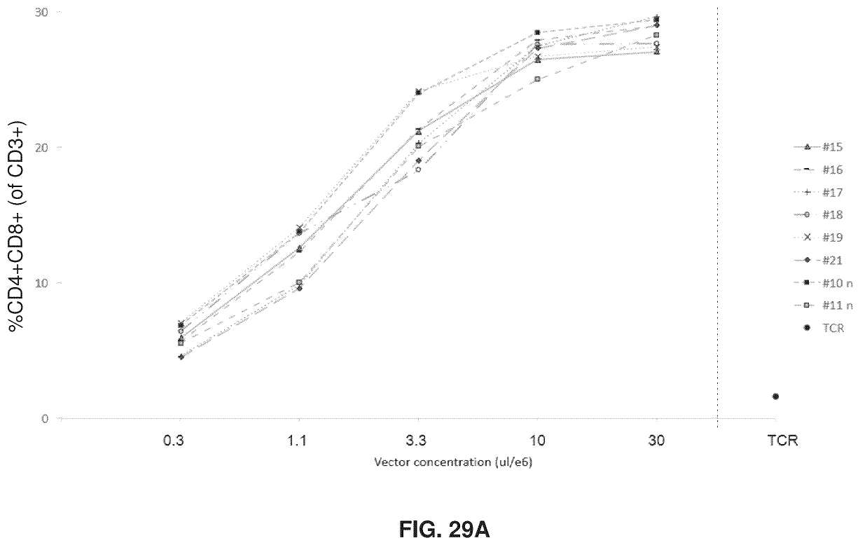

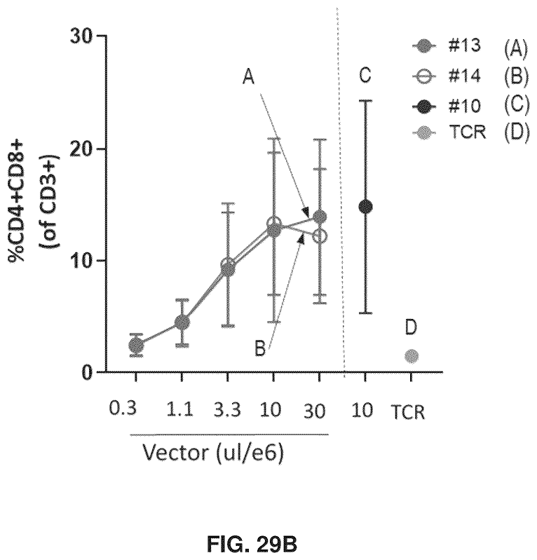

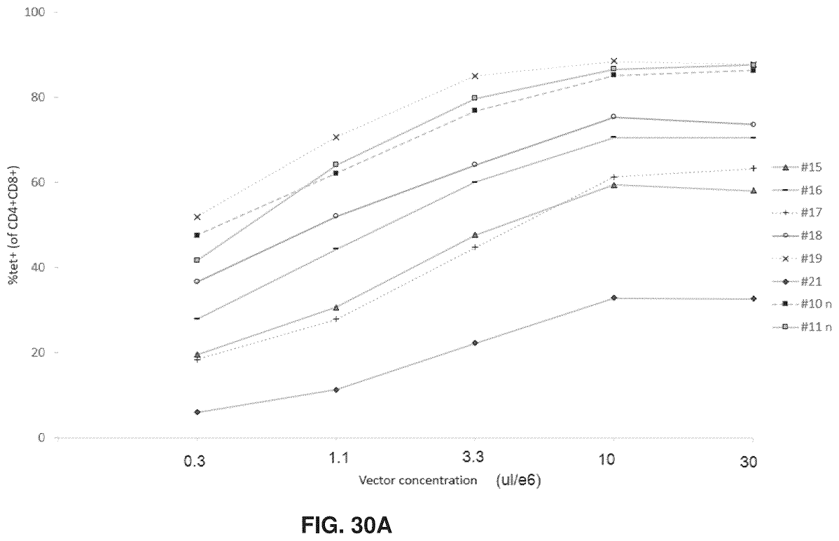

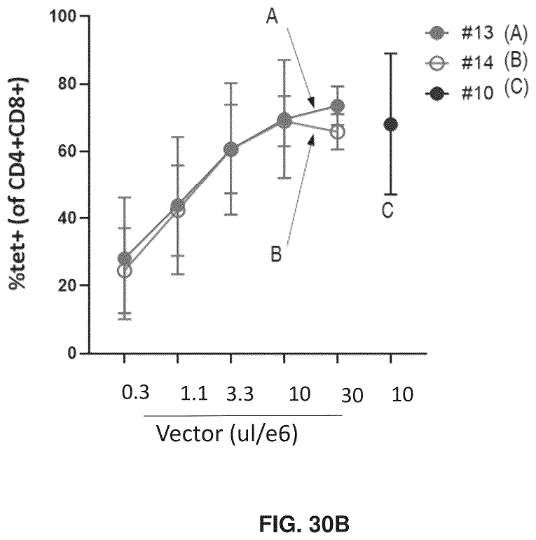

FIG. 29A and FIG. 29B shows % CD8+CD4+ cells transduced with Construct #10, #10n, #11, #13-#21 at 0.3 μl, 1.1 μl, 3.3 μl, 10 μl or 30 μl per 1×10⁶ cells. These results show comparable frequencies of CD8+CD4+ cells obtained by transduction with all vectors tested. Construct #8 (TCR only) serves as negative control. FIG. 30A and FIG. 30B shows % Tet of CD8+CD4+ cells from transduced with Construct #10, #10n, #11, #13-#21 at 0.3 μl, 1.1 μl, 3.3 μl, 10 μl or 30 μl per 1×10⁶ cells. These results show that there was a trend towards higher frequencies of CD4+CD8+tet+ in CD8β1 isoforms (Constructs #10 and #18) compared to CD8β3 isoforms (Construct #16) and CD8β5 isoforms (Constructs #15 and #17). FACS analysis was gated on live singlets, followed by CD3+, followed by CD4+CD8+, and followed by CD4+CD8+ Tet+.

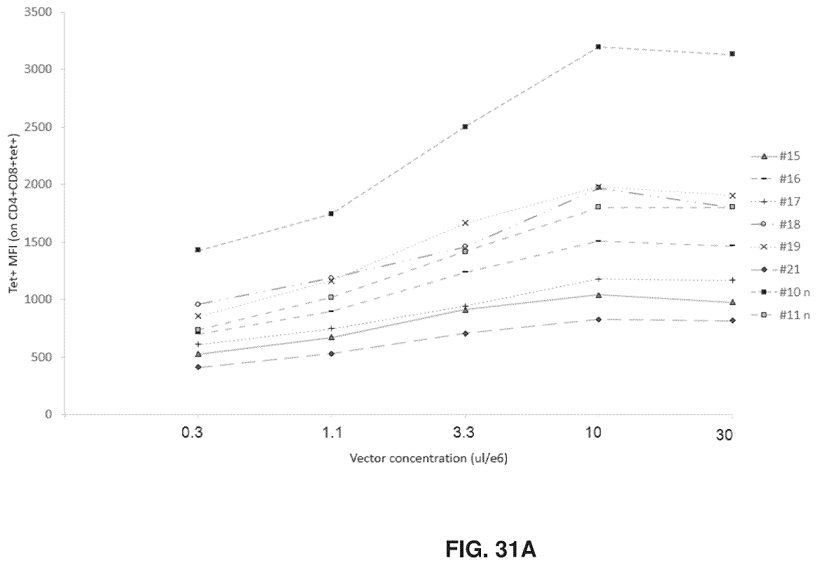

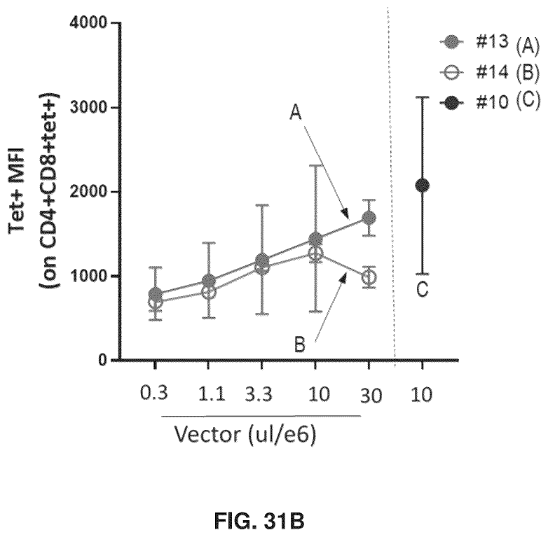

FIG. 31A and FIG. 31B shows Tet MFI of CD8+CD4+ Tet+ cells from transduced with Construct #10, #10n, #11, #13-#21 at 0.3 μl, 1.1 μl, 3.3 μl, 10 μl or 30 μl per 1×10⁶ cells. These results show a trend towards higher tetramer MFI on CD4+CD8+ Tet+ population in CD8β1 isoforms (Constructs #10 and #18) compared to CD8β3 isoforms (Construct #16) and CD8β5 isoforms (Constructs #15 and #17).

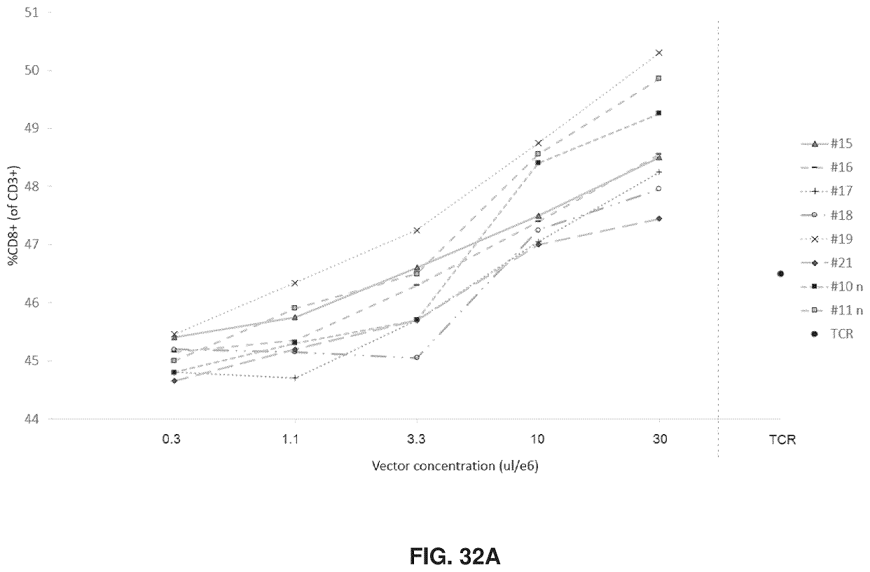

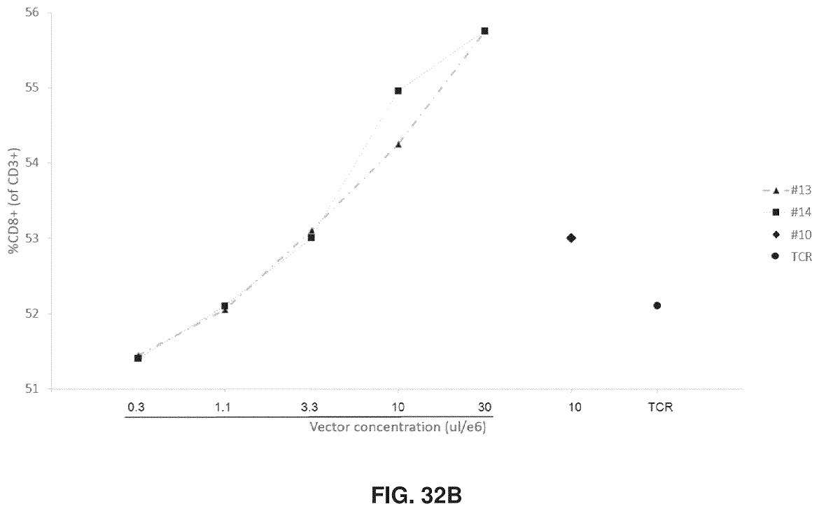

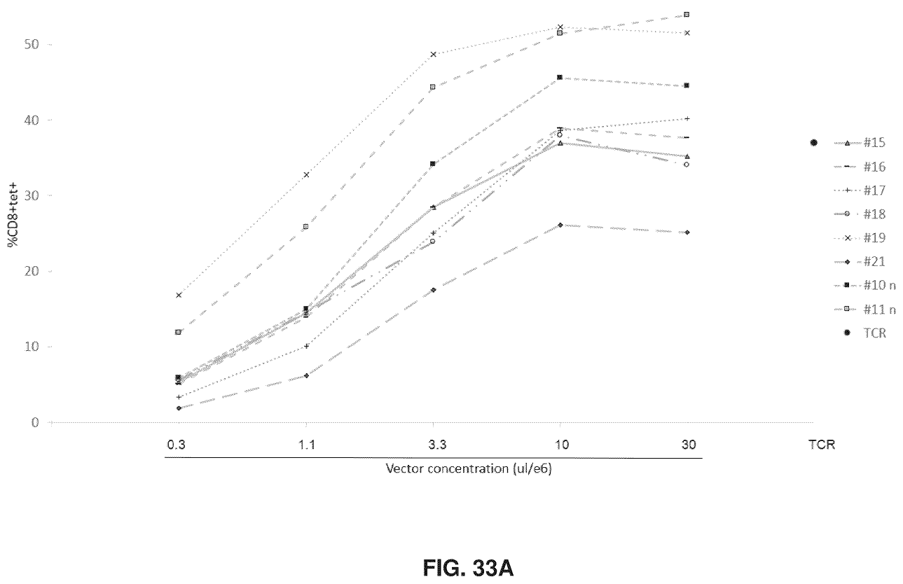

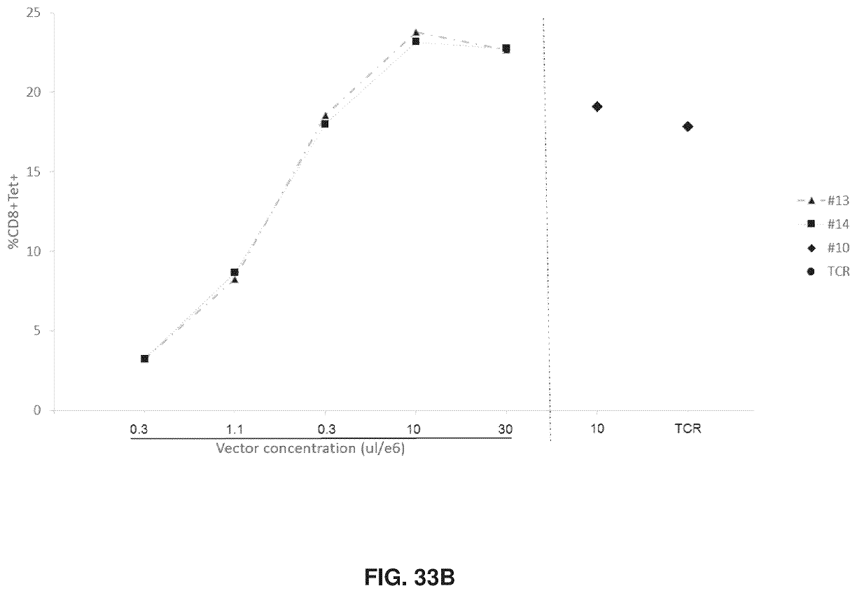

FIG. 32A and FIG. 32B show CD8 frequencies (% CD8+CD4− of CD3+) in cells transduced with Construct #10, #10n, #11, #13-#21 at 0.3 μl, 1.1 μl, 3.3 μl, 10 μl or 30 μl per 1×10⁶ cells. These results show no difference in the CD8 frequencies among the constructs. FIG. 33A and FIG. 33B shows % CD8+ Tet+ (of CD3+) cells transduced with Construct #10, #10n, #11, #13-#21 at 0.3 μl, 1.1 μl, 3.3 μl, 10 μl or 30 μl per 1×10⁶ cells. These results show slightly higher frequencies of CD8+ Tet+ (of CD3+) in cells transduced with Construct #10 than those transduced with the other constructs. FACS analysis was gated on live singlets, followed by CD3+, followed by CD8+CD4−, and followed by Tet+.

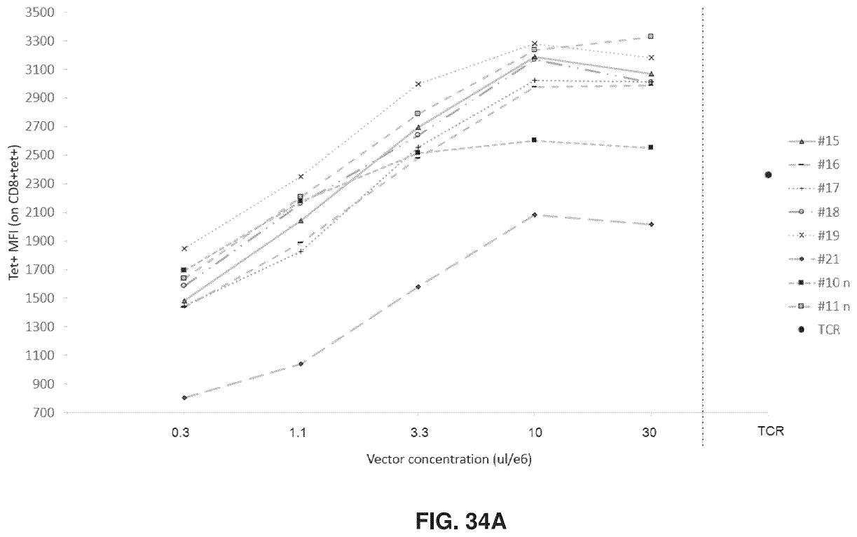

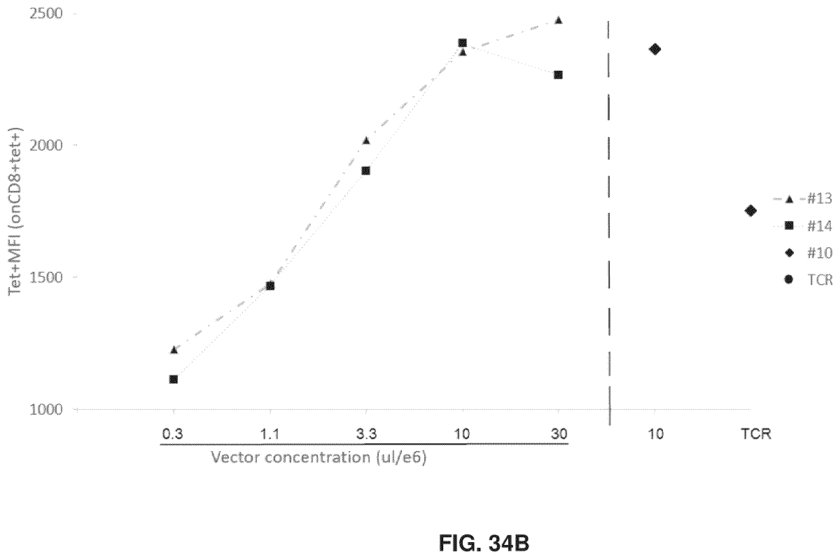

FIG. 34A and FIG. 34B shows Tet MFI of CD8+ Tet+ cells transduced with Construct #10, #10n, #11, #13-#21 at 0.3 μl, 1.1 μl, 3.3 μl, 10 μl or 30 μl per 1×10⁶ cells. These results show tetramer MFI of CD8+tet+ cells was comparable among CD8β1 (Constructs #18 and #10), CD8β5 (Constructs #15 and #17), and CD8β3 (Construct #16) isoforms, while Construct #21 expressed lower tetramer MFI.

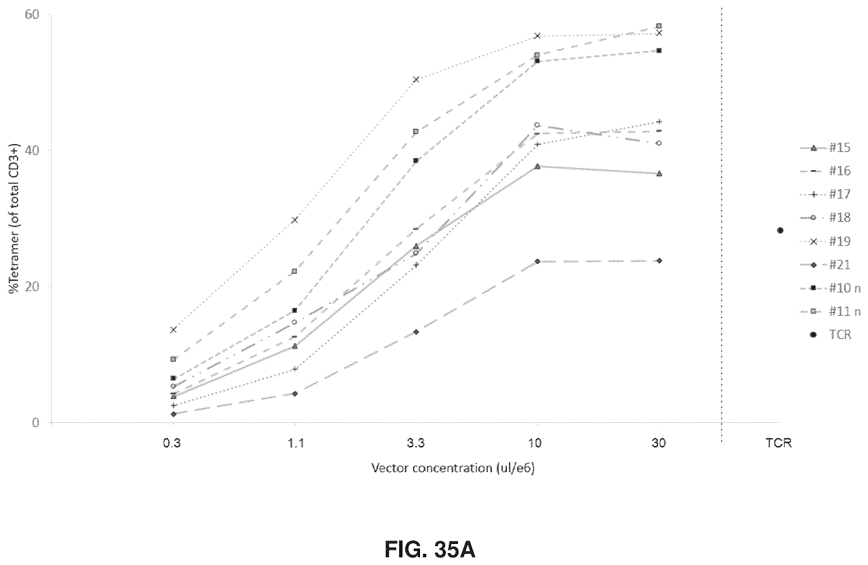

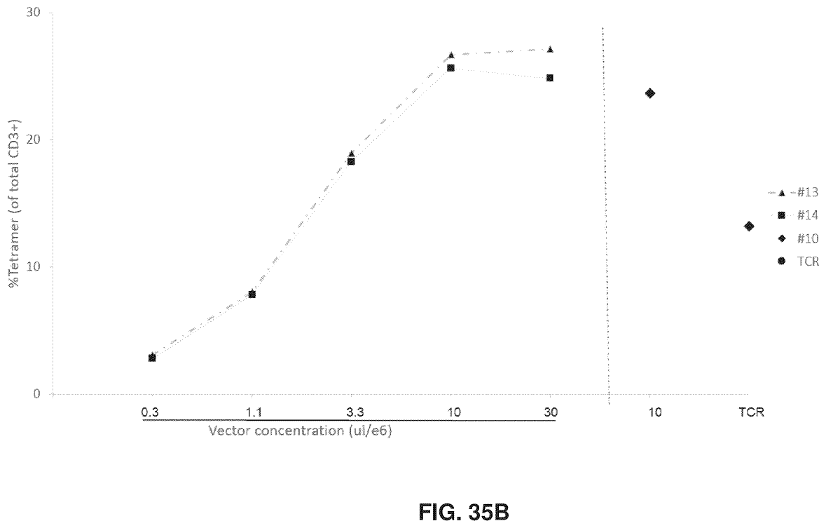

FIG. 35A and FIG. 35B shows % Tet+ of CD3+ cells transduced with Construct #10, #10n, #11, #13-#21 at 0.3 μl, 1.1 μl, 3.3 μl, 10 μl or 30 μl per 1×10⁶ cells. These results show higher frequencies of CD3+Tet+ in cells transduced with Construct #10 (CD8β1) compared to those transduced with CD8β3 (Construct #16) and CD8β5 (Constructs #15 and #17). FACS analysis was gated on live singlets, followed by CD3+, and followed by Tet+.

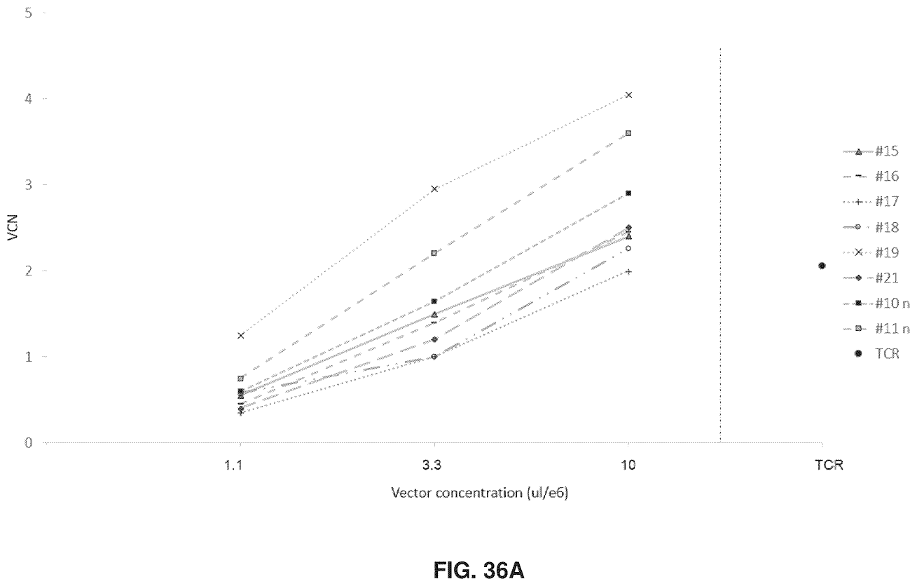

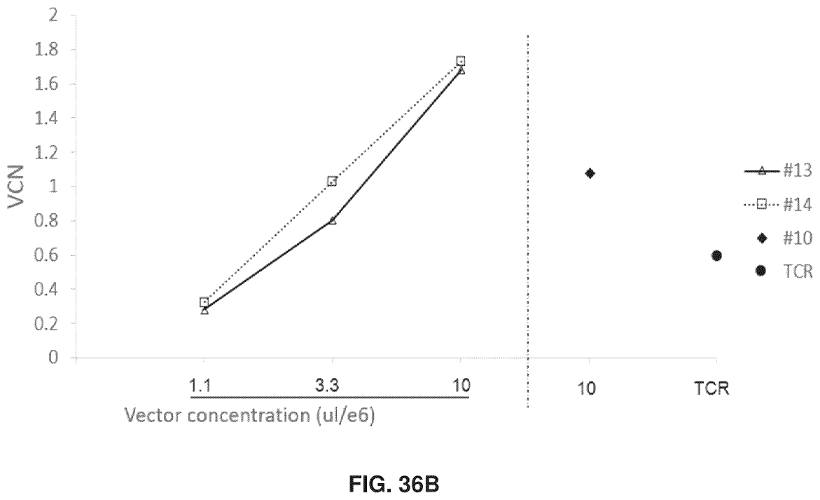

FIG. 36A and FIG. 36B shows vector copy number (VCN) of cells transduced with Construct #10, #10n, #11, #13-#21 at 0.3 μl, 1.1 μl, 3.3 μl, 10 μl or 30 μl per 1×10⁶ cells. These results show comparable ability of all constructs to integrate and express CD8/TCR genes.

In sum, these results show (1) viral vectors with CD8β1, CD8β3 and CD8β5 isoforms had good transducing titers; (2) all constructs were capable of successful manufacturing (e.g., high viability, fold expansions in the range of 6-12); (3) frequencies of CD3+tet+ among CD8β3 isoforms: CD8β1 (Construct #10) was greater than CD8β3 (Construct #16) and CD8β5 (Constructs #15 and #17), with Construct #21 showing the lowest values; (4) frequency of CD3+tet+ in Constructs #11 and #19 (m1CD8α (SEQ ID NO: 7)) showed the highest values; and (5) saturation in % CD3+tet+, % CD8+tet+ and % CD4+CD8+tet+ observed at 10 μl/e6. Optimal vector dose ranges between 3.3-10 μl/e6 for all constructs.

### Example 8

### Mid-Scale Vector Screening (Constructs #13-#19)

### T Cell Manufacturing

### Activation/Transduction

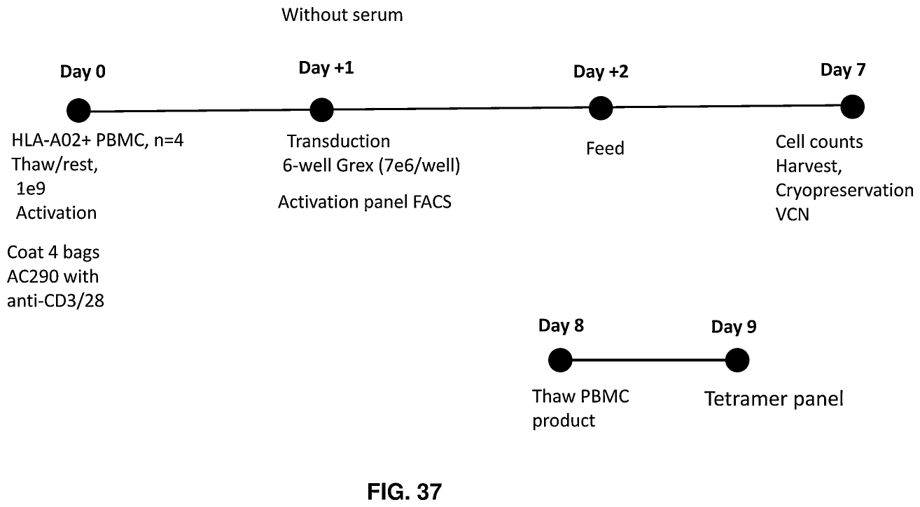

FIG. 37 shows that, on Day +0, PBMCs obtained from four HLA-A02+ donors were thawed and rested. Cells were activated in bags (AC290) coated with anti-CD3 and anti-CD28 antibodies in the absence of serum. On Day +1, activated PBMCs were transduced with viral vectors, e.g., Constructs #8, #10n, #11n, and #13-#19, in G-Rex® 6-well plates at about 7×10⁶ cells/well in the absence of serum. The amounts of virus used for transduction are shown in Table 9.

TABLE 9

| Constructs | Virus Volume/1 × 10⁶ cells |
|---|---|
| #13-19 | 2.5 μl and 5 μl |
| #10n and #11n | 2.5 μl and 5 μl |
| #8 (TCR) | 2.5 μl |
| NT | — |

### Expansion

FIG. 37 shows that, on Day +2, transduced PBMCs were expanded in the absence of serum. On Day +7, cells were harvested for subsequent analysis, e.g., FACS-Tetramer and vector copy number (VCN) and were cryopreserved. Fold expansion on Day +7 was comparable for all constructs (approximately 30-fold expansion). Viabilities of cells is greater than 90% on Day +7.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+ Tetramer(Tet)+ and CD8+ Tet+.

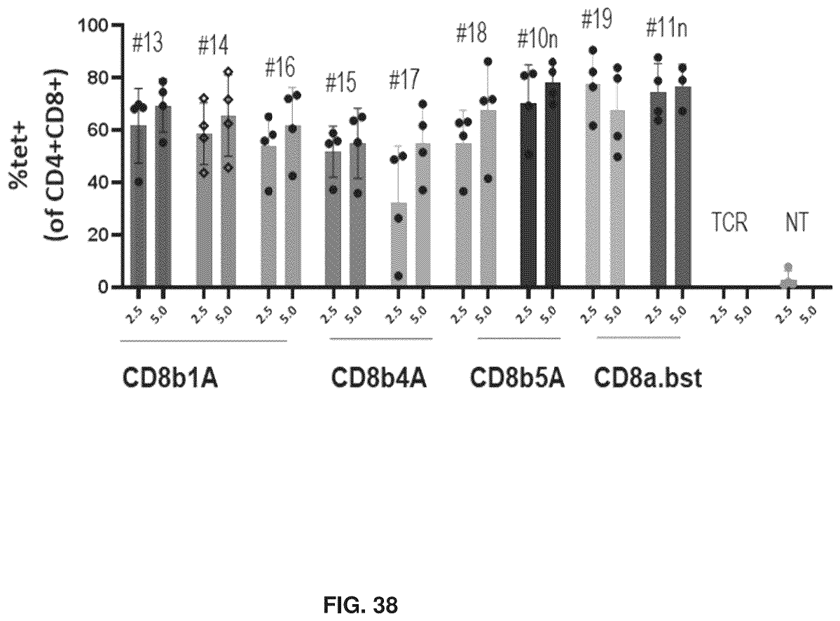

Similar to results described in Example 6, comparable frequencies of CD8+CD4+ cells were obtained by transduction with Construct #10n, #11n, #13-#19 at 2.5 μl or 5.0 μl per 1×10⁶ cells. Construct #8 (TCR only) serves as negative control. FIG. 38 shows % Tet of CD8+CD4+ cells transduced with Construct #10n, #11n, #13-#19 at 2.5 μl or 5.0 μl per 1×10⁶ cells. Similar to results described in Example 6, these results show that there was a trend towards higher frequencies of CD4+CD8+tet+ in CD8β1 isoforms (Construct #10n) compared to CD8β3 isoforms (Constructs #13, #14, #16) and CD8β5 isoforms (Constructs #15 and #17). FACS analysis was gated on live singlets, followed by CD3+, followed by CD4+CD8+, and followed by Tet+.

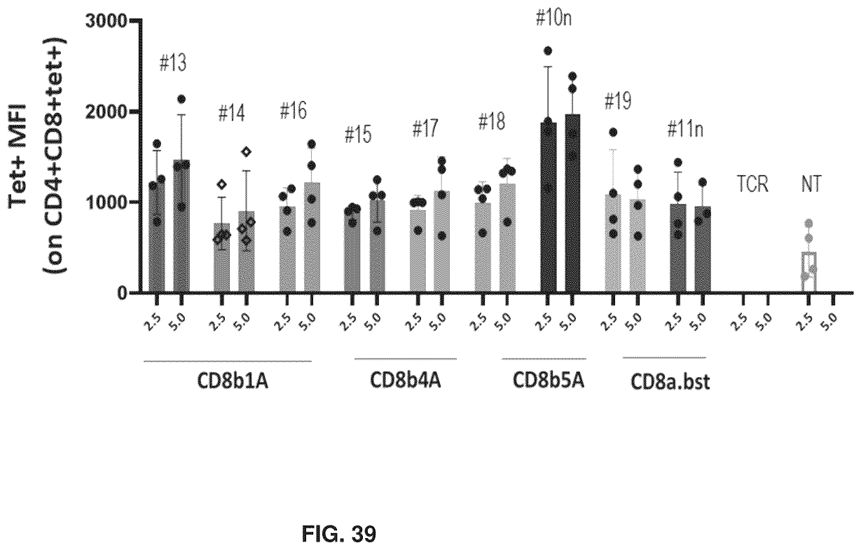

FIG. 39 shows Tet MFI of CD8+CD4+ Tet+ cells from transduced with Construct #10n, #11n, #13-#19 at 2.5 μl or 5.0 μl per 1×10⁶ cells. These results show higher tetramer MFIs on CD4+CD8+ Tet+ population in CD8β1 isoforms (Construct #10n) compared to CD8β3 isoforms (Construct #13) and CD8β5 isoforms (Constructs #15 and #17).

Similar to results described in Example 6, results show no difference in the CD8 frequencies (% CD8+CD4− of CD3+) in cells transduced with Construct #10n, #11n, #13-#19 at 2.5 μl or 5.0 μl per 1×10⁶ cells among the constructs (data not shown). Comparable frequencies of CD8+ Tet+ (of CD3+) in cells transduced with Construct #10n, #11n, #13-#19 at 2.5 μl or 5.0 μl per 1×10⁶ cells (data not shown). FACS analysis was gated on live singlets, followed by CD3+, followed by CD8+CD4−, and followed by Tet+.

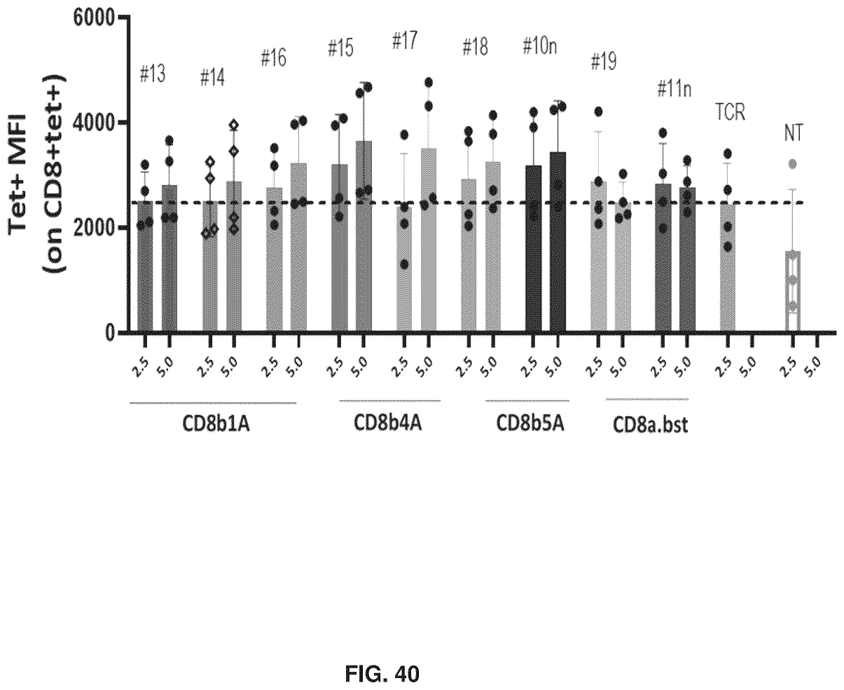

FIG. 40 shows Tet MFI of CD8+ Tet+ cells transduced with Construct #10n, #11n, #13-#19 at 2.5 μl or 5.0 μl per 1×10⁶ cells. These results show tetramer MFI of CD8+tet+ cells was comparable among CD8β1 (Constructs #18 and #10) and CD8β5 (Construct #15) isoforms, while CD8β3 (Constructs #13, #14, and #16) isoforms expressed lower tetramer MFI.

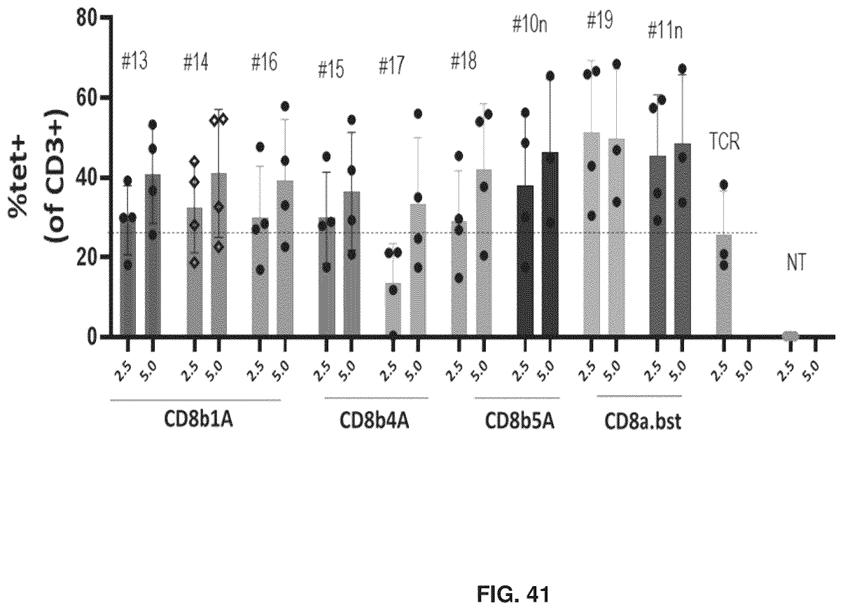

FIG. 41 shows % Tet+ of CD3+ cells transduced with Construct #10n, #11n, #13-#19 at 2.5 μl or 5.0 μl per 1×10⁶ cells. These results show slightly higher frequencies of CD3+Tet+ in cells transduced with Construct #10 (CD8β1) compared to those transduced with CD8β3 (Constructs #13, #14, and #16) and CD8β5 (Construct #15). FACS analysis was gated on live singlets, followed by CD3+, and followed by Tet+. Slightly higher total CD3+tet+ cell counts were observed in PBMC transduced with Construct #10 CD8β1) compared to those transduced with CD8β3 (Constructs #13, #14, and #16) and CD8β5 (Construct #15) (data not shown).

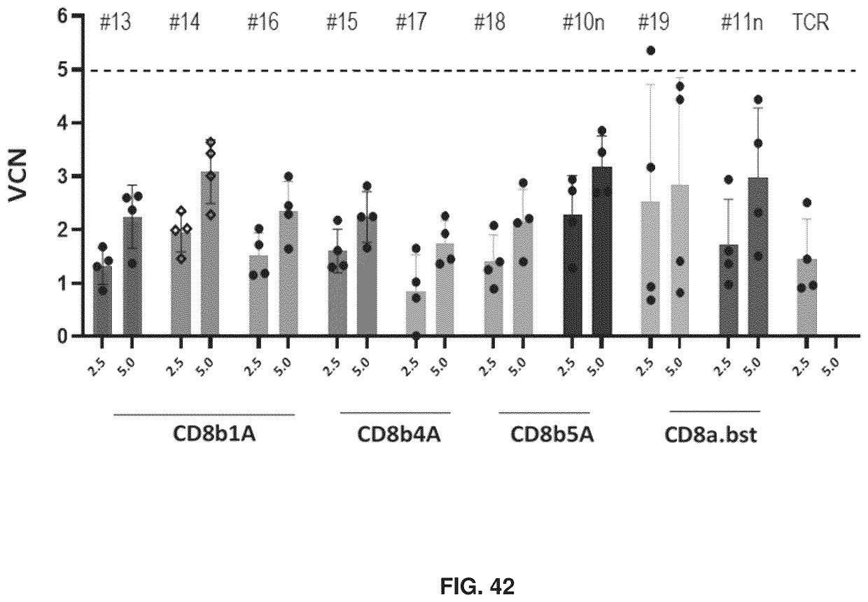

FIG. 42 shows vector copy number (VCN) of cells transduced with Construct #10n, #11n, #13-#19 at 2.5 μl or 5.0 μl per 1×10⁶ cells. These results show vector copies per cell remained below 5 in PBMC product derived using each individual construct at vector dose of 2.5 μl or 5.0 μl per 1×10⁶ cells.

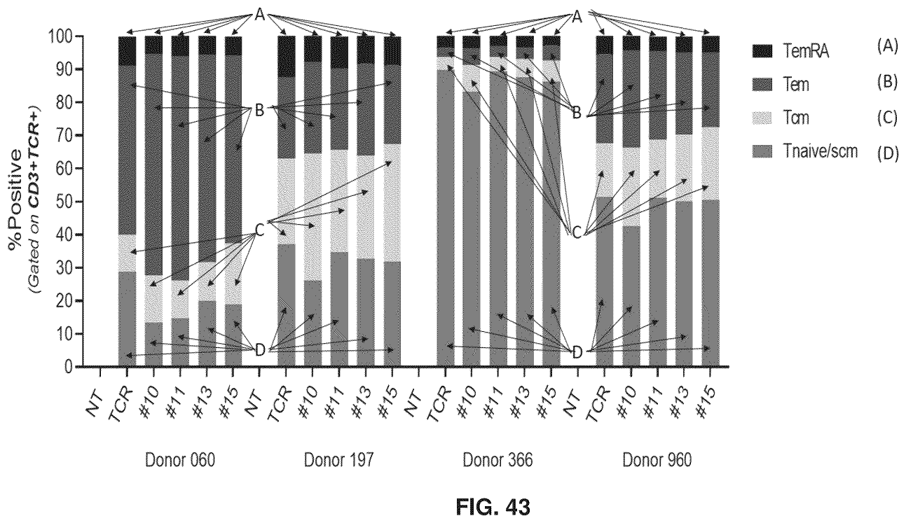

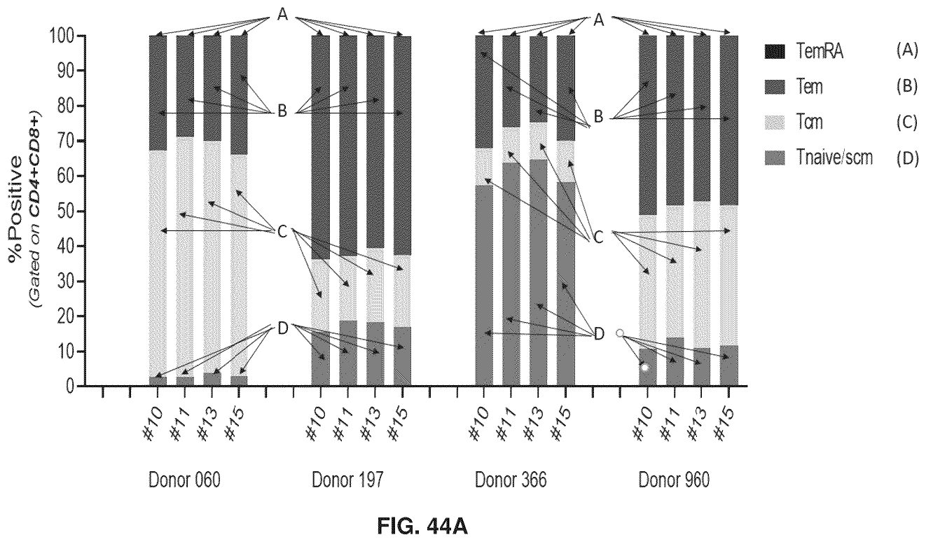

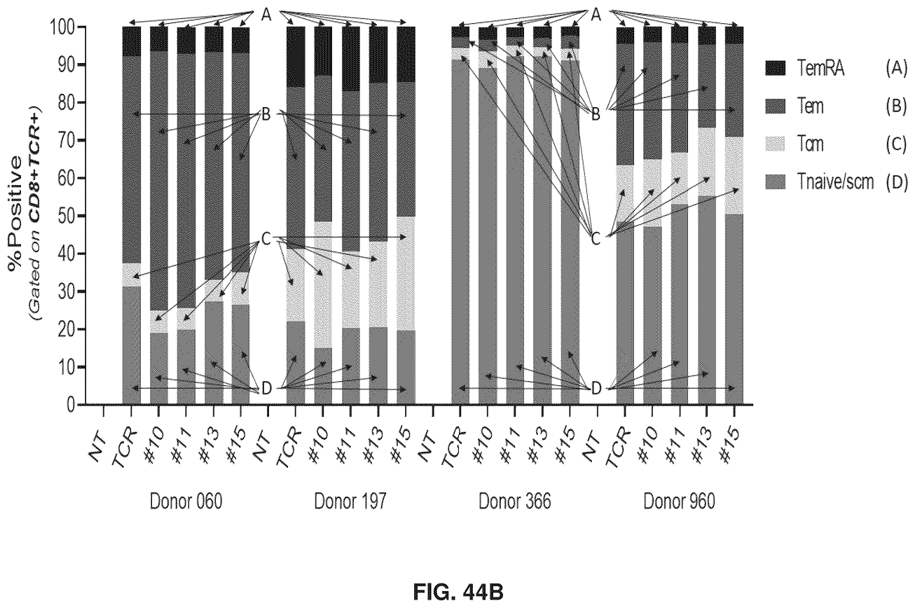

FIG. 43 shows the % T cell subsets in cells transduced with Construct #10, #11, #13, and #15 for each donor. Construct #8 (TCR only) and non-transduced cells were used as controls. These results show that TCR-only condition has slightly more naïve cells compared to the other constructs, consistent with lower fold-expansion. FIG. 44A and FIG. 44B shows % T cell subsets in cells transduced with Construct #10, #11, #13, and #15 for each donor. Construct #8 (TCR only) and non-transduced cells were used as controls. FACS analysis was gated on CD4+CD8+ for FIG. 44A and on CD4−CD8+ TCR+ for FIG. 44B. These results show donor-to-donor variability between frequencies of T cell memory subsets but little difference in the frequencies of Tₙₐᵢᵥₑ and T_{cm} between constructs.

In sum, these results show (1) viability and fold expansions were comparable among all constructs at day 7; (2) slightly higher frequency of CD3+tet+ observed in CD8β1 (Construct #10) compared to CD8β3 (Constructs #13, #14, and #16) and CD8β5 (Constructs #15 and #17); (3) vector copies per cell <5 for majority of the constructs at 2.5-5 μl/10⁶ dose; and (4) donor-to-donor variability between frequencies of T cell memory subsets but generally, Construct #10 has less naïve but more Tcm cells than the other β isoform constructs.

### Example 9

### Tumor Death Assay—Constructs #10, #11, #13 & #15

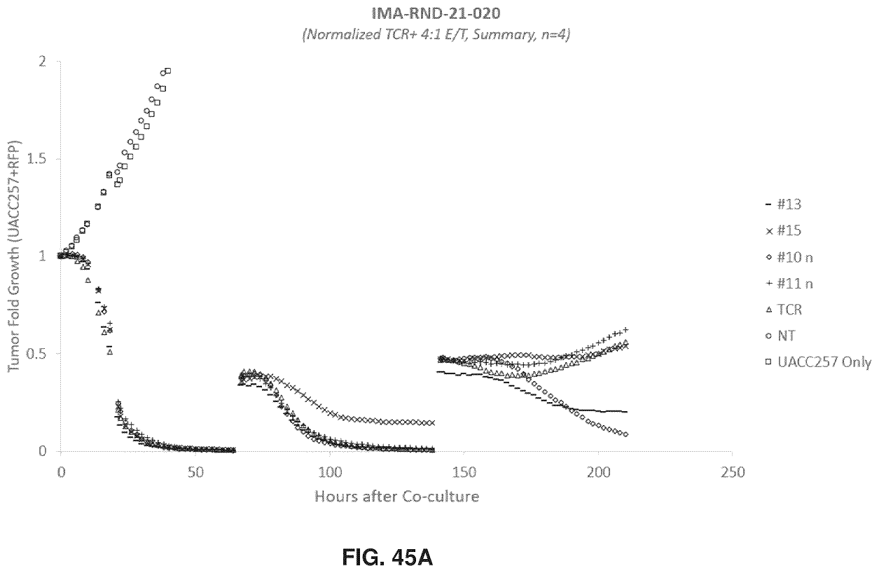

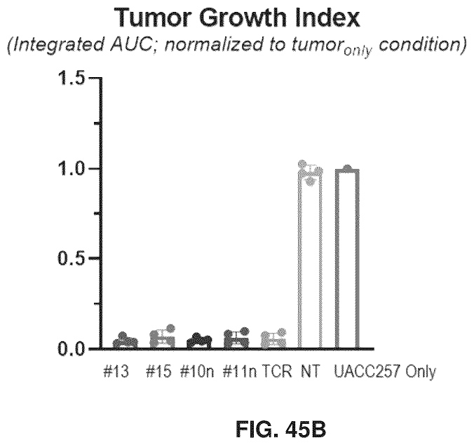

FIGS. 45A and 45B depicts data showing that Constructs #13 and #10 are comparable to TCR-only in mediating cytotoxicity against UACC257 target positive cells lines expressing high levels of antigen (1081 copies per cell). Construct #15 was also effective but slower in killing compared to Constructs #13 and #10. The effector:target ratio used to generate these results was 4:1. Similar results were obtained with a 2:1 effector:target ratio (data not shown).

### Example 10

### IFNγ Secretion Assay—Constructs #10, #11, #13 & #15

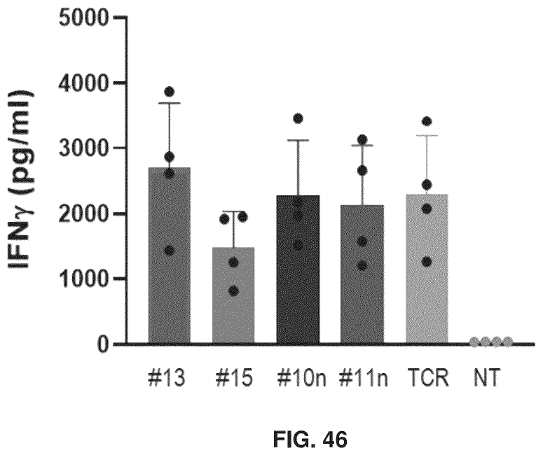

IFNγ secretion was measured in the UACC257 cells line. FIG. 46 shows IFNγ secretion in response in UACC257 cell line was higher with Construct #13 compared to Construct #10. IFNγ quantified in the supernatants from Incucyte plates. The effector:target ratio used to generate these results was 4:1. Similar results were obtained with a 2:1 effector:target ratio (data not shown).

### Example 11

### ICI Marker Expression—Constructs #10, #11, #13 & #15

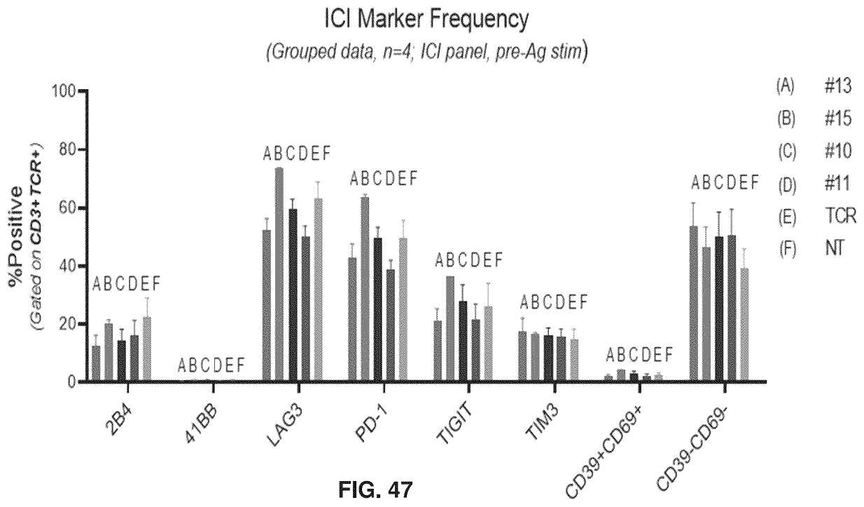

ICI marker frequency (2B4, 41BB, LAG3, PD-1, TIGIT, TIM3, CD39+CD69+, and CD39−CD69−) was measured. FIG. 47 shows Construct #15 has higher expression of LAG3, PD-1, and TIGIT compared to other constructs, followed by Construct #10.

### Example 12

### Cytokine Expression—Constructs #10, #11, #13 & #15

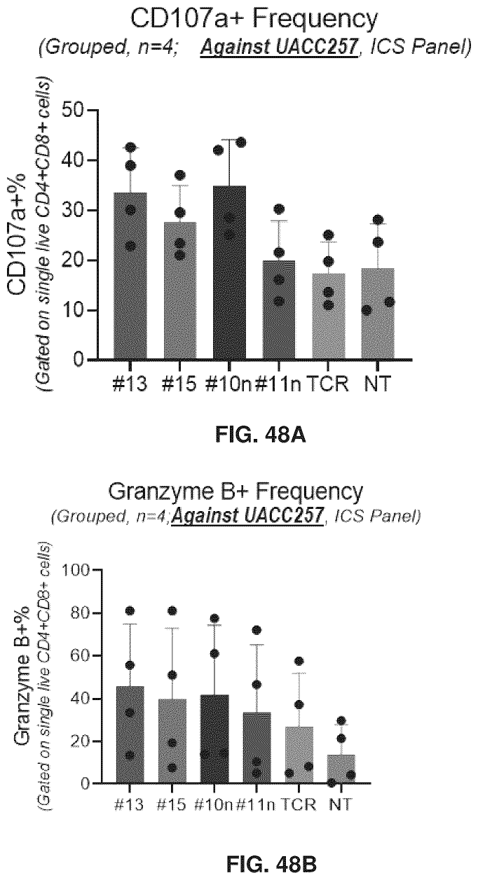

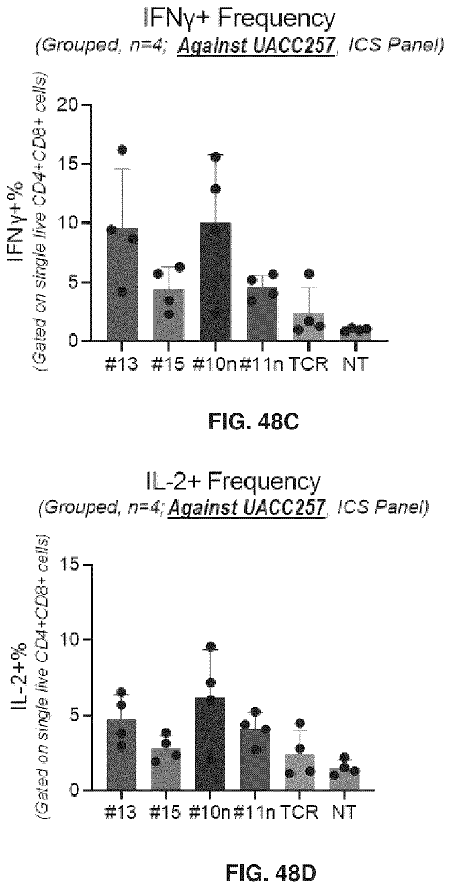

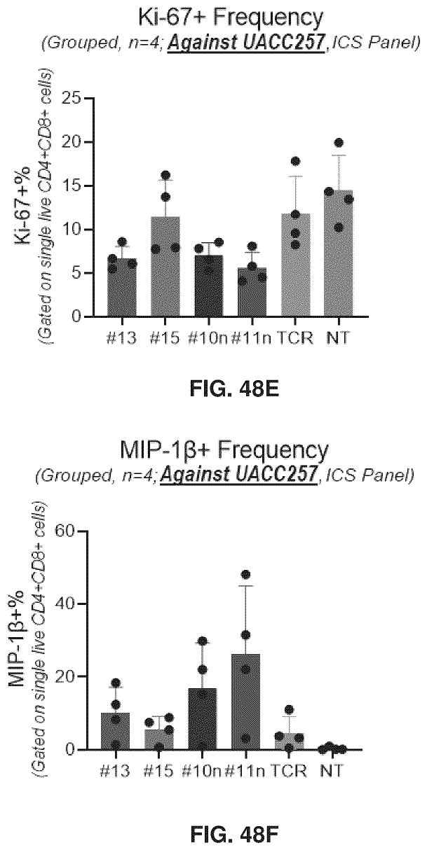

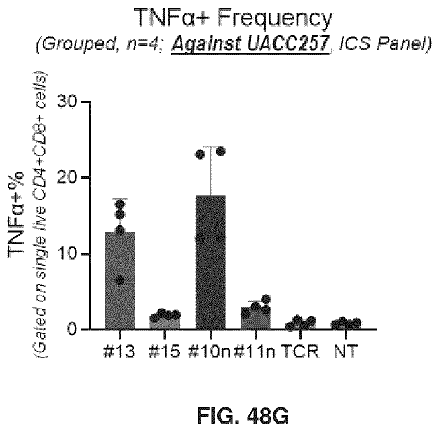

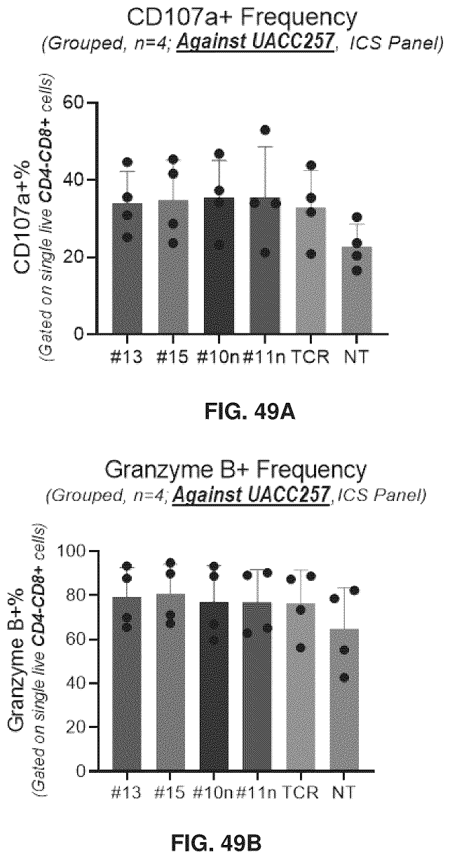

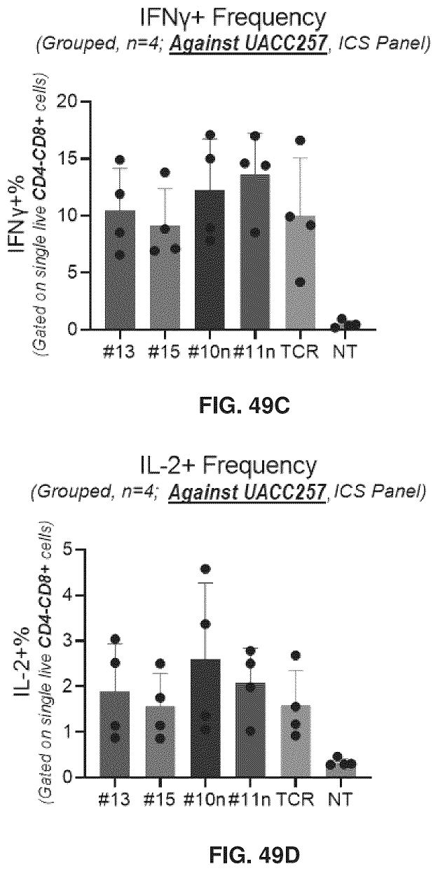

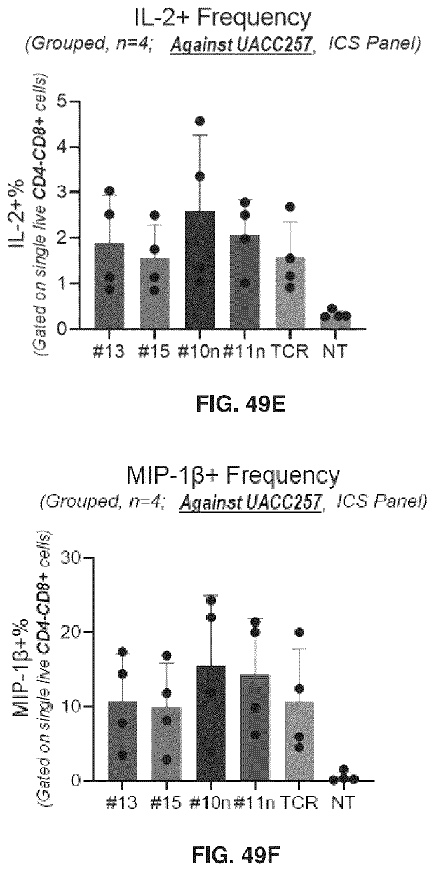

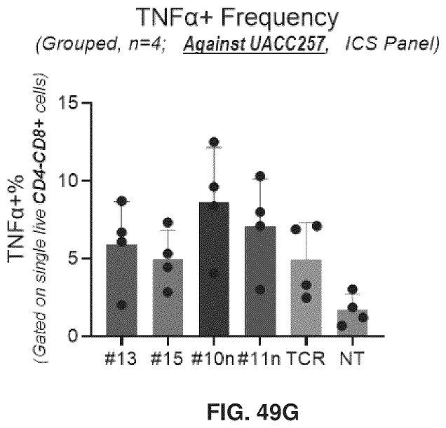

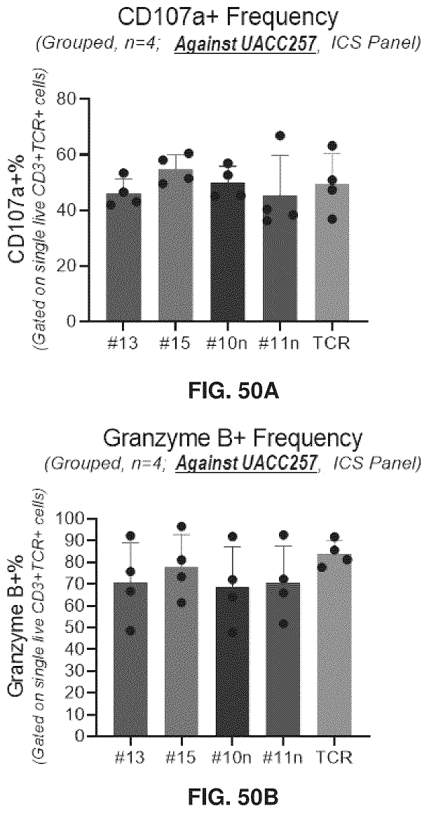

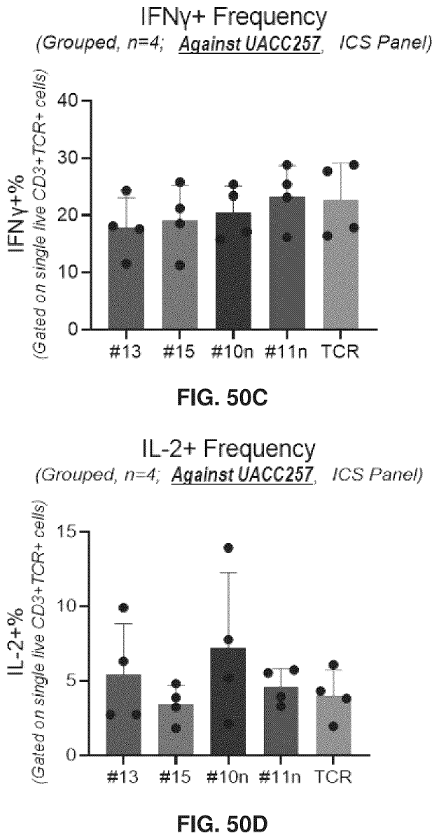

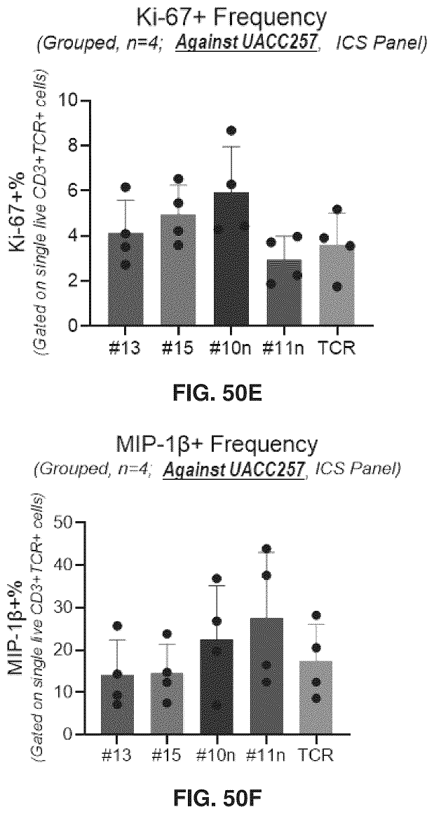

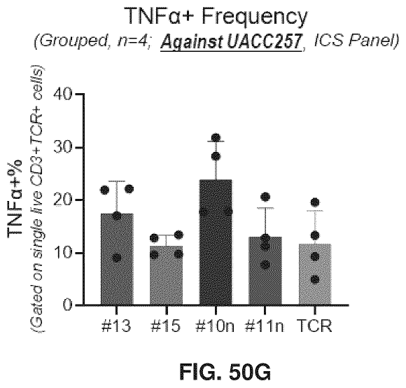

Expression of various cytokines was measured in UACC257 cells co-cultured at a 4:1 E:T ratio with PBMC transduced with Constructs #10, #11, #13, and #15. FIG. 48A-48G show increased expression of IFNγ, IL-2, and TNFα with CD4+CD8+ cells transduced with construct #10 (WT signal peptide, CD8β1) compared to other constructs. FACS analysis was gated on CD3+CD4+CD8+ cells against UACC257, 4:1 E:T. FIG. 49A-49G show increased expression of IFNγ, IL-2, MIP-1β, and TNFα with CD4−CD8+ cells transduced with construct #10 (WT signal peptide, CD8β1) compared to other constructs. FACS analysis was gated on CD3+CD4−CD8+ cells against UACC257, 4:1 E:T. FIG. 50A-50G show increased expression of IL-2 and TNFα with CD3+TCR+ cells transduced with construct #10 (WT signal peptide, CD8β1) compared to other constructs. MIP-1βexpression is highest in Construct #11 (similar results when gated on CD4+CD8+ cells). FACS analysis was gated on CD3+TCR+ cells against UACC257, 4:1 E:T.

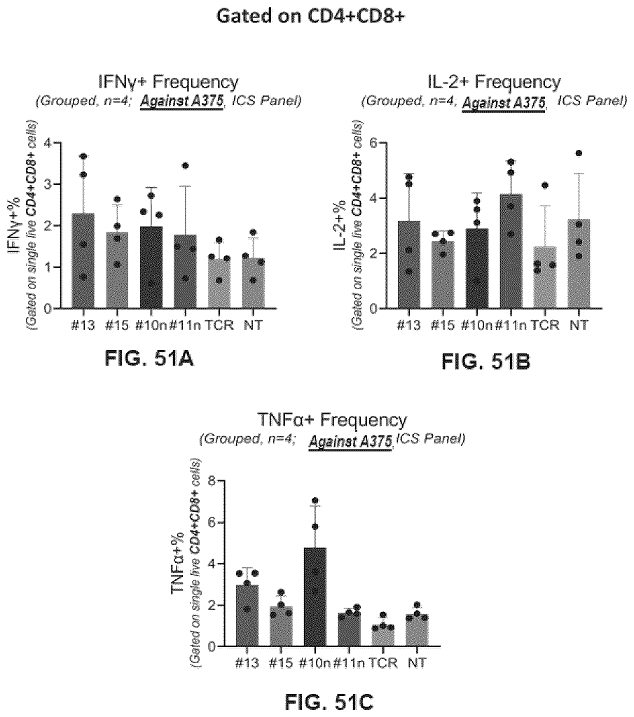

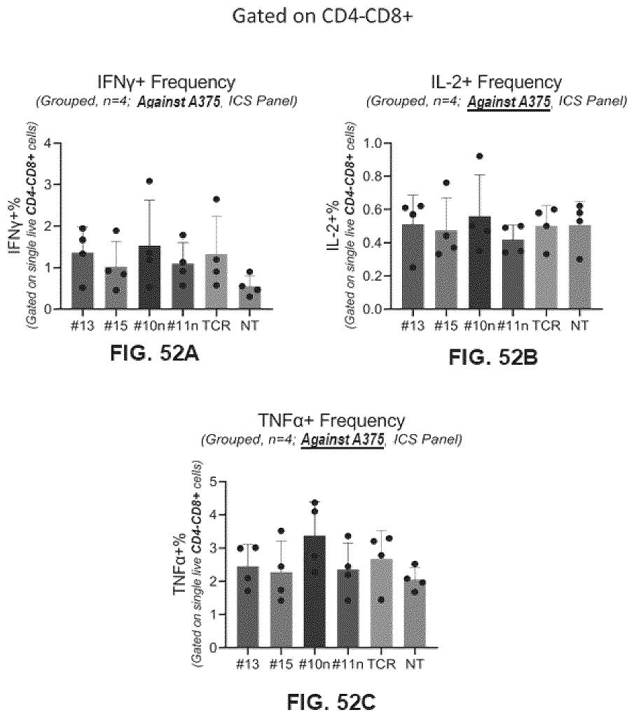

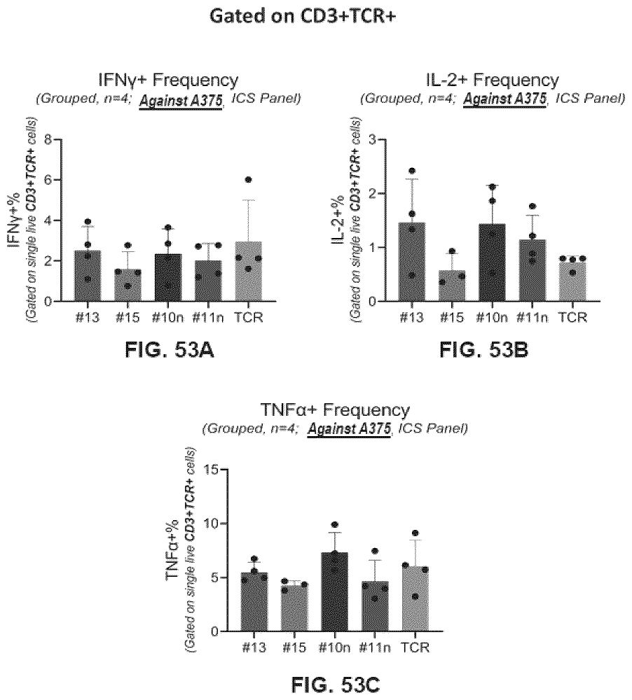

Expression of various cytokines was measured in A375 cells co-cultured at a 4:1 E:T ratio with PBMC transduced with Constructs #10, #11, #13, and #15. FIG. 51A-51C show results from FACS analysis gated on CD4+CD8+ cells against A375, 4:1 E:T. FIG. 52A-52C show results from FACS analysis gated on CD4−CD8+ cells against A375, 4:1 E:T. FIG. 53A-53C show results from FACS analysis gated on CD3+TCR+ cells against A375, 4:1 E:T. Overall, results were more variable when cells are co-cultured with A375+RFP, but similar trends are observed compared to activation by UACC257+RFP.

### Example 13

### Large-Scale Vector Screening (Constructs #10, #11, #13, #16, #18, #19)

### T Cell Manufacturing

### Activation/Transduction

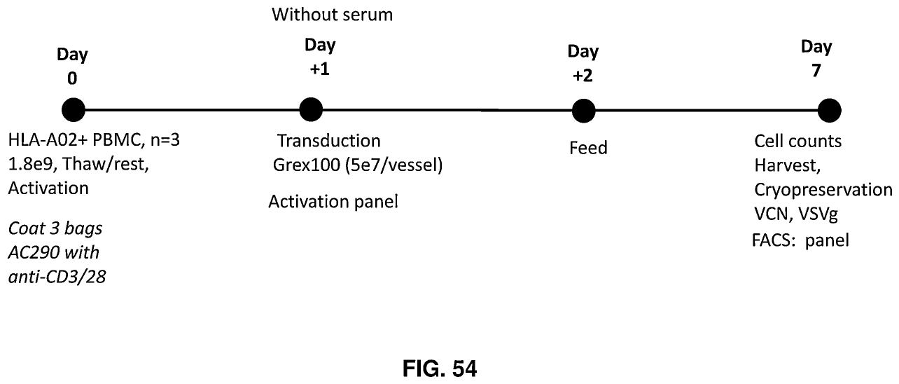

FIG. 54 shows that, on Day +0, PBMCs obtained from three HLA-A02+ donors were thawed and rested. Cells were activated in bags (AC290) coated with anti-CD3 and anti-CD28 antibodies in the absence of serum. On Day +1, activated PBMCs were transduced with viral vectors, e.g., Constructs #8, #10n, #11n, #13, #16, #18, and #19 in G-Rex® 100 cell culture vessels at about 5×10⁷ cells/vessel in the absence of serum. The amounts of virus used for transduction are shown in Table 10.

TABLE 10

| Constructs | Virus Volume/1 × 10⁶ cells | |
|---|---|---|
| #13, #16, #18, #10n | 5 | μl |
| #19 and #11n | 2.5 | μl |
| #8 (TCR) | 2.5 | μl |

| | |
|---|---|
| NT | — |

### Expansion

FIG. 54 shows that, on Day +2, transduced PBMCs were expanded in the absence of serum. On Day +7, cells were harvested for subsequent analysis, e.g., FACS-Tetramer and vector copy number (VCN) and were cryopreserved. Fold expansion on Day +7 was comparable for all constructs (approximately 30-fold expansion). Viabilities of cells is greater than 90% on Day +7.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+ Tetramer(Tet)+ and CD8+ Tet+.

Tumor death assays and cytokine expression in the presence and absence of autologous immature dendritic cells was also measured.

The results were consistent with the prior examples and are summarized in Table 11.

TABLE 11

| | | | | | | TCR only |
|---|---|---|---|---|---|---|
| | | Construct | Construct | Construct | Construct | Construct |
| | Parameters | #10 | #13 | #11 | #19 | #8 |
| Manufacturing | Viabilities | >90% | >90% | >90% | >90% | >90% |
| | Fold Expansion d7 | 28.7 ± 11% | 28.6 ± 11% | 31.6 ± 13% | 29.6 ± 13% | 30.1 ± 11% |
| | Transgene expression | 46.9 ± 12% | 42 ± 9.8% | 41 ± 12% | 48.2 ± 14% | 22.8 ± 8% |
| | (% CD3 + Tet+), | | | | | |
| | mean ± SD | | | | | |
| | Vector Copy Number | 3.3 ± 0.6% | 2.6 ± 0.7% | 2.0 ± 0.8% | 3.1 ± 1.8% | 1.7 ± 0.7% |
| Functionality | Multiple rounds of | +++ | +++ | +++ | +++ | +++ |
| | killing with UACC | | | | | |
| | Cytokine secretion | +++ | +++ | ++ | ++ | ++ |
| | (24 h, with UACC); | | | | | |
| | IFN-g, TNF-a, IL-2 | | | | | |
| | Cytokine secretion; | +++ | +++ | + | + | +/− |
| | CD4 + CD8 + TCR+ | | | | | |
| | (16 h, UACC); ICS | | | | | |
| | DC licensing assay | +++ | +++ | + | + | + |
| | (PBMC product) | | | | | |
| | IL-12, TNF-a & IL-6 | | | | | |
| | 3D Spheroid Assay | +++ | N/A | +++ | N/A | ++ |

### Example 14

### DC Licensing by CD4 Cells Expressing Constructs of the Present Disclosure

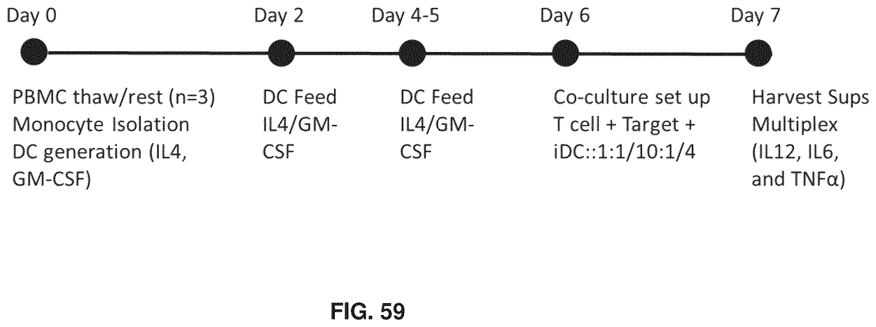

FIG. 59 shows a scheme of determining the levels of cytokine secretion by dendritic cells (DC) in the presence of PBMCs transduced with constructs of the present disclosure and in the presence of target cells, e.g., UACC257 cells. Briefly, Day 0, PBMCs (n=3) were thawed and rested, followed by monocyte isolation and autologous immature DCs (iDC) generation in the presence of IL-4 and GM-CSF; Day 2 and Day 4-5, DC were fed in the presence of IL-4 and GM-CSF; Day 6, iDC (+DC) were co-cultured with PBMC transduced with Construct #13, #16, #10n, #18, #11n, or #19 (Effector) and UACC257 cells (Target) at a ratio of Effector:Target:iDC=1:1/10:1/4 or without iDC (−DC), PBMCs transduced with TCR only, PBMCs without transduction (NT), PBMCs treated with iDC and LPS, and iDC only serve as controls; and Day 7 (after co-culturing for 24 hours), supernatants from the co-cultures were harvested, followed by cytokine profiling including, e.g., IL-12, IL-6, and TNF-a, using Multiplex.

Increased secretion of pro-inflammatory cytokines in tri-cocultures of autologous immature dendritic cells, UACC257 tumor cell line, and CD4+ T cell product expressing CD8αβ heterodimer and TCR (Construct #10) compared with that expressing CD8α* homodimer, in which the stalk region is replaced with CD8β stalk region, and TCR (Construct #11).

To determine the ability of CD4+ T cells expressing Constructs #10 or #11 to license DC, bulk PBMCs were transduced with Constructs #10 or #11, followed by selection of CD8+ and CD4+ cells from the product. Tri-cocultures of PBMCs, CD8+CD4− selected-product, or CD4+CD8+ selected-product with UACC257 tumor cell line in the presence or absence of autologous immature dendritic cells (iDCs) for 24 h followed by cytokine quantification of IL-12, TNF-α and IL-6 using Multiplex; iDCs alone or with LPS as controls, N=4-7, mean±SD, P values based on 2 way ANOVA.

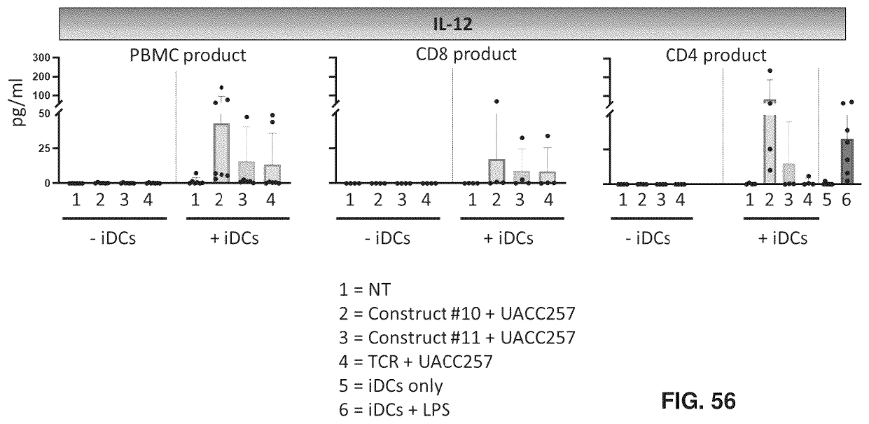

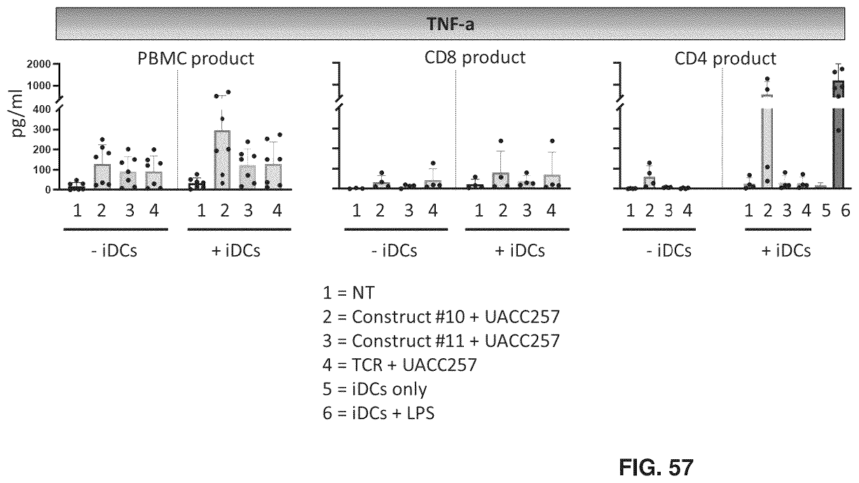

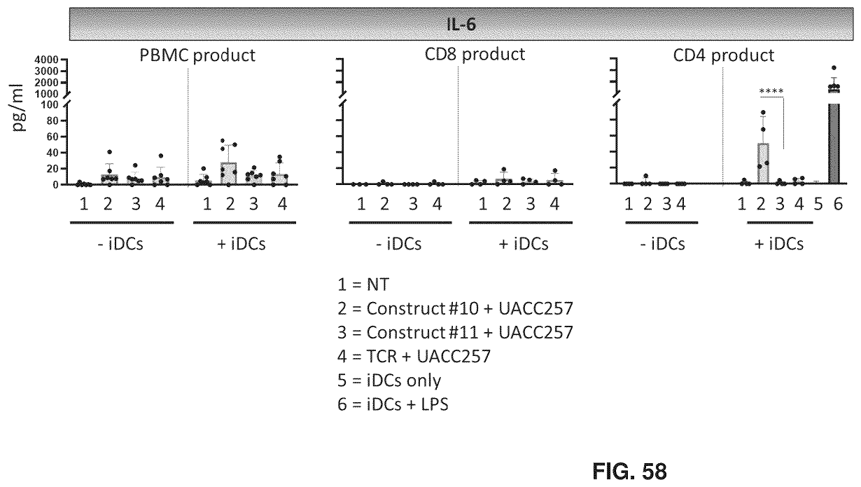

In the presence of immature dendritic cells (iDCs) and UACC257 cells, CD4+ T cells expressing Construct #10 (CD4+CD8+ T cells) performed better by inducing higher levels of IL-12 (FIG. 56), TNF-α (FIG. 57), and IL-6 (FIG. 58) secreted by dendritic cells (DC) than CD4+ T cells expressing Construct #11. On the other hand, the levels of IL-12, TNF-α, and IL-6 were comparable between CD8+ T cells expressing Constructs #10 and #11 (CD8+CD4− T cells). These results suggest that CD4+ T cells expressing CD8αβ heterodimer and TCR (Construct #10) may be a better product than CD4+ T cells expressing CD8α* homodimer and TCR (Construct #11) in DC licensing. The negative controls include the cytokine levels obtained (1) in the absence of iDCs (−iDCs), (2) in the presence of non-transduced T cells (NT)+UACC257 cells, and (3) in the presence of T cells transduced with TCR only (TCR)+UACC257 cells. The positive control includes the cytokine levels obtained from iDCs treated with lipopolysaccharide (LPS), which can activate DC.

### Example 15

### Assessment of DC Maturation and Cytokine Secretion by PBMC Products in Response to UACC257 Targets

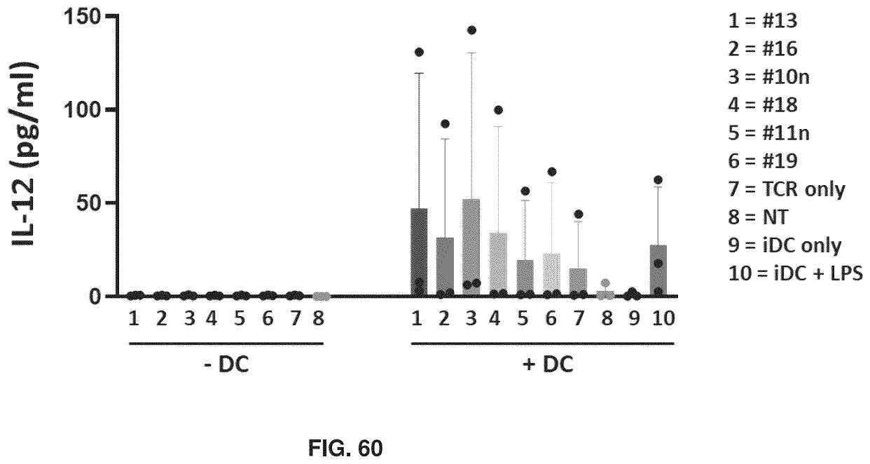

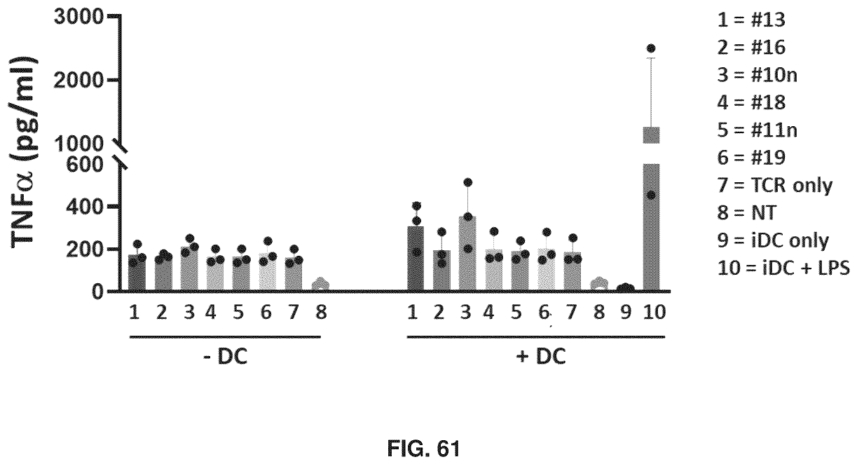

FIG. 60 shows IL-12 secretion levels induced by co-culturing PBMCs transduced with constructs of the present disclosure in the presence or absence of iDC and target cells, e.g., UACC257 cells. For example, IL-12 secretion was increased by co-culturing PBMCs transduced with Constructs #10 and 13 in the presence of iDC (+DC) and UACC257, as compared with that by co-culturing PBMCs transduced with TCR only. Increase of IL-12 secretion suggests (1) polarization towards Th1 cell-mediated immunity including TNF-α production (see, FIG. 61), (2) T cell proliferation, (3) IFN-γ production, and (4) cytolytic activity of cytotoxic T lymphocytes (CTLs).

FIG. 61 shows TNF-α secretion levels induced by co-culturing PBMCs transduced with constructs of the present disclosure in the presence or absence of iDC and target cells, e.g., UACC257 cells. For example, TNF-α secretion was increased by co-culturing PBMCs transduced with Constructs #10 and 13 in the presence of iDC (+DC) and UACC257, as compared with that by co-culturing PBMCs transduced with TCR only.

The increased IL-6 secretion (in addition to IL-12, TNF-α) may signify dendritic cell maturation, which may be augmented by CD40-CD40L interactions between CD4+ T cells and DCs. DC maturation and subsequent cytokine secretion may aid in modulation of the proinflammatory environment.

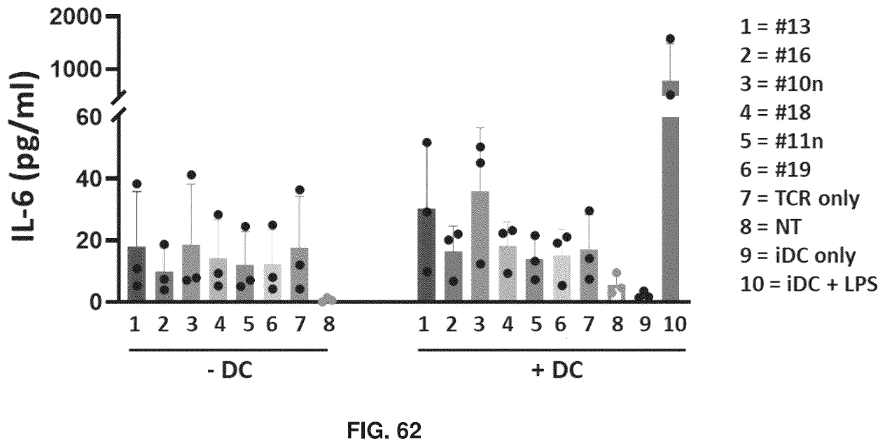

FIG. 62 shows IL-6 secretion levels induced by co-culturing PBMCs transduced with constructs of the present disclosure in the presence or absence of iDC and target cells, e.g., UACC257 cells. For example, IL-6 secretion was increased by co-culturing PBMCs transduced with Constructs #10 and 13 in the presence of iDC (+DC) and UACC257, as compared with that by co-culturing PBMCs transduced with TCR only.

These results show that PBMC products containing CD4+ T cells co-expressing transgenic TCR and CD8 co-receptor (CD8αβ heterodimer or CD8α homodimer) may license DCs in the microenvironment through antigen cross presentation to modulate the tumor microenvironment by, e.g., increasing IL-12, IL-6, and TNF-α secretion.

Table 12 shows comparison between constructs based on manufacturability and functionality.

TABLE 12

| | | Construct | Construct | Construct | Construct | |
|---|---|---|---|---|---|---|
| | Parameters | #10 | #13 | #11 | #19 | TCR only |
| Manufacturability | Viabilities | >90% | >90% | >90% | >90% | >90% |
| | Fold expansion | 28.7 ± 11% | 28.6 ± 11% | 31.6 ± 13% | 29.6 ± 13% | 30.1 ± 11% |
| | on Day 7 | | | | | |
| | Transgene | 46.9 ± 12% | 42 ± 9.8% | 41 ± 12% | 48.2 ± 14% | 22.8 ± 8% |
| | expression | | | | | |
| | (% CD3 + Tet+) | | | | | |
| | mean ± SD | | | | | |
| | Vector copy | 3.3 ± 0.6% | 2.6 ± 0.7% | 2.0 ± 0.8% | 3.1 ± 1.8% | 1.7 ± 0.7% |
| | number | | | | | |
| Functionality | Multiple rounds | +++ | +++ | +++ | +++ | +++ |
| | of killing with | | | | | |
| | UACC257 cells | | | | | |
| | Cytokine | +++ | +++ | ++ | ++ | ++ |
| | secretion (24 h, | | | | | |
| | with UACC257 | | | | | |
| | cells); IFN-γ, | | | | | |
| | TNF-α, IL-2 | | | | | |
| | Cytokine | +++ | +++ | + | + | +/− |
| | secretion; | | | | | |
| | CD4 + CD8 + TC | | | | | |
| | R + (16 h with | | | | | |
| | UACC257 | | | | | |
| | cells); ICS | | | | | |
| | DC licensing | +++ | +++ | + | + | + |
| | assay (PBMC | | | | | |
| | product) | | | | | |
| | IL-12, TNF-α, | | | | | |
| | and IL-6 | | | | | |
| | 3D spheroid | +++ | N/A | +++ | N/A | ++ |
| | assay | | | | | |

Notes:
“+++” = best response;
“++” = good response;
“+” = average response;
“+/−” = poor response.

Table 13 shows construct comparison and ranking (the smaller the number the better).

TABLE 13

| | Construct | Construct | Construct | Construct |
|---|---|---|---|---|
| Parameters | #10 | #13 | #11 | #19 |
| Manufacturability | 1 | 1 | 1 | 1 |
| Functionality PBMC | 1 | 1 | 2 | 2 |
| Functionality CD8 | 1 | 1 | 1 | 1 |
| Functionality CD4 | 1 | 1 | 3 | 3 |
| Time delay* | 1 | 1 | 1 | 1 |
| Total | 5 | 5 | 8 | 8 |

*Time delay here refers to any delay from, for example, GMP Vector manufacturing or any delay due to incomplete data set, which may add delay in implementation of constructs in clinical trials.

In sum, while manufacturability in terms of, e.g., viability, fold expansion, transgene expression, and vector copy number, may be equally good, as ranked 1, among cells transduced with Construct #10, #11, #13, or #19, functionality in terms of, e.g., cell killing, cytokine secretion, DC licensing, and 3D spheroid forming ability, of cells transduced with Construct #10 and #13 may be better, as ranked 1, than those transduced with Construct #11 and #19, as ranked 1-3.

### Example 16

### EC50 Assays

To determine the efficacy of T cells transduced with constructs of the present disclosure, e.g., Constructs #10 and #11, against target cells, EC50s were determined based on the levels of IFNγ produced by the transduced cells in the presence of PRAME peptide-pulsed T2 cells.

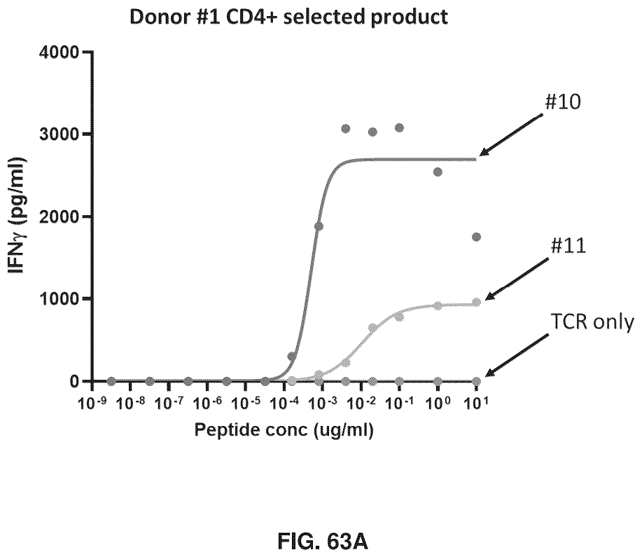

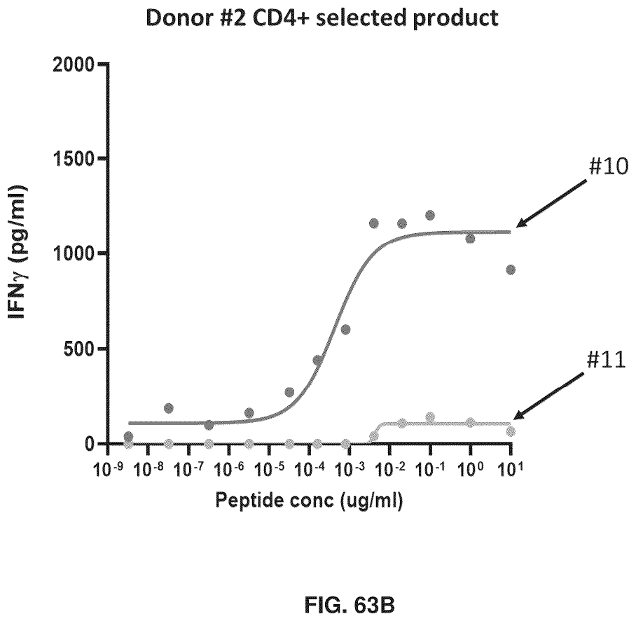

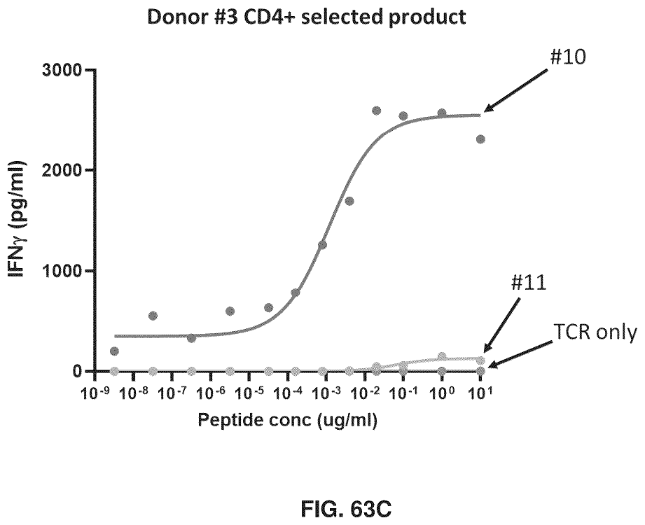

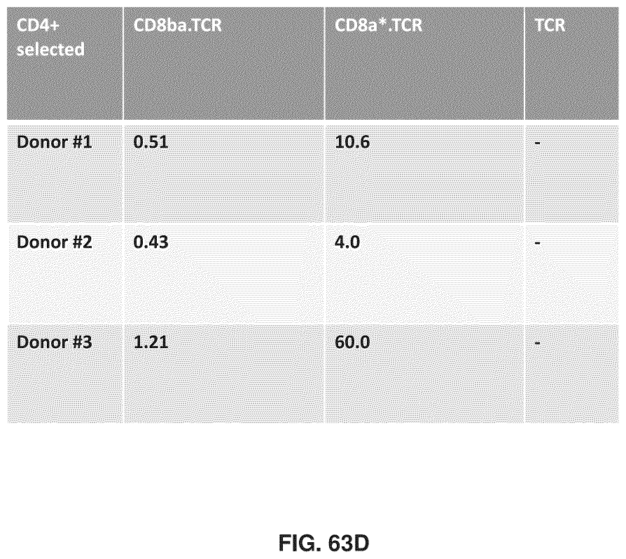

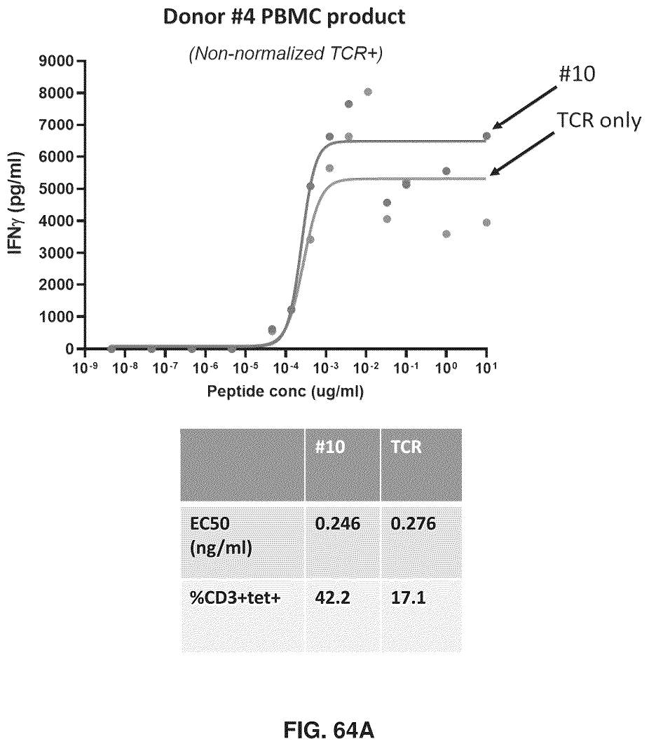

For example, to compare EC50s of CD4+ selected T cells transduced with Construct #10 (CD8αβ-TCR), Construct #11 (m1CD8α-TCR), or Construct #8 (TCR only), CD4+ selected products (TCR+ normalized) were co-cultured with PRAME peptide-pulsed T2 cells at defined concentrations at E:T ratio of 1:1 for 24 h. IFNγ levels were quantified in the supernatants after 24 h. FIGS. 63A-63C show IFNγ levels produced by the transduced CD4+ selected T cells obtained from Donor #1, #2, and #3, respectively. In general, CD4+ selected T cells transduced with Construct #10 were more sensitive to PRAME antigen as compared with that transduced with Construct #11 (m1CD8α TCR+CD4 T cells), as indicated by lower EC50 values (ng/ml) of CD4+ selected T cells transduced with Construct #10 than that transduced with Construct #11 (FIG. 63D). No response was observed among TCR+CD4+ cells (FIGS. 63A-63D). These results suggest that CD8αβ heterodimer may impart increased avidity to CD8αβ TCR+CD4+ T cells as compared to m1CD8α homodimer, leading to better efficacy against target cells.

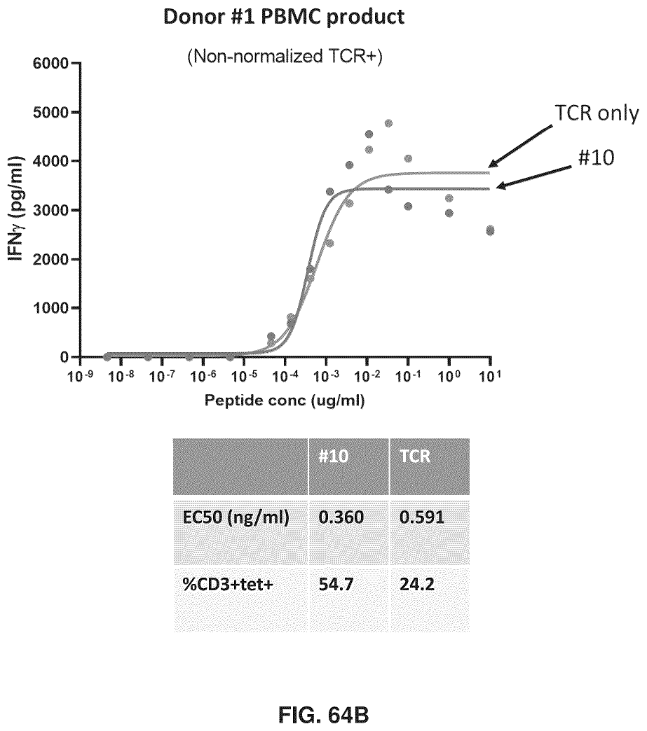

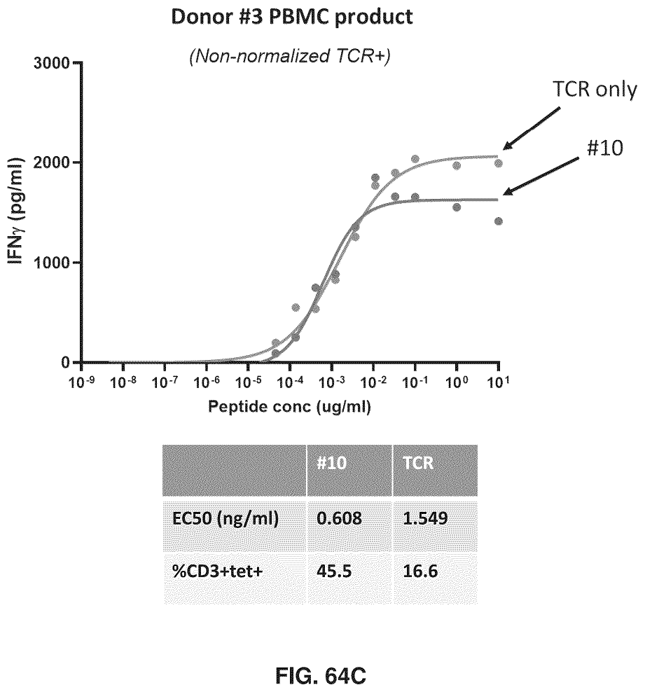

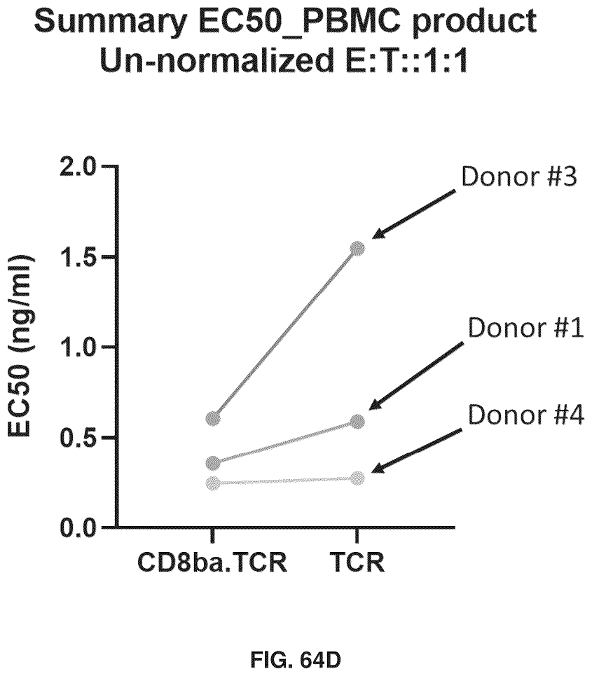

Similar experiments were performed using PBMC obtained from Donor #1, #3, and #4. Briefly, PBMC products (TCR+ non-normalized) were co-cultured with PRAME peptide-pulsed T2 cells at defined concentrations at E:T ratio of 1:1 for 24 h. IFNγ levels were quantified in the supernatants after 24 h. FIGS. 64A-64C show IFNγ levels produced by the transduced PBMC obtained from Donor #4, #1, and #3, respectively. Donor-to-donor variability was observed in the EC50 values. For example, while Donor #3 (FIGS. 64C and 64D) shows lower EC50 of PBMC transduced with Construct #10 as compared with that transduced with TCR only, Donors #1 (FIG. 64B) and #4 (FIG. 64A) show comparable EC50s between Construct #10 and TCR only (FIG. 64D). Thus, the increased avidity and efficacy observed in CD4+ selected T cell products expressing TCR and CD8αβ heterodimer as compared with that expressing TCR only may be obtained but to lesser extent when using PBMC products.

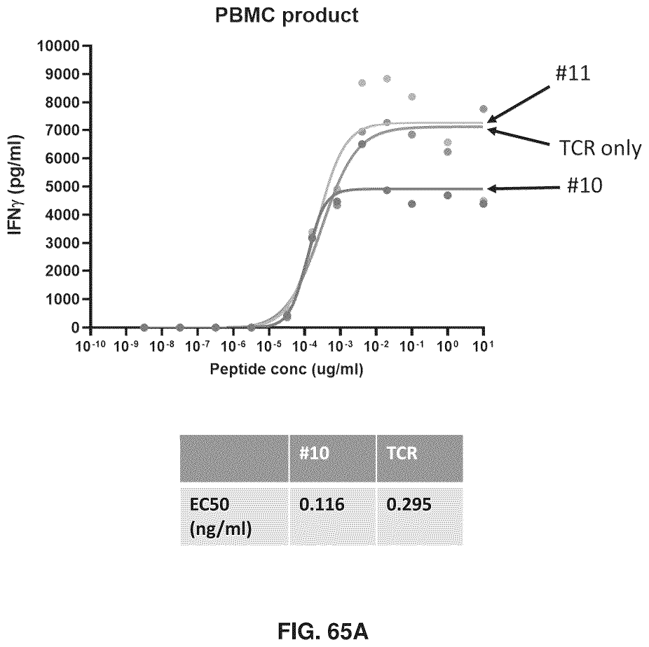

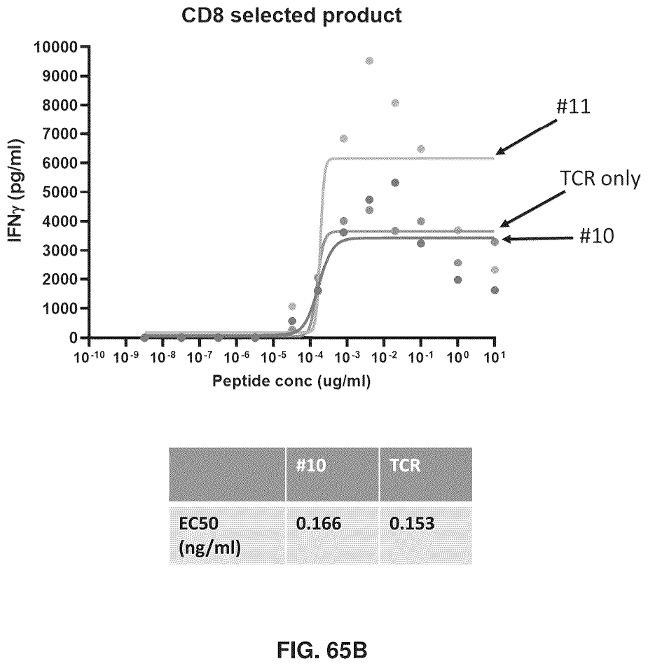

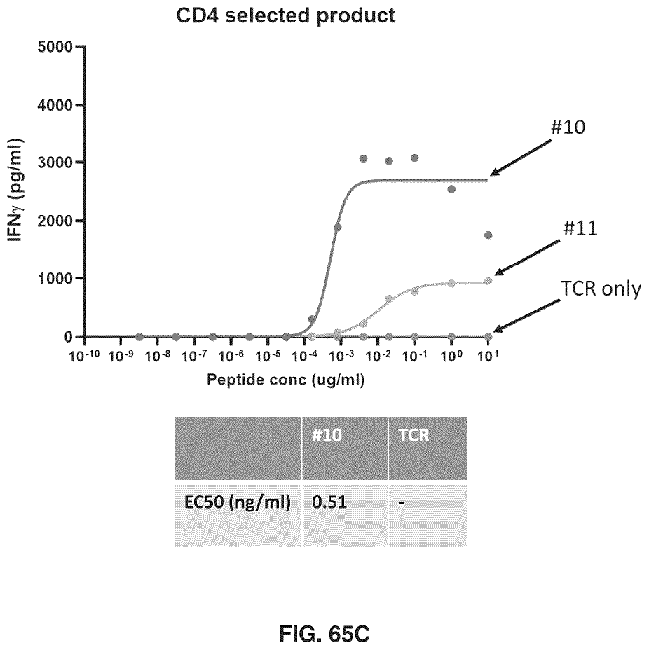

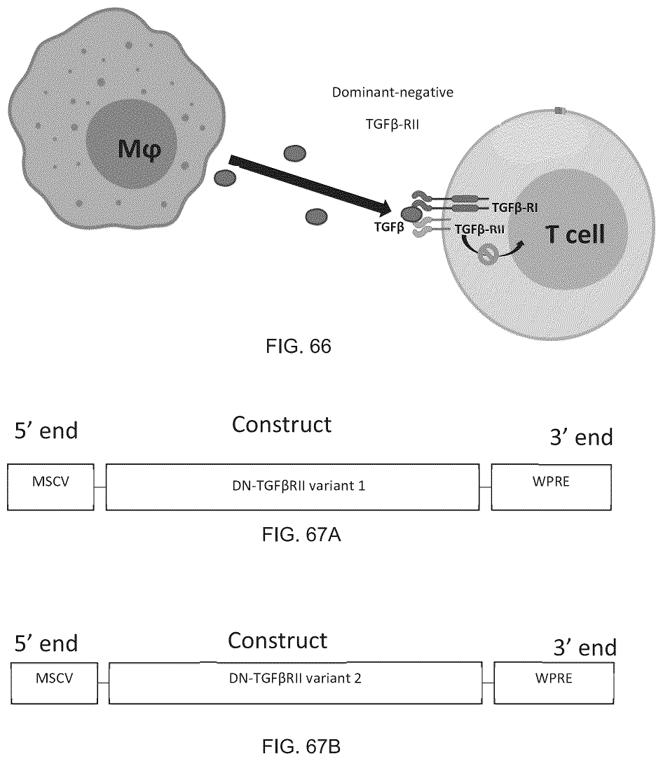

To compare EC50s of different T cell products obtained from the same donor, PBMC products, CD8+ selected products, and CD4+ selected products obtained from a single donor were co-cultured with PRAME peptide-pulsed T2 cells (TCR+ normalized) at defined concentrations at E:T ratio of 1:1 for 24 h. IFNγ levels were quantified in the supernatants after 24 h. FIGS. 65A-65C show that IFNγ levels produced by PBMC products (FIG. 65A), CD8+ selected products (FIG. 65B), and CD4+ selected products (FIG. 65C), respectively. Consistently, EC50 of CD4+ selected T cells transduced with Construct #10 was lower than that transduced with Construct #11 or TCR only (FIG. 65C), while EC50s of the transduced PBMC and CD8+ selected T cells were comparable between Construct #10 and TCR only transduction. Thus, the increased avidity and efficacy observed in CD4+ selected T cell products expressing TCR and CD8αβ heterodimer as compared with that expressing TCR and m1CD8α homodimer or with that expressing TCR only may be obtained but to lesser extent when using PBMC products or CD8+ selected T cell products.

### Example 17

### Cell Manufacturing—Small Scale

### Isolation, Rest, and Activation

Donor peripheral blood mononuclear cells (PBMC) were isolated from healthy donor leukapheresis and cryopreserved. PBMCs were later (on day D−1) thawed in Complete TexMACS medium (TexMACS (Miltenyi Biotec)+5% human AB serum), washed, and resuspended in Complete TexMACS and treated with benzonase nuclease (50 U/mL), and rested in Gas Permeable Rapid Expansion (G-Rex) or tissue culture-treated flasks overnight at 37° C.

On day D+0, following overnight rest, PBMC were counted, concentration-adjusted, and added to tissue culture bags coated with immobilized anti-CD3 & anti-CD28 antibodies for activation. Cells were activated overnight (16-20 hr) at 37° C.

### Transduction

On day D+1, following activation, cells were removed from the activation bags, washed, and counted. They were then added to G-Rex vessels containing a transduction master mix composed of unsupplemented TexMACS medium, protamine sulfate (10 μg/mL), interleukin (IL)-7 (10 ng/mL), and IL-15 (50 ng/mL). For cells to be transduced, lentiviral supernatant was added to the cells.

To produce cells expressing TCR only, lentivirus encoding a TCR against PRAME-004 (SLLQHLIGL) was added at 2.5 μL per million activated PBMC. This construct also comprised WPRE, as illustrated in FIG. 6, Construct #8. Unless otherwise noted, TCR-only cells in all further Examples herein were transduced with addition of lentivirus at 2.5 μL per million per cell. To produce cells expressing TCR and dominant negative TGFβRII variant 1 (dnTGFβRIIvar1) (SEQ ID NO: 305, encoded by SEQ ID NO: 306), lentivirus encoding dnTGFβRIIvar1 was also added at 0.31 (more specifically, 0.313) μL, 0.63 (more specifically, 0.625) μL, 1.25 μL, 2.5 μL, 3.75 μL, 5.0 μL, 6.25 μL, 7.5 μL, 8.75 μL, or 10.0 μL per million activated PBMC. To produce cells expressing TCR and dominant negative TGFβRII variant 2 (dnTGFβRIIvar2) (SEQ ID NO: 307, encoded by SEQ ID NO: 308), lentivirus encoding dnTGFβRIIvar2 was also added at 0.31 (more specifically, 0.313) μL, 0.63 (more specifically, 0.625) μL, 1.25 μL, 2.5 μL, 3.75 μL, 5.0 μL, 6.25 μL, 7.5 μL, 8.75 μL, or 10.0 μL per million activated PBMC.

To produce cells expressing dnTGFβRIIvar1 only, lentivirus encoding dnTGFβRIIvar1 (SEQ ID NO: 312) was added at 0.31 (more specifically, 0.313) μL, 0.63 (more specifically, 0.625) μL, 1.25 μL, 2.5 μL, 3.75 μL, 5.0 μL, 6.25 μL, 7.5 μL, 8.75 μL, or 10.0 μL per million activated PBMC. To produce cells expressing dnTGFβRIIvar2 only, lentivirus encoding dnTGFβRIIvar2 (SEQ ID NO: 313) was added at 0.31 (more specifically, 0.313) μL, 0.63 (more specifically, 0.625) μL, 1.25 μL, 2.5 μL, 3.75 μL, 5.0 μL, 6.25 μL, 7.5 μL, 8.75 μL, or 10.0 μL per million activated PBMC.

Vectors encoding dnTGFβRIIvar1 also comprise an MSCV promoter and WPRE (SEQ ID NO: 312). Vectors encoding dnTGFβRIIvar2 also comprise an MSCV promoter and WPRE (SEQ ID NO: 313).

For non-transduced (NT) cells, no lentivirus was added.

### Media/Cytokine Addition

The next day (˜24 hr) following transduction (on D+2), Complete TexMACS medium containing IL-7 (10 ng/ml) and IL-15 (50 ng/mL) were added to the vessel maximum volume, and cells were allowed to expand for 6 more days.

### Harvest and Cryopreservation

On day 7 (D+7), cells were harvested, washed, concentrated, and cryopreserved in CryoStor CS5 from BioLife Solutions.

### Characterization of Cell Products:

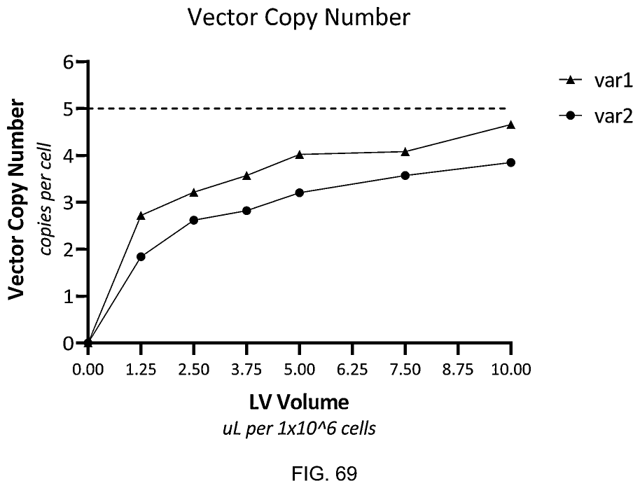

FIG. 69 shows vector copy number in cells transduced only with vectors encoding dnTGFβRIIvar1 (“var1”) or dnTGFβRIIvar2 (“var2”). Vector copy number (copies per cell) (Y axis) is plotted against the volume (in μL per 1×10⁶ cells) of lentivirus vector used to transduce the cells (X axis). Data are represented as mean. Vector copy number was assessed using quantitative PCR (qPCR) for the viral psi packaging element.

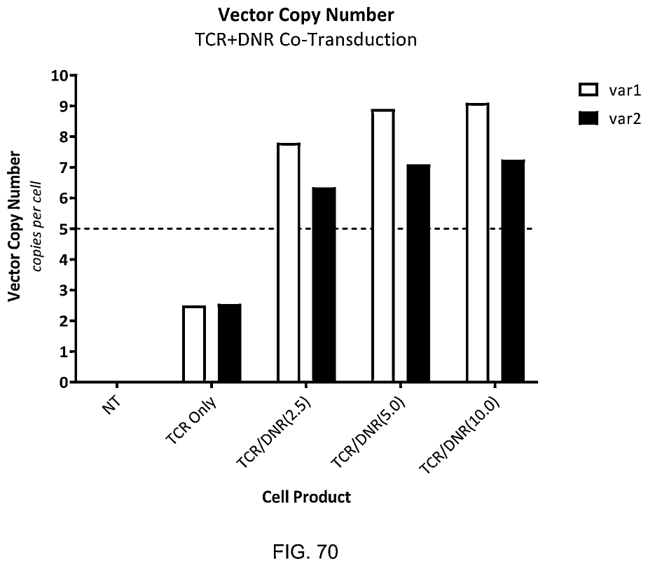

FIG. 70 shows vector copy number (copies per cell) in non-transduced cells (“NT”) and in cells transduced (i) only with vector encoding TCR at 2.5 μL per million cells (“TCR only”, both bars), (ii) with vector encoding TCR at 2.5 μL per million cells and with vector encoding dnTGFβRIIvar1 at 2.5 μL per million cells (“TCR/DNR(2.5)”, white bar), (iii) with vector encoding TCR at 2.5 μL per million cells and with vector encoding dnTGFβRIIvar1 at 5.0 μL per million cells (“TCR/DNR(5.0)”, white bar), (iv) with vector encoding TCR at 2.5 μL per million cells and with vector encoding dnTGFβRIIvar1 at 10.0 μL per million cells (“TCR/DNR(10.0)”, white bar), (v) with vector encoding TCR at 2.5 μL per million cells and with vector encoding dnTGFβRIIvar2 at 2.5 μL per million cells (“TCR/DNR(2.5)”, black bar), (vi) with vector encoding TCR at 2.5 μL per million cells and with vector encoding dnTGFβRIIvar2 at 5.0 μL per million cells (“TCR/DNR(5.0)”, black bar), or (vii) with vector encoding TCR at 2.5 μL per million cells and with vector encoding dnTGFβRIIvar2 at 10.0 μL per million cells (“TCR/DNR(10.0)”, black bar). Vector copy number (copies per cell) (Y axis) is shown. Data are represented as mean. These data show that TCR and dnTGFβRII lentiviral vectors contribute additively to vector copy number.

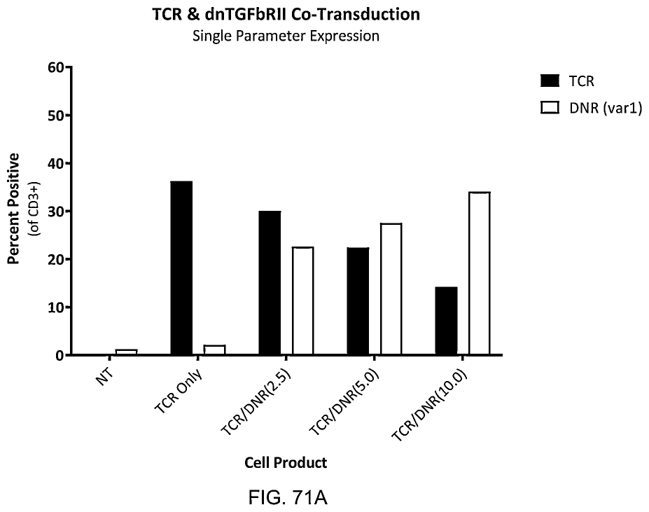

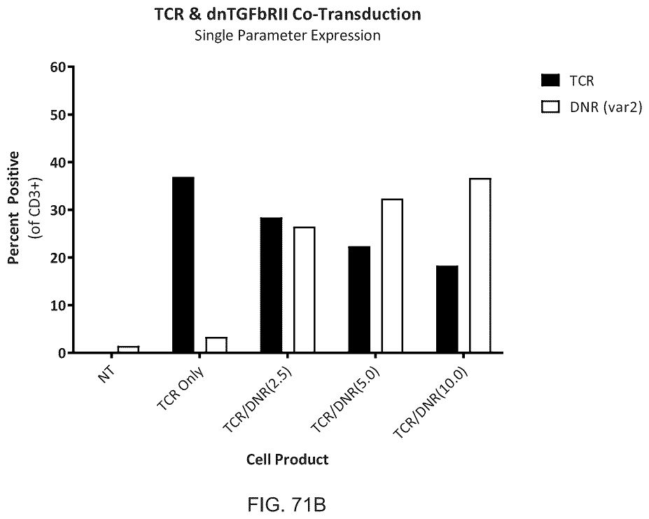

FIG. 71A shows percentage of CD3+ cells positive for TCR (black bars) or for dnTGFβRIIvar1 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar1 at 2.5 μL per million cells (“TCR/DNR(2.5)”), (iii) with vector encoding TCR at 2.5 μL per million cells and with vector encoding dnTGFβRIIvar1 at 5.0 μL per million cells (“TCR/DNR(5.0)”), or (iv) with vector encoding TCR at 2.5 μL per million cells and with vector encoding dnTGFβRIIvar1 at 10.0 μL per million cells (“TCR/DNR(10.0)”). Data are represented as mean.

Because antibody for TGFβRII cannot discriminate between wild-type TGFβRII and dnTGFβRII, the gating here was set on the non-transduced cells, so white bars are TGFβRII expression above baseline, but are likely to be dnTGFβRII and are used to represent dnTGFβRII. This applies to all flow cytometry measurements of dnTGFβRII set forth herein.

FIG. 71B shows percentage of CD3+ cells positive for TCR (black bars) or for dnTGFβRIIvar2 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar2 at 2.5 μL per million cells (“TCR/DNR(2.5)”), (iii) with vector encoding TCR at 2.5 μL per million cells and with vector encoding dnTGFβRIIvar2 at 5.0 μL per million cells (“TCR/DNR(5.0)”), or (iv) with vector encoding TCR at 2.5 μL per million cells and with vector encoding dnTGFβRIIvar2 at 10.0 μL per million cells (“TCR/DNR(10.0)”). Data are represented as mean.

The data in FIGS. 71A and 71B show that TCR expression decreases as DNR dose increases, suggesting competition between the two constructs during transduction.

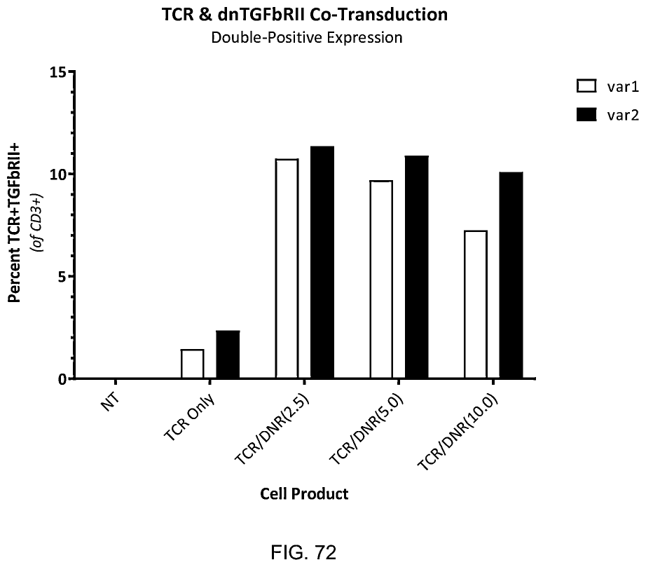

FIG. 72 shows percentage of CD3+ cells that are double-positive for TCR and dnTGFβRIIvar1 (white bars) or for TCR and dnTGFβRIIvar2 (black bars). Cells were non-transduced (“NT”) or transduced (i) only with vector encoding TCR at 2.5 μL per million cells (“TCR Only”, both bars), (ii) with vector encoding TCR at 2.5 μL per million cells and with vector encoding dnTGFβRIIvar1 at 2.5 μL per million cells (“TCR/DNR(2.5)”, white bar), (iii) with vector encoding TCR at 2.5 μL per million cells and with vector encoding dnTGFβRIIvar1 at 5.0 μL per million cells (“TCR/DNR(5.0)”, white bar), (iv) with vector encoding TCR at 2.5 μL per million cells and with vector encoding dnTGFβRIIvar1 at 10.0 μL per million cells (“TCR/DNR(10.0)”, white bar), (v) with vector encoding TCR at 2.5 μL per million cells and with vector encoding dnTGFβRIIvar2 at 2.5 μL per million cells (“TCR/DNR(2.5)”, black bar), (vi) with vector encoding TCR at 2.5 μL per million cells and with vector encoding dnTGFβRIIvar2 at 5.0 μL per million cells (“TCR/DNR(5.0)”, black bar), or (vii) with vector encoding TCR at 2.5 μL per million cells and with vector encoding dnTGFβRIIvar2 at 10.0 μL per million cells (“TCR/DNR(10.0)”, black bar). Data are represented as mean. These data show that the maximum TCR+TGFβRII+ double-positive cells was achieved at lowest TGFβRII vector dose tested.

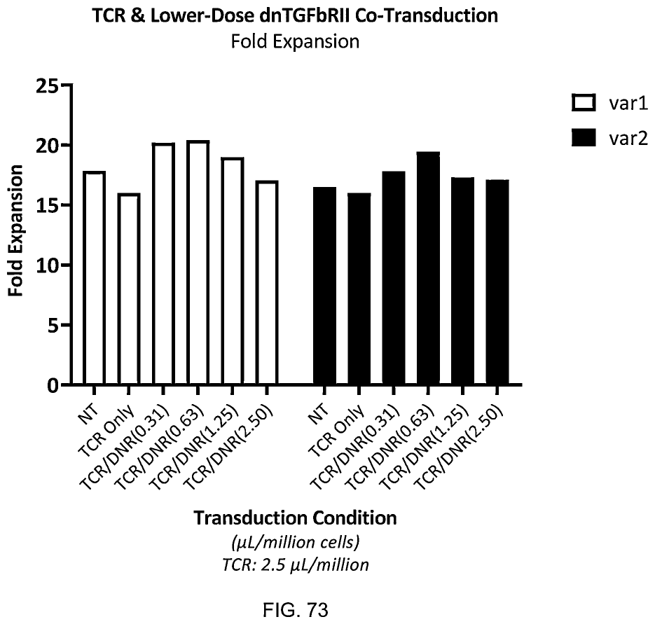

FIG. 73 shows fold expansion of non-transduced cells (“NT”, both bars) and of cells transduced with (i) only with vector encoding TCR at 2.5 μL per million cells (“TCR Only”, both bars), (ii) with vector encoding TCR at 2.5 μL per million cells and with vector encoding dnTGFβRIIvar1 at 0.31 μL per million cells (“TCR/DNR(0.31)”, white bar), (iii) with vector encoding TCR at 2.5 μL per million cells and with vector encoding dnTGFβRIIvar1 at 0.63 μL per million cells (“TCR/DNR(0.63)”, white bar), (iv) with vector encoding TCR at 2.5 μL per million cells and with vector encoding dnTGFβRIIvar1 at 1.25 μL per million cells (“TCR/DNR(1.25)”, white bar), (v) with vector encoding TCR at 2.5 μL per million cells and with vector encoding dnTGFβRIIvar1 at 2.50 μL per million cells (“TCR/DNR(2.50)”, white bar), (vi) with vector encoding TCR at 2.5 μL per million cells and with vector encoding dnTGFβRIIvar2 at 0.31 μL per million cells (“TCR/DNR(0.31)”, black bar), (vii) with vector encoding TCR at 2.5 μL per million cells and with vector encoding dnTGFβRIIvar2 at 0.63 μL per million cells (“TCR/DNR(0.63)”, black bar), (viii) with vector encoding TCR at 2.5 μL per million cells and with vector encoding dnTGFβRIIvar2 at 1.25 μL per million cells (“TCR/DNR(1.25)”, black bar), or (ix) with vector encoding TCR at 2.5 μL per million cells and with vector encoding dnTGFβRIIvar2 at 2.50 μL per million cells (“TCR/DNR(2.50)”, black bar). Data are represented as mean. These data show that cell fold expansion is similar in non-transduced cells, cells transduced with TCR only, and cells transduced with TCR and low doses of dnTGFβRII.

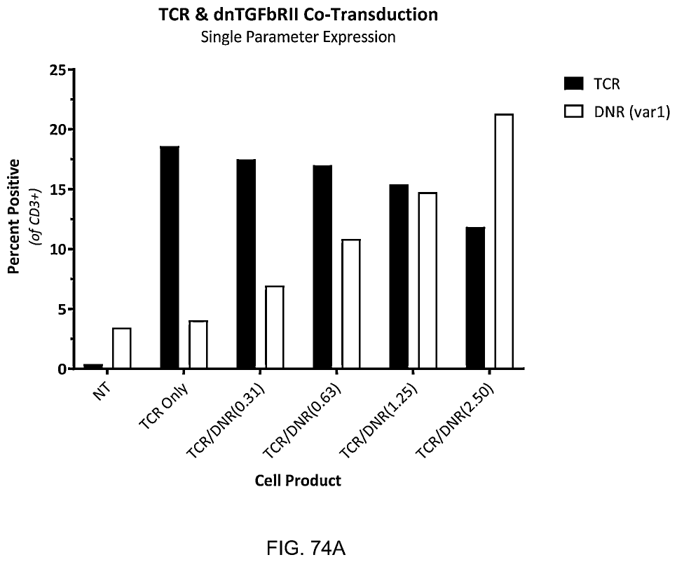

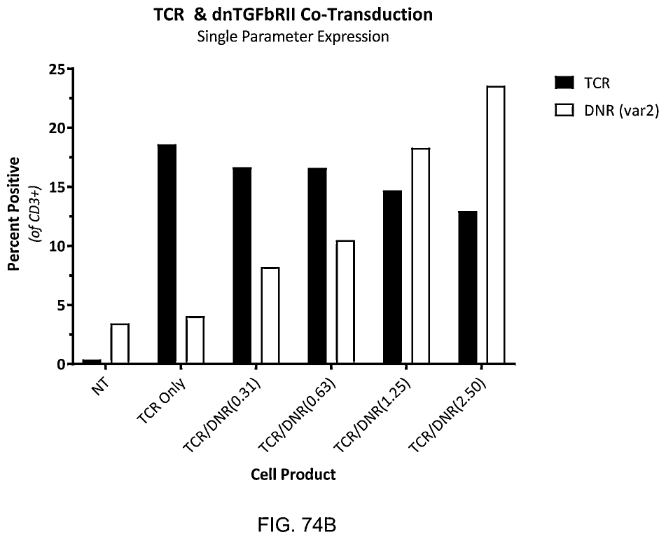

FIG. 74A shows percentage of CD3+ cells positive for TCR (black bars) or for dnTGFβRIIvar1 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar1 at 0.31 μL per million cells (“TCR/DNR(0.31)”), (iii) with vector encoding TCR at 2.5 μL per million cells and with vector encoding dnTGFβRIIvar1 at 0.63 μL per million cells (“TCR/DNR(0.63)”), (iv) with vector encoding TCR at 2.5 μL per million cells and with vector encoding dnTGFβRIIvar1 at 1.25 μL per million cells (“TCR/DNR(1.25)”), or (v) with vector encoding TCR at 2.5 μL per million cells and with vector encoding dnTGFβRIIvar1 at 2.50 μL per million cells (“TCR/DNR(2.50)”). Data are represented as mean.

FIG. 74B shows percentage of CD3+ cells positive for TCR (black bars) or for dnTGFβRIIvar2 (white bars). Cells were non-transduced (“NT”) or transduced (i) only with vector encoding TCR at 2.5 μL per million cells (“TCR Only”), (ii) with vector encoding TCR at 2.5 μL per million cells and with vector encoding dnTGFβRIIvar2 at 0.31 μL per million cells (“TCR/DNR(0.31)”), (iii) with vector encoding TCR at 2.5 μL per million cells and with vector encoding dnTGFβRIIvar2 at 0.63 μL per million cells (“TCR/DNR(0.63)”), (iv) with vector encoding TCR at 2.5 μL per million cells and with vector encoding dnTGFβRIIvar2 at 1.25 μL per million cells (“TCR/DNR(1.25)”), or (v) with vector encoding TCR at 2.5 μL per million cells and with vector encoding dnTGFβRIIvar2 at 2.50 μL per million cells (“TCR/DNR(2.50)”). Data are represented as mean.

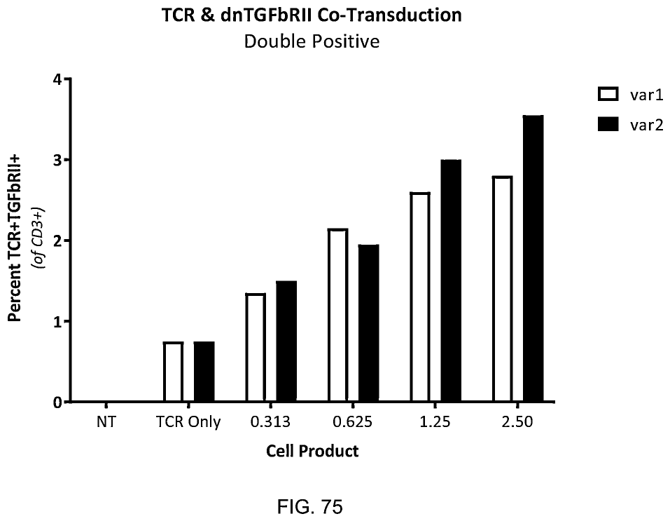

The data in FIGS. 74A and 74B show similar expression of TCR by cells also transduced with dnTGFβRIIvar1 at 0.31, 0.63, 1.25, or 2.50 μL per million cells The data show that the relative reduction in dnTGFβRIIvar1 expression resulting from reducing dnTGFβRII LV volume below 2.5 μL per million cells may not justify the slight increase in TCR expression; the optimal dose for co-transduction with TCR and DNR to balance expression of both constructs appears to be 2.5 μL and 2.5 μL, respectively. This is reinforced when evaluating TCR+dnTGFβRII+ double-positive expression, in FIG. 75.

FIG. 75 shows percentage of CD3+ cells double-positive for TCR and dnTGFβRIIvar1 (white bars) or for TCR and dnTGFβRIIvar2 (black bars). Cells were non-transduced (“NT”) or transduced (i) only with vector encoding TCR at 2.5 μL per million cells (“TCR Only”, both bars), (ii) with vector encoding TCR at 2.5 μL per million cells and with vector encoding dnTGFβRIIvar1 at 0.313 μL per million cells (“TCR/DNR(0.313)”, white bar), (iii) with vector encoding TCR at 2.5 μL per million cells and with vector encoding dnTGFβRIIvar1 at 0.625 μL per million cells (“TCR/DNR(0.625)”, white bar), (iv) with vector encoding TCR at 2.5 μL per million cells and with vector encoding dnTGFβRIIvar1 at 1.25 μL per million cells (“TCR/DNR(1.25)”, white bar), (v) with vector encoding TCR at 2.5 μL per million cells and with vector encoding dnTGFβRIIvar1 at 2.50 μL per million cells (“TCR/DNR(1.25)”, white bar), (vi) with vector encoding TCR at 2.5 μL per million cells and with vector encoding dnTGFβRIIvar2 at 0.313 μL per million cells (“TCR/DNR(0.313)”, black bar), (vii) with vector encoding TCR at 2.5 μL per million cells and with vector encoding dnTGFβRIIvar2 at 0.625 μL per million cells (“TCR/DNR(0.625)”, black bar), or (viii) with vector encoding TCR at 1.25 μL per million cells and with vector encoding dnTGFβRIIvar2 at 10.0 μL per million cells (“TCR/DNR(1.25)”, black bar), or (ix) with vector encoding TCR at 1.25 μL per million cells and with vector encoding dnTGFβRIIvar2 at 2.5 μL per million cells (“TCR/DNR(2.5)”, black bar). Data are represented as mean. These data show that the percent of double-positive cells maximized at 2.5 μL each TCR and dnTGFβRII.

### Example 18

### Transduction with Transduction Enhancers

Donor PBMC were thawed, rested, and activated in tissue culture bags coated with immobilized anti-CD3 & anti-CD28 antibodies as described previously in this Example. Transduction enhancers were tested to determine if co-transduction could be improved. Protamine sulfate (PS) is a highly cationic soluble polypeptide that is added to culture to increase viral particle-to-cell interactions. PS is an FDA-approved drug typically used for binding excess heparin in the blood. LentiBOOST® (LB), a product of SIRION Biotech, is a polaxamer-based non-cytotoxic transduction enhancer that functions by facilitating fusion of lentivirus particles with cell membranes, independent of receptors.

PS and LB were tested in conjunction to determine the effect of each. They were used at 10 μg/mL and 1 mg/mL, respectively. As a control, cells were also transduced with no enhancer.

1×10⁶ activated PBMC per well were plated in in 500 μL unsupplemented TexMACS medium containing 10 ng/mL IL-7, 50 ng/mL IL-15, and transduction enhancer (if used) in a G-Rex24 well tissue culture vessel. Cells (i) were not transduced or (ii) were transduced with 2.5 μL of TCR-encoding lentivirus (LV) per million cells and 2.5 μL dnTGFβRIIvar1-encoding lentivirus per million cells. Cells were incubated overnight at 37° C.

Two methods of co-transduction were tested: Either both LV constructs were added simultaneously to the activated PBMC (“Mixed”) or TCR-only LV was added for 6-8 hours followed by the addition of the dnTGFβRII construct (“Serial”) to determine if LV competition could be reduced without the need to activate the PBMC a second time.

Additionally, some conditions tested a “spinoculation” step to determine if centrifugation of cells in the transduction mixture would facilitate LV particle-T cell interactions. In this case, only the Mixed conditions were tested. Cells in these conditions were initially aliquoted into a standard (non-G-Rex) 24-well tissue culture plate along with their respective LV constructs and enhancer. The plates were centrifuged at 800×g for 90 minutes at 20° C./room temperature. Following spinoculation, the cells were incubated at 37° C.

The following day, cells transduced in the G-Rex vessels were supplemented with 6.5 mL Complete TexMACS containing IL-7 and IL-15 and placed back into incubation until harvest. Cells in standard (non-G-Rex) spinoculation plates were transferred to fresh G-Rex24 vessels and similarly supplemented with 6.5 mL Complete TexMACS with cytokines until harvest.

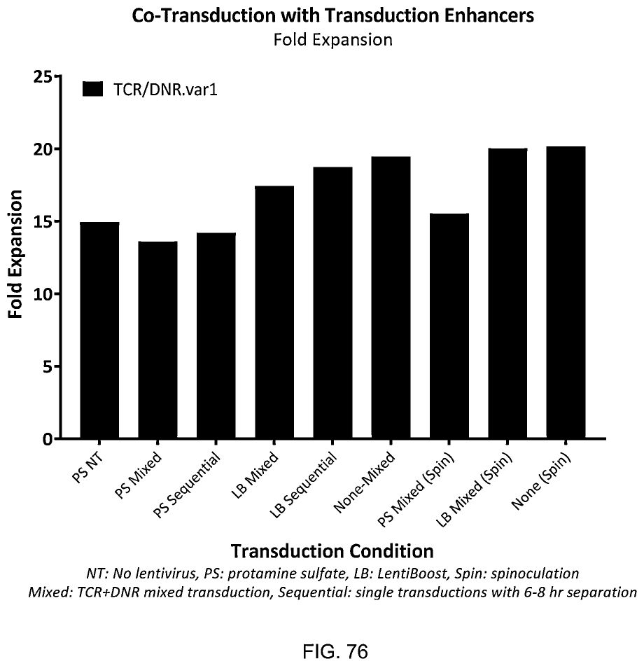

Cells were harvested and counted on day 6 following transduction. Fold expansion was calculated by dividing the resultant harvested cell number with the cell number with which transduction was initiated (1×10⁶). FIG. 76 shows fold expansion of non-transduced cells (“PS NT”) and cells transduced with TCR (2.5 μL per million cells) and dnTGFβRIIvar1 (2.5 μL per million cells) (all other bars). “PS NT” non-transduced cells were incubated with PS. “PS Mixed” transduced cells were transduced with TCR and dnTGFβRIIvar1 simultaneously with PS added. “PS Sequential” transduced cells were transduced with TCR and dnTGFβRIIvar1 sequentially with PS added. “LB Mixed” transduced cells were transduced with TCR and dnTGFβRIIvar1 simultaneously with LB added. “LB Sequential” transduced cells were transduced with TCR and dnTGFβRIIvar1 sequentially with LB added. “None Mixed” transduced cells were transduced with TCR and dnTGFβRIIvar1 simultaneously with no enhancer added. “PS Mixed (Spin)” transduced cells were transduced with TCR and dnTGFβRIIvar1 simultaneously with PS added and were spinoculated. “LB Mixed (Spin)” transduced cells were transduced with TCR and dnTGFβRIIvar1 simultaneously with LB added and were spinoculated. “None (Spin)” transduced cells were transduced with TCR and dnTGFβRIIvar1 simultaneously with no enhancer added and were spinoculated. These data show that LentiBoost® or no enhancer performed better than protamine sulfate.

Harvested cells were evaluated by flow cytometry using a panel of antibodies for the surface antigens CD3, CD4, CD8, and TGFβRII, as well as a TCR-specific tetramer. Percent TCR-positive and percent TGFβRII-positive cells were reported as single parameters as a proportion of CD3+ lymphocytes. Similarly, the percent of co-expressing (TCR+TGFβRII+ double-positive) cells were reported as a proportion of CD3+ lymphocytes.

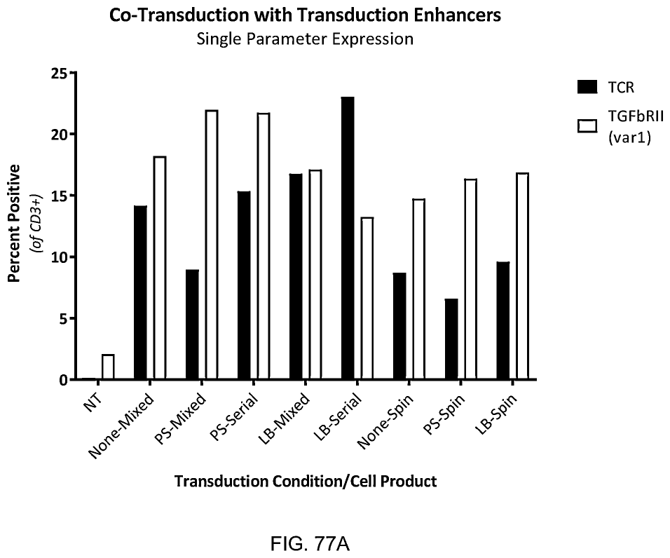

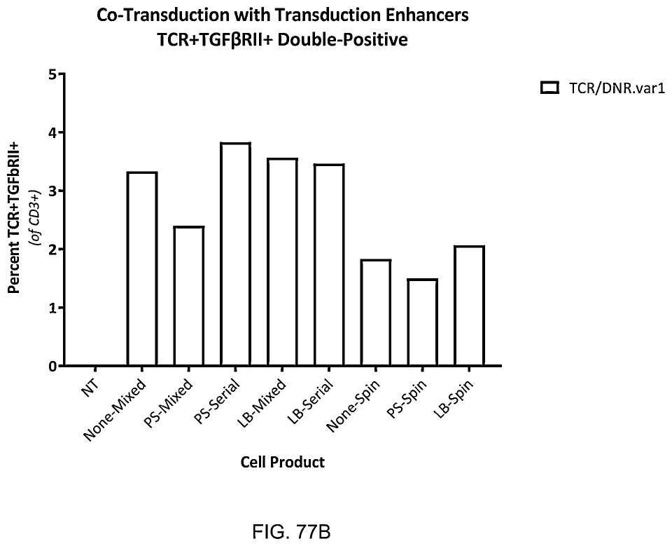

FIG. 77A shows percentage of CD3+ cells that were positive for TCR (black bars) or for dnTGFβRIIvar1 (white bars). Non-transduced cells (“NT”) are shown as a control. “None-Mixed” transduced cells were transduced with TCR and dnTGFβRIIvar1 simultaneously with no enhancer added. “PS Mixed” transduced cells were transduced with TCR and dnTGFβRIIvar1 simultaneously with PS added. “PS Serial” transduced cells were transduced with TCR and dnTGFβRIIvar1 sequentially with PS added. “LB Mixed” transduced cells were transduced with TCR and dnTGFβRIIvar1 simultaneously with LB added. “LB Serial” transduced cells were transduced with TCR and dnTGFβRIIvar1 sequentially with LB added. “None (Spin)” transduced cells were transduced with TCR and dnTGFβRIIvar1 simultaneously with no enhancer added and were spinoculated. “PS (Spin)” transduced cells were transduced with TCR and dnTGFβRIIvar1 simultaneously with PS added and were spinoculated. “LB (Spin)” transduced cells were transduced with TCR and dnTGFβRIIvar1 simultaneously with LB added and were spinoculated. Data are represented as mean.

FIG. 77B shows percentage of CD3+ cells that were double-positive for TCR and dnTGFβRIIvar1. Non-transduced cells (“NT”) are shown as a control. “None-Mixed” transduced cells were transduced with TCR and dnTGFβRIIvar1 simultaneously with no enhancer added. “PS Mixed” transduced cells were transduced with TCR and dnTGFβRIIvar1 simultaneously with PS added. “PS Serial” transduced cells were transduced with TCR and dnTGFβRIIvar1 sequentially with PS added. “LB Mixed” transduced cells were transduced with TCR and dnTGFβRIIvar1 simultaneously with LB added. “LB Serial” transduced cells were transduced with TCR and dnTGFβRIIvar1 sequentially with LB added. “None (Spin)” transduced cells were transduced with TCR and dnTGFβRIIvar1 simultaneously with no enhancer added and were spinoculated. “PS (Spin)” transduced cells were transduced with TCR and dnTGFβRIIvar1 simultaneously with PS added and were spinoculated. “LB (Spin)” transduced cells were transduced with TCR and dnTGFβRIIvar1 with LB added and were spinoculated. Data are represented as mean.

The data in FIGS. 77A and 77B show that LentiBOOST® or no enhancer with mixed transduction provided best fold expansion and double-positive TCR+dnTGFβRII+ expression.

### Example 19

### Cell Manufacturing—Mid-Scale

PBMC from two healthy donors sufficient for initiation of transduction in several G-Rex10M wells were thawed, rested, and activated as described in Example 17.

16-20 hours following activation, cells were transduced using the TCR-encoding lentiviral (LV) at a dose of 2.5 μL per million activated cells and dnTGFβRIIvar1-encoding LV at a dose of 2.5 μL per million activated cells, in unsupplemented (serum-free) TexMACS medium containing 10 ng/mL IL-7 and 50 ng/mL IL-15 and no transduction enhancer. Transduction cell density was targeted at 5×10⁶ per well in 2.5 mL (2×10⁶/mL; 5×10⁵ per cm²). Number of cells post-activation were sufficient for seeding 1 non-transduced well and 4 transduced wells. For Donor 1, five wells were initiated (1 NT, 4 transduced) at 4.32×10⁶ per well; similarly for Donor 2, five wells were initiated (1 NT, 4 transduced) at 5.20×10⁶ per well. Non-transduced cells were similarly prepared. Actual numbers of cells seeded per well were 4.32×10⁶ or 5.20×10⁶; the transduction master mix volume was scaled accordingly to normalize the cytokine and LV concentrations.

Cells were incubated with transduction mixture overnight at 37° C. About 24 hours following transduction, cells were supplemented with Complete TexMACS containing IL-7 and IL-15 to a maximum volume of 40 mL per well and incubated at 37° C. until harvest. Cells were harvested on day 6 following transduction, counted, evaluated for phenotype, and cryopreserved.

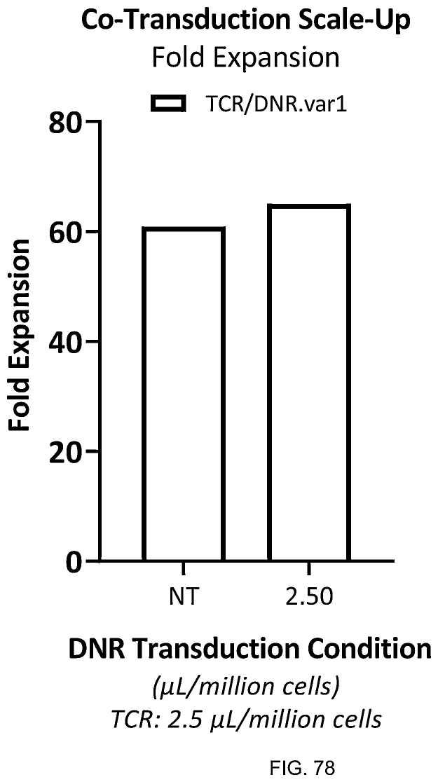

FIG. 78 shows fold expansion (Y Axis) of non-transduced (“NT”) cells and cells transduced with TCR (2.5 μL/million cells) and dnTGFβRIIvar1 (2.5 μL/million cells) (“2.50”). Data are represented as mean.

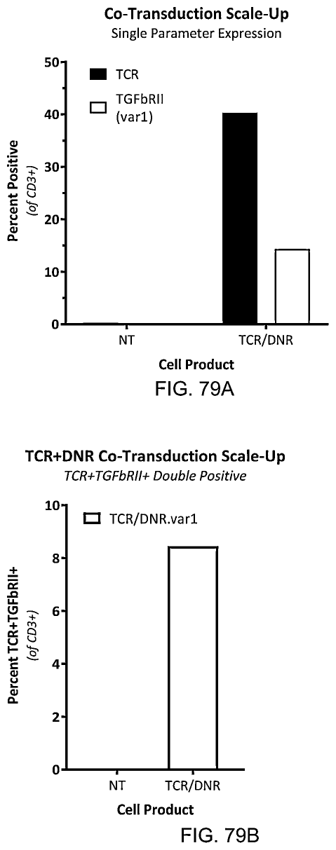

FIG. 79A shows percentage of non-transduced CD3+ cells (“NT”) and CD3+ cells transduced with TCR and dnTGFβRIIvar1 (“TCR/DNR”) that were positive for TCR (black bar) or for dnTGFβRIIvar1 (white bar). Data are represented as mean.

FIG. 79B shows percentage of non-transduced CD3+ cells (“NT”) and CD3+ cells transduced with TCR and dnTGFβRIIvar1 (“TCR/DNR”) that were double-positive for TCR and dnTGFβRIIvar1. Data are represented as mean.

### Cell Sorting

Cells generated from the mid-scale scale-up were sorted on a Miltenyi Biotech Tyto® cell sorter. Briefly, cells were thawed and washed, and about 1.5×10⁷ were stained for surface TGFβRII and for TCR-specific tetramer. Following staining, cells were washed counted, concentration-adjusted to approximately 5×10⁶/mL in 5 mL of Tyto® Buffer (PBS, 1% human serum albumin, 100 U/mL benzonase nuclease), filtered, and added to the Input chamber of a Tyto® cartridge.

Cells were sorted on the Tyto® in two runs per donor to produce the following fractions:

TCR+DNR+: Run 1 started with the freshly labeled, unsorted cells. The sort gate was set on TCR+TGFβRII+ double-positive cells. The resultant flow-through was depleted of TCR+TGFβRII+.

TCR+: Run 2 started with the flow-through from Run 1, which was returned to the input chamber of the Tyto® cartridge. The sort gate was set on TCR+ single-positive cells. The resultant flow through was depleted of TCR+TGFβRII+ cells and TCR+TGFβRII− cells. The resultant cells, depleted of TCR+TGFβRII+ cells and TCR+TGFβRII− cells, served as a “non-transduced” control in further experiments and are referred to as “waste”.

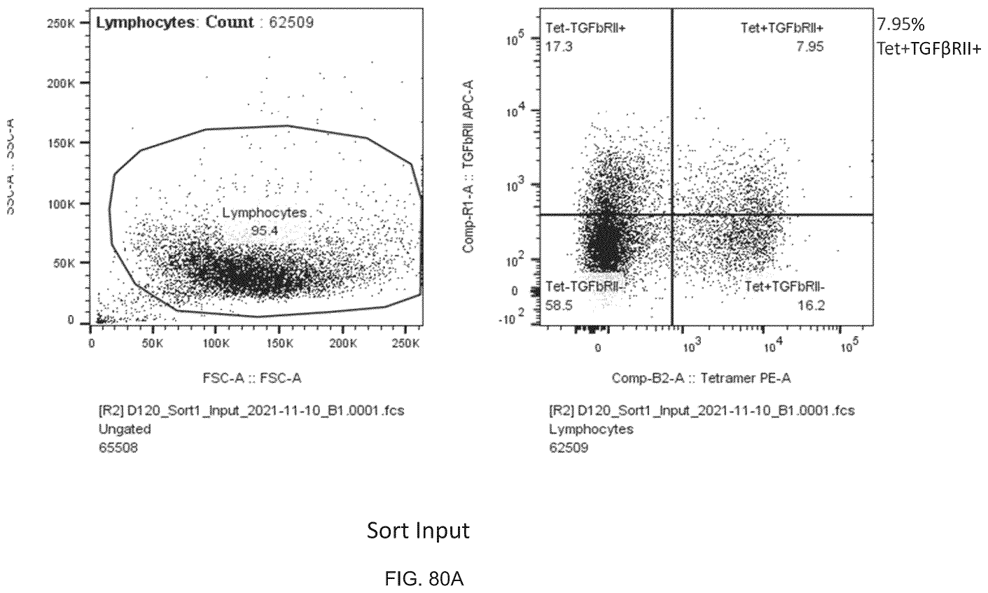

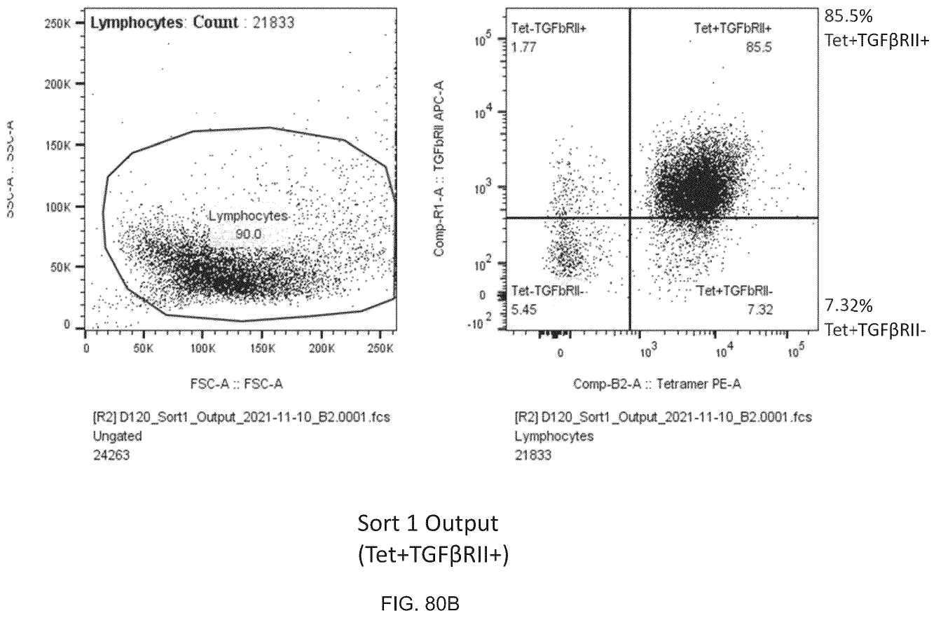

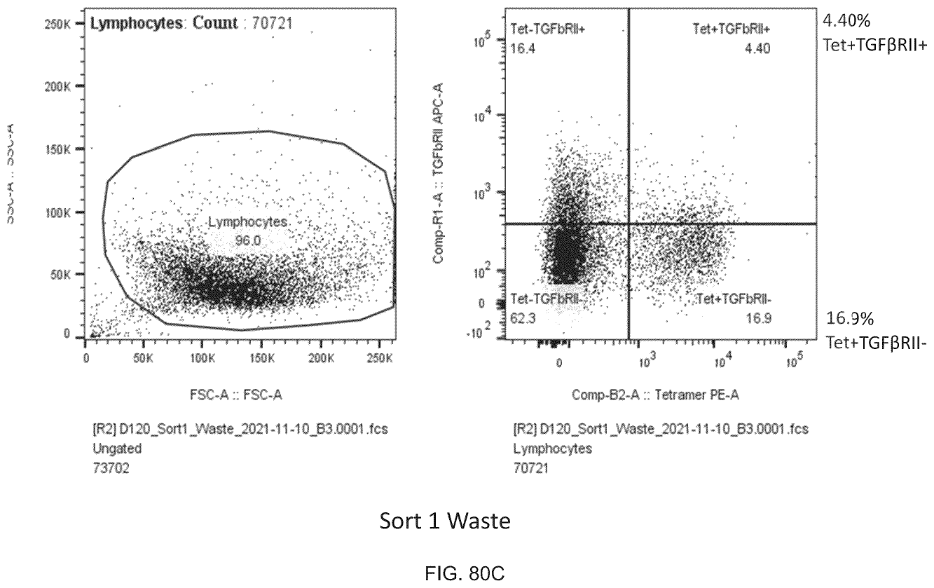

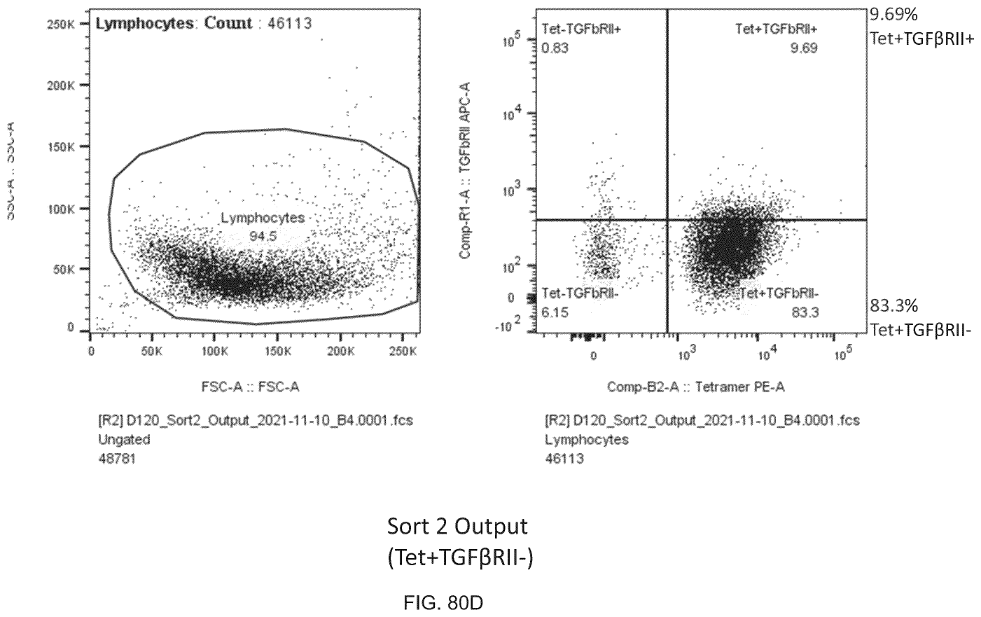

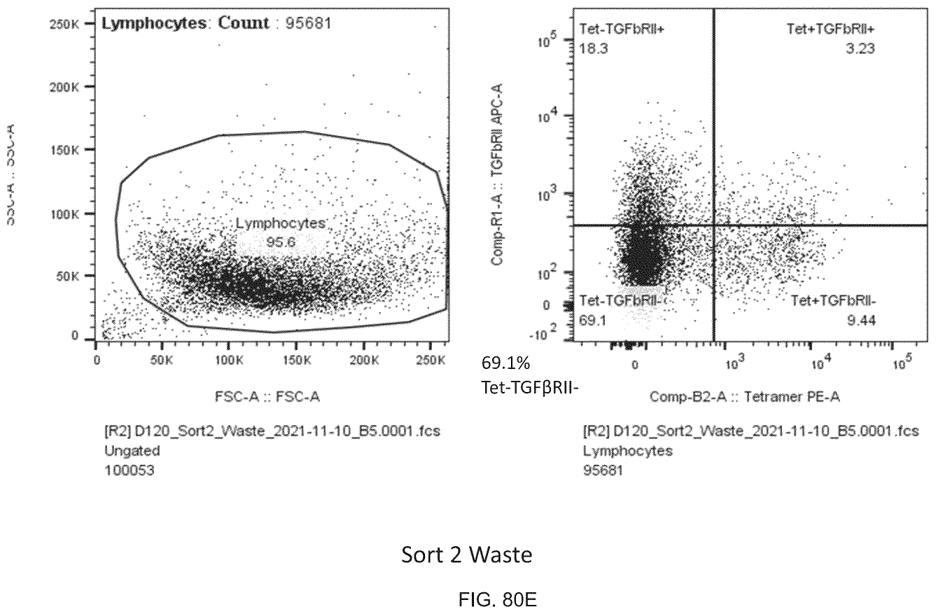

FIGS. 80A-80E show the sorting scheme and plots of resultant cell fractions for one donor (D120). It can be seen that the Sort 1 input (FIG. 80A) comprised a mix of TCR+dnTGFβRII+ cells, TCR+dnTGFβRII− cells, TCR-dnTGFβRII+ cells, and TCR-dnTGFβRII− cells. Collected Sort 1 output (FIG. 80B) comprised predominantly TCR+dnTGFβRII+ cells. The flow-through cells from Sort 1 (“Sort 1 Waste”) (FIG. 80C) were sorted a second time, producing Sort 2 output cells (FIG. 80D) comprising predominantly TCR+dnTGFβRII− cells, which were collected. The flow-through cells from Sort 2 (“Sort 2 Waste” or “waste”) (FIG. 80E) comprised predominantly TCR-dnTGFβRII− cells. For each of FIGS. 80A-80E, the left plot (FSC×SSC) shows the parent gate for the plot on the right; TCR/DNR expression as a proportion of “all lymphocytes”. Tet+ cells are TCR+ cells, while Tet− cells are TCR− cells.

A small sample of each post-sort fraction was evaluated on the Miltenyi Biotech MACSQuant® to assess for sort purity.

### Example 20

### Tumor Death Assays

Cells prepared as in Example 17 were thawed, washed, and resuspended in TexMACS medium supplemented with 5% by volume human AB serum (“Complete TexMACS”). Benzonase nuclease was added to a concentration of 50 U/mL and the cells were incubated (rested) overnight in tissue culture-treated flasks at 37° C.

UACC257 tumor cells were harvested using 0.05% trypsin, washed, and counted. Red fluorescent protein (RFP)-labeled tumor cells (“UACC257-RFP”) were plated at 5,000 to 10,000 per well in a flat-bottomed 96-well plate in 100 μL of Complete TexMACS. Plates were placed in an incubator at 37° C. UACC257 cells express high levels of the antigen PRAME (preferentially expressed antigen in melanoma).

Transduced cells (effector) and tumor cells (target) were co-cultured in 96 well tissue culture plates. Effector/target co-culture plates were placed into an IncuCyte ZOOM imager at 37° C. and 5% CO₂ and imaged every 2 hours for the duration of 3 to 12 days. Data was exported from the IncuCyte ZOOM software into Microsoft Excel and GraphPad Prism for further analysis. Fold tumor growth (RFP+ cell count) was normalized to 0 hr timepoint.

### Example 21

### Tumor Death Assays with TGF-β/Galunisertib

TCR-only transduced cells from one donor (prepared as in Example 17) were co-cultured with UACC257 tumor cells, as described in Example 20, at an effector:target (E:T) ratio of 5:1. Galunisertib (GAL, also known as LY2157299) is a small molecule inhibitor of TGF-β receptor type I (TGFβRI). TGF-β1 and/or GAL were added to some co-cultures TGF-131 was added at the initiation of coculture for all assays where it was used. Depending on the assay, subsequent additions of TGF-β1 were performed concurrent with fresh tumor cell additions (every 3-4 days) or daily as specified. TGF-β1 was not added (“0 ng/mL”) or was added at concentrations of 2, 8, 32, or 128 ng/mL (data not shown for 2 and 32 ng/mL for graph clarity. The data suggest that TGF-β1 at moderate concentrations (in the range of 2-32 ng/mL) all show similar effects. No TGF-β1 (0 ng/mL) and high concentration TGF-β1 (128 ng/mL) are shown to illustrate low and high endpoints, with 8 ng/mL shown both as a mid-point and because it lies in between the 5-10 ng/mL concentrations used in subsequent experiments. GAL was not added or was added at a concentration of 5 μM (“+GAL”). UACC257 cells cultured alone (with no addition of TGF-β1 or GAL) were used as a control.

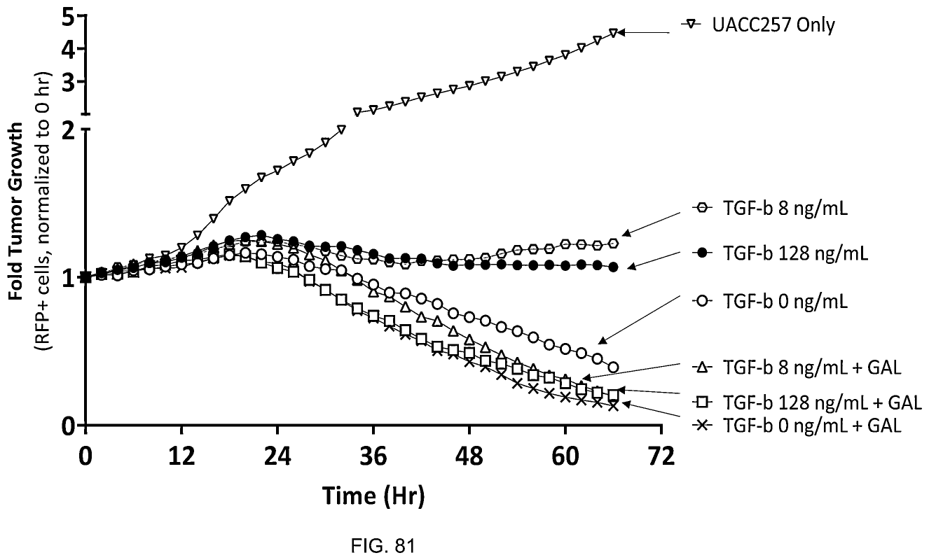

FIG. 81 shows tumor fold growth of UACC257-RFP cells, normalized to 0 hours. Shown are UACC257 cells only (“UACC257 Only”, open downward triangles) and UACC257 cells co-cultured with TCR-only transduced cells, where the co-cultures were treated with (i) 8 ng/mL TGF-β1 (“TGF-b 8 ng/mL”, open octagons), (ii) 128 ng/mL TGF-β1 (“TGF-b 128 ng/mL”, solid circles), (iii) no addition of TGF-β1 or GAL (“TGF-b 0 ng/mL”, open circles), (iv) 8 ng/mL TGF-β1 plus 5 μM GAL (“TGF-b 8 ng/mL+GAL”, open upward triangles), (v) 128 ng/mL TGF-β1 plus 5 μM GAL (“TGF-b 128 ng/mL+GAL”, open squares), or (vi) 5 μM GAL only (“TGF-b 0 ng/mL+GAL”, X's). Data are represented as mean of replicate wells from a single donor. These data show that addition of exogenous TGF-β1 to the co-culture reduced the anti-tumor cytotoxicity of the effector cells, as represented by a relative increase in fold tumor growth exhibited in the co-cultures where 8 ng/mL or 128 ng/mL TGF-β1 was added without the addition of GAL, as compared to the co-culture where neither TGF-β1 nor GAL were added. These data also show that blocking TGF-β1 signaling by addition of GAL rescues or enhances ability of effector cells to kill tumor cells, as shown in the co-cultures to which GAL was added. These data suggest that the anti-tumor toxicity of the effector cells in the tumor microenvironment may be reduced by the presence of TGF-β in the tumor microenvironment.

### Example 22

### TGF-β/Galunisertib Cell Trace Proliferation Assay

Transduced cells from one donor (prepared as in Example 17) were thawed, washed, and resuspended in TexMACS medium supplemented with 5% by volume human AB serum (“Complete TexMACS”). Benzonase nuclease was added to a concentration of 50 U/mL and the cells were incubated (rested) overnight in tissue culture-treated flasks at 37° C.

UACC257 tumor cells were harvested using 0.05% trypsin, washed, and counted. Red fluorescent protein (RFP)-labeled tumor cells were plated at 5,000 tumor cells per well in 100 μL in triplicate in a 96-well plate in Complete TexMACS. Plates were placed in an incubator at 37° C.

On the day of co-culture, effector T cells were counted, washed, and resuspended in PBS containing a CellTrace Violet proliferation dye at 1:1000 dilution (1 μL dye per mL PBS) and incubated for 20 minutes at 37° C. After labeling incubation, Complete TexMACS with 5% human AB serum was added in excess to bind remaining free dye and incubated for another 5 minutes at 37° C.

Labeled effector T cells were then washed, counted, and resuspended in Complete TexMACS and added in 1 mL per well to previously prepared tumor targets for a total of 2 mL per well at an effector:target (E:T) ratio of 5:1.

At the initiation of co-culture, TGF-β1 was not added (0 ng/mL) or was added at 2, 8, 32, or 128 ng/mL. TGF-β signaling inhibitor GAL was either not added (“−GAL”) or added at a concentration of 5 μM (“+GAL”). Cells were harvested after 3 days of co-culture. The cells were indeed stained immediately following harvest with a panel of surface antibodies against CD3, CD4, and CD8 (in addition to already being labeled with the CellTrace Violet dye) to determine any differential proliferative effects of the treatments on T cell subsets. Proliferation modeling and statistics were generated using the Proliferation Modeling feature of FlowJo.

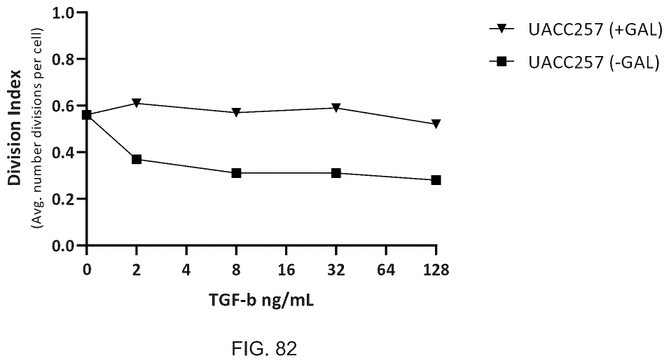

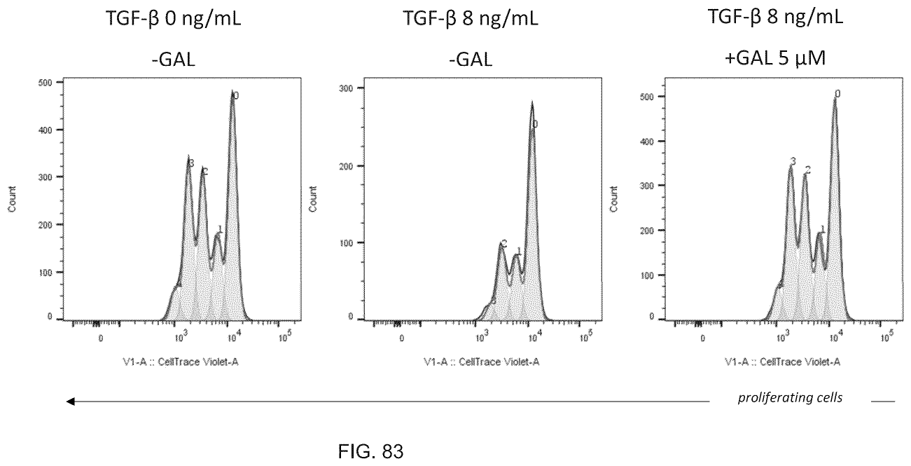

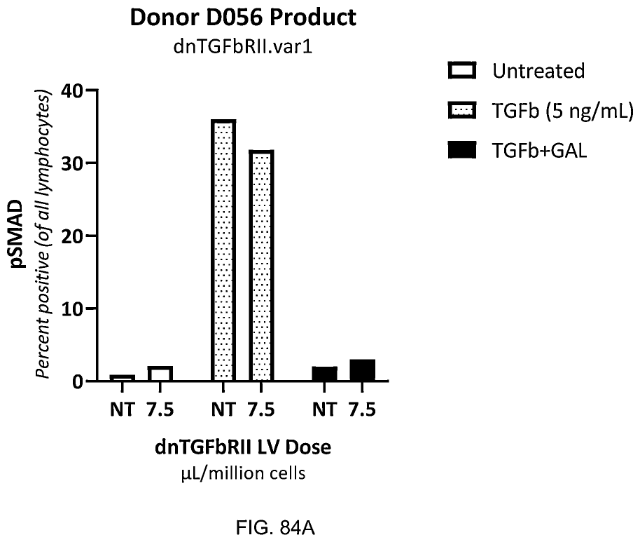

Proliferation plots were reported on CD3+ lymphocytes, and statistics reported as Division Index, which is the average number of divisions per cell within the specified gate, inclusive of cells that both did and did not divide. FIG. 82 shows the Division Index (average number of divisions per cell) of effector T cells (transduced with TCR only) co-cultured with UACC257 cells in the presence (“+GAL”) or absence (“−GAL”) of GAL a concentration of 5 μM, and the absence of TGF-β1 (0 ng/mL) or the presence of TGF-β1 at 2, 8, 32, or 128 ng/mL. Data are represented as mean of replicate wells from a single donor. FIG. 83 shows the proliferation of effector cells (transduced with TCR only) cocultured with UACC257 tumor cells with no addition of TGF-β1 or GAL (“TGF-β 0 ng/mL −GAL”), with the addition of 8 ng/mL TGF-β1 and no addition of GAL (“TGF-β 8 ng/mL −GAL”), and with the addition of 8 ng/mL TGF-β1 and 5 μM GAL (“TGF-β 8 ng/mL+GAL 5 μM”). Cell count (on the Y axis) ranges from 0 to 500 for the “TGF-β 0 ng/mL −GAL” plot, from 0 to 300 for the “TGF-β 8 ng/mL −GAL” plot, and from 0 to 500 for the “TGF-β 8 ng/mL+GAL 5 μM” plot. The brightest peak is labelled as “0,” or undivided. Each subsequent division/daughter generation is labeled 1, 2, 3, etc. and represents the number of divisions cells within that peak have undergone. Data are represented as mean of replicate wells from a single donor. These data show that addition of exogenous TGF-β1 to co-cultures suppresses proliferation of effector T cells responding to target-bearing UACC257 cells. Proliferation of effector T cells can be rescued by addition of the TGF-β signaling inhibitor, GAL.

### Example 23

### Phospho-SMAD Intracellular Staining Assay

SMAD is a signal transducer in the TGFβ signaling pathway. SMAD is phosphorylated in response to binding of TGFβ to its receptor and facilitates expression of TGFβ-responsive genes.

Non-transduced cells and cells expressing dnTGFβvar1 or dnTGFβvar2 (prepared as in Example 17) were thawed and washed in TexMACS medium supplemented with 5% by volume human AB serum (“Complete TexMACS”). They were subsequently resuspended in unsupplemented TexMACS to rest under serum starvation overnight at 37° C. After overnight rest, effector cells were counted and the concentration was adjusted as necessary and 100 μL aliquots were added into a flat-bottomed 96-well tissue culture plate. To avoid stimulating SMAD phosphorylation from the manipulation of cells during counting/plating, cells were further rested about 2 hours in serum-free TexMACS at 37° C.

After 2 hours, 50 μL of a 3× solution of TGF-β1 (15 ng/mL) and/or GAL (15 μM) was added to the plated effector cells for final 1× concentrations of 5 ng/mL TGF-β1 and/or 5 μM GAL, respectively. For untreated cells, 50 μL of untreated medium was added. Alternatively, tumor line supernatant+/−GAL was added to the effector cells to determine if tumor-secreted TGF-β was active in tumor cell culture. Data not shown, data was inconclusive. Treated cells were then incubated for 1-2 hours at 37° C.

Following cytokine treatment, cells were prepared for intracellular staining with reagents from Becton Dickinson, following recommended protocols. Briefly, cells were fixed with the addition of an equal volume (150 μL) of pre-warmed 1×BD Lyse/Fix Buffer for 10-15 minutes at 37° C. Following fixation, cells were centrifuged and the supernatant was decanted from the plate Immediately, cells were resuspended in 200 μL of pre-chilled PhosFlow Perm Buffer III and incubated for 30 minutes at 4° C.

Following permeabilization, cells were washed twice with Flow Buffer (PBS, 1% FBS, 0.1% sodium azide). Cells were then resuspended in 50 μL of Flow Buffer containing labeled antibodies for pSMAD2/3 or isotype and appropriate surface antigens and incubated for 30 minutes at 20° C./room temperature. Following staining, cells were washed with Flow Buffer and resuspended in Fixation/Running Buffer (PBS, 1% FBS, 1% paraformaldehyde) and acquired on a cytometer. Cytometry data files were analyzed in FlowJo. pSMAD+ percentages were reported as a proportion of all live lymphocytes.

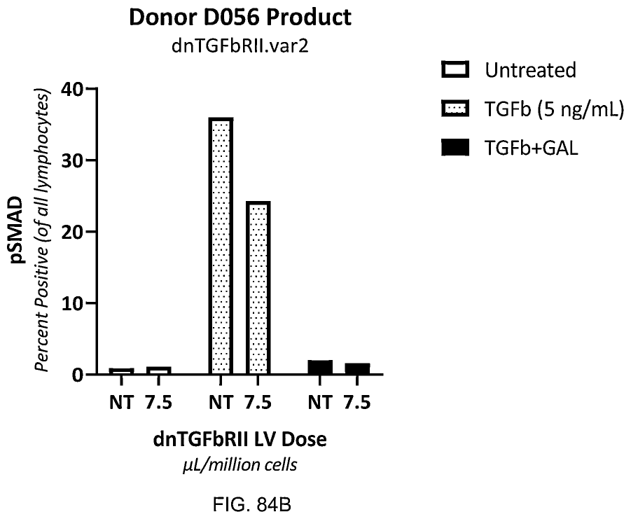

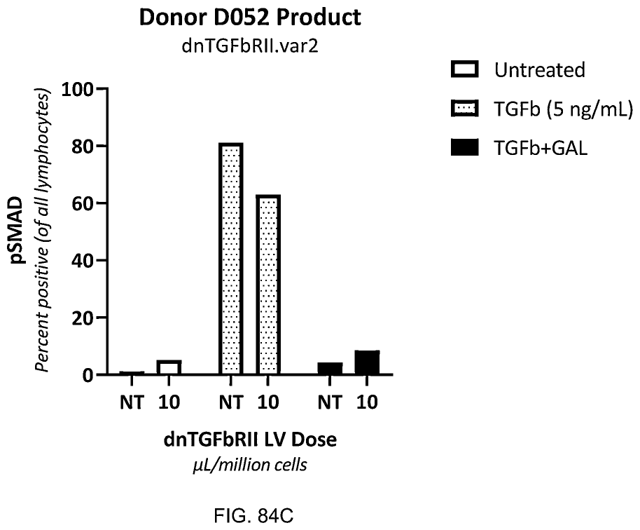

FIG. 84A shows the percentage of all lymphocytes that were positive for pSMAD (Y axis). Lymphocytes are from a first donor. Results are shown for non-transduced cells (“NT”) and for cells transduced with vector encoding dnTGFβRIIvar1 at 7.5 μL per million cells (“7.5”). Cells were untreated (white bars), treated with 5 ng/mL TGFβ (ticked bars), or treated with treated with 5 ng/mL TGFβ and 5 μM GAL (black bars). Data are represented as mean of replicate wells from a single donor.

FIG. 84B shows the percentage of all lymphocytes that were positive for pSMAD (Y axis). Lymphocytes are from the first donor. Results are shown for non-transduced cells (“NT”) and for cells transduced with vector encoding dnTGFβRIIvar2 at 7.5 μL per million cells (“7.5”). Cells were untreated (white bars), treated with 5 ng/mL TGFβ (ticked bars), or treated with treated with 5 ng/mL TGFβ and 5 μM GAL (black bars). Data are represented as mean of replicate wells from a single donor.

FIG. 84C shows the percentage of all lymphocytes that were positive for pSMAD (Y axis). Lymphocytes are from a second donor. Results are shown for non-transduced cells (“NT”) and for cells transduced with vector encoding dnTGFβRIIvar2 at 10 μL per million cells (“10”). Cells were untreated (white bars), treated with 5 ng/mL TGFβ (ticked bars), or treated with treated with 5 ng/mL TGFβ and 5 μM GAL (black bars). Data are represented as mean of replicate wells from a single donor.

The data in FIGS. 84A-84C show that cells transduced with dnTGFβRIIvar1 or dnTGFβRIIvar2 show reduced phosphorylated SMAD when exposed to TGF-β, as compared to non-transduced cells.

### Example 24

### Tumor Death Assays

Cells from one donor (D120) prepared and sorted as in Example 19 were co-cultured with UACC257-RFP tumor cells, as described in Example 20, at an effector:target (E:T) ratio of 4:1 or 1:1 (data not shown; data showed the same differential trend when comparing TCR+dnTGFβRII+ to TCR+ only cells. Overall cytotoxicity/tumor control was reduced as compared to the 4:1 E:T assay, as is expected when fewer effector cells are added to the culture).

Transduced cells (effector) and tumor cells (target) were co-cultured in 96 well tissue culture plates. Starting number of tumor cells per well was 10,000 cells.

TGF-β1 at a concentration of 10 ng/mL was added to the co-cultures added either (i) at co-culture initiation and concurrently with tumor cell challenges, denoted as “TGFb” or (ii) at co-culture initiation and daily, denoted as “TGFb Daily”.

Effector/target co-culture plates were placed into an IncuCyte imager at 37° C. and 5% CO₂ and imaged every 2 hours for the duration of 3 to 12 days. Data was exported from the IncuCyte ZOOM software into Microsoft Excel and GraphPad Prism for further analysis. Fold tumor growth (RFP+ cell count) was normalized to 0 hr timepoint.

Co-culture plates were removed at day 3 or 4 following the initiation of co-culture, and 50% of the supernatant was removed using a micropipettor. Complete TexMACS medium containing the same number of tumor target cells (10,000 cells) as at assay initiation were added to bring each well to full volume. Addition of tumor cells may be referred to as a “challenge”, a “stimulation”, or an “add-back”. Cells were placed back in the IncuCyte until the next timepoint (3 to 4 days after tumor addition).

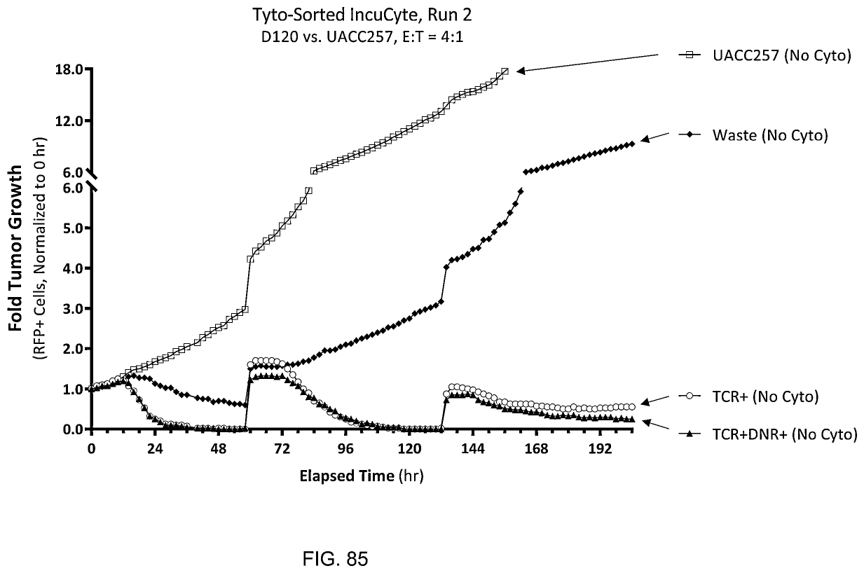

FIG. 85 shows fold growth of UACC257-RFP tumor cells normalized to 0 hours. Tumor cells were added back to the co-cultures at approximately 60 hours and at approximately 134 hours after the initiation of co-culture. Growth of UACC257-RFP cells cultured alone (“UACC257 (No Cyto)”) is shown as a control (open squares). Growth of UACC257-RFP cells co-cultured with cells depleted of TCR+dnTGFβRII+ cells and TCR+dnTGFβRII− cells (“Waste (No Cyto)”) is shown (solid diamonds). Growth of UACC257-RFP cells co-cultured with TCR+ cells depleted of TCR+dnTGFβRII+ cells (“TCR+(No Cyto)”) is shown (open circles). Growth of UACC257-RFP cells co-cultured with TCR+dnTGFβRII+ cells (“TCR+DNR+(No Cyto)”) is shown (solid triangles). Effector:Target (E:T) ratio was 4:1. No TGF-β1 was added to the cell cultures. Data are represented as mean of replicate wells from a single donor. These data show that each of TCR+ cells and TCR+dnTGFβRII+ cells are able to mediate killing of tumor cells over three tumor challenges.

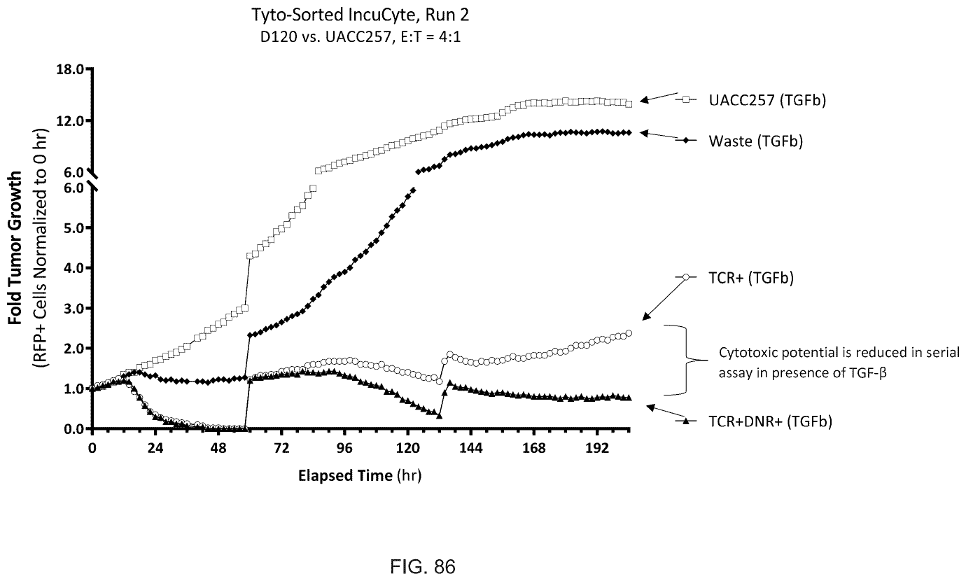

FIG. 86 shows fold growth of UACC257-RFP tumor cells normalized to 0 hours. Tumor cells were added back to the co-cultures at approximately 60 hours and at approximately 134 hours after the initiation of co-culture. Growth of UACC257-RFP cells cultured alone (“UACC257 (TGFb)”) is shown as a control (open squares). Growth of UACC257-RFP cells co-cultured with cells depleted of TCR+dnTGFβRII+ cells and TCR+dnTGFβRII− cells (“Waste (TGFb)”) is shown (solid diamonds). Growth of UACC257-RFP cells co-cultured with TCR+ cells depleted of TCR+dnTGFβRII+ cells (“TCR+(TGFb)”) is shown (open circles). Growth of UACC257-RFP cells co-cultured with TCR+dnTGFβRII+ cells (“TCR+DNR+(TGFb)”) is shown (solid triangles). Effector:Target (E:T) ratio was 4:1. TGF-β1 at a concentration of 10 ng/mL was added to each of cell cultures at the initiation of culture and at the times that tumor cells were added back. Data are represented as mean of replicate wells from a single donor. These data show that ability of effector cells to kill target cells was compromised by the addition of exogenous TGF-β1, but that TCR+dnTGFβRII+ cells maintained their killing ability better than did TCR+ cells. Thus, the data show that reduction of effector cell killing ability by addition of exogenous TGF-β1 can be rescued by expression of dnTGFβRII.

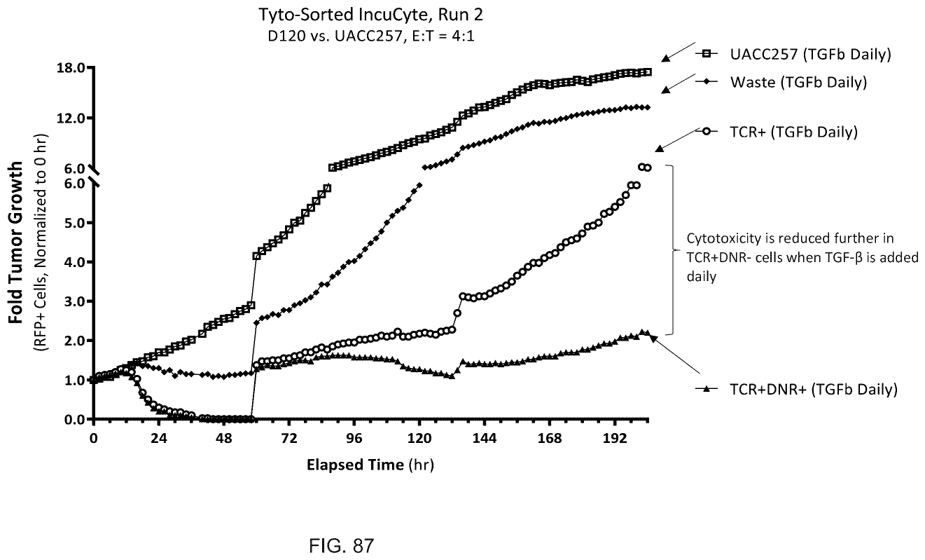

FIG. 87 shows fold growth of UACC257-RFP tumor cells normalized to 0 hours. Tumor cells were added back to the co-cultures at approximately 60 hours and at approximately 134 hours after the initiation of co-culture. Growth of UACC257-RFP cells cultured alone (“UACC257 (TGFb Daily)”) is shown as a control (open squares). Growth of UACC257-RFP cells co-cultured with cells depleted of TCR+dnTGFβRII+ cells and TCR+dnTGFβRII− cells (“Waste (TGFb Daily)”) is shown (solid diamonds). Growth of UACC257-RFP cells co-cultured with TCR+ cells depleted of TCR+dnTGFβRII+ cells (“TCR+(TGFb Daily)”) is shown (open circles). Growth of UACC257-RFP cells co-cultured with TCR+dnTGFβRII+ cells (“TCR+DNR+(TGFb Daily)”) is shown (solid triangles). Effector:Target (E:T) ratio was 4:1. TGF-β1 at a concentration of 10 ng/mL was added to each of cell cultures daily, including on the day of co-culture initiation. Data are represented as mean of replicate wells from a single donor. These data show that ability of effector cells to kill target cells was compromised by the addition of exogenous TGF-β1, but that TCR+dnTGFβRII+ cells maintained their killing ability better than did TCR+ cells. Thus, the data show that reduction of effector cell killing ability by addition of exogenous TGF-β1 can be rescued by expression of dnTGFβRII.

### Example 25

### TGF-β Cell Trace Proliferation Assay

UACC257 tumor cells were harvested using 0.05% trypsin, washed, and counted. Red fluorescent protein (RFP)-labeled tumor cells were plated at 30,000 cells per well in a flat-bottomed 24-well plate in 1 mL of Complete TexMACS. Plates were placed in an incubator at 37° C.

On the day of co-culture, cells from two donors prepared and sorted as in Example 19 were counted, washed, and resuspended in PBS containing a CellTrace Violet proliferation dye at 1:1000 dilution (1 μL dye per mL PBS) and incubated for 20 minutes at 37° C. After labeling incubation, Complete TexMACS with 5% human AB serum was added in excess to bind remaining free dye and incubated for another 5 minutes at 37° C.

Labeled effector T cells were then washed, counted, and resuspended in Complete TexMACS and 120,000 per well were added in 1 mL per well to previously prepared tumor targets for a total of 2 mL per well at an effector:target (E:T) ratio of 4:1.

TGF-β1 was added once to some co-cultures at 10 ng/mL upon the initiation of co-culture.

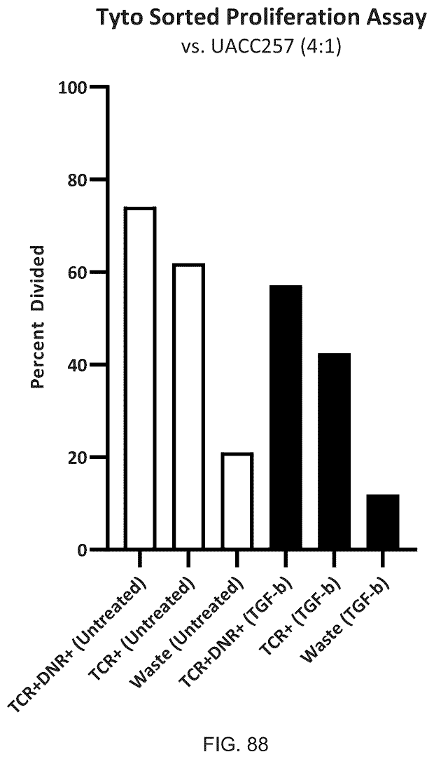

Cells were harvested after 3 days of co-culture. Proliferation modeling and statistics were generated using the Proliferation Modeling feature of FlowJo. Data are reported as “Percent Divided” (the percentage of the number of cells that had undergone at least one division) as a proportion of live CD3+CD8+ lymphocytes. FIG. 88 shows percentage of cells divided. Shown are (i) TCR+dnTGFβRII+ cells untreated with TGF-β1 (“TCR+DNR+(Untreated)”), (ii) TCR+ cells depleted of TCR+dnTGFβRII+ cells, untreated with TGF-β1 (“TCR+(Untreated)”), (iii) TCR-dnTGFβRII− cells untreated with TGF-β1 (“Waste (Untreated)”), (iv) TCR+dnTGFβRII+ cells treated with 10 ng/mL TGF-β1 (“TCR+DNR+(TGF-b)”), (v) TCR+ cells depleted of TCR+dnTGFβRII+ cells and treated with 10 ng/mL TGF-β1 (“TCR+(TGF-b)”), and (vi) TCR-dnTGFβRII− cells treated with 10 ng/mL TGF-β1 (“Waste (TGF-b)”). An average of cells from two donors is shown. Data are represented as mean. These data show that a larger proportion of TCR+dnTGFβRII+ cells divided in response to co-culture with tumor cells, as compared to TCR+ cells or TCR-dnTGFβRII− cells. This effect was seen both with and without addition of exogenous TGF-β1, suggesting that the cells may be affected by TGF-β1 autocrine signaling.

### Example 26

### Fold Expansion and Construct Expression

Products were manufactured as previously described with some minor updates:

CD8+ and CD4+ selected T cells (rather than isolated whole PBMC from leukapheresis product) were used as starting material.

Briefly, isolated PBMC from leukapheresis product are selected for CD8+ and CD4+ cells using immunomagnetic beads in the CliniMACS (Miltenyi Biotec) according to manufacturer's instructions. The resulting selected CD8+ and CD4+ cells are cryopreserved and stored in vapor phase liquid nitrogen until they are needed for activation.

During post-thaw rest, activation, and transduction, cells were suspended in TexMACS medium containing 2% by volume Physiologix XF SR (Nucleus Biologics). This xeno-free serum replacement is meant to eliminate serum components which might interfere with transduction.

For the Day +2 feed and the remainder of the expansion, TexMACS plus 5% by volume heat-inactivated human AB serum is used.

The transduction master mix includes added deoxynucleotides (50 μM) to increase lentiviral activity once inside the target cells.

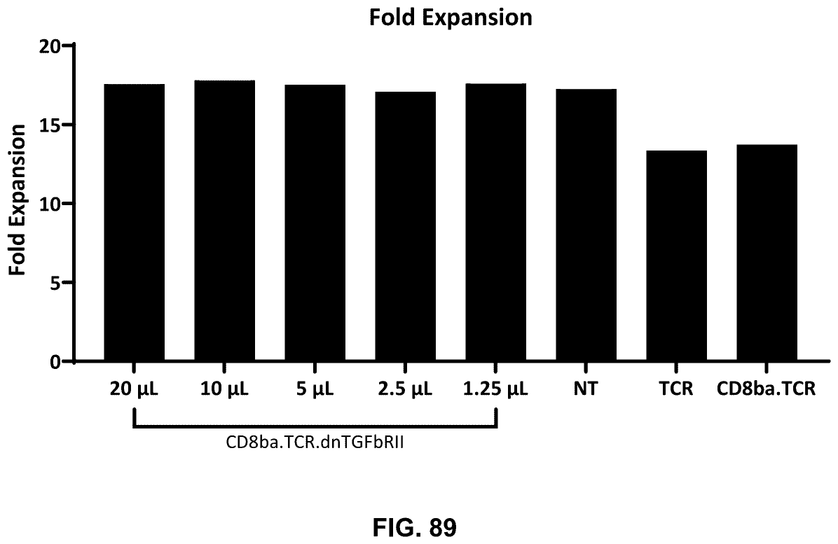

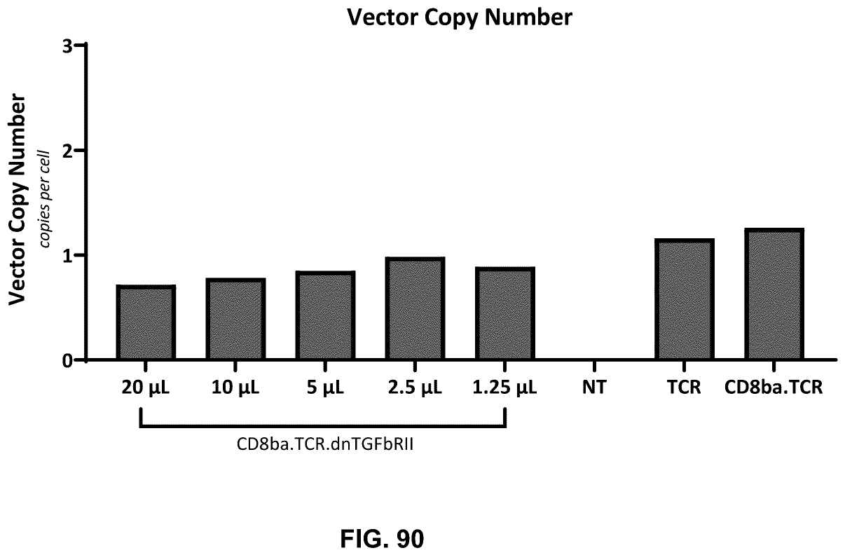

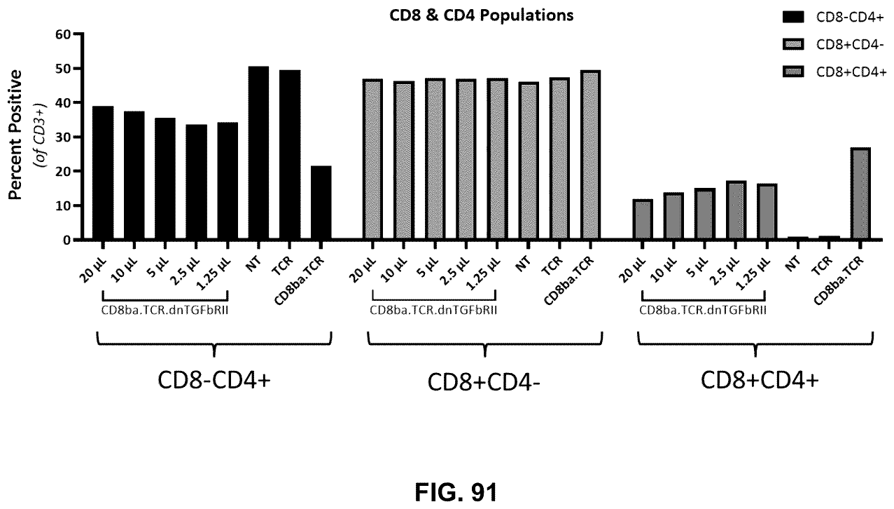

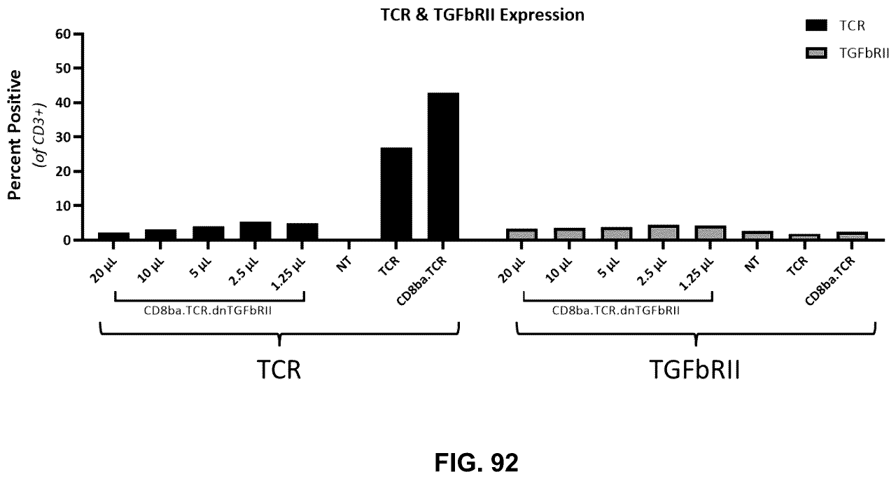

The volume of lentivirus constructs encoding CD8βα.TCR.dnTGFβRII (CD8ba.TCR.dnTGFbRII) used for transduction was titrated from 20, 10, 5, 2.5, and 1.25 μL per 1×10⁶ activated cells. TCR-only (TCR) and CD8βα.TCR (CD8ba.TCR) transduced cells were used as controls, as well as non-transduced cells (NT). Products were evaluated by flow cytometry for expression of CD3, CD8, CD4, TGFβRII, and TCR as previously described. Results are shown in FIGS. 89, 91 and 92. CD8βα.TCR.dnTGFβRII constructs conferred CD8 expression on CD4+ T cells.

### Vector Copy Number

The number of integrated vector copies was determined by qPCR. DNA was extracted from pellets of product cells manufactured as described in Example 26 using the QIAprep Spin Miniprep DNA purification kit (Qiagen), following the manufacturer's instructions. qPCR was performed using TaqMan Fast Advanced master mix and TaqMan viral psi-FAM and albumin-VIC primers/probes (Life Technologies) and analyzed on a QuantStudio 6 Pro qPCR machine (Applied Biosystems). VCN was determined by the formula:

$${VCN}_{sample} = 2 \times \frac{Mean psi value}{Mean albumin value}$$

Results are shown in FIG. 89. CD8βα.TCR.dnTGFβRII constructs did not affect fold expansion.

### Mid-Scale Manufacturing

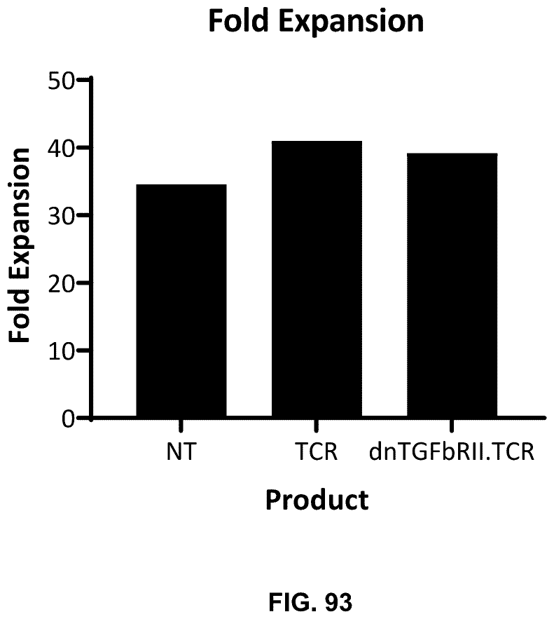

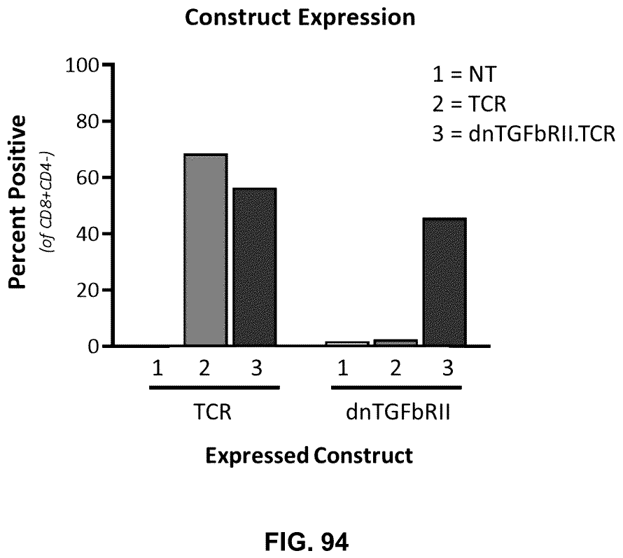

A mid-scale scaleup was performed as described in Example 19. Results are shown in FIGS. 93 and 94.

### Example 27

### Phospho-SMAD Intracellular Staining

Cells generated using the manufacturing method as described in Example 26 were thawed and washed in Hanks Balanced Salt Solution (HBSS) supplemented with 10% by volume human AB serum. They were subsequently resuspended in unsupplemented TexMACS and approximately 100 μL per sample were added to 5 mL round bottomed FACS tubes to rest under serum starvation overnight at 37° C.

After overnight rest, 50 μL of concentrated treatment solution (either 3×TGF-β1 or vehicle) was added to stimulate the cells for a final concentration of 10 ng/mL TGF-β1 (or 0 ng/mL for vehicle). Cells were allowed incubated under treatment at 37° C. for 1-2 hours.

Following cytokine treatment, cells were prepared for intracellular staining with reagents from Becton Dickinson, following recommended protocols. Briefly, cells fixed with the addition of 3 mL of pre-warmed 1×BD Lyse/Fix buffer for 10-15 minutes at 37° C.

Following fixation, cells were centrifuged and the supernatant was decanted from the tubes; then, they were washed once with 3 mL Flow Buffer (PBS without calcium or magnesium, 1% fetal bovine serum, and 0.1% sodium azide).

Following wash, the flow buffer was decanted and 1 mL of pre-chilled (−80° C.) PhosFlow Perm Buffer III was slowly added to each tube while gently vortexing. Cells were then permeabilized for 30 minutes on ice.

Following permeabilization, cells were washed twice with Flow Buffer. Cells were then resuspended in 50 μL of Flow Buffer containing labeled antibodies for pSMAD2/3 or isotype and appropriate surface antigens and incubated for 30 minutes at 20° C./room temperature.

Following staining, cells were washed with Flow Buffer and resuspended in Fixation/Running Buffer (PBS, 1% FBS, and 1% paraformaldehyde) and acquired on a cytometer.

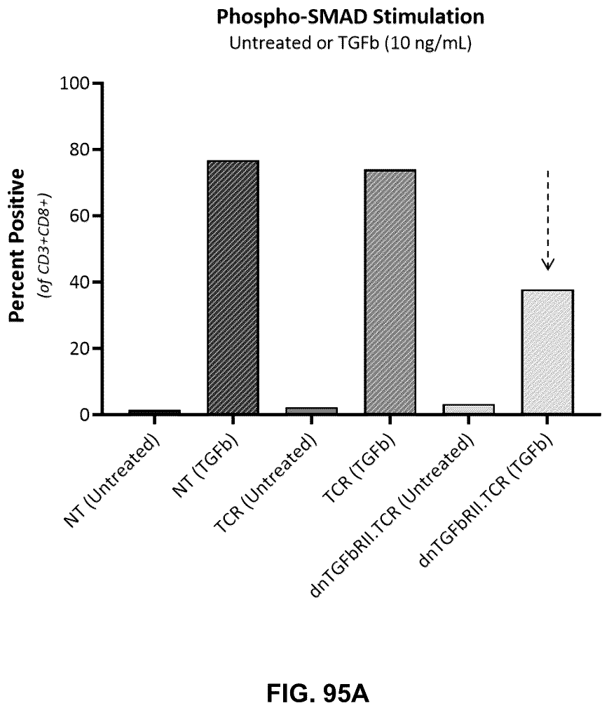

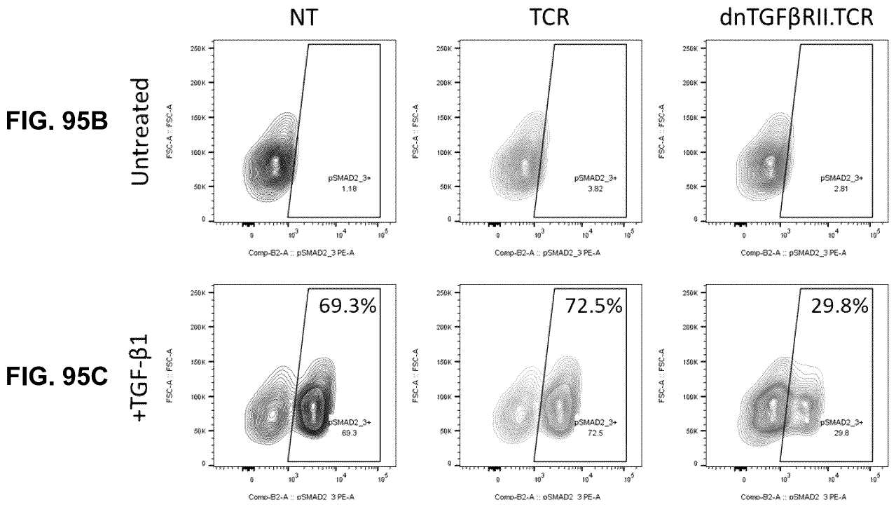

Cytometry data files were analyzed in FlowJo. Results are shown in FIG. 95A-C. Cells containing dnTGFβRII.TCR resisted signaling through TGF-β receptor complex, resulting in lower levels of phosphorylated SMAD after exposure to TGF-β1.

### Example 28

### Co-Culture Stimulation Proliferation Assays

Effector T cell product was thawed and rested overnight as previously described.

UACC257 cells were trypsinized, washed, and counted. 45,000 tumor cells were plated in 1 mL Complete TexMACS per well in a 24-well tissue culture plate and allowed to adhere for approximately 4 hours at 37° C.

A portion of rested T cells were labeled with CellTrace Violet as previously described and added in 1 mL per well to wells already containing tumor cells for a final E:T ratio of 4:1 (normalized to TCR+%). Unlabeled duplicates were added to remaining wells at the same ratio.

After 3 days (Tumor Challenge #1), CellTrace-labeled wells were harvested evaluated for proliferation metrics as previously described. The remaining wells were re-seeded with 45,000 tumor cells (Tumor Challenge #2) and returned to the incubator.

On D+6, remaining replicates were harvested and labeled with CellTrace Violet before being returned to their respective wells and re-seeded with an additional 45,000 tumor cells (Tumor Challenge #3). 3 days later (Day +9), Tumor Challenge #3 cells were harvested and evaluated for proliferation metrics.

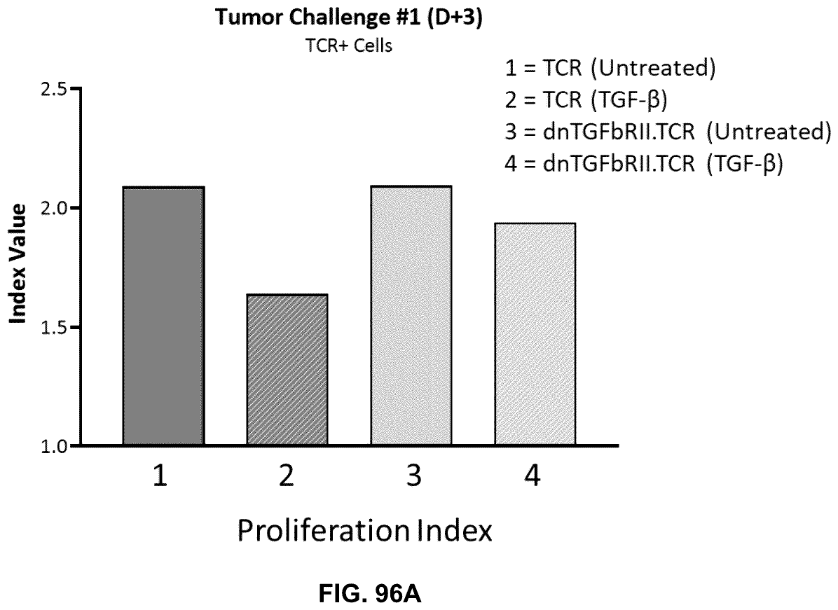

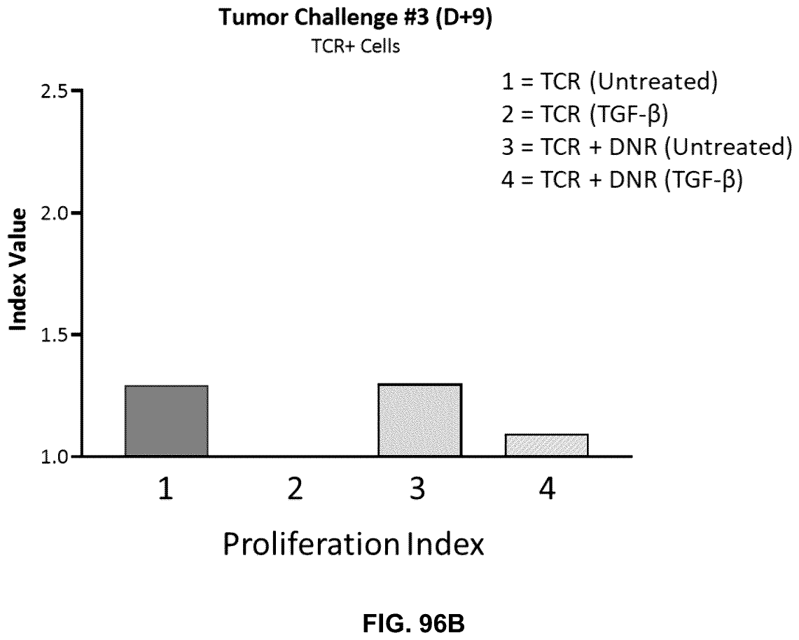

The above was duplicated with TGF-β (10 ng/mL) added concurrent with each tumor addition. Thus, the differences in proliferation metrics could be compared between the first and third tumor additions and between untreated and TGF-β-treated conditions at both time points. Results are shown in FIG. 96A-B. dnTGFβRII.TCR cells were resistant to inhibition by TGF-β during first challenge and survive 3 tumor challenges in the presence of TGF-β.

### Example 29

### Serial IncuCyte Cytotoxicity Assay

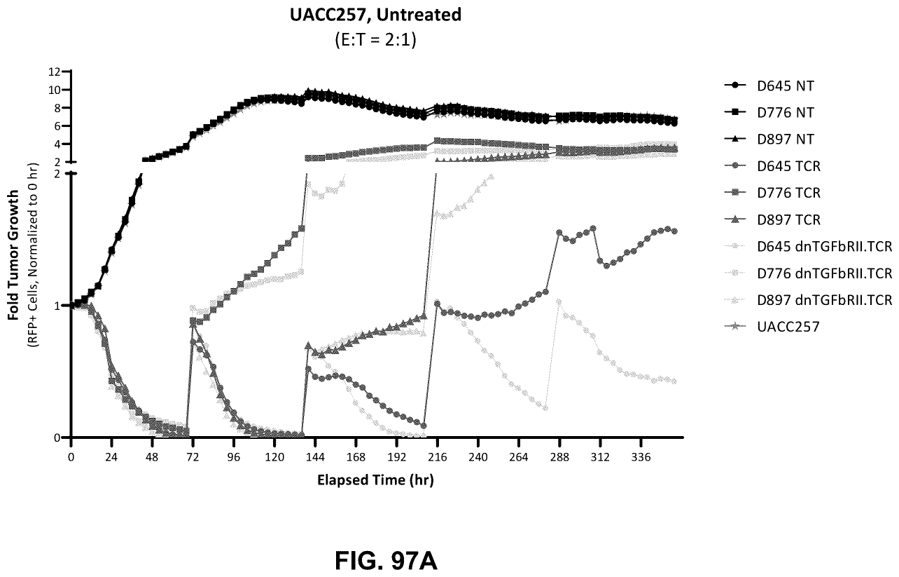

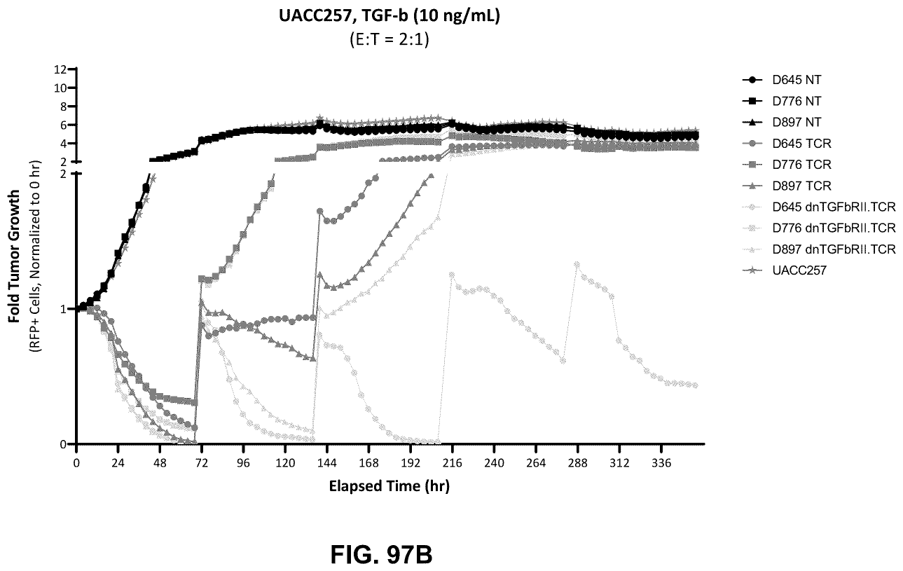

IncuCyte assays were performed as previously described at 2:1 E:T ratio with UACC257 tumor cells. Effector numbers were normalized to TCR+ percentage. Duplicate assays were performed in which cocultures were either untreated (vehicle) or treated with 10 ng/mL TGF-β1, which was added concurrently with each tumor addition. Results are shown in FIG. 97A-B.

### Example 30

### Cytokine Secretion

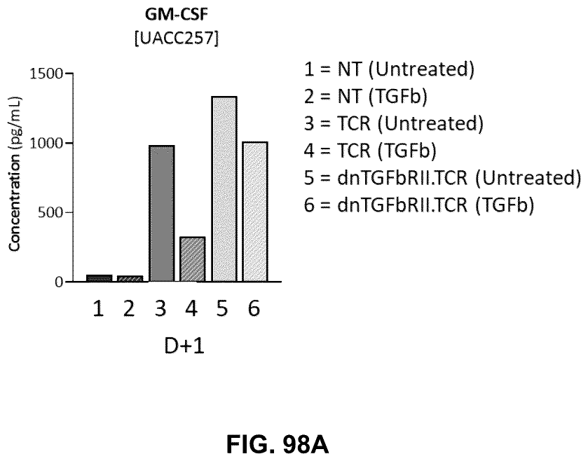

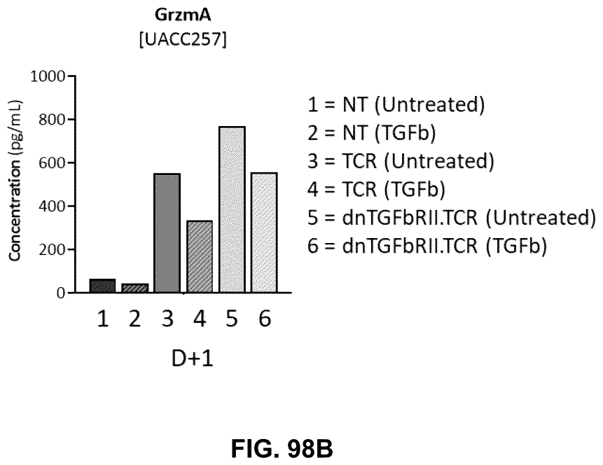

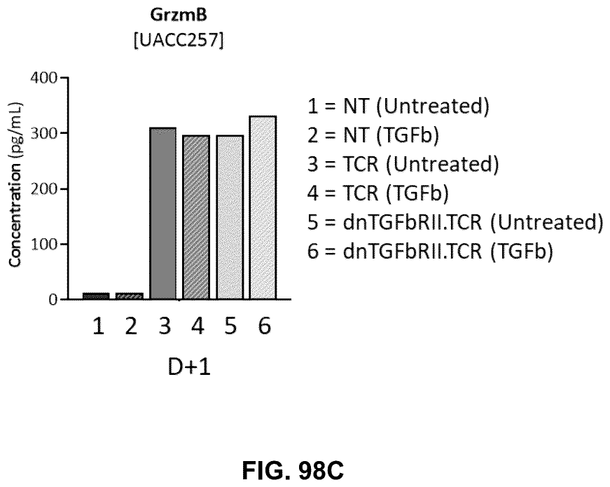

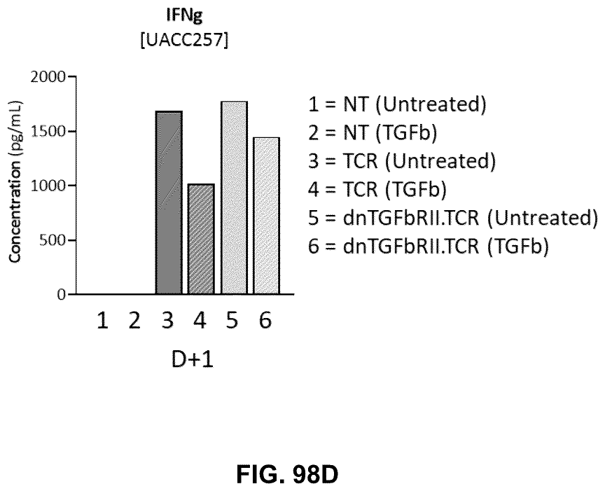

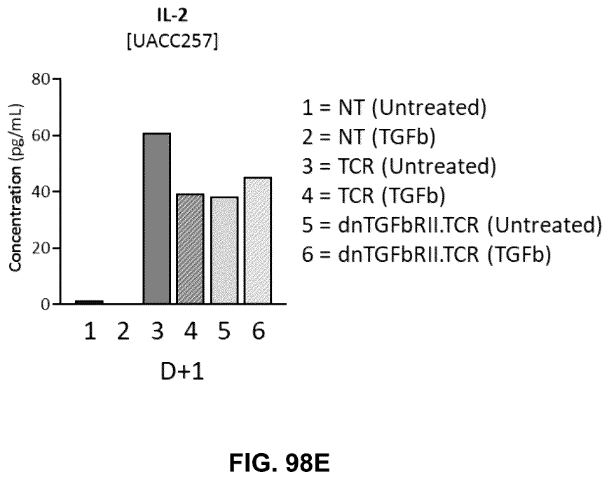

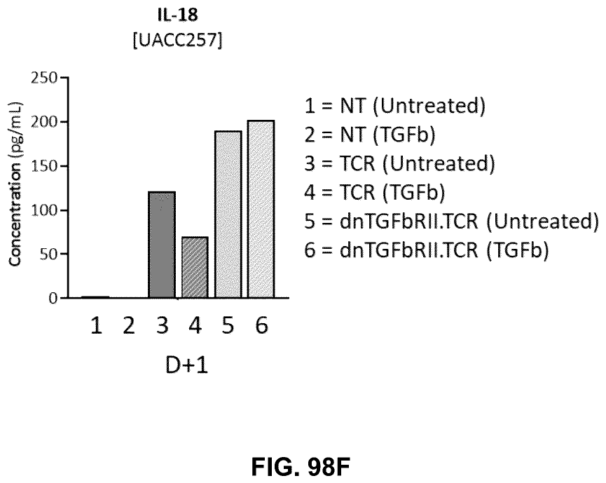

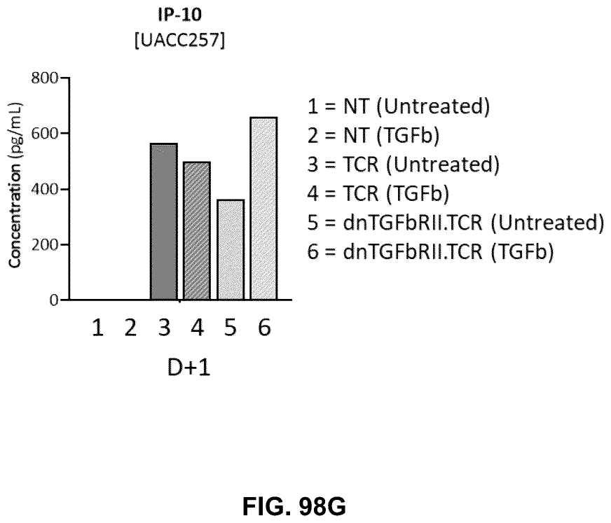

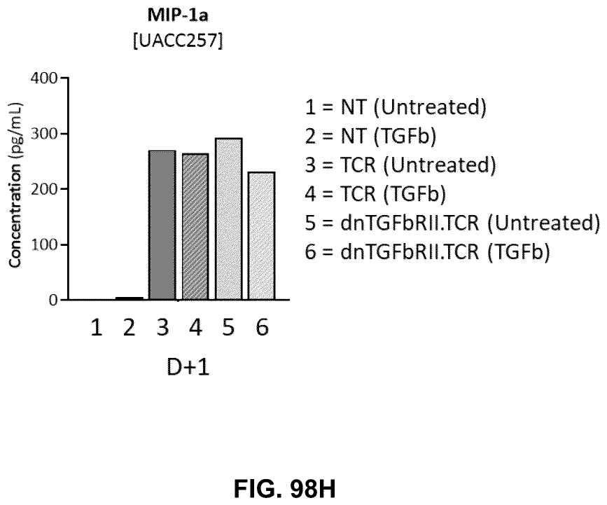

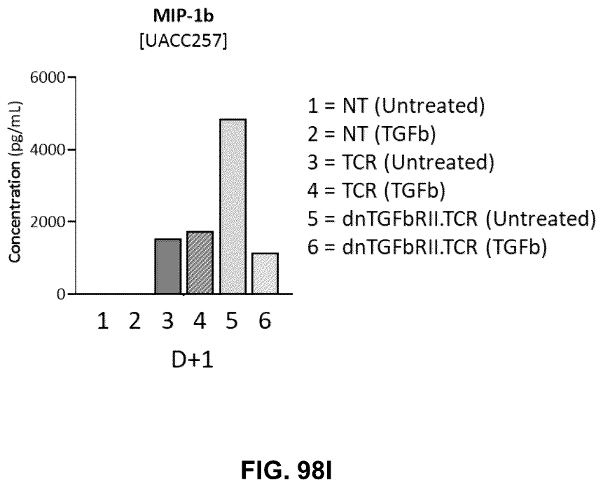

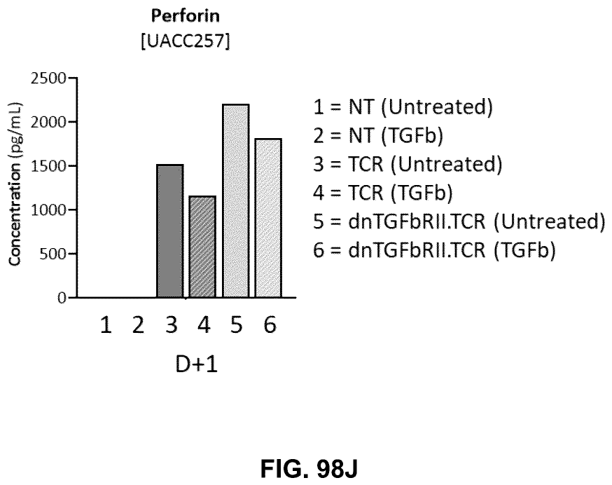

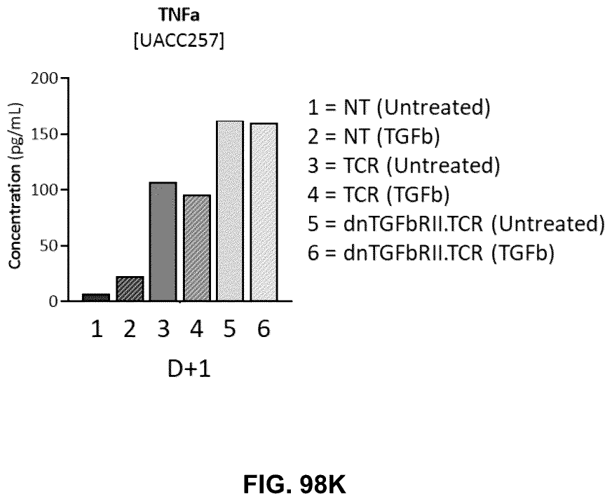

Cytokine concentrations in supernatants harvested from IncuCyte Cytotoxicity Assays (Example 29) were assessed using the Luminex® platform (ThermoFisher Scientific) according to the manufacturer's instructions. Results are shown in FIG. 98A-K. dnTGFβRII.TCR cells showed comparable or improved cytokine secretion.

The invention may be characterized by the following aspects:
1. A nucleic acid encoding a polypeptide comprising (i) SEQ ID NO: 305 or a sequence at least about 95%, at least about 96%, at least about 97%, at least about 98%, at least about 99%, or about 100% identical to SEQ ID NO: 305; (ii) SEQ ID NO: 307 or a sequence at least about 95%, at least about 96%, at least about 97%, at least about 98%, at least about 99%, or about 100% identical to SEQ ID NO: 307; or (iii) both (i) and (ii).
2. A nucleic acid comprising (i) SEQ ID NO: 306 or a sequence at least about 80%, at least about 85%, at least about 90%, at least about 95%, at least about 96%, at least about 97%, at least about 98%, at least about 99%, or about 100% identical to SEQ ID NO: 306; (ii) SEQ ID NO: 308 or a sequence at least about 80%, at least about 85%, at least about 90%, at least about 95%, at least about 96%, at least about 97%, at least about 98%, at least about 99%, or about 100% identical to SEQ ID NO: 308; or (iii) both (i) and (ii).
3. A nucleic acid comprising (i) SEQ ID NO: 312 or a sequence at least about 80%, at least about 85%, at least about 90%, at least about 95%, at least about 96%, at least about 97%, at least about 98%, at least about 99%, or about 100% identical to SEQ ID NO: 312; (ii) SEQ ID NO: 313 or a sequence at least about 80%, at least about 85%, at least about 90%, at least about 95%, at least about 96%, at least about 97%, at least about 98%, at least about 99%, or about 100% identical to SEQ ID NO: 313; or (iii) both (i) and (ii).
4. A vector comprising the nucleic acid of any one of aspects 1-3.
5. The vector of aspect 4, wherein the vector further comprises a post-transcriptional regulatory element (PRE) sequence selected from Woodchuck PRE (WPRE) (SEQ ID NO: 264), Woodchuck PRE (WPRE) mutant 1 (SEQ ID NO: 256), Woodchuck PRE (WPRE) mutant 2 (SEQ ID NO: 257), and hepatitis B virus (HBV) PRE (HPRE) (SEQ ID NO: 366).
6. The vector of aspect 5, wherein the post-transcriptional regulatory element (PRE) sequence is the Woodchuck PRE (WPRE) mutant 1 comprising the nucleic acid sequence of SEQ ID NO: 256.
7. The vector of aspect 5, wherein the post-transcriptional regulatory element (PRE) sequence is the Woodchuck PRE (WPRE) mutant 2 comprising the nucleic acid sequence of SEQ ID NO: 257.
8. The vector of any one of aspects 4-7, wherein the vector further comprises a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, or Murine Stem Cell Virus (MSCV) promoter.
9. The vector of aspect 8, wherein the promoter is a Murine Stem Cell Virus (MSCV) promoter.
10. The vector of any one of aspects 4-9, wherein the vector is a viral vector or a non-viral vector.
11. The vector of aspect 10, wherein the vector is a viral vector.
12. The vector of aspect 10 or aspect 11, wherein the viral vector is selected from adenoviruses, poxviruses, alphaviruses, arenaviruses, filoviruses, rhabdoviruses, retroviruses, lentiviruses, herpesviruses, paramyxoviruses, picornaviruses, and combinations thereof.
13. The vector of any one of aspects 10-12, wherein the viral vector is pseudotyped with an envelope protein of a virus selected from a native feline endogenous virus (RD114), a version of RD114 (RD114TR), gibbon ape leukemia virus (GALV), a version of GALV (GALV-TR), amphotropic murine leukemia virus (MLV 4070A), baculovirus (GP64), vesicular stomatitis virus (VSV-G), fowl plague virus (FPV), Ebola virus (EboV), baboon retroviral envelope glycoprotein (BaEV), and lymphocytic choriomeningitis virus (LCMV).
14. The vector of any one of aspects 4-13, wherein the vector is a lentiviral vector.
15. The vector of any one of aspects 4-14, wherein the vector further comprises a nucleic acid encoding a chimeric antigen receptor (CAR).
16. A T cell or natural killer (NK) cell (i) transduced with the nucleic acid of any one of aspects 1-3 or (ii) comprising the vector of any one of aspects 4-15.
17. The T cell or natural killer (NK) cell of aspect 16, wherein the cell is an αβ T cell, a γδ T cell, a natural killer T cell, a natural killer (NK) cell, or any combination thereof.
18. The T cell or natural killer (NK) cell of aspect 17, wherein the αβ T cell is a CD4+ T cell.
19. The T cell or natural killer (NK) cell of aspect 17, wherein the αβ T cell is a CD8+ T cell.
20. The T cell or natural killer (NK) cell of aspect 17, wherein the γδ T cell is a Vγ9Vδ2+ T cell.
21. The nucleic acid of any one of aspects 1-3 further comprising a nucleic acid sequence encoding at least one TCR polypeptide, at least one CD8 polypeptide, or at least one TCR polypeptide and at least one CD8 polypeptide.
22. A vector comprising the nucleic acid of aspect 21.
23. The vector of aspect 22, wherein the vector further comprises a post-transcriptional regulatory element (PRE) selected from a Woodchuck PRE (WPRE), Woodchuck PRE (WPRE) mutant 1, Woodchuck PRE (WPRE) mutant 2, or hepatitis B virus (HBV) PRE (HPRE) (SEQ ID NO: 366).
24. The vector of aspect 23, wherein the post-transcriptional regulatory element (PRE) is a Woodchuck PRE (WPRE) mutant 1 comprising the nucleotide sequence of SEQ ID NO: 256.
25. The vector of aspect 23, wherein the post-transcriptional regulatory element (PRE) is a Woodchuck PRE (WPRE) mutant 2 comprising the nucleotide sequence of SEQ ID NO: 257.
26. The vector of any one of aspects 22-25, wherein the vector further comprises a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, or Murine Stem Cell Virus (MSCV) promoter.
27. The vector of aspect 26, wherein the promoter is a Murine Stem Cell Virus (MSCV) promoter.
28. The vector of any one of aspects 22-27, wherein the vector is a viral vector or a non-viral vector.
29. The vector of aspect 28, wherein the vector is a viral vector.
30. The vector of aspect 28 or aspect 29, wherein the viral vector is selected from adenoviruses, poxviruses, alphaviruses, arenaviruses, flaviviruses, rhabdoviruses, retroviruses, lentiviruses, herpesviruses, paramyxoviruses, picornaviruses, and combinations thereof.
31. The vector of aspect 28 or 29, wherein the viral vector is pseudotyped with an envelope protein of a virus selected from the native feline endogenous virus (RD114), a version of RD114 (RD114TR), gibbon ape leukemia virus (GALV), a version of GALV (GALV-TR), amphotropic murine leukemia virus (MLV 4070A), baculovirus (GP64), vesicular stomatitis virus (VSV-G), fowl plague virus (FPV), Ebola virus (EboV), baboon retroviral envelope glycoprotein (BaEV), and lymphocytic choriomeningitis virus (LCMV).
32. The vector of any one of aspects 22-31, wherein the vector is a lentiviral vector.
33. The vector of any one of aspects 22-32, wherein the vector further comprises a nucleic acid encoding a chimeric antigen receptor (CAR).
34. A T cell and/or natural killer (NK) cell (i) transduced with the nucleic acid of aspect 21 or (ii) comprising the vector of any one of aspects 22-33.
35. The T cell and/or natural killer (NK) cell of aspect 34, wherein the T cell is an αβ T cell, a γδ T cell, and/or a natural killer T cell.
36. The T cell and/or natural killer (NK) cell of aspect 35, wherein the αβ T cell is a CD4+ T cell.
37. The T cell and/or natural killer (NK) cell of aspect 35, wherein the αβ T cell is a CD8+ T cell.
38. The T cell and/or natural killer (NK) cell of aspect 35, wherein the γδ T cell is a Vγ9Vδ2+ T cell.
39. A composition comprising the T cell and/or natural killer (NK) cell of any one of aspects 34-38.
40. The composition of aspect 39, wherein the composition is a pharmaceutical composition.
41. The composition of aspect 39 or aspect 40, wherein the composition further comprises an adjuvant, excipient, carrier, diluent, buffer, stabilizer, or a combination thereof.
42. The composition of aspect 41, wherein the adjuvant is an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof.
43. The composition of aspect 41 or aspect 42, wherein the adjuvant is IL-2, IL-7, IL-12, IL-15, IL-21, or any combination thereof.
44. A method of preparing T cells and/or natural killer cells for immunotherapy comprising:
isolating T cells and/or natural killer cells from a blood sample of a human subject,
activating the isolated T cells and/or natural killer cells,
transducing the activated T cells and/or natural killer cells with the nucleic acid of aspect 21 or the vector of any one of aspects 22-33, and
expanding the transduced T cells and/or natural killer cells.
   45. The method of aspect 44, further comprising isolating CD4+CD8+ T cells from the transduced T cells and/or natural killer cells and expanding the isolated CD4+CD8+ transduced T cells.
   46. The method of aspect 44 or aspect 45, wherein the blood sample comprises peripheral blood mononuclear cells (PMBC).
   47. The method of any one of aspects 44-46, wherein the activating comprises contacting the T cells and/or natural killer cells with an anti-CD3 and an anti-CD28 antibody.
   48. The method of any one of aspects 44-47, wherein the T cell is a CD4+ T cell.
   49. The method of any one of aspects 44-47, wherein the T cell is a CD8+ T cell.
   50. The method of aspect 44-49, wherein the T cell is a γδ T cell or an αβ T cell.
   51. The method of any one of aspects 44-50, wherein the activation and/or expanding are in the presence of a combination of IL-2 and IL-15 and optionally with zoledronate.
   52. A method of treating a patient who has cancer, comprising administering to the patient the composition of any one of aspects 39-43, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   53. A method of eliciting an immune response in a patient who has cancer, comprising administering to the patient the composition of any one of aspects 39-43, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   54. The method of aspect 52 or 53, wherein the T cell and/or natural killer (NK) cell kills cancer cells that present a peptide in a complex with an MHC molecule on a cell surface.
   55. A nucleic acid comprising:
(a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) a nucleic acid sequence encoding at least one dominant negative TGFβ Receptor II (dnTGFβRII) polypeptide,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, or 91 and 92;
wherein the CD8 α chain is SEQ ID NO: 7, 258, 259, 262, or a variant thereof;
wherein the CD8 β chain is SEQ ID NO: 8, 9, 10, 11, 12, 13, or 14; and
wherein at least one of the at least one dnTGFβRII polypeptide is selected from (i) SEQ ID NO: 305 or a sequence at least about 95%, at least about 96%, at least about 97%, at least about 98%, at least about 99%, or about 100% identical thereto or (ii) SEQ ID NO: 307 or a sequence at least about 95%, at least about 96%, at least about 97%, at least about 98%, at least about 99%, or about 100% identical thereto.
   56. A nucleic acid comprising:
(a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) a nucleic acid sequence encoding at least one dominant negative TGFβ Receptor II (dnTGFβRII) polypeptide,
wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303;
wherein the CD8 α chain is SEQ ID NO: 7, 258, 259, 262, or a variant thereof;
wherein the CD8 β chain is SEQ ID NO: 8, 9, 10, 11, 12, 13, or 14; and
wherein the at least one dnTGFβRII polypeptide is selected from (i) SEQ ID NO: 305 or a sequence at least about 95%, at least about 96%, at least about 97%, at least about 98%, at least about 99%, or about 100% identical thereto or (ii) SEQ ID NO: 307 or a sequence at least about 95%, at least about 96%, at least about 97%, at least about 98%, at least about 99%, or about 100% identical thereto.
   57. A nucleic acid comprising: (a) a nucleic acid sequence at least about 80% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide.
   58. A nucleic acid comprising: (a) a nucleic acid sequence at least about 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide.
   59. The nucleic acid of aspect 57 or aspect 58, wherein the nucleic acid sequence or sequences encoding at least one of the at least one dominant negative TGFβ Receptor II (dnTGFβRII) polypeptide is selected from (i) SEQ ID NO: 306 or a sequence at least about 90%, 91%, 92%, 93%, 94%, 95%, 96%, 97%, 98%, 99%, or 100% identical thereto or (ii) SEQ ID NO: 308 or a sequence at least about 90%, 91%, 92%, 93%, 94%, 95%, 96%, 97%, 98%, 99%, or 100% identical thereto.
   60. A vector comprising the nucleic acid of any one of aspects 54-58.
   61. A vector comprising N1, N2, N3, N4, N5, L1, L2, L3, and L4, in any order, wherein N1 comprises a nucleic acid sequence encoding a CD8β chain and is present or absent, N2 comprises a nucleic acid sequence encoding a CD8α chain, N3 comprises a nucleic acid sequence encoding a TCRβ chain, N4 comprises a nucleic acid sequence encoding a TCRα chain, and N5 comprises a nucleic acid sequence encoding at least one dominant negative TGFβ Receptor II (dnTGFβRII) polypeptide; and wherein L1-L4 each comprises a nucleic acid sequence encoding at least about one linker, wherein each of L1-L4 is independently the same or different, and wherein each of L1-L4 is independently present or absent.
   62. The vector of aspect 61, comprising Formula I or Formula II:

5′-N1-L1-N2-L2-N3-L3-N4-L4-N5-3′ [I]

5′-N5-L1-N1-L2-N2-L3-N3-L4-N4-3′ [II].

63. The vector of aspect 61 or aspect 62, wherein N1 comprises a nucleic acid sequence encoding SEQ ID NO: 8, 9, 10, 11, 12, 13, or 14.
64. The vector of any one of aspects 61-63, wherein N2 comprises a nucleic acid sequence encoding SEQ ID NO: 7, 258, 259, 262, or a variant thereof.
65. The vector of any one of aspects 61-64, wherein N4 and N3 comprise a nucleic acid sequence encoding SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92.
66. The vector of any one of aspects 61-65, wherein N5 comprises a nucleic acid sequence encoding (i) SEQ ID NO: 305 or a sequence at least about 95%, at least about 96%, at least about 97%, at least about 98%, at least about 99%, or about 100% identical to SEQ ID NO: 305 or (ii) SEQ ID NO: 307 or a sequence at least about 95%, at least about 96%, at least about 97%, at least about 98%, at least about 99%, or about 100% identical to SEQ ID NO: 307.
67. The vector of any one of aspects 61-66, wherein the vector further comprises (i) a nucleic acid encoding a 2A peptide or an internal ribosome entry site (IRES) positioned between N1 and L1, between L1 and N2, between N2 and L2, between L2 and N3, between N3 and L3, between L3 and N4, between N4 and L4, between L4 and N5, or any combination thereof or (ii) a nucleic acid encoding a 2A peptide or an internal ribosome entry site (IRES) positioned between N5 and L1, between L1 and N1, between N1 and L2, between L2 and N2, between N2 and L3, between L3 and N3, between N3 and L4, between L4 and N4, or any combination thereof.
68. The vector of aspect 67, wherein the 2A peptide is P2A (SEQ ID NO: 93), T2A (SEQ ID NO: 94), E2A (SEQ ID NO: 95), or F2A (SEQ ID NO: 96).
69. The vector of aspect 67, wherein the IRES is selected from the group consisting of IRES from picornavirus, IRES from flavivirus, IRES from pestivirus, IRES from retrovirus, IRES from lentivirus, IRES from insect RNA virus, and IRES from cellular mRNA.
70. The vector of any one of aspects 61-69, wherein the vector further comprises (i) a nucleic acid encoding a furin positioned between N1 and L1, between L1 and N2, between N2 and L2, between L2 and N3, between N3 and L3, between L3 and N4, between N4 and L4, between L4 and N5, or any combination thereof or (ii) a nucleic acid encoding a furin positioned between N5 and L1, between L1 and N1, between N1 and L2, between L2 and N2, between N2 and L3, between L3 and N3, between N3 and L4, between L4 and N4, or any combination thereof.
71. The vector of any one of aspects 60-70, wherein the vector further comprises a post-transcriptional regulatory element (PRE) selected from a Woodchuck PRE (WPRE), Woodchuck PRE (WPRE) mutant 1, Woodchuck PRE (WPRE) mutant 2, or hepatitis B virus (HBV) PRE (HPRE) (SEQ ID NO: 366).
72. The vector of aspect 71, wherein the post-transcriptional regulatory element (PRE) is a Woodchuck PRE (WPRE) mutant 1 comprising the nucleic acid sequence of SEQ ID NO: 256.
73. The vector of aspect 71, wherein the post-transcriptional regulatory element (PRE) is a Woodchuck PRE (WPRE) mutant 2 comprising the nucleic acid sequence of SEQ ID NO: 257.
74. The vector of any one of aspects 60-73, wherein the vector further comprises a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, or Murine Stem Cell Virus (MSCV) promoter.
75. The vector of aspect 74, wherein the promoter is a Murine Stem Cell Virus (MSCV) promoter.
76. The vector of any one of aspects 60-75, wherein the vector is a viral vector or a non-viral vector.
77. The vector of aspect 76, wherein the vector is a viral vector.
78. The vector of aspect 76 or aspect 77, wherein the viral vector is selected from adenoviruses, poxviruses, alphaviruses, arenaviruses, flaviviruses, rhabdoviruses, retroviruses, lentiviruses, herpesviruses, paramyxoviruses, picornaviruses, and combinations thereof.
79. The vector of aspect 77 or 78, wherein the viral vector is pseudotyped with an envelope protein of a virus selected from the native feline endogenous virus (RD114), a version of RD114 (RD114TR), gibbon ape leukemia virus (GALV), a version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), and lymphocytic choriomeningitis virus (LCMV).
80. The vector of any one of aspects 60-79, wherein the vector is a lentiviral vector.
81. The vector of any one of aspects 60-80, wherein the vector further comprises a nucleic acid encoding a chimeric antigen receptor (CAR).
82. A T cell and/or natural killer (NK) cell transduced with the nucleic acid of any one of aspects 55-59.
83. A T cell and/or natural killer (NK) cell transduced with the vector of any one of aspects 60-81.
84. A T cell and/or natural killer (NK) cell comprising:
(a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) at least one dominant negative TGFβ Receptor II (dnTGFβRII) polypeptide,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92;
wherein the CD8 α chain is SEQ ID NO: 7, 258, 259, 262, or a variant thereof;
wherein the CD8 β chain is SEQ ID NO: 8, 9, 10, 11, 12, 13, or 14; and
wherein at least one of the at least one dominant negative TGFβ Receptor II (dnTGFβRII) polypeptide is selected from (i) SEQ ID NO: 305 or a sequence at least about 95%, at least about 96%, at least about 97%, at least about 98%, at least about 99%, or about 100% identical to SEQ ID NO: 305 or (ii) SEQ ID NO: 307 or a sequence at least about 95%, at least about 96%, at least about 97%, at least about 98%, at least about 99%, or about 100% identical to SEQ ID NO: 307.
   85. A T cell and/or natural killer (NK) cell comprising:
(a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) at least one dominant negative TGFβ Receptor II (dnTGFβRII) polypeptide,
wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303;
wherein the CD8 α chain is SEQ ID NO: 7, 258, 259, 262, or a variant thereof; and
wherein, if present, the CD8 β chain is SEQ ID NO: 8, 9, 10, 11, 12, 13, or 14.
   86. The T cell and/or natural killer (NK) cell of aspect 85, wherein at least one of the at least one dominant negative TGFβ Receptor II (dnTGFβRII) polypeptide comprises SEQ ID NO: 305 or a sequence at least about 95%, at least about 96%, at least about 97%, at least about 98%, at least about 99%, or about 100% identical thereto.
   87. The T cell and/or natural killer (NK) cell of aspect 85, wherein at least one of the at least one dominant negative TGFβ Receptor II (dnTGFβRII) polypeptide comprises SEQ ID NO: 307 or a sequence at least about 95%, at least about 96%, at least about 97%, at least about 98%, at least about 99%, or about 100% identical thereto.
   88. The T cell and/or natural killer (NK) cell of any one of aspects 82-87, wherein the T cell is an αβ T cell, a γδ T cell, and/or a natural killer T cell.
   89. The T cell and/or natural killer (NK) cell of aspect 88, wherein the αβ T cell is a CD4+ T cell.
   90. The T cell and/or natural killer (NK) cell of aspect 88, wherein the αβ T cell is a CD8+ T cell.
   91. The T cell and/or natural killer (NK) cell of aspect 88, wherein the γδ T cell is a Vγ9Vδ2+ T cell.
   92. A composition comprising the T cell and/or natural killer (NK) cell of any one of aspects 82-91.
   93. The composition of aspect 92, wherein the composition is a pharmaceutical composition.
   94. The composition of aspect 92 or aspect 93, wherein the composition further comprises an adjuvant, excipient, carrier, diluent, buffer, stabilizer, or a combination thereof.
   95. The composition of aspect 94, wherein the adjuvant is an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof.
   96. The composition of aspect 94 or aspect 95, wherein the adjuvant is IL-2, IL-7, IL-12, IL-15, IL-21, or any combination thereof.
   97. A method of preparing T cells and/or natural killer cells for immunotherapy comprising:
isolating T cells and/or natural killer cells from a blood sample of a human subject,
activating the isolated T cells and/or natural killer cells,
transducing the activated T cells and/or natural killer cells with the nucleic acid of any one of aspects 55-59 or the vector of any one of aspects 60-81, and
expanding the transduced T cells and/or natural killer cells.
   98. The method of aspect 97, further comprising isolating CD4+CD8+ T cells from the transduced T cells and/or natural killer cells and expanding the isolated CD4+CD8+ transduced T cells.
   99. The method of aspect 97 or aspect 98, wherein the blood sample comprises peripheral blood mononuclear cells (PMBC).
   100. The method of any one of aspects 97-99, wherein the activating comprises contacting the T cells and/or natural killer cells with an anti-CD3 and an anti-CD28 antibody.
   101. The method of any one of aspects 97-100, wherein the T cell is a CD4+ T cell.
   102. The method of any one of aspects 97-100, wherein the T cell is a CD8+ T cell.
   103. The method of aspect 97-102, wherein the T cell is a γδ T cell or an αβ T cell.
   104. The method of any one of aspects 97-103, wherein the activation and/or expanding are in the presence of a combination of IL-2 and IL-15 and optionally with zoledronate.
   105. A method of treating a patient who has cancer, comprising administering to the patient the composition of any one of aspects 92-96, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   106. A method of eliciting an immune response in a patient who has cancer, comprising administering to the patient the composition of any one of aspects 92-96, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   107. The method of aspect 105 or 106, wherein the T cell and/or natural killer (NK) cell kills cancer cells that present a peptide in a complex with an MHC molecule on a cell surface.
   108. A nucleic acid comprising:
(a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) a nucleic acid sequence encoding at least one dominant negative TGFβ Receptor II (dnTGFβRII) polypeptide,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92;
wherein the CD8 α chain is SEQ ID NO: 7, 258, 259, 262, or a variant thereof;
wherein the CD8 β chain is SEQ ID NO: 8, 9, 10, 11, 12, 13, or 14; and
wherein at least one of the at least one dnTGFβRII polypeptide is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical to thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto.
   109. A nucleic acid comprising:
(a) a nucleic acid sequence encoding (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) a nucleic acid sequence encoding at least one dominant negative TGFβ Receptor II (dnTGFβRII) polypeptide,
wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303;
wherein the CD8 α chain is SEQ ID NO: 7, 258, 259, 262, or a variant thereof;
wherein the CD8 β chain is SEQ ID NO: 8, 9, 10, 11, 12, 13, or 14; and
wherein at least one of the at least one dnTGFβRII polypeptide is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical or (ii) SEQ ID NO: 313 or a sequence at least about 80%, at least about 85%, at least about 90%, at least about 95%, at least about 96%, at least about 97%, at least about 98%, at least about 99%, or about 100% identical thereto.
   110. A nucleic acid comprising: (a) a nucleic acid sequence at least about 80% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide.
   111. A nucleic acid comprising: (a) a nucleic acid sequence at least about 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301 and (b) a nucleic acid sequence or sequences encoding at least one dominant negative TGFβ Receptor II (dnTGFβRII) polypeptide.
   112. The nucleic acid of aspect 110 or aspect 111, wherein the nucleic acid sequence encoding at least one of the at least one dominant negative TGFβ Receptor II (dnTGFβRII) polypeptide also comprises an MSCV promoter and a WPRE sequence and is selected from (i) SEQ ID NO: 312 or a sequence at least about 80%, at least about 85%, at least about 90%, at least about 95%, at least about 96%, at least about 97%, at least about 98%, at least about 99%, or about 100% identical thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto.
   113. A vector comprising the nucleic acid of any one of aspects 108-112.
   114. The vector of aspect 113, wherein the vector is a viral vector or a non-viral vector.
   115. The vector of aspect 114, wherein the vector is a viral vector.
   116. The vector of aspect 113 or 114, wherein the viral vector is selected from adenoviruses, poxviruses, alphaviruses, arenaviruses, flaviviruses, rhabdoviruses, retroviruses, lentiviruses, herpesviruses, paramyxoviruses, picornaviruses, and combinations thereof.
   117. The vector of aspect 115 or 116, wherein the viral vector is pseudotyped with an envelope protein of a virus selected from the native feline endogenous virus (RD114), a version of RD114 (RD114TR), gibbon ape leukemia virus (GALV), a version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), and lymphocytic choriomeningitis virus (LCMV).
   118. The vector of any one of aspects 113-117, wherein the vector is a lentiviral vector.
   119. The vector of any one of aspects 113-118, wherein the vector further comprises a nucleic acid encoding a chimeric antigen receptor (CAR).
   120. A T cell and/or natural killer (NK) cell transduced with the nucleic acid of any one of aspects 108-112 or comprising the vector of any one of aspects 113-119.
   121. A T cell and/or natural killer (NK) cell comprising:
(a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) at least one dominant negative TGFβ Receptor II (dnTGFβRII) polypeptide,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92;
wherein the CD8 α chain is SEQ ID NO: 7, 258, 259, 262, or a variant thereof;
wherein the CD8 β chain is SEQ ID NO: 8, 9, 10, 11, 12, 13, or 14; and
is encoded by a nucleic acid sequence also comprising an MSCV promoter and a WPRE sequence and selected from (i) SEQ ID NO: 312 or a sequence at least about 80%, at least about 85%, at least about 90%, at least about 95%, at least about 96%, at least about 97%, at least about 98%, at least about 99%, or about 100% identical to thereto or (ii) SEQ ID NO: 313 or a sequence at least about 80%, at least about 85%, at least about 90%, at least about 95%, at least about 96%, at least about 97%, at least about 98%, at least about 99%, or about 100% identical thereto.
   122. A T cell and/or natural killer (NK) cell comprising:
(a) (i) a T-cell receptor (TCR) comprising an α chain and a β chain and a CD8 polypeptide comprising an α chain and a β chain, or (ii) a TCR comprising an α chain and a β chain and a CD8 polypeptide comprising an α chain without a β chain, and
(b) at least one dominant negative TGFβ Receptor II (dnTGFβRII) polypeptide,
wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303;
wherein the CD8 α chain is SEQ ID NO: 7, 258, 259, 262, or a variant thereof; and
wherein, if present, the CD8 β chain is SEQ ID NO: 8, 9, 10, 11, 12, 13, or 14.
   123. The T cell and/or natural killer (NK) cell of aspect 122, wherein at least one of the at least one dominant negative TGFβ Receptor II (dnTGFβRII) polypeptide is encoded by a nucleic acid sequence that also comprises an MSCV promoter and a WPRE sequence and that is selected from SEQ ID NO: 312 or a sequence at least about 80%, at least about 85%, at least about 90%, at least about 95%, at least about 96%, at least about 97%, at least about 98%, at least about 99%, or about 100% identical to thereto.
   124. The T cell and/or natural killer (NK) cell of aspect 122, wherein at least one of the at least one dominant negative TGFβ Receptor II (dnTGFβRII) polypeptide is encoded by a nucleic acid sequence that also comprises an MSCV promoter and a WPRE sequence and that is selected from SEQ ID NO: 313 or a sequence at least about 80%, at least about 85%, at least about 90%, at least about 95%, at least about 96%, at least about 97%, at least about 98%, at least about 99%, or about 100% identical to thereto.
   125. The T cell and/or natural killer (NK) cell of any one of aspects 120-124, wherein the T cell is an αβ T cell, a γδ T cell, and/or a natural killer T cell.
   126. The T cell and/or natural killer (NK) cell of aspect 125, wherein the αβ T cell is a CD4+ T cell.
   127. The T cell and/or natural killer (NK) cell of aspect 125, wherein the αβ T cell is a CD8+ T cell.
   128. The T cell and/or natural killer (NK) cell of aspect 125, wherein the γδ T cell is a Vγ9Vδ2+ T cell.
   129. A composition comprising the T cell and/or natural killer (NK) cell of any one of aspects 120-128.
   130. The composition of aspect 129, wherein the composition is a pharmaceutical composition.
   131. The composition of aspect 129 or aspect 130, wherein the composition further comprises an adjuvant, excipient, carrier, diluent, buffer, stabilizer, or a combination thereof.
   132. The composition of aspect 131, wherein the adjuvant is an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof.
   133. The composition of aspect 131 or aspect 132, wherein the adjuvant is IL-2, IL-7, IL-12, IL-15, IL-21, or any combination thereof.
   134. A method of preparing T cells and/or natural killer cells for immunotherapy comprising:
isolating T cells and/or natural killer cells from a blood sample of a human subject,
activating the isolated T cells and/or natural killer cells,
transducing the activated T cells and/or natural killer cells with the nucleic acid of any one of aspects 108-112 or the vector of any one of aspects 113-119, and
expanding the transduced T cells and/or natural killer cells.
   135. The method of aspect 134, further comprising isolating CD4+CD8+ T cells from the transduced T cells and/or natural killer cells and expanding the isolated CD4+CD8+ transduced T cells.
   136. The method of aspect 134 or aspect 135, wherein the blood sample comprises peripheral blood mononuclear cells (PMBC).
   137. The method of any one of aspects 134-136, wherein the activating comprises contacting the T cells and/or natural killer cells with an anti-CD3 and an anti-CD28 antibody.
   138. The method of any one of aspects 134-138, wherein the T cell is CD4+ T cell.
   139. The method of any one of aspects 134-138, wherein the T cell is CD8+ T cell.
   140. The method of aspect 134-139, wherein the T cell is γδ T cell or αβ T cell.
   141. The method of any one of aspects 134-140, wherein the activation and/or expanding are in the presence of a combination of IL-2 and IL-15 and optionally with zoledronate.
   142. A method of treating a patient who has cancer, comprising administering to the patient the composition of any one of aspects 129-133, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   143. A method of eliciting an immune response in a patient who has cancer, comprising administering to the patient the composition of any one of aspects 129-133, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   144. The method of aspect 142 or 143, wherein the T cell and/or natural killer (NK) cell kills cancer cells that present a peptide in a complex with an MHC molecule on a cell surface.
   145. A method of increasing persistence, longevity, functionality, naivety, capacity to kill antigen-presenting cells, or a combination thereof, of T cells and/or natural killer (NK) cell, comprising:
isolating T cells and/or natural killer (NK) cells from a blood sample of a human subject,
activating the isolated T cells and/or natural killer (NK) cells,
transducing the activated T cells and/or natural killer (NK) cells with the nucleic acid of any one of aspects 21, 55-59, or 108-112 or the vector of any one of aspects 22-33, 60-81, or 113-119, or a combination thereof, to obtain transduced T cells and/or natural killer (NK) cells, and
obtaining the transduced T cells or natural killer (NK) cells,
wherein the persistence, longevity, functionality, naivety, capacity to kill antigen-presenting cells, or a combination thereof of the transduced T cells and/or natural killer (NK) cells is increased as compared with that of control cells.
   146. The method of aspect 145, further comprising expanding the transduced T cells and/or natural killer (NK) cells.
   147. The method of aspect 145 or aspect 146, wherein the control cells comprise non-transduced T cells and/or natural killer (NK) cells, T cells and/or natural killer (NK) cells transduced with TCR only, or a combination thereof.
   148. The method of aspect 145 or aspect 146, wherein the control cells comprise non-transduced T cells and/or natural killer (NK) cells, T cells and/or natural killer (NK) cells transduced with TCR only, T cells and/or natural killer (NK) cells transduced with TCR and CD8 only, or a combination thereof.
   149. The method of any one of aspects 145-148, wherein the persistence, longevity, functionality, naivety, capacity to kill antigen-presenting cells, or a combination thereof of the transduced T cells and/or natural killer (NK) cells and the control cells is determined after one challenge with antigen-presenting cells, two challenges with antigen-presenting cells, three challenges with antigen-presenting cells, four challenges with antigen-presenting cells, five challenges with antigen-presenting cells, six challenges with antigen-presenting cells, seven challenges with antigen-presenting cells, or more challenges with antigen-presenting cells.
   150. The method of any one of aspects 145-148, wherein the persistence, longevity, functionality, naivety, capacity to kill antigen-presenting cells, or a combination thereof of the transduced T cells and/or natural killer (NK) cells and the control cells is determined after two challenges with antigen-presenting cells, after three challenges with antigen-presenting cells, or after more challenges with antigen-presenting cells.
   151. The method of any one of aspects 145-148, wherein the transduced T cells and/or natural killer (NK) cells and the control cells are cultured in the presence of exogenous TGF-β, optionally TGF-β1.
   152. The method of aspect 151, wherein the exogenous TGF-β, optionally TGF-β1, is added to cell cultures daily.
   153. The method of aspect 151, wherein the exogenous TGF-β, optionally TGF-β1, is added to cell cultures at the same time or times that tumor cells are added to cell cultures.
   154. A method of increasing interferon γ (IFNγ) secretion by T cells and/or natural killer (NK) cells, comprising:
isolating T cells and/or natural killer (NK) cells from a blood sample of a human subject,
activating the isolated T cells and/or natural killer (NK) cells,
transducing the activated T cells and/or natural killer (NK) cells with the nucleic acid of any one of aspects 21, 55-59, or 108-112 or the vector of any one of aspects 22-33, 60-81, or 113-119, or a combination thereof, to obtain transduced T cells and/or natural killer (NK) cells, and
obtaining the transduced T cells or natural killer (NK) cells,
wherein the IFNγ secretion of the transduced T cells and/or natural killer (NK) cells is increased as compared with that of control cells.
   155. The method of aspect 154, further comprising expanding the transduced T cells and/or natural killer (NK) cells.
   156. The method of aspect 154 or aspect 155, wherein the control cells comprise non-transduced T cells and/or natural killer (NK) cells, T cells and/or natural killer (NK) cells transduced with TCR only, or a combination thereof.
   157. The method of 154 or aspect 155, wherein the control cells comprise non-transduced T cells and/or natural killer (NK) cells, T cells and/or natural killer (NK) cells transduced with TCR only, T cells and/or natural killer (NK) cells transduced with TCR and CD8 only, or a combination thereof.
   158. The method of any one of aspects 154-157, wherein the IFNγ secretion by the transduced T cells and/or natural killer (NK) cells and the control cells is determined after one challenge with antigen-presenting cells, two challenges with antigen-presenting cells, three challenges with antigen-presenting cells, four challenges with antigen-presenting cells, five challenges with antigen-presenting cells, six challenges with antigen-presenting cells, seven challenges with antigen-presenting cells, or more challenges with antigen-presenting cells.
   159. The method of any one of aspects 154-157, wherein the IFNγ secretion by the transduced T cells and/or natural killer (NK) cells and the control cells is determined after two challenges with antigen-presenting cells, after three challenges with antigen-presenting cells, or after more challenges with antigen-presenting cells.
   160. The method of any one of aspects 154-159, wherein the transduced T cells and/or natural killer (NK) cells and the control cells are cultured in the presence of exogenous TGF-β, optionally TGF-β1.
   161. The method of aspect 160, wherein the exogenous TGF-β, optionally TGF-β1, is added to cell cultures daily.
   162. The method of aspect 160, wherein the exogenous TGF-β, optionally TGF-β1, is added to cell cultures at the same time or times that tumor cells are added to cell cultures.
   163. The method of any one of aspects 145-162, wherein the antigen presenting cells present an antigen on a cell surface, and the transduced T cells and/or natural killer (NK) cells and the control cells are capable of killing the antigen presenting cells.
   164. The method of aspect 163, wherein the antigen comprises a peptide.
   165. The method of aspect 164, wherein the antigen comprising a peptide is in a complex with an MHC molecule on the cell surface.
   166. A transduced T cell and/or natural killer (NK) cell produced by the method of any one of aspects 145-166.
   167. The transduced T cell and/or natural killer (NK) cell of aspect 166, wherein the T cell is an αβ T cell, a γδ T cell, and/or a natural killer T cell.
   168. The transduced T cell and/or natural killer (NK) cell of aspect 167, wherein the αβ T cell is a CD4+ T cell.
   169. The transduced T cell and/or natural killer (NK) cell of aspect 167, wherein the αβ T cell is a CD8+ T cell.
   170. The transduced T cell and/or natural killer (NK) cell of aspect 167, wherein the γδ T cell is a Vγ9Vδ2+ T cell.
   171. A composition comprising the transduced T cell and/or natural killer (NK) cell of any one of aspects 167-170.
   172. The composition of aspect 171, wherein the composition is a pharmaceutical composition.
   173. The composition of aspect 171 or aspect 172, wherein the composition further comprises an adjuvant, excipient, carrier, diluent, buffer, stabilizer, or a combination thereof.
   174. The composition of aspect 173, wherein the adjuvant is an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof.
   175. The composition of aspect 173 or aspect 174, wherein the adjuvant is IL-2, IL-7, IL-12, IL-15, IL-21, or any combination thereof.
   176. A method of treating a patient who has cancer, comprising administering to the patient the composition of any one of aspects 171-175, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   177. A method of eliciting an immune response in a patient who has cancer, comprising administering to the patient the composition of any one of aspects 171-175, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
   178. The method of aspect 176 or 177, wherein the T cell and/or natural killer (NK) cell kills cancer cells that present a peptide in a complex with an MHC molecule on a cell surface.
   179. A nucleic acid encoding a fusion polypeptide of Formula III:

N-terminus-P6-PL-P7-C-terminus [III],
wherein P6 and P7 are each independently a first and second polypeptides and PL is a linker, wherein PL comprises SEQ ID NO: 320 or 322 or a sequence at least about 95%, at least about 96%, at least about 97%, at least about 98%, at least about 99%, or about 100% identical to SEQ ID NO: 320 or 322.
   180. A nucleic acid comprising formula IV:

5′-N6-NL-N7-3′ [IV],
wherein N6 and N7 each independently encode a first and second polypeptides and NL encodes a linker, wherein NL comprises SEQ ID NO: 321 or 323 or a sequence at least about 95%, at least about 96%, at least about 97%, at least about 98%, at least about 99%, or about 100% identical to SEQ ID NO: 321 or 323.
   181. A polypeptide, polypeptides, or fusion polypeptide encoded by the nucleic acid of any one of aspects 1-3, 21, 55-59, 108-112, or 179-180.
   182. The polypeptide, polypeptides, or fusion polypeptide of aspect 181 wherein the polypeptide is isolated, recombinant, or both isolated and recombinant.
   183. A T cell and/or natural killer (NK) cell comprising a polypeptide comprising an amino acid sequence at least about 95%, 96%, 97%, 98%, 99%, or 100% identical to the amino acid sequence of SEQ ID NO: 305 or 307 and (a) at least one TCR polypeptide comprising an α chain and a β chain, (b) at least one CD8 polypeptide comprising (i) an α chain, (ii) a β chain, or (iii) an α chain and a β chain or (c) at least one TCR polypeptide comprising an α chain and a β chain and at least one CD8 polypeptide comprising (i) an α chain, (ii) a β chain, or (iii) an α chain and a β chain.
   184. The T cell and/or natural killer (NK) cell of aspect 183, wherein the T cell is an αβ T cell, a γδ T cell, and/or a natural killer T cell.
   185. The T cell and/or natural killer (NK) cell of aspect 184, wherein the αβ T cell is a CD4+ T cell.
   186. The T cell and/or natural killer (NK) cell of aspect 184, wherein the αβ T cell is a CD8+ T cell.
   187. The T cell and/or natural killer (NK) cell of aspect 184, wherein the γδ T cell is a Vγ9Vδ2+ T cell.
   188. The nucleic acid of any one of aspects 1-3, 21, 55-59, 108-112, or 179-180, wherein the nucleic acid is isolated, recombinant, or both isolated and recombinant.
   189. The vector of any one of aspects 4-15, 22-33, 60-81, or 113-119, wherein the vector is isolated, recombinant, or both isolated and recombinant.
   190. The T cell and/or natural killer (NK) cell of any one of aspects 16-20, 34-38, 82-91, 120-128, 167-171, or 184-188, wherein the T cell and/or natural killer (NK) cell is isolated, recombinant, engineered, or any combination thereof.

What is claimed is:

1. A nucleic acid encoding a polypeptide comprising
(i) SEQ ID NO: 305 or a sequence at least about 95%, at least about 96%, at least about 97%, at least about 98%, at least about 99%, or about 100% identical to SEQ ID NO: 305;
(ii) SEQ ID NO: 307 or a sequence at least about 95%, at least about 96%, at least about 97%, at least about 98%, at least about 99%, or about 100% identical to SEQ ID NO: 307; or
(iii) both (i) and (ii).

2. The nucleic acid of claim 1 comprising
(i) SEQ ID NO: 306 or a sequence at least about 80%, at least about 85%, at least about 90%, at least about 95%, at least about 96%, at least about 97%, at least about 98%, at least about 99%, or about 100% identical to SEQ ID NO: 306;
(ii) SEQ ID NO: 308 or a sequence at least about 80%, at least about 85%, at least about 90%, at least about 95%, at least about 96%, at least about 97%, at least about 98%, at least about 99%, or about 100% identical to SEQ ID NO: 308; or
(iii) both (i) and (ii).

3. The nucleic acid of claim 1, further comprising a nucleic acid sequence encoding at least one TCR polypeptide, at least one CD8 polypeptide, or at least one TCR polypeptide and at least one CD8 polypeptide.

4. The nucleic acid of claim 1, comprising
(i) SEQ ID NO: 312 or a sequence at least about 80%, at least about 85%, at least about 90%, at least about 95%, at least about 96%, at least about 97%, at least about 98%, at least about 99%, or about 100% identical to SEQ ID NO: 312;
(ii) SEQ ID NO: 313 or a sequence at least about 80%, at least about 85%, at least about 90%, at least about 95%, at least about 96%, at least about 97%, at least about 98%, at least about 99%, or about 100% identical to SEQ ID NO: 313; or
(iii) both (i) and (ii).

5. The nucleic acid of claim 3,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, or 91 and 92, in particular wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303;
wherein the CD8α chain is SEQ ID NO: 7, 258, 259, 262, or a variant thereof;
wherein the CD8β chain is SEQ ID NO: 8, 9, 10, 11, 12, 13, or 14.

6. The nucleic acid of claim 3, wherein
the nucleic acid sequence encoding the at least one TCR polypeptide and at least one CD8 polypeptide comprises at least about 80%, 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301.

7. A vector comprising the nucleic acid of claim 1.

8. The vector of claim 7, further comprising a post-transcriptional regulatory element (PRE) selected from a Woodchuck PRE (WPRE), Woodchuck PRE (WPRE) mutant 1, Woodchuck PRE (WPRE) mutant 2, or hepatitis B virus (HBV) PRE (HPRE) (SEQ ID NO: 366).

9. The vector of claim 7, wherein the vector is a viral vector or a non-viral vector.

10. The vector of claim 9, wherein the viral vector is selected from adenoviruses, poxviruses, alphaviruses, arenaviruses, flaviviruses, rhabdoviruses, retroviruses, lentiviruses, herpesviruses, paramyxoviruses, picornaviruses, and combinations thereof, in particular a lentiviral vector.

11. A vector comprising N1, N2, N3, N4, N5, L1, L2, L3, and L4, in any order, wherein
N1 comprises a nucleic acid sequence encoding a CD8β chain and is present or absent, wherein the CD8β chain is SEQ ID NO: 8, 9, 10, 11, 12, 13, or 14,
N2 comprises a nucleic acid sequence encoding a CD8α chain,
wherein the CD8α chain is SEQ ID NO: 7, 258, 259, 262, or a variant thereof,
N3 comprises a nucleic acid sequence encoding a TCRβ chain,
N4 comprises a nucleic acid sequence encoding a TCRα chain,
wherein the TCR α chain and the TCR β chain are selected from SEQ ID NO: 15 and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, or 91 and 92, in particular wherein the TCR α chain and the TCR β chain are selected from SEQ ID NO: 15 and 16, 57 and 58, 59 and 60, 61 and 62, 63 and 64, 65 and 66, 67 and 68, 69 and 70, and 71 and 303; and
N5 comprises a nucleic acid sequence encoding at least one dominant negative TGFβ Receptor II (dnTGFβRII) polypeptide comprising
(i) SEQ ID NO: 305 or a sequence at least about 95%, at least about 96%, at least about 97%, at least about 98%, at least about 99%, or about 100% identical to SEQ ID NO: 305;
(ii) SEQ ID NO: 307 or a sequence at least about 95%, at least about 96%, at least about 97%, at least about 98%, at least about 99%, or about 100% identical to SEQ ID NO: 307; or
(iii) both (i) and (ii), and
wherein L1-L4 each comprises a nucleic acid sequence encoding at least about one linker, wherein each of L1-L4 is independently the same or different, and wherein each of L1-L4 is independently present or absent.

12. The vector of claim 11, comprising Formula I or Formula II:
5′-N1-L1-N2-L2-N3-L3-N4-L4-N5-3′ [I]
5′-N5-L1-N1-L2-N2-L3-N3-L4-N4-3′ [II].

13. The vector of claim 11, further comprising
(i) a nucleic acid encoding a 2A peptide or an internal ribosome entry site (IRES) positioned between N1 and L1, between L1 and N2, between N2 and L2, between L2 and N3, between N3 and L3, between L3 and N4, between N4 and L4, between L4 and N5, or any combination thereof or
(ii) a nucleic acid encoding a 2A peptide or an internal ribosome entry site (IRES) positioned between N5 and L1, between L1 and N1, between N1 and L2, between L2 and N2, between N2 and L3, between L3 and N3, between N3 and L4, between L4 and N4, or any combination thereof,
wherein the 2A peptide is P2A (SEQ ID NO: 93), T2A (SEQ ID NO: 94), E2A (SEQ ID NO: 95), or F2A (SEQ ID NO: 96).

14. A method of preparing T cells and/or natural killer cells for immunotherapy comprising:
isolating T cells and/or natural killer cells from a blood sample of a human subject,
activating the isolated T cells and/or natural killer cells,
transducing the activated T cells and/or natural killer cells with the nucleic acid of claim 1, and
expanding the transduced T cells and/or natural killer cells.

15. A method of increasing persistence, longevity, functionality, naivety, capacity to kill antigen-presenting cells, IFN-γ secretion or a combination thereof, of T cells and/or natural killer (NK) cell, comprising:
isolating T cells and/or natural killer (NK) cells from a blood sample of a human subject,
activating the isolated T cells and/or natural killer (NK) cells,
transducing the activated T cells and/or natural killer (NK) cells with the nucleic acid of claim 1, or a combination thereof, to obtain transduced T cells and/or natural killer (NK) cells, and
obtaining the transduced T cells and/or natural killer (NK) cells,
wherein the persistence, longevity, functionality, naivety, capacity to kill antigen-presenting cells, IFN-γ secretion or a combination thereof of the transduced T cells and/or natural killer (NK) cells is increased as compared with that of control cells.

16. The method of claim 15, wherein the control cells comprise non-transduced T cells and/or natural killer (NK) cells, T cells and/or natural killer (NK) cells transduced with TCR only, T cells and/or natural killer (NK) cells transduced with TCR and CD8 only, or a combination thereof.

17. A T cell and/or natural killer (NK) cell transduced with the nucleic acid of claim 1.

18. A composition comprising the T cell and/or natural killer (NK) cell of claim 17.

19. The composition of claim 18, further comprising an adjuvant selected from an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or any combination thereof, in particular wherein the adjuvant is IL-2, IL-7, IL-12, IL-15, IL-21, and any combination thereof.

20. A method of treating and/or eliciting an immune response in a patient who has cancer, comprising administering to the patient the composition of claim 18, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.


SEQUENCE LISTING (367 sequences)

SEQ ID NO: 1 - protein, 114 aa, synthetic construct
  REGION 1..114: note = CD8alpha Ig-like domain-1
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 2 - protein, 38 aa, synthetic construct
  REGION 1..38: note = CD8beta stalk region
SVVDFLPTTAQPTKKSTLKKRVCRLPRPETQKGPLCSP

SEQ ID NO: 3 - protein, 21 aa, synthetic construct
  REGION 1..21: note = CD8alpha transmembrane domain
IYIWAPLAGTCGVLLLSLVIT

SEQ ID NO: 4 - protein, 32 aa, synthetic construct
  REGION 1..32: note = CD8alpha cytoplasmic tail
LYCNHRNRRRVCKCPRPVVKSGDKPSLSARYV

SEQ ID NO: 5 - protein, 205 aa, synthetic construct
  REGION 1..205: note = m1CD8alpha (signal-less)
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 6 - protein, 21 aa, synthetic construct
  REGION 1..21: note = Signal peptide
MALPVTALLLPLALLLHAARP

SEQ ID NO: 7 - protein, 226 aa, synthetic construct
  REGION 1..226: note = m1CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 8 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 9 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 10 - protein, 189 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 11 - protein, 200 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 12 - protein, 225 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 13 - protein, 225 aa, Homo sapiens; identical to SEQ ID NO: 12

SEQ ID NO: 14 - protein, 222 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 15 - protein, 273 aa, synthetic construct
  REGION 1..273: note = R11KEA alpha chain
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 311 aa, synthetic construct
  REGION 1..311: note = R11KE beta chain
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 274 aa, synthetic construct
  REGION 1..274: note = R20P1H7 alpha chain
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 314 aa, synthetic construct
  REGION 1..314: note = R20P1H7 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 272 aa, synthetic construct
  REGION 1..272: note = R7P1D5 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 310 aa, synthetic construct
  REGION 1..310: note = R7P1D5 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 277 aa, synthetic construct
  REGION 1..277: note = R10P2G12 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 313 aa, synthetic construct
  REGION 1..313: note = R10P2G12 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 271 aa, synthetic construct
  REGION 1..271: note = R10P1A7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESKETRLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 317 aa, synthetic construct
  REGION 1..317: note = R10P1A7 beta chain
MLLLLLLLGPGISLLLPGSLAGSGLGAWSQHPSVWICKSGTSVKIECRSLDFQATTMFWYRQFPKQSLMLMATSNEGSKATYEQGVEKDKFLINHASLTLSTLTVTSAHPEDSSFYICSARAGGHEQFFGPGTRLTVLEDLKNVFPPEVAVFEPSEAEISHTQKATLVCLATGFYPDHVELSWVWNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 271 aa, synthetic construct
  REGION 1..271: note = R4P1D10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFHDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 308 aa, synthetic construct
  REGION 1..308: note = R4P1D10 beta chain
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVASAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 274 aa, synthetic construct
  REGION 1..274: note = R4P3F9 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 308 aa, synthetic construct
  REGION 1..308: note = R4P3F9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 271 aa, synthetic construct
  REGION 1..271: note = R4P3H3 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 314 aa, synthetic construct
  REGION 1..314: note = R4P3H3 beta chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLTSGGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 270 aa, synthetic construct
  REGION 1..270: note = R36P3F9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 314 aa, synthetic construct
  REGION 1..314: note = R36P3F9 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTSGGLSGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 272 aa, synthetic construct
  REGION 1..272: note = R52P2G11 alpha chain
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 312 aa, synthetic construct
  REGION 1..312: note = R52P2G11 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGSPDGNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 279 aa, synthetic construct
  REGION 1..279: note = R53P2A9 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYNSYAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 310 aa, synthetic construct
  REGION 1..310: note = R53P2A9 beta chain
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLDGTSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 271 aa, synthetic construct
  REGION 1..271: note = R26P1A9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCLIGASGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 310 aa, synthetic construct
  REGION 1..310: note = R26P1A9 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYFGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 276 aa, synthetic construct
  REGION 1..276: note = R26P2A6 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 311 aa, synthetic construct
  REGION 1..311: note = R26P2A6 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASTTPDGTDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 274 aa, synthetic construct
  REGION 1..274: note = R26P3H1 alpha chain
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 310 aa, synthetic construct
  REGION 1..310: note = R26P3H1 beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRAEGGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 43 - protein, 273 aa, synthetic construct
  REGION 1..273: note = R35P3A4 alpha chain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASPTGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 311 aa, synthetic construct
  REGION 1..311: note = R35P3A4 beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLGGASQEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 266 aa, synthetic construct
  REGION 1..266: note = R37P1C9 alpha chain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 309 aa, synthetic construct
  REGION 1..309: note = R37P1C9 beta chain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 277 aa, synthetic construct
  REGION 1..277: note = R37P1H1 alpha chain
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFGYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 311 aa, synthetic construct
  REGION 1..311: note = R37P1H1 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSNEGQGWEAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 268 aa, synthetic construct
  REGION 1..268: note = R42P3A9 alpha chain
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVHNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 322 aa, synthetic construct
  REGION 1..322: note = R42P3A9 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLGQGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 276 aa, synthetic construct
  REGION 1..276: note = R43P3F2 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 323 aa, synthetic construct
  REGION 1..323: note = R43P3F2 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGTSGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 273 aa, synthetic construct
  REGION 1..273: note = R43P3G5 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 311 aa, synthetic construct
  REGION 1..311: note = R43P3G5 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRLPSRTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 55 - protein, 270 aa, synthetic construct
  REGION 1..270: note = R59P2E7 alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 321 aa, synthetic construct
  REGION 1..321: note = R59P2E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGLGTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - protein, 273 aa, synthetic construct
  REGION 1..273: note = R11P3D3 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 311 aa, synthetic construct
  REGION 1..311: note = R11P3D3 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 59 - protein, 275 aa, synthetic construct
  REGION 1..275: note = R16P1C10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 311 aa, synthetic construct
  REGION 1..311: note = R16P1C10 beta chain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 274 aa, synthetic construct
  REGION 1..274: note = R16P1E8 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 309 aa, synthetic construct
  REGION 1..309: note = R16P1E8 beta chain
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 63 - protein, 273 aa, synthetic construct
  REGION 1..273: note = R17P1A9 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 314 aa, synthetic construct
  REGION 1..314: note = R17P1A9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - protein, 277 aa, synthetic construct
  REGION 1..277: note = R17P1D7 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 313 aa, synthetic construct
  REGION 1..313: note = R17P1D7 beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 67 - protein, 273 aa, synthetic construct
  REGION 1..273: note = R17P1G3 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 311 aa, synthetic construct
  REGION 1..311: note = R17P1G3 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 274 aa, synthetic construct
  REGION 1..274: note = R17P2B6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 319 aa, synthetic construct
  REGION 1..319: note = R17P2B6 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 273 aa, synthetic construct; identical to SEQ ID NO: 15
  REGION 1..273: note = R11P3D3KE alpha chain

SEQ ID NO: 72 - DNA, 819 bp, synthetic construct
  misc_feature 1..819: note = R11KEA alpha chain nucleic acid sequence
  source 1..819: mol_type = other DNA
atggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcc

SEQ ID NO: 73 - DNA, 933 bp, synthetic construct
  misc_feature 1..933: note = R11KEA beta chain nucleic acid sequence
  source 1..933: mol_type = other DNA
atggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcgggga

SEQ ID NO: 74 - protein, 307 aa, synthetic construct
  REGION 1..307: note = R39P1C12 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 75 - protein, 270 aa, synthetic construct
  REGION 1..270: note = R39P1F5 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 311 aa, synthetic construct
  REGION 1..311: note = R39P1F5 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 272 aa, synthetic construct
  REGION 1..272: note = R40P1C2 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 311 aa, synthetic construct
  REGION 1..311: note = R40P1C2 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 79 - protein, 271 aa, synthetic construct
  REGION 1..271: note = R41P3E6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 310 aa, synthetic construct
  REGION 1..310: note = R41P3E6 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 81 - protein, 270 aa, synthetic construct
  REGION 1..270: note = R43P3G4 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 311 aa, synthetic construct
  REGION 1..311: note = R43P3G4 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 281 aa, synthetic construct
  REGION 1..281: note = R44P3B3 alpha chain
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 311 aa, synthetic construct
  REGION 1..311: note = R44P3B3 beta chain
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 272 aa, synthetic construct
  REGION 1..272: note = R44P3E7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 320 aa, synthetic construct
  REGION 1..320: note = R44P3E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 87 - protein, 273 aa, synthetic construct
  REGION 1..273: note = R49P2B7 alpha chain
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 312 aa, synthetic construct
  REGION 1..312: note = R49P2B7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 89 - protein, 276 aa, synthetic construct
  REGION 1..276: note = R55P1G7 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 309 aa, synthetic construct
  REGION 1..309: note = R55P1G7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 270 aa, synthetic construct
  REGION 1..270: note = R59P2A7 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 307 aa, synthetic construct
  REGION 1..307: note = R59P2A7 beta chain
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 93 - protein, 19 aa, synthetic construct
  REGION 1..19: note = P2A
ATNFSLLKQAGDVEENPGP

SEQ ID NO: 94 - protein, 18 aa, synthetic construct
  REGION 1..18: note = T2A
EGRGSLLTCGDVEENPGP

SEQ ID NO: 95 - protein, 20 aa, synthetic construct
  REGION 1..20: note = E2A
QCTNYALLKLAGDVESNPGP

SEQ ID NO: 96 - protein, 22 aa, synthetic construct
  REGION 1..22: note = F2A
VKQTLNFDLLKLAGDVESNPGP

SEQ ID NO: 97 - protein, 559 aa, synthetic construct
  REGION 1..559: note = RD114TR
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

SEQ ID NO: 98 - protein, 10 aa, Homo sapiens
YLYDSETKNA

SEQ ID NO: 99 - protein, 9 aa, Homo sapiens
HLMDQPLSV

SEQ ID NO: 100 - protein, 9 aa, Homo sapiens
GLLKKINSV

SEQ ID NO: 101 - protein, 9 aa, Homo sapiens
FLVDGSSAL

SEQ ID NO: 102 - protein, 10 aa, Homo sapiens
FLFDGSANLV

SEQ ID NO: 103 - protein, 9 aa, Homo sapiens
FLYKIIDEL

SEQ ID NO: 104 - protein, 11 aa, Homo sapiens
FILDSAETTTL

SEQ ID NO: 105 - protein, 9 aa, Homo sapiens
SVDVSPPKV

SEQ ID NO: 106 - protein, 8 aa, Homo sapiens
VADKIHSV

SEQ ID NO: 107 - protein, 9 aa, Homo sapiens
IVDDLTINL

SEQ ID NO: 108 - protein, 9 aa, Homo sapiens
GLLEELVTV

SEQ ID NO: 109 - protein, 10 aa, Homo sapiens
TLDGAAVNQV

SEQ ID NO: 110 - protein, 10 aa, Homo sapiens
SVLEKEIYSI

SEQ ID NO: 111 - protein, 9 aa, Homo sapiens
LLDPKTIFL

SEQ ID NO: 112 - protein, 9 aa, Homo sapiens
YTFSGDVQL

SEQ ID NO: 113 - protein, 9 aa, Homo sapiens
YLMDDFSSL

SEQ ID NO: 114 - protein, 9 aa, Homo sapiens
KVWSDVTPL

SEQ ID NO: 115 - protein, 11 aa, Homo sapiens
LLWGHPRVALA

SEQ ID NO: 116 - protein, 11 aa, Homo sapiens
KIWEELSVLEV

SEQ ID NO: 117 - protein, 9 aa, Homo sapiens
LLIPFTIFM

SEQ ID NO: 118 - protein, 9 aa, Homo sapiens
FLIENLLAA

SEQ ID NO: 119 - protein, 11 aa, Homo sapiens
LLWGHPRVALA

SEQ ID NO: 120 - protein, 9 aa, Homo sapiens
FLLEREQLL

SEQ ID NO: 121 - protein, 9 aa, Homo sapiens
SLAETIFIV

SEQ ID NO: 122 - protein, 9 aa, Homo sapiens
TLLEGISRA

SEQ ID NO: 123 - protein, 9 aa, Homo sapiens
KIQEILTQV

SEQ ID NO: 124 - protein, 10 aa, Homo sapiens
VIFEGEPMYL

SEQ ID NO: 125 - protein, 9 aa, Homo sapiens
SLFESLEYL

SEQ ID NO: 126 - protein, 9 aa, Homo sapiens
SLLNQPKAV

SEQ ID NO: 127 - protein, 9 aa, Homo sapiens
GLAEFQENV

SEQ ID NO: 128 - protein, 9 aa, Homo sapiens
KLLAVIHEL

SEQ ID NO: 129 - protein, 9 aa, Homo sapiens
TLHDQVHLL

SEQ ID NO: 130 - protein, 11 aa, Homo sapiens
TLYNPERTITV

SEQ ID NO: 131 - protein, 9 aa, Homo sapiens
KLQEKIQEL

SEQ ID NO: 132 - protein, 10 aa, Homo sapiens
SVLEKEIYSI

SEQ ID NO: 133 - protein, 11 aa, Homo sapiens
RVIDDSLVVGV

SEQ ID NO: 134 - protein, 9 aa, Homo sapiens
VLFGELPAL

SEQ ID NO: 135 - protein, 9 aa, Homo sapiens
GLVDIMVHL

SEQ ID NO: 136 - protein, 9 aa, Homo sapiens
FLNAIETAL

SEQ ID NO: 137 - protein, 9 aa, Homo sapiens
ALLQALMEL

SEQ ID NO: 138 - protein, 9 aa, Homo sapiens
ALSSSQAEV

SEQ ID NO: 139 - protein, 11 aa, Homo sapiens
SLITGQDLLSV

SEQ ID NO: 140 - protein, 9 aa, Homo sapiens
QLIEKNWLL

SEQ ID NO: 141 - protein, 9 aa, Homo sapiens
LLDPKTIFL

SEQ ID NO: 142 - protein, 9 aa, Homo sapiens
RLHDENILL

SEQ ID NO: 143 - protein, 9 aa, Homo sapiens
YTFSGDVQL

SEQ ID NO: 144 - protein, 9 aa, Homo sapiens
GLPSATTTV

SEQ ID NO: 145 - protein, 11 aa, Homo sapiens
GLLPSAESIKL

SEQ ID NO: 146 - protein, 9 aa, Homo sapiens
KTASINQNV

SEQ ID NO: 147 - protein, 9 aa, Homo sapiens
SLLQHLIGL

SEQ ID NO: 148 - protein, 9 aa, Homo sapiens
YLMDDFSSL

SEQ ID NO: 149 - protein, 9 aa, Homo sapiens
LMYPYIYHV

SEQ ID NO: 150 - protein, 9 aa, Homo sapiens
KVWSDVTPL

SEQ ID NO: 151 - protein, 11 aa, Homo sapiens
LLWGHPRVALA

SEQ ID NO: 152 - protein, 9 aa, Homo sapiens
VLDGKVAVV

SEQ ID NO: 153 - protein, 9 aa, Homo sapiens
GLLGKVTSV

SEQ ID NO: 154 - protein, 9 aa, Homo sapiens
KMISAIPTL

SEQ ID NO: 155 - protein, 11 aa, Homo sapiens
GLLETTGLLAT

SEQ ID NO: 156 - protein, 9 aa, Homo sapiens
TLNTLDINL

SEQ ID NO: 157 - protein, 9 aa, Homo sapiens
VIIKGLEEI

SEQ ID NO: 158 - protein, 9 aa, Homo sapiens
YLEDGFAYV

SEQ ID NO: 159 - protein, 11 aa, Homo sapiens
KIWEELSVLEV

SEQ ID NO: 160 - protein, 9 aa, Homo sapiens
LLIPFTIFM

SEQ ID NO: 161 - protein, 10 aa, Homo sapiens
ISLDEVAVSL

SEQ ID NO: 162 - protein, 10 aa, Homo sapiens
KISDFGLATV

SEQ ID NO: 163 - protein, 11 aa, Homo sapiens
KLIGNIHGNEV

SEQ ID NO: 164 - protein, 9 aa, Homo sapiens
ILLSVLHQL

SEQ ID NO: 165 - protein, 9 aa, Homo sapiens
LDSEALLTL

SEQ ID NO: 166 - protein, 13 aa, Homo sapiens
VLQENSSDYQSNL

SEQ ID NO: 167 - protein, 11 aa, Homo sapiens
HLLGEGAFAQV

SEQ ID NO: 168 - protein, 9 aa, Homo sapiens
SLVENIHVL

SEQ ID NO: 169 - protein, 9 aa, Homo sapiens
YTFSGDVQL

SEQ ID NO: 170 - protein, 9 aa, Homo sapiens
SLSEKSPEV

SEQ ID NO: 171 - protein, 10 aa, Homo sapiens
AMFPDTIPRV

SEQ ID NO: 172 - protein, 9 aa, Homo sapiens
FLIENLLAA

SEQ ID NO: 173 - protein, 9 aa, Homo sapiens
FTAEFLEKV

SEQ ID NO: 174 - protein, 9 aa, Homo sapiens
ALYGNVQQV

SEQ ID NO: 175 - protein, 9 aa, Homo sapiens
LFQSRIAGV

SEQ ID NO: 176 - protein, 11 aa, Homo sapiens
ILAEEPIYIRV

SEQ ID NO: 177 - protein, 9 aa, Homo sapiens
FLLEREQLL

SEQ ID NO: 178 - protein, 10 aa, Homo sapiens
LLLPLELSLA

SEQ ID NO: 179 - protein, 9 aa, Homo sapiens
SLAETIFIV

SEQ ID NO: 180 - protein, 11 aa, Homo sapiens
AILNVDEKNQV

SEQ ID NO: 181 - protein, 9 aa, Homo sapiens
RLFEEVLGV

SEQ ID NO: 182 - protein, 9 aa, Homo sapiens
YLDEVAFML

SEQ ID NO: 183 - protein, 11 aa, Homo sapiens
KLIDEDEPLFL

SEQ ID NO: 184 - protein, 9 aa, Homo sapiens
KLFEKSTGL

SEQ ID NO: 185 - protein, 11 aa, Homo sapiens
SLLEVNEASSV

SEQ ID NO: 186 - protein, 10 aa, Homo sapiens
GVYDGREHTV

SEQ ID NO: 187 - protein, 10 aa, Homo sapiens
GLYPVTLVGV

SEQ ID NO: 188 - protein, 9 aa, Homo sapiens
ALLSSVAEA

SEQ ID NO: 189 - protein, 9 aa, Homo sapiens
TLLEGISRA

SEQ ID NO: 190 - protein, 9 aa, Homo sapiens
SLIEESEEL

SEQ ID NO: 191 - protein, 9 aa, Homo sapiens
ALYVQAPTV

SEQ ID NO: 192 - protein, 10 aa, Homo sapiens
KLIYKDLVSV

SEQ ID NO: 193 - protein, 9 aa, Homo sapiens
ILQDGQFLV

SEQ ID NO: 194 - protein, 9 aa, Homo sapiens
SLLDYEVSI

SEQ ID NO: 195 - protein, 9 aa, Homo sapiens
LLGDSSFFL

SEQ ID NO: 196 - protein, 10 aa, Homo sapiens
VIFEGEPMYL

SEQ ID NO: 197 - protein, 9 aa, Homo sapiens
ALSYILPYL

SEQ ID NO: 198 - protein, 9 aa, Homo sapiens
FLFVDPELV

SEQ ID NO: 199 - protein, 11 aa, Homo sapiens
SEWGSPHAAVP

SEQ ID NO: 200 - protein, 9 aa, Homo sapiens
ALSELERVL

SEQ ID NO: 201 - protein, 9 aa, Homo sapiens
SLFESLEYL

SEQ ID NO: 202 - protein, 9 aa, Homo sapiens
KVLEYVIKV

SEQ ID NO: 203 - protein, 10 aa, Homo sapiens
VLLNEILEQV

SEQ ID NO: 204 - protein, 9 aa, Homo sapiens
SLLNQPKAV

SEQ ID NO: 205 - protein, 9 aa, Homo sapiens
KMSELQTYV

SEQ ID NO: 206 - protein, 11 aa, Homo sapiens
ALLEQTGDMSL

SEQ ID NO: 207 - protein, 11 aa, Homo sapiens
VIIKGLEEITV

SEQ ID NO: 208 - protein, 9 aa, Homo sapiens
KQFEGTVEI

SEQ ID NO: 209 - protein, 9 aa, Homo sapiens
KLQEEIPVL

SEQ ID NO: 210 - protein, 9 aa, Homo sapiens
GLAEFQENV

SEQ ID NO: 211 - protein, 9 aa, Homo sapiens
NVAEIVIHI

SEQ ID NO: 212 - protein, 9 aa, Homo sapiens
ALAGIVTNV

SEQ ID NO: 213 - protein, 12 aa, Homo sapiens
NLLIDDKGTIKL

SEQ ID NO: 214 - protein, 10 aa, Homo sapiens
VLMQDSRLYL

SEQ ID NO: 215 - protein, 9 aa, Homo sapiens
KVLEHVVRV

SEQ ID NO: 216 - protein, 9 aa, Homo sapiens
LLWGNLPEI

SEQ ID NO: 217 - protein, 9 aa, Homo sapiens
SLMEKNQSL

SEQ ID NO: 218 - protein, 9 aa, Homo sapiens
KLLAVIHEL

SEQ ID NO: 219 - protein, 10 aa, Homo sapiens
ALGDKFLLRV

SEQ ID NO: 220 - protein, 11 aa, Homo sapiens
FLMKNSDLYGA

SEQ ID NO: 221 - protein, 10 aa, Homo sapiens
KLIDHQGLYL

SEQ ID NO: 222 - protein, 12 aa, Homo sapiens
GPGIFPPPPPQP

SEQ ID NO: 223 - protein, 9 aa, Homo sapiens
ALNESLVEC

SEQ ID NO: 224 - protein, 9 aa, Homo sapiens
GLAALAVHL

SEQ ID NO: 225 - protein, 9 aa, Homo sapiens
LLLEAVWHL

SEQ ID NO: 226 - protein, 9 aa, Homo sapiens
SIIEYLPTL

SEQ ID NO: 227 - protein, 9 aa, Homo sapiens
TLHDQVHLL

SEQ ID NO: 228 - protein, 9 aa, Homo sapiens
SLLMWITQC

SEQ ID NO: 229 - protein, 11 aa, Homo sapiens
FLLDKPQDLSI

SEQ ID NO: 230 - protein, 10 aa, Homo sapiens
YLLDMPLWYL

SEQ ID NO: 231 - protein, 9 aa, Homo sapiens
GLLDCPIFL

SEQ ID NO: 232 - protein, 9 aa, Homo sapiens
VLIEYNFSI

SEQ ID NO: 233 - protein, 11 aa, Homo sapiens
TLYNPERTITV

SEQ ID NO: 234 - protein, 9 aa, Homo sapiens
AVPPPPSSV

SEQ ID NO: 235 - protein, 9 aa, Homo sapiens
KLQEELNKV

SEQ ID NO: 236 - protein, 11 aa, Homo sapiens
KLMDPGSLPPL

SEQ ID NO: 237 - protein, 9 aa, Homo sapiens
ALIVSLPYL

SEQ ID NO: 238 - protein, 9 aa, Homo sapiens
FLLDGSANV

SEQ ID NO: 239 - protein, 10 aa, Homo sapiens
ALDPSGNQLI

SEQ ID NO: 240 - protein, 9 aa, Homo sapiens
ILIKHLVKV

SEQ ID NO: 241 - protein, 9 aa, Homo sapiens
VLLDTILQL

SEQ ID NO: 242 - protein, 9 aa, Homo sapiens
HLIAEIHTA

SEQ ID NO: 243 - protein, 9 aa, Homo sapiens
SMNGGVFAV

SEQ ID NO: 244 - protein, 9 aa, Homo sapiens
MLAEKLLQA

SEQ ID NO: 245 - protein, 9 aa, Homo sapiens
YMLDIFHEV

SEQ ID NO: 246 - protein, 11 aa, Homo sapiens
ALWLPTDSATV

SEQ ID NO: 247 - protein, 9 aa, Homo sapiens
GLASRILDA

SEQ ID NO: 248 - protein, 9 aa, Homo sapiens
ALSVLRLAL

SEQ ID NO: 249 - protein, 9 aa, Homo sapiens
SYVKVLHHL

SEQ ID NO: 250 - protein, 9 aa, Homo sapiens
VYLPKIPSW

SEQ ID NO: 251 - protein, 9 aa, Homo sapiens
NYEDHFPLL

SEQ ID NO: 252 - protein, 9 aa, Homo sapiens
VYIAELEKI

SEQ ID NO: 253 - protein, 12 aa, Homo sapiens
VHFEDTGKTLLF

SEQ ID NO: 254 - protein, 9 aa, Homo sapiens
VLSPFILTL

SEQ ID NO: 255 - protein, 9 aa, Homo sapiens
HLLEGSVGV

SEQ ID NO: 256 - DNA, 607 bp, synthetic construct
  misc_feature 1..607: note = WPREmut1
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 257 - DNA, 581 bp, synthetic construct
  misc_feature 1..581: note = WPREmut2
  source 1..581: mol_type = other DNA
gagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 258 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 259 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 260 - protein, 47 aa, synthetic construct
  REGION 1..47: note = CD8alpha stalk
KPTTTPAPRPPTPAPTIASQPLSLRPEACRPAAGGAVHTRGLDFACD

SEQ ID NO: 261 - protein, 114 aa, synthetic construct
  REGION 1..114: note = CD8alpha Ig-like domain-2
SQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPA

SEQ ID NO: 262 - protein, 226 aa, synthetic construct
  REGION 1..226: note = m2CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 263 - DNA, 367 bp, synthetic construct
  misc_feature 1..367: note = MSCV promoter
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcact

SEQ ID NO: 264 - DNA, 607 bp, synthetic construct
  misc_feature 1..607: note = WPRE
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 265 - skipped

SEQ ID NO: 266 - protein, 4 aa, synthetic construct
  REGION 1..4: note = Linker
SGSG

SEQ ID NO: 267 - DNA, 4320 bp, synthetic construct
  misc_feature 1..4320: note = CD8b3.CD8a.TCR.WPREmut2 construct nucleotide sequence
  source 1..4320: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 268 - protein, 1110 aa, synthetic construct
  REGION 1..1110: note = CD8b3.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - DNA, 4314 bp, synthetic construct
  misc_feature 1..4314: note = CD8b1(S19).CD8a.TCR.WPREmut2 construct nucleotide sequence
  source 1..4314: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 270 - protein, 1108 aa, synthetic construct
  REGION 1..1108: note = CD8b1(S19).CD8a.TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 271 - DNA, 4428 bp, synthetic construct
  misc_feature 1..4428: note = CD8b4.CD8a.TCR.WPREmut2 construct nucleotide sequence
  source 1..4428: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 272 - protein, 1146 aa, synthetic construct
  REGION 1..1146: note = CD8b4.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 273 - DNA, 4308 bp, synthetic construct
  misc_feature 1..4308: note = CD8b1(S19).CD8a(S19).TCR.WPREmut2 construct nucleotide sequence
  source 1..4308: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 274 - protein, 1106 aa, synthetic construct
  REGION 1..1106: note = CD8b1(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 275 - DNA, 4416 bp, synthetic construct
  misc_feature 1..4416: note = CD8b4(S19).CD8a(S19).TCR.WPREmut2 construct nucleotide sequence
  source 1..4416: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 276 - protein, 1142 aa, synthetic construct
  REGION 1..1142: note = CD8b4(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - DNA, 4407 bp, synthetic construct
  misc_feature 1..4407: note = CD8b5(S19).CD8a(S19).TCR.WPREmut2 construct nucleotide sequence
  source 1..4407: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 278 - protein, 1139 aa, synthetic construct
  REGION 1..1139: note = CD8b5(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 279 - DNA, 3573 bp, synthetic construct
  misc_feature 1..3573: note = CD8a(S19).CD8bstalk.TCR.WPREmut2 construct nucleotide sequence
  source 1..3573: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 280 - protein, 861 aa, synthetic construct
  REGION 1..861: note = CD8a(S19).CD8bstalk.TCR.WPREmut2 amino acid sequence
MEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 281 - DNA, 4284 bp, synthetic construct
  misc_feature 1..4284: note = TCRba.CD8a.CD8b1.2.WPREmut2 construct nucleotide sequence
  source 1..4284: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgacctagccagttcagagtgtcccccctggaccggacctggaacctgggagagacagtggaactgaagtgccaggtgctgctgagcaaccccaccagcggctgctcttggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtgcgggccaagagatctggcagcggcgccaccaatttcagcctgctgaaacaggccggcgacgtggaagagaaccctggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtctgaaaggtaaggtttaccaagagcctttgtcaccgaacgcttgtatggacacgaccgctatcctccaaccgcataggtcttgtctcactcacgggtcatgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 282 - protein, 1098 aa, synthetic construct
  REGION 1..1098: note = TCRba.CD8a.CD8b1.2.WPREmut2 amino acid sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 283 - DNA, 3606 bp, synthetic construct
  misc_feature 1..3606: note = WT-SPa/CD8a-Ig like domain 1/CD8bstalk/CD4 TM+IC/TCRba/WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 284 - protein, 872 aa, synthetic construct
  REGION 1..872: note = WT-SPa/CD8a-Ig like domain 1/CD8bstalk/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 285 - DNA, 3579 bp, synthetic construct
  misc_feature 1..3579: note = CD8a.CD8bstalk.TCR.WRPEmut2 construct nucleotide sequence
  source 1..3579: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 286 - protein, 863 aa, synthetic construct
  REGION 1..863: note = CD8a.CD8bstalk.TCR.WRPEmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 287 - DNA, 3606 bp, synthetic construct
  misc_feature 1..3606: note = CD8a.TCR.WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 288 - protein, 872 aa, synthetic construct
  REGION 1..872: note = CD8a.TCR.WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 289 - DNA, 3633 bp, synthetic construct
  misc_feature 1..3633: note = WT-SPa/CD8a-Ig like domain 1(co)/CD8astalk(co)/CD4 TM+IC/TCRba/WPREmut2 construct nucleotide sequence
  source 1..3633: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 290 - protein, 881 aa, synthetic construct
  REGION 1..881: note = WT-SPa/CD8a-Ig like domain 1(co)/CD8astalk(co)/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 291 - DNA, 4419 bp, synthetic construct
  misc_feature 1..4419: note = PTE.CD8ab.TCR.WPREmut2 construct nucleotide sequence
  source 1..4419: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 292 - protein, 1143 aa, synthetic construct
  REGION 1..1143: note = PTE.CD8ab.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 293 - protein, 21 aa, synthetic construct
  REGION 1..21: note = Signal peptide
MRPRLWLLLAAQLTVLHGNSV

SEQ ID NO: 294 - protein, 19 aa, synthetic construct
  REGION 1..19: note = Signal peptide
MEFGLSWLFLVAILKGVQC

SEQ ID NO: 295 - DNA, 3631 bp, synthetic construct
  misc_feature 1..3631: note = CD8a.TCR.WPRE construct nucleotide sequence
  source 1..3631: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 296 - protein, 872 aa, synthetic construct; identical to SEQ ID NO: 288
  REGION 1..872: note = CD8a.TCR.WPRE amino acid sequence

SEQ ID NO: 297 - DNA, 4419 bp, synthetic construct; identical to SEQ ID NO: 291
  misc_feature 1..4419: note = CD8ba.TCR.WPRE nucleotide sequence
  source 1..4419: mol_type = other DNA

SEQ ID NO: 298 - protein, 1143 aa, synthetic construct; identical to SEQ ID NO: 292
  REGION 1..1143: note = CD8ba.TCR.WPRE amino acid sequence

SEQ ID NO: 299 - DNA, 7751 bp, synthetic construct
  misc_feature 1..7751: note = TCR.WPRE nucleotide sequence
  source 1..7751: mol_type = other DNA
aaacgccagcaacgcggcctttttacggttcctggccttttgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagaggcctctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaatctgcagaattcgcccttatcgattgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggatctggcagcggcgccaccaatttcagcctgctgaaacaggccggcgacgtggaagagaaccctggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgtacgtgtacattacgcgcctaggatttaaatccactcctgacaactactctttaatgcacgaggtgtcagtaggtgaaggagtcgtagttgtcgataagcagcaacatcttactacactggtctcaactccggatccgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 300 - protein, 607 aa, synthetic construct
  REGION 1..607: note = TCR.WPRE amino acid sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGSGSGATNFSLLKQAGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 301 - DNA, 3828 bp, synthetic construct
  misc_feature 1..3828: note = CD8aNCAM1Fusion.TCR.WPREmut2 nucleotide sequence
  source 1..3828: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccggccatcgtgggcatcctgatcgtgatcttcgtgctgctgctggtggtggtggacatcacctgctacttcctgaacaagtgcggcctgttcatgtgcatcgccgtgaacctgtgcggcaaggccggccccggcgccaagggcaaggacatggaggagggcaaggccgccttcagcaaggacgagagcaaggagcccatcgtggaggtgaggaccgaggaggagaggacccccaaccacgacggcggcaagcacaccgagcccaacgagaccacccccctgaccgagcccgagaagggccccgtggaggccaagcccgagtgccaggagaccgagaccaagcccgcccccgccgaggtgaagaccgtgcccaacgacgccacccagaccaaggagaacgagagcaaggcccgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 302 - protein, 946 aa, synthetic construct
  REGION 1..946: note = CD8aNCAM1Fusion.TCR.WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKARAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 303 - protein, 311 aa, synthetic construct; identical to SEQ ID NO: 16
  REGION 1..311: note = R11P3D3KE beta chain

SEQ ID NO: 304 - protein, 273 aa, synthetic construct
  REGION 1..273: note = R39P1C12 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 305 - protein, 224 aa, synthetic construct
  REGION 1..224: note = Dominant Negative TGFbetaRII var1 Amino Acid Sequence
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 306 - DNA, 672 bp, synthetic construct
  misc_feature 1..672: note = Dominant Negative TGFbetaRII var1 Nucleic Acid Sequence
  source 1..672: mol_type = other DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 307 - protein, 199 aa, synthetic construct
  REGION 1..199: note = Dominant Negative TGFbetaRII var2 Amino Acid Sequence
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 308 - DNA, 597 bp, synthetic construct
  misc_feature 1..597: note = Dominant Negative TGFbetaRII var2 Nucleic Acid Sequence
  source 1..597: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 309 - DNA, 729 bp, synthetic construct
  misc_feature 1..729: note = CD8beta1 Nucleic Acid Sequence, codon optimized
  source 1..729: mol_type = other DNA
atgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacc

SEQ ID NO: 310 - DNA, 705 bp, synthetic construct
  misc_feature 1..705: note = CD8alpha1 Nucleic Acid Sequence, codon optimized
  source 1..705: mol_type = other DNA
atggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtg

SEQ ID NO: 311 - DNA, 678 bp, synthetic construct
  misc_feature 1..678: note = m1CD8alpha Nucleic Acid Sequence, codon optimized
  source 1..678: mol_type = other DNA
atggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtg

SEQ ID NO: 312 - DNA, 1687 bp, synthetic construct
  misc_feature 1..1687: note = MSCV Pro- Dominant Negative TGFbetaRII var1 - WPRE Nucleic Acid Sequence
  source 1..1687: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcatagaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 313 - DNA, 1612 bp, synthetic construct
  misc_feature 1..1612: note = MSCV Pro- Dominant Negative TGFbetaRII var2 - WPRE Nucleic Acid Sequence
  source 1..1612: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcatagaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 314 - protein, 15 aa, synthetic construct
  REGION 1..15: note = Linker Amino Acid Sequence
GGGGSGGGGSGGGGS

SEQ ID NO: 315 - DNA, 45 bp, synthetic construct
  misc_feature 1..45: note = Linker Nucleic Acid Sequence
  source 1..45: mol_type = other DNA
ggcggcggcggcagcggcggcggcggcagcggcggcggcggcagc

SEQ ID NO: 316 - protein, 18 aa, synthetic construct
  REGION 1..18: note = Linker Amino Acid Sequence
KESGSVSSEQLAQFRSLD

SEQ ID NO: 317 - DNA, 54 bp, synthetic construct
  misc_feature 1..54: note = Linker Nucleic Acid Sequence
  source 1..54: mol_type = other DNA
aaagagtccggctccgtgtcctccgaacagctggcgcagtttcgttccctggat

SEQ ID NO: 318 - protein, 14 aa, synthetic construct
  REGION 1..14: note = Linker Amino Acid Sequence
EGKSSGSGSESKST

SEQ ID NO: 319 - DNA, 42 bp, synthetic construct
  misc_feature 1..42: note = Linker Nucleic Acid Sequence
  source 1..42: mol_type = other DNA
gaaggcaaatcctccggctccggctccgaatccaaatccacc

SEQ ID NO: 320 - protein, 31 aa, synthetic construct
  REGION 1..31: note = Linker Amino Acid Sequence
SGGGSGGGGSGGGGSGGGGSGGGGSGGGTLQ

SEQ ID NO: 321 - DNA, 93 bp, synthetic construct
  misc_feature 1..93: note = Linker Nucleic Acid Sequence
  source 1..93: mol_type = other DNA
tctggtggtggttctggtgggggtggctctggcggcgggggatcaggcggaggagggtccggaggcggaggctctggtgggggtactctacag

SEQ ID NO: 322 - protein, 21 aa, synthetic construct
  REGION 1..21: note = Linker Amino Acid Sequence
SGGGSGGGGSGGGGSGGGTLQ

SEQ ID NO: 323 - DNA, 63 bp, synthetic construct
  misc_feature 1..63: note = Linker Nucleic Acid Sequence
  source 1..63: mol_type = other DNA
tctggtggtggttctggtgggggtggctctggcggcgggggatctggtgggggtactctacag

SEQ ID NO: 324 - protein, 26 aa, synthetic construct
  REGION 1..26: note = Linker Amino Acid Sequence
SGGGSGGGGSGGGGSGGGGSGGGSLQ

SEQ ID NO: 325 - DNA, 78 bp, synthetic construct
  misc_feature 1..78: note = Linker Nucleic Acid Sequence
  source 1..78: mol_type = other DNA
agcggcggcggcagcggcggcggcggcagcggcggcggcggcagcggcggcggcggcagcggcggcggcagcctacag

SEQ ID NO: 326 - protein, 20 aa, synthetic construct
  REGION 1..20: note = Linker Amino Acid Sequence
SGGSGGGGSGGGSGGGGSLQ

SEQ ID NO: 327 - protein, 8 aa, synthetic construct
  REGION 1..8: note = Linker Amino Acid Sequence
GSGSGSGS

SEQ ID NO: 328 - protein, 11 aa, synthetic construct
  REGION 1..11: note = Linker Amino Acid Sequence
GGSGGSGGSGG

SEQ ID NO: 329 - protein, 5 aa, synthetic construct
  REGION 1..5: note = Linker Amino Acid Sequence
GGSGG

SEQ ID NO: 330 - protein, 15 aa, synthetic construct
  REGION 1..15: note = Linker Amino Acid Sequence
GGGGSGGGGSGGGGS

SEQ ID NO: 331 - protein, 8 aa, synthetic construct
  REGION 1..8: note = Linker Amino Acid Sequence
GGGGGGGG

SEQ ID NO: 332 - protein, 6 aa, synthetic construct
  REGION 1..6: note = Linker Amino Acid Sequence
GGGGGG

SEQ ID NO: 333 - protein, 5 aa, synthetic construct
  REGION 1..5: note = Linker Amino Acid Sequence
GGGGS

SEQ ID NO: 334 - protein, 10 aa, synthetic construct
  REGION 1..10: note = Linker Amino Acid Sequence
GGGGSGGGGS

SEQ ID NO: 335 - protein, 11 aa, synthetic construct
  REGION 1..11: note = Linker Amino Acid Sequence
GGSGGHMGSGG

SEQ ID NO: 336 - protein, 15 aa, synthetic construct
  REGION 1..15: note = Linker Amino Acid Sequence
EAAAKEAAAKEAAAK

SEQ ID NO: 337 - protein, 10 aa, synthetic construct
  REGION 1..10: note = Linker Amino Acid Sequence
EAAAKEAAAK

SEQ ID NO: 338 - protein, 5 aa, synthetic construct
  REGION 1..5: note = Linker Amino Acid Sequence
EAAAK

SEQ ID NO: 339 - protein, 46 aa, synthetic construct
  REGION 1..46: note = Linker Amino Acid Sequence
AEAAAKEAAAKEAAAKEAAAKALEAEAAAKEAAAKEAAAKEAAAKA

SEQ ID NO: 340 - protein, 5 aa, synthetic construct
  REGION 1..5: note = Linker Amino Acid Sequence
PAPAP

SEQ ID NO: 341 - protein, 12 aa, synthetic construct
  REGION 1..12: note = Linker Amino Acid Sequence
AEAAAKEAAAKA

SEQ ID NO: 342 - protein, 17 aa, synthetic construct
  REGION 1..17: note = Linker Amino Acid Sequence
VSQTSKLTRAETVFPDV

SEQ ID NO: 343 - protein, 6 aa, synthetic construct
  REGION 1..6: note = Linker Amino Acid Sequence
PLGLWA

SEQ ID NO: 344 - protein, 6 aa, synthetic construct
  REGION 1..6: note = Linker Amino Acid Sequence
RVLAEA

SEQ ID NO: 345 - protein, 10 aa, synthetic construct
  REGION 1..10: note = Linker Amino Acid Sequence
EDVVCCSMSY

SEQ ID NO: 346 - protein, 8 aa, synthetic construct
  REGION 1..8: note = Linker Amino Acid Sequence
GGIEGRGS

SEQ ID NO: 347 - protein, 10 aa, synthetic construct
  REGION 1..10: note = Linker Amino Acid Sequence
TRHRQPRGWE

SEQ ID NO: 348 - protein, 10 aa, synthetic construct
  REGION 1..10: note = Linker Amino Acid Sequence
AGNRVRRSVG

SEQ ID NO: 349 - protein, 9 aa, synthetic construct
  REGION 1..9: note = Linker Amino Acid Sequence
RRRRRRRRR

SEQ ID NO: 350 - protein, 4 aa, synthetic construct
  REGION 1..4: note = Linker Amino Acid Sequence
GFLG

SEQ ID NO: 351 - protein, 16 aa, synthetic construct
  REGION 1..16: note = Linker Amino Acid Sequence
GGGGSLVPRGSGGGGS

SEQ ID NO: 352 - protein, 34 aa, synthetic construct
  REGION 1..34: note = Linker Amino Acid Sequence
APAPAPAPAPAPAPAPAPAPAPAPAPAPAPAPAP

SEQ ID NO: 353 - protein, 32 aa, synthetic construct
  REGION 1..32: note = Linker Amino Acid Sequence
APAPAPAPAPAPAPAPAPAPAPAPAPAPAPAP

SEQ ID NO: 354 - protein, 30 aa, synthetic construct
  REGION 1..30: note = Linker Amino Acid Sequence
APAPAPAPAPAPAPAPAPAPAPAPAPAPAP

SEQ ID NO: 355 - protein, 28 aa, synthetic construct
  REGION 1..28: note = Linker Amino Acid Sequence
APAPAPAPAPAPAPAPAPAPAPAPAPAP

SEQ ID NO: 356 - protein, 26 aa, synthetic construct
  REGION 1..26: note = Linker Amino Acid Sequence
APAPAPAPAPAPAPAPAPAPAPAPAP

SEQ ID NO: 357 - protein, 24 aa, synthetic construct
  REGION 1..24: note = Linker Amino Acid Sequence
APAPAPAPAPAPAPAPAPAPAPAP

SEQ ID NO: 358 - protein, 22 aa, synthetic construct
  REGION 1..22: note = Linker Amino Acid Sequence
APAPAPAPAPAPAPAPAPAPAP

SEQ ID NO: 359 - protein, 20 aa, synthetic construct
  REGION 1..20: note = Linker Amino Acid Sequence
APAPAPAPAPAPAPAPAPAP

SEQ ID NO: 360 - protein, 18 aa, synthetic construct
  REGION 1..18: note = Linker Amino Acid Sequence
APAPAPAPAPAPAPAPAP

SEQ ID NO: 361 - protein, 16 aa, synthetic construct
  REGION 1..16: note = Linker Amino Acid Sequence
APAPAPAPAPAPAPAP

SEQ ID NO: 362 - protein, 14 aa, synthetic construct
  REGION 1..14: note = Linker Amino Acid Sequence
APAPAPAPAPAPAP

SEQ ID NO: 363 - protein, 12 aa, synthetic construct
  REGION 1..12: note = Linker Amino Acid Sequence
APAPAPAPAPAP

SEQ ID NO: 364 - protein, 10 aa, synthetic construct
  REGION 1..10: note = Linker Amino Acid Sequence
APAPAPAPAP

SEQ ID NO: 365 - skipped

SEQ ID NO: 366 - DNA, 723 bp, synthetic construct
  misc_feature 1..723: note = Hepatitis B Virus (HBV) Post-Transcriptional Regulatory Element Nucleic Acid Sequence (HPRE)
  source 1..723: mol_type = other DNA
taaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtc

SEQ ID NO: 367 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK